(19) United States
(12) Patent Application Publication
(10) Pub. No.: US 2025/0327027 A1
(43) Pub. Date: Oct. 23, 2025

(54) METHODS TO GENERATE ENHANCED TUMOR INFILTRATING LYMPHOCYTES THROUGH MICROFLUIDIC DELIVERY

(71) Applicant: STEMCELL TECHNOLOGIES CANADA INC., VANCOUVER (CA)

(72) Inventors: Jonathan B. GILBERT, Watertown, MA (US); Devin BRIDGEN, Watertown, MA (US); Michael Finnan MALONEY, Watertown, MA (US); Emrah Ilker OZAY, Watertown, MA (US); Scott Mcnabb LOUGHHEAD, Watertown, MA (US); Rebecca Jean SILVER, Watertown, MA (US); Ashley BRATE, Watertown, MA (US); Murillo SILVA, Watertown, MA (US); Nader Ahmad EL-SAYES, Watertown, MA (US)

(21) Appl. No.: 18/293,181

(22) PCT Filed: Jul. 28, 2022
(86) PCT No.: PCT/US2022/074273

§ 371 Date: Jan. 29, 2024

Related U.S. Application Data
(60) Provisional application No. 63/227,253, filed on Jul. 29, 2021.

(51) Int. Cl. C12N 5/0783; C07K 14/54; C07K 14/55; C07K 14/705

(52) U.S. Cl. CPC C12N 5/0636; C07K 14/5418; C07K 14/5434; C07K 14/55; C07K 14/705; C07K 14/70532; C12N 2501/2302; C12N 2501/2312; C12N 2501/2315; C12N 2501/515; C12N 2510/00

(57) ABSTRACT

The present application provides TILs comprising agents that enhance activity and/or proliferative capacity of the TILs, methods of manufacturing such TILs, and methods of using such modified TILs for enhancing an immune response.

### CROSS-REFERENCE TO RELATED APPLICATION

This PCT application claims the priority benefit of U.S. Provisional Application No. 63/227,253, filed Jul. 29, 2021, which is incorporated herein by reference in its entirety.

### REFERENCE TO SEQUENCE LISTING SUBMITTED ELECTRONICALLY VIA EFS WEB

The content of the electronically submitted sequence listing (7771-75455US00_SequenceListing.xml; Size: 30,475 bytes; and Date of Creation: Aug. 27, 2024) submitted in this application is incorporated herein by reference in its entirety.

### FIELD OF THE DISCLOSURE

The present disclosure relates generally to TILs comprising agents that enhance activity and/or proliferative capacity of the TILs, methods of manufacturing such TILs, and methods of using such modified TILs for enhancing an immune response.

### BACKGROUND OF THE DISCLOSURE

There are various challenges associated with the development of Tumor Infiltrating Lymphocyte (TIL) therapies. While TIL therapies have shown significant solid tumor activity in patients, current TIL compositions require patient lymphodepletion and maintenance in high dose IL-2 after cell infusion to support clinical activity. Ex vivo engineering of the TIL product with mRNA could enhance potency, expand the potential patient population, and potentially allow for repeat dosing and concomitant treatment with other therapies.

All references cited herein, including patent applications and publications, are incorporated by reference in their entirety. The patent publications WO 2016070136, US 20180142198, WO 2017008063, US 20180201889, WO 2019178005, and WO 2019178006 and WO 2020176789 are hereby expressly incorporated by reference in their entirety.

### BRIEF SUMMARY OF THE DISCLOSURE

In some aspects, the disclosure provides a method of modulating the activity and/or proliferative capacity of tumor-infiltrating lymphocytes (TILs), comprising modifying the TILs to increase expression of one or more co-stimulatory molecules, one or more anti-apoptotic factors, and/or one or more cytokines. In some aspects, the disclosure provides, a method of modulating the activity and/or proliferative capacity of tumor-infiltrating lymphocytes (TILs), comprising modifying the TILs to increase expression of one or more of co-stimulatory molecules. In some aspects, the co-stimulatory molecule is B7-H2 (ICOSL), B7-1 (CD80), B7-2 (CD86), CD70, LIGHT, HVEM, CD40, 4-1BBL, OX40L, TL1A, GITRL, CD30L, TIM4, SLAM, CD48, CD58, CD155, or CD112 In some aspects, the co-stimulatory molecule is CD86. In some aspects, the disclosure provides a method of modulating the activity and/or proliferative capacity of TILs, comprising modifying the TILs to increase expression of one or more cytokines.

In some aspects of the methods described herein, the TILs are modified to comprise a chimeric membrane-bound cytokine. In some aspects, the chimeric membrane-bound cytokine is a fusion protein comprising the cytokine and a transmembrane domain. In some aspects, the cytokine is joined to the transmembrane domain by a peptide linker. In some aspects, the peptide linker is (G4S)3 (SEQ ID NO: 3) or (EAAAK)3 (SEQ ID NO: 4). In some aspects, the cytokine is a Type I cytokine. In some aspects, the cytokine is IL-15, IL-12, IL-2, IL-7, IFN α, IFN β, or IL-21 or functional variant thereof. In some aspects, the cytokine is IL-2 or a functional variant thereof, IL-7 or a functional variant thereof, IL-15 or a functional variant thereof, and/or IL-12 or a functional variant thereof. In some aspects, the cytokine is a membrane-bound IL-2. In some aspects, the cytokine is membrane-bound IL-7. In some aspects, the cytokine is membrane-bound IL-15. In some aspects, the cytokine is membrane-bound IL-12. In some aspects, the chimeric membrane-bound cytokine comprises the amino acid sequence of any one of SEQ ID NOs: 7-10 and 13. Some aspects of the present disclosure relates to a method of modulating the activity and/or proliferative capacity of TILs, comprising modifying the TILs to express or increase the expression of one or more anti-apoptotic factors. In some aspects, the anti-apoptotic factor comprises Bcl-2.

In some aspects of the disclosure, the modified TILs comprise increased expression of one or more cytokines and/or one or more of co-stimulatory molecules, wherein the TILs are prepared by a process comprising a) passing a cell suspension comprising input TILs through a cell-deforming constriction, wherein a diameter of the constriction is a function of a diameter of the input TILs in the suspension, thereby causing perturbations of the input TILs large enough for one or more nucleic acids encoding one or more cytokines and/or one or more nucleic acids encoding one or more co-stimulatory molecules to pass through to form perturbed input TILs; and b) incubating the perturbed input TILs with the one or more nucleic acids encoding one or more cytokines and/or one or more nucleic acids encoding one or more co-stimulatory molecules to allow the one or more nucleic acids encoding one or more cytokines and/or one or more nucleic acids encoding one or more co-stimulatory molecules to enter the perturbed input TILs; wherein the nucleic acids are expressed, thereby generating the modified TILs comprising the one or more cytokines and/or the one or more co-stimulatory molecules. In some aspects of the disclosure, the modified TILs comprises increased expression of one or more cytokines and/or one or more of co-stimulatory molecules, wherein the TILs are prepared by a process comprising: a) passing a cell suspension comprising input TILs through a cell-deforming constriction, wherein a diameter of the constriction is a function of a diameter of the input TILs in the suspension, thereby causing perturbations of the input TILs large enough for the one or more nucleic acids encoding one or more cytokines and/or the one or more co-stimulatory molecules to pass through to form perturbed input TILs; and b) incubating the perturbed input TILs with the one or more nucleic acids encoding one or more cytokines and/or the one or more co-stimulatory molecules to enter the perturbed input TILs; wherein the nucleic acids are expressed thereby generating the modified TILs comprising the one or more cytokines and/or the one or more co-stimulatory molecules. In some aspects, the method comprises: (a) incubating the TILs with the nucleic acid encoding the chimeric membrane-bound cytokine before, during and/or after passing the cell suspension through the cell-deforming constriction (b) incubating the TILs with the nucleic acid encoding the chimeric membrane-bound cytokine and the co-stimulatory molecule before, during and/or after passing the cell suspension through the cell-deforming constriction; (c) incubating the TILs with the nucleic acid encoding the chimeric membrane-bound cytokine and the nucleic acid encoding the co-stimulatory molecule before, during and/or after passing the cell suspension through the cell-deforming constriction, or (d) incubating the TILs with the nucleic acid encoding the chimeric membrane-bound cytokine and the nucleic acid encoding the one or more co-stimulatory molecules before, during and/or after passing the cell suspension through the cell-deforming constriction. In some aspects, the method comprises (a) incubating the TILs with the nucleic acid encoding the chimeric membrane-bound cytokine before passing the cell suspension through the cell-deforming constriction (b) incubating the TILs with the nucleic acid encoding the chimeric membrane-bound cytokine and the co-stimulatory molecule before passing the cell suspension through the cell-deforming constriction; (c) incubating the TILs with the nucleic acid encoding the chimeric membrane-bound cytokine and the nucleic acid encoding the co-stimulatory molecule before passing the cell suspension through the cell-deforming constriction; or (d) incubating the TILs with the nucleic acid encoding the chimeric membrane-bound cytokine and the nucleic acid encoding the one or more co-stimulatory molecules before passing the cell suspension through the cell-deforming constriction. In some aspects, one or more of the nucleic acids is mRNA

In some aspects, modifying the TILs to increase expression of one or more co-stimulatory molecules, one or more anti-apoptotic factors, and/or one or more cytokines comprise passing a cell suspension comprising the TILs through a cell-deforming constriction, thereby causing perturbations of the TILs such that one or more nucleic acids encoding the one or more co-stimulatory molecules, one or more anti-apoptotic factors, and/or one or more nucleic acids encoding the one or more cytokines enter the TILs through the perturbations when contacted with the TILs. In some aspects, the modifying further comprises contacting the TILs with the one or more nucleic acids encoding the one or more co-stimulatory molecules, the one or more nucleic acids encoding the one or more anti-apoptotic factors, and/or the one or more nucleic acids encoding the one or more cytokines. In some aspects, the method comprises contacting the TILs with the one or more nucleic acids encoding the one or more co-stimulatory molecules, the one or more nucleic acids encoding the one or more anti-apoptotic factors, and/or the one or more nucleic acids encoding the one or more cytokines during the passing of the cell suspension through the cell-deforming constriction. In some aspects, the method comprises contacting the TILs with the one or more nucleic acids encoding the one or more co-stimulatory molecules, the one or more nucleic acids encoding the one or more anti-apoptotic factors, and/or the one or more nucleic acids encoding the one or more cytokines before passing the cell suspension through the cell-deforming constriction. In some aspects, the method comprises contacting the TILs with the one or more nucleic acids encoding the one or more co-stimulatory molecules, the one or more nucleic acids encoding the one or more anti-apoptotic factors, and/or the one or more nucleic acids encoding the one or more cytokines after the cell suspension passes through the cell-deforming constriction.

In some aspects of the disclosure, the modified TILs have increased expression of one or more of T-bet, EOMES, TCF1, CD127, CD103, CD45RO, CCR5, and CD62L compared to corresponding TILs that are not modified (“reference TILs”). In some aspects, the modified TILs have increased expression of one or more of T-bet, EOMES, TCF1, CD127, CD103, CID45RO, CCR5, and CD62L when cultured in the absence of exogenous cytokines, as compared to corresponding TILs that are not modified. In some aspects, the expression of one or more of T-bet, EOMES, TCF1, CD127, CD103, CD45RO, CCR5, and CD62L in the modified TILs is increased by more than about 1.2-fold, 1.5-fold, 1.8-fold, 2-fold, 3-fold, 4-fold, 5-fold, 8-fold, or more than 10-fold as compared to corresponding TILs that are not modified.

In some aspects, the modified TILs exhibit increased proliferation compared to corresponding TILs that are not modified. In some aspects, the modified TILs exhibit increased proliferation when cultured in media not comprising exogenous cytokines, as compared to corresponding TILs that are not modified. In some aspects, after the modifying, the TILs exhibit increased in vivo persistence as compared to reference TILs, which comprise corresponding TILs that have not been modified. In some aspects, after the modifying, the TILs exhibit the increased in vivo persistence in the absence of an exogenous cytokine as compared to the reference TILs. In some aspects, after the modifying, the TILs exhibit increased viability as compared to reference TILs, which comprise corresponding TILs that have not been modified. In some aspects, after the modifying, the TILs exhibit the increased viability in the absence of an exogenous cytokine as compared to the reference TILs. In some aspects, after the modifying, the TILs produce greater amount of IFN-γ upon anti-CD3 stimulation as compared to reference TILs, which comprise corresponding TILs that have not been modified. In some aspects, after the modifying, the TILs are capable of killing tumor cells in the absence of an exogenous cytokine. In some aspects, after the modifying, the TILs exhibit increased survival as compared to reference TILs, which comprise corresponding TILs that have not been modified. In some aspects, after the modifying, the TILs exhibit the increased survival in the absence of an exogenous cytokine as compared to the reference TILs. In some aspects, the exogenous cytokine is IL-2 and/or IL-12; optionally wherein the exogenous cytokine is IL-2.

In some aspects, after the modifying, the TILs exhibit an increased expression of a membrane-bound IL-2 as compared to reference TILs, which comprise corresponding TILs that have not been modified. In some aspects, after the modifying, the TILs exhibit an increased expression of a membrane-bound IL-12 as compared to reference TILs, which comprise corresponding TILs that have not been modified. In some aspects, after the modifying, the TILs exhibit an increased expression of CD86 as compared to reference TILs, which comprise corresponding TILs that have not been modified. In some aspects, after the modifying, the TILs exhibit an increased expression of membrane-bound IL-7 as compared to reference TILs, which comprise corresponding TILs that have not been modified. In some aspects, after the modifying, the TILs exhibit an increased expression of membrane-bound Il-15 as compared to reference TILs, which comprise corresponding TILs that have not been modified. In some aspects, after the modifying, the TILs exhibit an increased expression of Bcl-2 as compared to reference TILs, which comprise corresponding TILs that have not been modified.

In some aspects, the method of modulating the activity and/or proliferative capacity of TILs provided herein comprises comprise expanding the TILs prior to the modifying. In some aspects, expanding the TILs comprise culturing the TILs with one or more agents, wherein the agents are capable of expanding the TILs such that the number of TILs is increased as compared to the number of the TILs prior to the expansion.

In some aspects, the one or more agents comprise an exogenous cytokine, an antibody, a feeder cell, or combinations thereof. In some aspects, the exogenous cytokine comprises exogenous IL-15 and/or exogenous IL-2 In some aspects, the exogenous cytokine does not comprise exogenous IL-15 and/or exogenous IL-2. In some aspects, the antibody comprises an anti-CD3 antibody. In some aspects, the anti-CD3 antibody is not OKT3.

In some aspects, expanding the TILs comprise a first expansion phase and a second expansion phase. In some aspects, the first expansion phase comprises culturing the TILs in the presence of an exogenous cytokine. In some aspects, the exogenous cytokine does not comprise exogenous IL-2 and/or exogenous IL-15. In some aspects, the exogenous cytokine comprises exogenous IL-15 and/or exogenous IL-2. In some aspects, the first expansion phase comprises culturing the TILs in the presence of an anti-CD3 antibody. In some aspects, the anti-CD3 antibody is not OKT3.

In some aspects, the second expansion phase comprises culturing the TILs in the presence of an exogenous cytokine, an anti-CD3 antibody, a feeder cell, or a combination thereof. In some aspects, the exogenous cytokine does not comprise exogenous IL-2 and/or exogenous IL-15. In some aspects, the exogenous cytokine comprises exogenous IL-2 and/or exogenous IL-15 in some aspects, the anti-CD3 antibody is not OKT3.

In some aspects, the first expansion phase occurs prior to the second expansion phase. In some aspects, the first expansion phase is for at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, or at least about 20 days. In some aspects, the first expansion phase is for between about 7 days to about 14 days.

In some aspects, the second expansion phase is for at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, or at least about 20 days. In some aspects, the second expansion phase is for between about 7 days to about 16 days. In some aspects, the second expansion phase is for between about 7 days to about 15 days. In some aspects, the second expansion phase is for between about 7 days to about 12 days.

In some aspects, the disclosure provides a composition comprising modified TILs which exhibit increased expression of one or more of co-stimulatory molecules, one or more anti-apoptotic factors, and/or one or more cytokines as compared to reference TILs, which comprise corresponding non-modified TILs. In some aspects, the disclosure provides a composition comprising modified TILs, wherein the TILs exhibit increased expression of one or more of co-stimulatory molecules as compared to reference TILs, which comprise corresponding non-modified TILs. In some aspects, the co-stimulatory molecule is B7-H2 (ICOSL), B7-1 (CD80), B7-2 (CD86), CD70, LIGHT, HVEM, CD40, 4-1BBL, OX40L, TL1A, GITRL, CD30L, TIM4, SLAM, CD48, CD58, CD155, or CD112. In some aspects, the co-stimulatory molecule is CD86. In some aspects, the TILs are modified to increase expression of one or more cytokines. Accordingly, some aspects of the present disclosure relates to a composition comprising modified TILs which exhibit increased expression of one or more cytokines as compared to reference TILs, which comprise corresponding non-modified TILs. In some aspects, the one or more cytokines comprise a chimeric membrane-bound cytokine. In some aspects, the disclosure provides a composition comprising modified TILs the TILs are modified to comprise a chimeric membrane-bound cytokine.

In some aspects of the compositions described herein, the chimeric membrane-bound cytokine is a fusion protein comprising the cytokine and a transmembrane domain. In some aspects, the cytokine is joined to the transmembrane domain by a peptide linker. In some aspects, the peptide linker is (G4S)3 (SEQ ID NO: 3) or (EAAAK)3 (SEQ ID NO 4). In some aspects, the cytokine is a Type I cytokine. In some aspects, the cytokine is IL-15, IL-12, IL-2, IL-7, IFN α, IFN β, or IL-21 or functional variant thereof. In some aspects, the cytokine is IL-2 or a functional variant thereof, IL-7 or a functional variant thereof, IL-15 or a functional variant thereof, and/or IL-12 or a functional variant thereof. In some aspects, the cytokine is a membrane-bound IL-2. In some aspects, the cytokine is a membrane-bound IL-7. In some aspects, the cytokine is a membrane-bound IL-15. In some aspects, the cytokine is a membrane-bound IL-12. In some aspects, the chimeric membrane-bound cytokine comprises the amino acid sequence of any one of SEQ ID NOs: 7-10 and 13. Also provided herein is a composition comprising modified TILs, wherein the modified TILs express or exhibit increased expression of one or more anti-apoptotic factors as compared to reference TILs, which comprise corresponding non-modified TILs. In some aspects, the anti-apoptotic factor comprise Bcl-2.

In some aspects of the compositions described herein, the modified TILs comprise increased expression of one or more cytokines and/or one or more of co-stimulatory molecules, wherein the modified TILs are prepared by a process comprising a) passing a cell suspension comprising input TILs through a cell-deforming constriction, wherein a diameter of the constriction is a function of a diameter of the input TILs in the suspension, thereby causing perturbations of the input TILs large enough for one or more nucleic acids encoding one or more cytokines and/or one or more nucleic acids encoding one or more co-stimulatory molecules to pass through to form perturbed input TILs; and b) incubating the perturbed input TILs with the one or more nucleic acids encoding one or more cytokines and/or one or more nucleic acids encoding one or more co-stimulatory molecules to allow the one or more nucleic acids encoding one or more cytokines and/or one or more nucleic acids encoding one or more co-stimulatory molecules to enter the perturbed input TILs; wherein the nucleic acids are expressed, thereby generating the modified TILs comprising the one or more cytokines and/or the one or more co-stimulatory molecules. In some aspects, the TILs comprises increased expression of one or more cytokines and/or one or more of co-stimulatory molecules, wherein the TILs are prepared by a process comprising: a) passing a cell suspension comprising input TILs through a cell-deforming constriction, wherein a diameter of the constriction is a function of a diameter of the input TILs in the suspension, thereby causing perturbations of the input TILs large enough for the one or more nucleic acids encoding one or more cytokines and/or the one or more co-stimulatory molecules to pass through to form perturbed input TILs; and b) incubating the perturbed input TILs with the one or more nucleic acids encoding one or more cytokines and/or the one or more co-stimulatory molecules to enter the perturbed input TILs; wherein the nucleic acids are expressed thereby generating the modified TILs comprising the one or more cytokines and/or the one or more co-stimulatory molecules. In some aspects, the process of preparing the modified TILs comprises: (a) incubating the TILs with the nucleic acid encoding the chimeric membrane-bound cytokine before, during and/or after passing the cell suspension through the cell-deforming constriction; (b) incubating the TILs with the nucleic acid encoding the chimeric membrane-bound cytokine and the co-stimulatory molecule before, during and/or after passing the cell suspension through the cell-deforming constriction; (c) incubating the TILs with the nucleic acid encoding the chimeric membrane-bound cytokine and the nucleic acid encoding the co-stimulatory molecule before, during and/or after passing the cell suspension through the cell-deforming constriction; or (d) incubating the TILs with the nucleic acid encoding the chimeric membrane-bound cytokine and the nucleic acid encoding the one or more co-stimulatory molecules before, during and/or after passing the cell suspension through the cell-deforming constriction. In some aspects, the process of preparing the modified TILs comprises: (a) incubating the TILs with the nucleic acid encoding the chimeric membrane-bound cytokine before passing the cell suspension through the cell-deforming constriction (b) incubating the TILs with the nucleic acid encoding the chimeric membrane-bound cytokine and the co-stimulatory molecule before passing the cell suspension through the cell-deforming constriction; (c) incubating the TILs with the nucleic acid encoding the chimeric membrane-bound cytokine and the nucleic acid encoding the co-stimulatory molecule before passing the cell suspension through the cell-deforming constriction; or (d) incubating the TILs with the nucleic acid encoding the chimeric membrane-bound cytokine and the nucleic acid encoding the one or more co-stimulatory molecules before passing the cell suspension through the cell-deforming constriction. In some aspects, one or more of the nucleic acids is mRNA.

In some aspects, a composition provided herein comprises modified TILs, wherein the modified TILs have been passed through a cell-deforming constriction, thereby causing perturbations of the TILs such that one or more nucleic acids encoding the one or more co-stimulatory molecules, one or more nucleic acids encoding the one or more anti-apoptotic factors, and/or one or more nucleic acids encoding the one or more cytokines entered the TILs through the perturbations when contacted with the TILs.

In some aspects of the compositions described herein, the modified TILs have increased expression of one or more of T-bet, EOMES, TCF1, CD127, CD103, CD45RO, CCR5, and CD62L compared to corresponding TILs that are not modified. In some aspects, the modified TILs have increased expression of one or more of T-bet, EOMES, TCF1, CD127, CD103, CD45RO, CCR5, and CD62L when cultured in the absence of exogenous cytokines, compared to corresponding TILs that are not modified. In some aspects, the expression of one or more of T-bet, EOMES, TCF1, CD127, CD103, CD45RO, CCR5, and CD62L in modified TILs is increased by more than about 1.2-fold, 1.5-fold, 1.8-fold, 2-fold, 3-fold, 4-fold, 5-fold, 8-fold, or more than 10-fold compared to the corresponding TILs that are not modified.

In some aspects, the modified TILs of the compositions exhibit increased proliferation compared to corresponding TILs that are not modified. In some aspects, the modified TILs exhibit increased proliferation when cultured in media not comprising exogenous cytokines, as compared to corresponding TILs that are not modified. For any of the compositions provided herein, in some aspects, the modified TILs exhibit one or more of the following properties as compared to reference TILs, which comprise corresponding non-modified TILs: (i) increased in vivo persistence, (ii) increased viability, (iii) increased ability to produce IFN-γ upon anti-CD3 stimulation, (iv) increased ability to kill tumor cells, (v) increased survival, or (vi) any combination of (i) to (v). In some aspects, the modified TILs exhibit one or more of the properties in the absence of an exogenous cytokine. In some aspects, the exogenous cytokine is IL-2 and/or IL-12; optionally wherein the exogenous cytokine is IL-2. In some aspects, the modified TILs exhibit increased expression of one or more of the following: (i) membrane-bound IL-2, (ii) membrane-bound IL-12, (iii) CD86, (iv) membrane-bound IL-7, (v) membrane-bound IL-15, (vi) Bcl-2, or (vii) any combination of (i) to (vi). In some aspects, a composition provided herein (e.g., described above) further comprises a pharmaceutically acceptable carrier.

In some aspects, the disclosure provides a method of modulating an immune response in an individual, comprising administering the modified TILs to an individual, wherein the modified TILs are prepared according to the method described herein. In some aspects, the disclosure provides a method of modulating an immune response in an individual, comprising administering the composition described herein.

In some aspects, the disclosure provides a method for treating a cancer, an infectious disease, or a viral-associated disease in an individual, comprising administering the modified TILs to an individual, wherein the modified TILs are prepared according to the method described herein. In some aspects, the disclosure provides a method for treating a cancer, an infectious disease, or a viral-associated disease in an individual, comprising administering the composition described herein. In some aspects, the method comprises multiple administration of the modified TILs, or multiple administration of the composition. In some aspects, the modified TILs is administered intravenously or intratumorally. In some aspects, the individual is a human. In some aspects, the modified TILs are administered prior to, concurrently with, or following administration of another therapy.

In some aspects, the disclosure provides a pharmaceutical composition for stimulating an immune response in an individual, wherein the composition comprises an effective amount of composition described herein. In some aspects, the disclosure provides a pharmaceutical composition for use as a medicine, wherein the pharmaceutical composition comprises an effective amount of composition described herein. In some aspects, the disclosure provides a pharmaceutical composition for treating a cancer, an infectious disease, or a viral-associated disease in an individual, wherein the pharmaceutical composition comprises an effective amount of composition described herein. In some aspects, the composition comprises modified TILs that are administered prior to, concurrently with, or following administration of another therapy.

In some aspects of the constriction-mediated delivery described herein, the width of the constriction is about 10% to about 99% of the mean diameter of the input TILs. In some aspects, the width of the constriction is about 3.5 μm to about 4.2 μm, or about 3.5 μm to about 4.8 μm, or about 3.5 μm to about 6 μm, or about 5 μm to about 12 μm, or about 12 μm to about 15 μm, or about 6 μm to about 12 μm, or about 8 μm to about 11 μm, or about 9 μm to about 11 μm. In some aspects, the width of the constriction is about 3 μm to about 5 μm. In some aspects, the width of the constriction is about 4 μm. In some aspects, the width of the constriction is about 4.5 μm. In some aspects, the cell suspension comprising the plurality of input TILs are passed through multiple constrictions wherein the multiple constrictions are arranged in series and/or in parallel.

In some aspects, the disclosure provides a kit for use in any of the methods described herein. In some aspects, the disclosure provides a kit comprising the composition described herein. In some aspects, the kit further comprises one or more of buffers, diluents, filters, needles, syringes, or package inserts with instructions for administering the composition to an individual.

Also provided herein is a method of producing TILs which exhibit one or more improved properties, comprising intracellularly delivering one or more nucleic acids encoding a cytokine, wherein the cytokine is capable of improving one or more properties of the TILs. Also provided herein is a method of producing TILs which exhibit one or more improved properties, comprising intracellularly delivering one or more nucleic acids encoding a co-stimulatory molecule, wherein the co-stimulatory molecule is capable of improving one or more properties of the TILs. The present disclosure further provides a method of producing TILs which exhibit one or more improved properties, comprising intracellularly delivering one or more nucleic acids encoding an anti-apoptotic factor, wherein the anti-apoptotic factor is capable of improving one or more properties of the TILs. Some aspects of the present disclosure relates to method of producing TILs which exhibit one or more improved properties, comprising intracellularly delivering one or more nucleic acids encoding a cytokine, one or more nucleic acids encoding an anti-apoptotic factor, and one or more nucleic acids encoding a co-stimulatory molecule, wherein the cytokine, anti-apoptotic factor, and/or the co-stimulatory molecule is capable of improving one or more properties of the TILs.

In any of the above methods, in some aspects, the one or more improved properties comprise: (i) increased proliferation; (ii) increased in vivo persistence, (iii) increased viability, (iv) increased ability to produce IFN-γ upon anti-CD3 stimulation, (v) increased ability to kill tumor cells, (vi) increased survival, or (vii) any combination of (i) to (vi).

In some aspects, a nucleic acid useful for the present disclosure encodes a cytokine, wherein the cytokine comprises IL-15, IL-12, IL-7, IL-2, IFN-α, IFN-β, or IL-21 or functional variant thereof. In some aspects, the cytokine comprises a membrane-bound cytokine. In some aspects, the cytokine is a membrane-bound IL-2. In some aspects, the cytokine is a membrane-bound IL-7. In some aspects, the cytokine is a membrane-bound IL-15. In some aspects, the cytokine is a membrane-bound IL-12 In some aspects, a nucleic acid useful the for the present disclosure encodes a co-stimulatory molecule, wherein the co-stimulatory molecule comprises B7-H2 (ICOSL), B7-1 (CD80), B7-2 (CD86), CD70, LIGHT, HVEM, CD40, 4-1BBL, OX40L, TL1A, GITRL, CD30L, TIM4, SLAM, CD48, CD58, CD155, or CD112. In some aspects, the nucleic acid encodes an anti-apoptotic factor, wherein the anti-apoptotic factor comprises Bcl-2.

In some aspects, the disclosure provides a method of producing TILs comprising a chimeric membrane-bound cytokine, the method comprising introducing a nucleic acid encoding the chimeric membrane-bound cytokine to the TILs. In some aspects, the TILs comprising the chimeric membrane-bound cytokine are prepared by: a) passing a cell suspension comprising input TILs through a cell-deforming constriction, wherein a diameter of the constriction is a function of a diameter of the input TILs in the suspension, thereby causing perturbations of the input TILs large enough for a nucleic acid encoding the chimeric membrane-bound cytokine to pass through to form a perturbed input TILs; and b) incubating the perturbed input TILs with the nucleic acid encoding the chimeric membrane-bound cytokine to allow the nucleic acid to enter the perturbed input TILs where the nucleic acid encoding the chimeric membrane-bound cytokine is expressed; thereby generating TILs comprising a chimeric membrane-bound cytokine. In some aspects, the method comprises incubating the TILs with the nucleic acid encoding the chimeric membrane-bound cytokine thereof before, during and/or after passing the cell suspension through the cell-deforming constriction. In some aspects, the method comprises incubating the TILs with the nucleic acid encoding the chimeric membrane-bound cytokine before passing the cell suspension through the cell-deforming constriction. In some aspects, the nucleic acid encoding the chimeric membrane-bound cytokine is an mRNA encoding the chimeric membrane-bound cytokine.

For any of the producing methods provided herein, in some aspects, intracellularly delivering the one or more nucleic acids comprise passing the TILs through a cell-deforming constriction, thereby causing perturbations in the TILs such that the one or more nucleic acids enter the TILs through the perturbations when contacted with the TILs. In some aspects, the method comprises contacting the TILs with the one or more nucleic acids. In some aspects, the nucleic acids are mRNA.

For any of the producing methods provided herein (e.g., such as those described above), the width of the constriction is about 10% to about 99% of the mean diameter of the input TILs. In some aspects, the width of the constriction is about 5 μm to about 12 μm, or about 6 μm to about 12 μm, or about 8 μm to about 11 μm, or about 9 μm to about 11 μm, or about 12 μm to about 15 μm. In some aspects, the width of the constriction is about 10 μm. In some aspects, the width of the constriction is about 8 μm. In some aspects, the width of the constriction is about 4 μm. In some aspects the width of the constriction is about 4.5 μm. In some aspects, the cell suspension comprising the plurality of input TILs are passed through multiple constrictions wherein the multiple constrictions are arranged in series and/or in parallel.

In some aspects, the disclosure provides the use of a pharmaceutical composition in the manufacture of a medicament for stimulating an immune response in an individual, wherein the pharmaceutical composition comprises an effective amount of composition described herein. In some aspects, the disclosure provides the use of a pharmaceutical composition in the manufacture of a medicament for treating a cancer, an infectious disease, or a viral-associated disease in an individual, wherein the pharmaceutical composition comprises an effective amount of composition described herein. In some aspects, the pharmaceutical composition is formulated for multiple administration. In some aspects, the pharmaceutical composition is administered intravenously or intratumorally. In some aspects, the individual is a human. In some aspects, the pharmaceutical composition is formulated for administration prior to, concurrently with, or following administration of another therapy.

### BRIEF DESCRIPTION OF THE DRAWINGS

FIG. 1A shows the viability of tumor infiltrating lymphocytes expressing membrane-bound IL-2 (“mbIL-2”), membrane-bound IL-12 (“mbIL-12”), or both mbIL-2 and mbIL-12, provided by constriction mediated delivery of mRNA, at various time points following delivery. No contact (“NC”, e.g. cells were not processed by constriction mediated delivery), empty squeeze (e.g., constriction mediated delivery was performed with an empty payload), and GFP provided by constricted mediated delivery of mRNA were used as controls in these experiments.

FIG. 1B shows the GFP expression in tumor infiltrating lymphocytes expressing membrane-bound IL-2 (“mbIL-2”), membrane-bound IL-12 (“mbIL-12”), or both mbIL-2 and mbIL-12, provided by constriction mediated delivery of mRNA, at various time points following delivery. No contact (“NC”), empty squeeze (e.g., constriction mediated delivery was performed with an empty payload), and GFP provided by constricted mediated delivery of mRNA, were used as controls in these experiments.

FIG. 1C shows the membrane-bound IL-2 (“mbIL-2”) expression in tumor infiltrating lymphocytes expressing mbIL-2, membrane-bound IL-12 (“mbIL-12”), or both mbIL-2 and mbIL-12, provided by constriction mediated delivery of mRNA, at various time points following delivery. No contact (“NC”), empty squeeze (e.g., constriction mediated delivery was performed with an empty payload), and GFP provided by constricted mediated delivery of mRNA were used as controls in these experiments.

FIG. 1D shows the membrane-bound IL-12 (“mbIL-12”) expression in tumor infiltrating lymphocytes expressing membrane-bound IL-2 (“mbIL-2”), mbIL-12, or both mbIL-2 and mbIL-12, provided by constriction mediated delivery of mRNA, at various time points following delivery. No contact (“NC”), empty squeeze (e.g., constriction mediated delivery was performed with an empty payload), and GFP provided by constricted mediated delivery of mRNA were used as controls in these experiments.

FIG. 2A shows the mean fluorescence intensity (“MFI”) for GFP expression in tumor infiltrating lymphocytes expressing membrane-bound IL-2 (“mbIL-2”), membrane-bound IL-12 (“mbIL-12”), or both mbIL-2 and mbIL-12, provided by constriction mediated delivery of mRNA, at various time points following delivery. No contact (“NC”), empty squeeze (e.g., constriction mediated delivery was performed with an empty payload), and GFP provided by constricted mediated delivery of mRNA, were used as controls in these experiments.

FIG. 2B shows the mean fluorescence intensity (“MFI”) for membrane-bound IL-2 (“mbIL-2”) expression in tumor infiltrating lymphocytes expressing mbIL-2, membrane-bound IL-12 (“mbIL-12”), or both mbIL-2 and mbIL-12, provided by constriction mediated delivery of mRNA, at various time points following delivery. No contact (“NC”), empty squeeze (e.g., constriction mediated delivery was performed with an empty payload), and GFP provided by constricted mediated delivery of mRNA were used as controls in these experiments.

FIG. 2C shows the mean fluorescence intensity (“MFI”) for membrane-bound IL-12 (“mbIL-12”) expression in tumor infiltrating lymphocytes expressing membrane-bound IL-2 (“mbIL-2”), mbIL-12, or both mbIL-2 and mbIL-12, provided by constriction mediated delivery of mRNA, at various time points following delivery. No contact (“NC”), empty squeeze (e.g., constriction mediated delivery was performed with an empty payload), and GFP provided by constricted mediated delivery of mRNA were used as controls in these experiments.

FIGS. 3A-3D show the viability of tumor infiltrating lymphocytes expressing membrane-bound IL-2 (“mbIL-2”), membrane-bound IL-12 (“mbIL-12”), or both mbIL-2 and mbIL-12, provided by constriction mediated delivery of mRNA, and thereafter cultured in media comprising different IL-2 concentrations (3000 IU/mL, 300 IU/mL, 30 IU/mL, or 0 IU/mL. No contact (“NC”) and empty squeeze (e.g., constriction mediated delivery was performed with an empty payload) were used as controls in these experiments.

FIGS. 4A-4D show the proliferation of tumor infiltrating lymphocytes expressing membrane-bound IL-2 (“mbIL-2”), membrane-bound IL-12 (“mbIL-12”), or both mbIL-2 and mbIL-12, provided by constriction mediated delivery of mRNA, and thereafter cultured in media comprising different IL-2 concentrations (3000 IU/m L, 300 IU/m L, 30 IU/m L, or 0 IU/mL. No contact (“NC”) and empty squeeze (e.g., constriction mediated delivery was performed with an empty payload) were used as controls in these experiments.

FIGS. 5A-5D show the CD39+ expression of tumor infiltrating lymphocytes expressing membrane-bound IL-2 (“mbIL-2”), membrane-bound IL-12 (“mbIL-12”), or both mbIL-2 and mbIL-12, provided by constriction mediated delivery of mRNA, and thereafter cultured in media comprising different IL-2 concentrations (3000 IU/mL, 300 IU/mL, 30 IU/mL, or 0 IU/mL. No contact (“NC”) and empty squeeze (e.g., constriction mediated delivery was performed with an empty payload) were used as controls in these experiments.

FIGS. 6A-6D show the CD39+CD69+ expression of tumor infiltrating lymphocytes expressing membrane-bound IL-2 (“mbIL-2”), membrane-bound IL-12 (“mbIL-12”), or both mbIL-2 and mbIL-12, provided by constriction mediated delivery of mRNA, and thereafter cultured in media comprising different IL-2 concentrations (3000 IU/mL, 300 IU/mL, 30 IU/mL, or 0 IU/mL. No contact (“NC”) and empty squeeze (e.g., constriction mediated delivery was performed with an empty payload) were used as controls in these experiments.

FIGS. 7A-7D show the CD62L+ expression of tumor infiltrating lymphocytes expressing membrane-bound IL-2 (“mbIL-2”), membrane-bound IL-12 (“mbIL-12”), or both mbIL-2 and mbIL-12, provided by constriction mediated delivery of mRNA, and thereafter cultured in media comprising different IL-2 concentrations (3000 IU/mL, 300 IU/mL, 30 IU/mL, or 0 IU/mL. No contact (“NC”) and empty squeeze (e.g., constriction mediated delivery was performed with an empty payload) were used as controls in these experiments.

FIGS. 8A-8H show the CD69+ expression of tumor infiltrating lymphocytes expressing membrane-bound IL-2 (“mbIL-2”), membrane-bound IL-12 (“mbIL-12”), or both mbIL-2 and mbIL-12, provided by constriction mediated delivery of mRNA, and thereafter cultured in media comprising different IL-2 concentrations (3000 IU/mL, 300 IU/mL, 30 IU/mL, or 0 IU/mL. No contact (“NC”) and empty squeeze (e.g., constriction mediated delivery was performed with an empty payload) were used as controls in these experiments.

FIGS. 9A-9D show the membrane-bound IL-2 (“mbIL-2”) expression in tumor infiltrating lymphocytes expressing mbIL-2, membrane-bound IL-12 (“mbIL-12”), or both mbIL-2 and mbIL-12, provided by constriction mediated delivery of mRNA, and thereafter cultured in media comprising different IL-2 concentrations (3000 IU/mL, 300 IU/mL, 30 IU/mL, or 0 IU/mL. No contact (“NC”) and empty squeeze (e.g., constriction mediated delivery was performed with an empty payload) were used as controls in these experiments.

FIGS. 10A-10D show membrane-bound IL-12 (“mbIL-12”) expression in tumor infiltrating lymphocytes expressing membrane-bound IL-2 (“mbIL-2”), mbIL-12, or both mbIL-2 and mbIL-12, provided by constriction mediated delivery of mRNA, and thereafter cultured in media comprising different IL-2 concentrations (3000 IU/mL, 300 IU/mL, 30 IIU/mL, or 0 IU/mL. No contact (“NC”) and empty squeeze (e.g., constriction mediated delivery was performed with an empty payload) were used as controls in these experiments.

FIGS. 11A-11D show expression of both membrane-bound IL-2 and IL-12 (“mbIL-2 mbIL-12”) in tumor infiltrating lymphocytes expressing membrane-bound IL-2 (“mbIL-2”), mbIL-12, or both mbIL-2 and mbIL-12, provided by constriction mediated delivery of mRNA, and thereafter cultured in media comprising different IL-2 concentrations (3000 IU/mL, 300 IU/mL, 30 IU/mL, or 0 IU/mL. No contact (“NC”) and empty squeeze (e.g., constriction mediated delivery was performed with an empty payload) were used as controls in these experiments.

FIGS. 12A-12D show the T-bet+ expression of tumor infiltrating lymphocytes expressing membrane-bound IL-2 (“mbIL-2”), membrane-bound IL-12 (“mbIL-12”), or both mbIL-2 and mbIL-12, provided by constriction mediated delivery of mRNA, and thereafter cultured in media comprising different IL-2 concentrations (3000 IU/mL, 300 IU/mL, 30 IU/mL, or 0 IU/mL. No contact (“NC”) and empty squeeze (e.g., constriction mediated delivery was performed with an empty payload) were used as controls in these experiments.

FIGS. 13A-13D show the Eomes+ and TCF-1+ expression of tumor infiltrating lymphocytes expressing membrane-bound IL-2 (“mbIL-2”), membrane-bound IL-12 (“mbIL-12”), or both mbIL-2 and mbIL-12, provided by constriction mediated delivery of mRNA, and thereafter cultured in media comprising different IL-2 concentrations (3000 IU/mL, 300 IU/mL, 30 IU/mL, or 0 IU/mL. No contact (“NC”) and empty squeeze (e.g., constriction mediated delivery was performed with an empty payload) were used as controls in these experiments.

FIGS. 14A-14B show the viability of tumor infiltrating lymphocytes expressing both membrane-bound IL-2 (“mbIL-2”) and membrane-bound IL-12 (“mbIL-12”) provided by constriction mediated delivery of mRNA at 0.1 mg/mL, 0.25 mg/mL, or 0.5 mg/mL, and tumor infiltrating lymphocytes expressing CD86 and mbIL-2, CD86 and mb-IL12, and CD86 and both mbIL-2 and mbIL-12, provided by constriction mediated delivery of mRNA at 0.25 mg/mL The tumor infiltrating lymphocytes were cultured in media comprising either 3000 IU/mL (FIG. 14A) or 0 IU/mL (FIG. 14B) IL-2 following mRNA delivery. No contact (“NC”) and empty squeeze (e.g., constriction mediated delivery was performed with an empty payload) were used as controls in these experiments.

FIGS. 15A-15B show the proliferation of tumor infiltrating lymphocytes expressing both membrane-bound IL-2 (“mbIL-2”) and membrane-bound IL-12 (“mbIL-12”) provided by constriction mediated delivery of mRNA at 0.1 mg/mL, 0.25 mg/mL, or 0.5 mg/mL, and tumor infiltrating lymphocytes expressing CD86 and mbIL-2, CD86 and mb-IL12, and CD86 and both mbIL-2 and mbIL-12, provided by constriction mediated delivery of mRNA at 0.25 mg/mL. The tumor infiltrating lymphocytes were cultured in media comprising either 3000 IU/mL (FIG. 15A) or 0 IU/mL (FIG. 15B) IL-2 following mRNA delivery. No contact (“NC”) and empty squeeze (e.g., constriction mediated delivery was performed with an empty payload) were used as controls in these experiments.

FIGS. 16A-16B show the CD39+ expression of tumor infiltrating lymphocytes expressing both membrane-bound IL-2 (“mbIL-2”) and membrane-bound IL-12 (“mbIL-12”) provided by constriction mediated delivery of mRNA at 0.1 mg/mL, 0.25 mg/mL, or 0.5 mg/mL, and tumor infiltrating lymphocytes expressing CD86 and mbIL-2. CD86 and mb-IL12, and CD86 and both mbIL-2 and mbIL-12, provided by constriction mediated delivery of mRNA at 0.25 mg/mL. The tumor infiltrating lymphocytes were cultured in media comprising either 3000 IUI/mL (FIG. 16A) or 0 IU/mL (FIG. 16B) IL-2 following mRNA delivery. No contact (“NC”) and empty squeeze (e.g., constriction mediated delivery was performed with an empty payload) were used as controls in these experiments.

FIGS. 17A-17B show the CD62L+ expression of tumor infiltrating lymphocytes expressing both membrane-bound IL-2 (“mbIL-2”) and membrane-bound IL-12 (“mbIL-12”) provided by constriction mediated delivery of mRNA at 0.1 mg/mL, 0.25 mg/mL, or 0.5 mg/mL, and tumor infiltrating lymphocytes expressing CD86 and mbIL-2, CD86 and mb-IL12, and CD86 and both mbIL-2 and mbIL-12, provided by constriction mediated delivery of mRNA at 0.25 mg/mL. The tumor infiltrating lymphocytes were cultured in media comprising either 3000 IU/mL (FIG. 17A) or 0 IU/mL (FIG. 17B) IL-2 following mRNA delivery. No contact (“NC”) and empty squeeze (e.g., constriction mediated delivery was performed with an empty payload) were used as controls in these experiments.

FIGS. 18A-18B show the CD86+ expression of tumor infiltrating lymphocytes expressing both membrane-bound IL-2 (“mbIL-2”) and membrane-bound IL-12 (“mbIL-12”) provided by constriction mediated delivery of mRNA at 0.1 mg/mL, 0.25 mg/mL, or 0.5 mg/mL, and tumor infiltrating lymphocytes expressing CD86 and mbIL-2, CD86 and mb-IL12, and CD86 and both mbIL-2 and mbIL-12, provided by constriction mediated delivery of mRNA at 0.25 mg/mL. The tumor infiltrating lymphocytes were cultured in media comprising either 3000 IU/mL (FIG. 18A) or 0 IU/mL (FIG. 18B) IL-2 following mRNA delivery. No contact (“NC”) and empty squeeze (e.g., constriction mediated delivery was performed with an empty payload) were used as controls in these experiments.

FIGS. 19A-19B show the membrane-bound IL-2 (“mbIL-2”) expression of tumor infiltrating lymphocytes expressing both mbIL-2 and membrane-bound IL-12 (“mbIL-12”) provided by constriction mediated delivery of mRNA at 0.1 mg/mL, 0.25 mg/mL, or 0.5 mg/mL, and tumor infiltrating lymphocytes expressing CD86 and mbIL-2, CD86 and mb-IL12, and CD86 and both mbIL-2 and mbIL-12, provided by constriction mediated delivery of mRNA at 0.25 mg/mL. The tumor infiltrating lymphocytes were cultured in media comprising either 3000 IU/mL (FIG. 19A) or 0 IU/mL (FIG. 19B) IL-2 following mRNA delivery. No contact (“NC”) and empty squeeze (e.g., constriction mediated delivery was performed with an empty payload) were used as controls in these experiments.

FIGS. 19C-19D show the mean fluorescence intensity (“MFI”) of membrane-bound IL-2 (“mbIL-2”) expression of tumor infiltrating lymphocytes expressing both mbIL-2 and membrane-bound IL-12 (“mbIL-12”) provided by constriction mediated delivery of mRNA at 0.1 mg/mL, 0.25 mg/mL, or 0.5 mg/mL, and tumor infiltrating lymphocytes expressing CD86 and mbIL-2, CD86 and mb-IL12, and CD86 and both mbIL-2 and mbIL-12, provided by constriction mediated delivery of mRNA at 0.25 mg/mL The tumor infiltrating lymphocytes were cultured in media comprising either 3000 IU/mL (FIG. 19C) or 0 IU/mL (FIG. 19D) IL-2 following mRNA delivery. No contact (“NC”) and empty squeeze (e.g., constriction mediated delivery was performed with an empty payload) were used as controls in these experiments.

FIGS. 20A-20B show the membrane-bound IL-12 (“mbIL-12”) expression of tumor infiltrating lymphocytes expressing both mbIL-12 and membrane-bound IL-2 (“mbIL-2”) provided by constriction mediated delivery of mRNA at 0.1 mg/mL, 0.25 mg/mL, or 0.5 mg/mL, and tumor infiltrating lymphocytes expressing CD86 and mbIL-2, CD86 and mb-IL12, and CD86 and both mbIL-2 and mbIL-12, provided by constriction mediated delivery of mRNA at 0.25 mg/mL. The tumor infiltrating lymphocytes were cultured in media comprising either 3000 IU/mL (FIG. 20A) or 0 IU/mL (FIG. 20B) IL-2 following mRNA delivery. No contact (“NC”) and empty squeeze (e.g., constriction mediated delivery was performed with an empty payload) were used as controls in these experiments.

FIGS. 20C-20D show the mean fluorescence intensity (“MFI”) of membrane-bound IL-12 (“mbIL-12”) expression of tumor infiltrating lymphocytes expressing both mbIL-12 and membrane-bound IL-2 (“mbIL-2”) provided by constriction mediated delivery of mRNA at 0.1 mg/mL, 0.25 mg/mL, or 0.5 mg/mL, and tumor infiltrating lymphocytes expressing CD86 and mbIL-2, CD86 and mb-IL12, and CD86 and both mbIL-2 and mbIL-12, provided by constriction mediated delivery of mRNA at 0.25 mg/mL. The tumor infiltrating lymphocytes were cultured in media comprising either 3000 IU/mL (FIG. 20C) or 0 IU/mL (FIG. 20D) IL-2 following mRNA delivery. No contact (“NC”) and empty squeeze (e.g., constriction mediated delivery was performed with an empty payload) were used as controls in these experiments.

FIGS. 21A-21B show the T-bet+ expression of tumor infiltrating lymphocytes expressing both membrane-bound IL-2 (“mbIL-2”) and membrane-bound IL-12 (“mbIL-12”) provided by constriction mediated delivery of mRNA at 0.1 mg/mL, 0.25 mg/mL, or 0.5 mg/mL, and tumor infiltrating lymphocytes expressing CD86 and mbIL-2. CD86 and mb-IL12, and CD86 and both mbIL-2 and mbIL-12, provided by constriction mediated delivery of mRNA at 0.25 mg/mL. The tumor infiltrating lymphocytes were cultured in media comprising either 3000 IU/mL (FIG. 21A) or 0 IU/mL (FIG. 21B) IL-2 following mRNA delivery. No contact (“NC”) and empty squeeze (e.g., constriction mediated delivery was performed with an empty payload) were used as controls in these experiments.

FIGS. 22A-22B show the Eomes+ expression of tumor infiltrating lymphocytes expressing both membrane-bound IL-2 (“mbIL-2”) and membrane-bound IL-12 (“mbIL-12”) provided by constriction mediated delivery of mRNA at 0.1 mg/mL, 0.25 mg/mL, or 0.5 mg/mL, and tumor infiltrating lymphocytes expressing CD86 and mbIL-2, CD86 and mb-IL12, and CD86 and both mbIL-2 and mbIL-12, provided by constriction mediated delivery of mRNA at 0.25 mg/mL. The tumor infiltrating lymphocytes were cultured in media comprising either 3000 IU/mL (FIG. 22A) or 0 IU/mL (FIG. 22B) IL-2 following mRNA delivery. No contact (“NC”) and empty squeeze (e.g., constriction mediated delivery was performed with an empty payload) were used as controls in these experiments.

FIGS. 23A-23B show the Eomes+ and TCF-1+ expression of tumor infiltrating lymphocytes expressing both membrane-bound IL-2 (“mbIL-2”) and membrane-bound IL-12 (“mbIL-12”) provided by constriction mediated delivery of mRNA at 0.1 mg/mL, 0.25 mg/mL, or 0.5 mg/mL, and tumor infiltrating lymphocytes expressing CD86 and mbIL-2, CD86 and mb-IL12, and CD86 and both mbIL-2 and mbIL-12, provided by constriction mediated delivery of mRNA at 0.25 mg/mL. The tumor infiltrating lymphocytes were cultured in media comprising either 3000 IU/mL (FIG. 23A) or 0 IU/mL (FIG. 23B) IL-2 following mRNA delivery. No contact (“NC”) and empty squeeze (e.g., constriction mediated delivery was performed with an empty payload, i.e., no payload) were used as controls in these experiments.

FIGS. 24A-24B show the expression kinetics of membrane-bound IL-2 and IL-12, respectively, in TILs squeeze processed with mRNA encoding membrane-bound IL-2 and/or membrane-bound IL-12. The expression of membrane-bound IL-2 and membrane-bound IL-12 are shown as the geometric mean of the fluorescence intensity as measured using flow cytometry. The different groups shown include TILs that were: (i) not modified (i.e., no squeeze processing and no mRNA) (“NC”), (ii) not squeeze processed but expanded in the presence of a high dose of IL-2 (“NC+rhIL2”), (iii) squeeze processed without any payload (“ES”), (iv) squeeze processed without any payload but expanded in presence of a high dose of IL-2 (“ES+rhIL-2”), (v) squeeze processed with mRNA encoding membrane-bound IL-2 (“mbIL2”), and (vi) squeeze processed with mRNA encoding membrane-bound IL-2 and mRNA encoding membrane-bound IL-12 (“mbIL2/12”).

FIGS. 24C-24D show CD62L expression and viability of TILs at various time points after squeeze processing with mRNA encoding membrane-bound IL-2 and/or membrane-bound IL-12. FIG. 24C provides the CD62L expression, which is shown as the geometric mean of the fluorescence intensity as measured using flow cytometry. FIG. 24D provides a comparison of the percentage of viable cells observed. The different groups shown are the same as those described in FIGS. 24A and 24B.

FIGS. 25A-25B show the expression of membrane-bound IL-2 and IL-12, respectively, in TILs. Squeeze processed TILs were cultured in vitro as shown in FIGS. 25A-B, or adoptively transferred into NSG mice. The animals received an administration of one of the following TILs: (i) squeeze processed without any payload (“ES” or “Control”), (ii) squeeze processed with mRNA encoding membrane-bound IL-2 (“mbIL2”), and (iii) squeeze processed with mRNA encoding membrane bound IL-2 and mRNA encoding membrane-bound IL-12 (“mbIL2/12”).

FIGS. 25C-25E provide comparison of the in vivo persistence of the squeeze-processed TILs described in FIGS. 25A-25B In FIG. 25C, the persistence of the TILs is shown as a percentage of total CD45+ cells that expressed human CD45 (i.e., squeeze-processed TILs) observed at different time points in the blood post adoptive transfer as measured using flow cytometry. In FIG. 25D, the persistence of the TILs is provided as a comparison of the percentage of total CD45+ cells that expressed human CD45 (i.e., squeeze-processed TILs) observed in the spleen at day 5 post adoptive transfer. FIG. 25E provides a comparison of the absolute cell count of the squeeze processed TILs observed in the spleen of animals from the different groups at day 5 post adoptive transfer.

FIGS. 25F-25G provide comparison of squeeze-processed TILs having a central memory phenotype, effector memory phenotype, or effector phenotype in the blood (FIG. 25F) or in the spleen (FIG. 25G) at day 5 post adoptive transfer.

FIGS. 26A-26D show mbIL-2 and mbIL-12 expression at various time points post-thaw. The TILs are the same as those described in FIGS. 26A and 26B. FIGS. 26A and 26C show mbIL-2 expression as percentage of total CD3+ T cells and as geometric mean of the fluorescence intensity, respectively. FIGS. 26B and 26D show mbIL-12 expression as percentage of total CD3+ T cells and as geometric mean of the fluorescence intensity, respectively.

FIGS. 27A and 27B provide comparison of IL-2 and IL-12 expression, respectively, in TILs from the different groups with (2^{nd} bar in each of the groups) or without (1^{st} bar in each of the groups) anti-CD3 antibody stimulation. The expression of both IL-2 and IL-12 are shown as geometric mean of the fluorescence intensity as measured using flow cytometry. The different groups were TILs that were (i) not modified (i.e., no squeeze processing and no mRNA) (“NC”), (ii) not squeeze processed but expanded in the presence of a high dose of IL-2 (“NC+rhIL2”), (iii) not squeeze processed but expanded in the presence of a high dose of IL-2 and IL-12 (“NC+rhIL2/12”), (iv) squeeze processed without any payload (“ES”), (v) squeeze processed with mRNA encoding membrane-bound IL-2 (“mbIL2”), and (vi) squeeze processed with mRNA encoding membrane-bound IL-2 and mRNA encoding membrane-bound IL-12 (“mbIL2/12”).

FIG. 27C provide comparison of the ability of TILs from the different groups to produce IFN-γ with (2^{nd} bar in each of the groups) or without (1^{st} bar in each of the groups) anti-CD3 antibody stimulation. In FIG. 27C, IFN-γ expression is shown as geometric mean of the fluorescence intensity as measured using flow cytometry. The different TIL groups are the same as described in FIGS. 27A and 27B.

FIGS. 28A and 28B show the expression kinetics of membrane-bound IL-2 and membrane-bound IL-7, respectively, in TILs squeeze processed with one of the following: (i) not modified (i.e., no squeeze processing and no mRNA) (“NC”), (ii) not squeeze processed but expanded in the presence of a high dose of IL-2 (“NC+IL-2”), (iii) squeeze processed without any payload (“ES”), (iv) squeeze processed with mRNA encoding membrane-bound IL-2 (“mbIL-2”), (v) squeeze processed with mRNA encoding membrane-bound IL-7 (“mbIL-7”) and (vi) squeeze processed with mRNA encoding membrane-bound IL-2 and mRNA encoding membrane-bound IL-7 (“mbIL2/7”).

FIG. 28C provides exemplary flow cytometry plots showing the expression of membrane-bound IL-7 and detection of IL-7 receptor on TILs that were either squeeze processed without any mRNA (left graph) or squeeze processed with mRNA encoding membrane-bound IL-7.

FIG. 28D provides a comparison of the percentage of viable cells observed in TILs from the different groups at various time points post squeeze processing. The different groups shown are the same as those described in FIGS. 28A and 28B.

FIG. 28E provides a comparison of pSTAT5 expression in TILs from the different groups at day 1 (left graph) or at day 2 (right graph) post squeeze processing. pSTAT5 expression is shown as a percentage of total cells that express pSTAT5. The different TIL groups shown are the same as those described in FIGS. 28A and 28B.

FIGS. 29A-29C provide comparison of membrane-bound IL-2 (FIG. 29A), membrane-bound IL-12 (FIG. 29B), and membrane-bound IL-7 (FIG. 29C) expression in the following groups of TILs. (i) not modified (i.e., no squeeze processing and no mRNA) (“NC”), (ii) not squeeze processed but expanded in the presence of a high dose of IL-2 (“NC+rhIL2”), (iii) not squeeze processed but expanded in the presence of a high dose of recombinant IL-2 and recombinant IL-12 (“NC+rhIL2/12”), (iv) squeeze processed without any payload (“ES”). (v) squeeze processed with mRNA encoding membrane-bound IL-2 (“mbIL2”), (vi) squeeze processed with mRNA encoding membrane-bound IL-2 and mRNA encoding membrane-bound IL-12 (“mbIL2/12”), and (vii) squeeze processed with mRNA encoding membrane-bound IL-2, mRNA encoding membrane-bound IL-7, and mRNA encoding membrane-bound IL-12 (“mbIL-2/7/12”). The expression of each of the membrane-bound cytokines is shown as the geometric mean of the fluorescence intensity as measured using flow cytometry.

FIGS. 29D-29H show the ability of the different TILs to kill tumor cells grown from the resected tumor the TILs are derived from in vitro. FIGS. 29D-29G provide representative co-culture fluorescent images of caspase-3/7 expression (green marker for cell death), tumor cells (labeled in red) In FIG. 29D, the tumor cells were cultured with unprocessed TILs (i.e., no squeeze processing and no mRNA). In FIG. 29E, the tumor cells were cultured with the unprocessed TILs in combination with recombinant IL-2 and IL-12. In FIG. 29F, the tumor cells were cultured with TILs squeeze processed with mRNA encoding a membrane-bound IL-2 and a mRNA encoding a membrane-bound IL-12. In FIG. 29G, the tumor cells were cultured with TILs squeeze processed with mRNA encoding a membrane-bound IL-2, mRNA encoding a membrane-bound IL-7, and mRNA encoding a membrane-bound IL-12. FIG. 29H provides a quantitative comparison of the results provided in FIGS. 29D-29G.

FIG. 29I provides a comparison of IFN-γ production by the different TILs as measured using ELISA. The different TIL groups are the same as that described in FIGS. 29D-29H.

FIGS. 30A-30B provide comparison of the expression of membrane-bound IL-2 and membrane-bound IL-7, respectively, in TILs from different groups at day 1 post squeeze processing. The expression of each of the membrane-bound cytokines is shown as the geometric mean of the fluorescence intensity as measured by using flow cytometry. The different TILs shown include: (i) unprocessed TILs (i.e., no squeeze processing and no mRNA) (“NC”), (ii) TILs squeeze processed with no mRNA (“ES”), (iii) TILs squeeze processed with mRNA encoding membrane-bound IL-2 (“mbIL-2”), (iv) TILs having under squeeze processing twice with mRNA encoding membrane-bound IL-2 (“mbIL-2 2×”), (v) TILs squeeze processed with mRNA encoding membrane-bound IL-7 (“mbIL-7”), and (vi) TILs squeeze processed with mRNA encoding membrane-bound IL-2 and mRNA encoding membrane-bound IL-7 (“mbIL-2/7”).

FIG. 30C shows pSTAT5 expression in TILs of the different groups at day 1 post-squeeze processing. pSTAT5 expression is shown as the geometric mean of the fluorescence intensity as measured using flow cytometry. The different TILs shown are the same as that described above in FIGS. 30A-30B, including unprocessed TILs cultured in recombinant IL-2 (“NC+rhIL-2”) or recombinant LL-7 (“NC+rhIL-7).

FIG. 30D provides a comparison of the percentage of viable cells observed at day 5 post-squeeze processing in the different TIL groups. The different TILs shown are the same as that described above in FIGS. 30A-30C.

FIGS. 31A-31C provide comparison of Bcl-2 and membrane-bound IL-7 expression in the following groups of TILs. (i) unprocessed TILs (“NC”), (ii) TILs squeeze processed with no mRNA and subsequently cultured with recombinant IL-7 (“ES+rhIL-7”), (iii) TILs squeeze processed with mRNA encoding membrane-bound IL-7, (iv) TILs squeeze processed with mRNA encoding Bcl-2, (v) TILs squeeze processed with mRNA encoding membrane-bound IL-7 and mRNA encoding Bcl-2 (“mbIL-7+Bcl-2”), (vi) TILs squeeze processed with mRNA encoding membrane-bound IL-2 (“mbIL-2”), (vii) TILs squeeze processed with mRNA encoding membrane-bound IL-2 and mRNA encoding Bcl-2 (“mbIL-2+Bcl-2”), and (viii) TILs squeeze processed with mRNA encoding membrane-bound IL-2, mRNA encoding membrane-bound IL-7, and mRNA encoding Bcl-2. Bcl-2 expression is shown both as a percentage of total TILs (FIG. 31A) and as geometric mean of the fluorescence intensity as measured using flow cytometry (FIG. 31B). FIG. 31C shows the expression of membrane-bound IL-7 as the geometric mean of the fluorescence intensity as measured using flow cytometry.

FIGS. 31D and 31E show the effect of Bcl-2 inhibitor Venetoclax and Mcl-1 inhibitor S63548 (targets which are downstream of IL-7R signaling) on the viability of the different TILs described in FIGS. 31A-31C. In FIG. 31D, the percentage of live TILs observed is provided as function of the inhibitor concentration. In FIG. 31E, the effect is shown as a percentage of TILs that are positive for Apotracker dye expression (marker for apoptosis).

FIGS. 32A-32F provide comparison of the expression of membrane-bound IL-2 (FIGS. 32A and 32B), membrane-bound IL-12 (FIGS. 32C and 32D), and CD62L expression (FIGS. 32E and 32F) in the following groups of TILs: (i) unprocessed TILs (“NC”), (ii) TILs squeeze processed without any mRNA (“ES”), (iii) TILs squeeze processed with mRNA encoding a membrane-bound IL-2, mRNA encoding a membrane-bound IL-12, and mRNA encoding CD86, with each of the mRNAs at a concentration of 250 μg/mL (“250 μg/mL Triple mRNA”), and (iv) TILs squeeze processed with the same three mRNAs of (iii) but with each of the mRNAs at a concentration of 500 μg/mL (“500 μg/mL Triple mRNA”). Expression of the different markers are shown both as a measure fluorescence intensity (FIGS. 32A, 32C, and 32E) and as a percentage of total cells (FIGS. 32B, 32D, and 32F).

FIG. 33A provides a representative flow cytometry plot showing the expression of membrane-bound IL-2 expression (y-axis) and CD25 expression (x-axis) in TILs squeeze processed with mRNA encoding membrane-bound IL-2.

FIGS. 33B and 33C provide comparison of CD25 expression on TILs from three different donors (TIL-1, TIL-2, and TIL-3) using either a non-blocking anti-CD25 antibody (FIG. 33B) or a blocking anti-CD25 antibody (FIG. 33C), in the following TIL groups: (i) squeeze processed without any mRNA (“ES”), (ii) squeezed processed with mRNA encoding membrane-bound IL-2 and sub-gated onto the fraction of mbIL-2-expressing TILs (“mbIL-2 positive”), and (iii) squeezed processed with mRNA encoding membrane-bound IL-2 and sub-gated onto the fraction of non-mbIL-2-expressing TILs (“mbIL-2 negative”).

FIG. 33D provides a comparison of available CD25 expressed on the TILs from the different groups at various time points post-squeeze processing. The different groups shown include: the same groups from FIG. 33C, plus TILs that were: (i) squeeze processed without any mRNA and cultured in recombinant IL-2 (“ES+IL-2”), (ii) squeeze processed with mRNA encoding membrane-bound IL-2 (“mbIL-2 total”), and (iii) squeeze processed with mRNA encoding membrane-bound IL-2 and cultured in recombinant IL-2 (“mbIL-2+IL-2”).

FIGS. 34A-34C provide comparison of the expression of GFP, membrane-bound IL-2, and membrane-bound IL-12, respectively, in cells isolated from the blood, spleen, liver, and lung of mice that received administration of one of the following TILs: (i) TILs squeeze processed without any mRNA (“ES”), (ii) TILs squeeze processed with mRNA encoding membrane-bound IL-2 (“mbIL2”), and (iii) TILs squeeze processed with mRNA encoding membrane-bound IL-2, mRNA encoding membrane-bound IL-12, and mRNA encoding GFP (“mbIL-2, mbIL12, GFP”).

FIG. 34D provides comparison of the percentage of the transferred TILs observed in various tissues (blood, spleen, liver, and lung) of the mice. The groups are the same as those described in FIGS. 34A-34C.

FIGS. 35A-35F show the expression of the following markers in the TILs at day 1 post squeeze processing: membrane-bound IL-2, membrane-bound IL-12, Ki67, CD39, CD62L, and CD69, respectively. The different TILs shown include: (i) unprocessed TILs (“NC”), (b) unprocessed TILs cultured in recombinant IL-2 (“NC+IL-2”), (c) unprocessed TILs cultured in recombinant IL-2 and IL-12 (“NC+IL-2/12”), (d) TILs squeeze processed with no mRNA (“ES”), (e) TILs squeeze processed with no mRNA and cultured in recombinant IL-2 (“ES+IL-2”), (f) TILs squeeze processed with no mRNA and cultured in recombinant IL-2 and IL-12 (“ES+IL-2/12”), (g) TILs squeeze processed with mRNA encoding membrane-bound IL-2 (“mbIL-2”), and (h) TILs squeeze processed with mRNA encoding membrane-bound IL-2 and mRNA encoding membrane-bound IL-12 (“mbIL-2/12”).

FIGS. 36A and 36B show the expression of membrane-bound IL-2 and membrane-bound IL-12, respectively, in two different TIL donors (“Donor 1” and “Donor 2”) at day 1 post squeeze processing. The different TIL groups shown include: (i) unprocessed TILs (“NC”), (ii) unprocessed TILs cultured in recombinant IL-2 (“NC+IL-2”), (iii) unprocessed TILs cultured in recombinant IL-2 and IL-12 (“NC+IL-2/12”), (iv) TILs squeeze processed with no mRNA (“ES”), (v) TILs squeeze processed with mRNA encoding membrane-bound IL-2 (“mbIL-2”), and (vi) TILs squeeze processed with mRNA encoding membrane-bound IL-2 and mRNA encoding membrane-bound IL-12 (“mbIL-2/12”).

FIGS. 36C and 36D show the proliferative capacity and viability, respectively, as measured at day 4 post squeeze delivery for the different TILs described in FIGS. 36A and 36B, 36E-36F show CD62L expression of TILs that were CD62L+ at day 0 (“Positive at Baseline”. FIG. 36E) or CD62L− at day 0 (“Negative at Baseline”, FIG. 36F) measured at day 4 post-squeeze delivery for the different TILs in FIGS. 36A-B. FIGS. 36G-H show geometric mean of the fluorescence intensity of proliferative dyes for TILs that were CD62L− at day 0 (“Negative at Baseline”. FIG. 36G) that are CD62L− (bar 1) or CD62L+ (bar 2) at day 4 post-squeeze delivery, or CD62L+ at day 0 (“Positive at Baseline”. FIG. 36F) that are CD62L− (bar 1) or CD62L+ (bar 2) at day 4 post-squeeze delivery for the different TILs in FIGS. 36A-B.

FIGS. 37A and 37B provide flow cytometry histogram plots showing the expression of membrane-bound IL-7 and Bcl-2, respectively, in the following groups of TILs: (i) unprocessed TILs (“NC”), (ii) TILs squeeze processed with no mRNA (“ES”), (iii) TILs squeeze processed with mRNA encoding membrane-bound IL-7 (“mbIL-7), (iv) TILs squeeze processed with mRNA encoding Bcl-2 (“BCL2”), and (v) TILs squeeze processed with mRNA encoding membrane-bound IL-7 and mRNA encoding Bcl-2 (“mbIL-7+BCL2”).

FIG. 37C shows the viability of the different TILs described in FIGS. 37A and 37B after 5 days of culture with varying concentrations of exogenous recombinant IL-7 protein.

FIG. 37D shows the effect of membrane-bound IL-7 protein expression on the dependency of TILs to IL-7 for viability.

FIGS. 38A-38) provide comparison of mbIL-2 and mbIL-12 expression in TILs after squeeze processing with mRNAs encoding mbIL-2 or mbIL-12 using constrictions having different diameters. As further described in Example 20, prior to squeeze processing, the TILs were expanded via REP processing that involved one of four different anti-CD3 antibody clones (OKT3, UCHT1, HIT3a, and SK7; see x-axis). FIGS. 38A and 38C show mbIL-2 expression as percentage of total CD3+ T cells and as geometric mean of the fluorescence intensity, respectively. FIGS. 38A and 38C show mbIL-12 expression as percentage of total CD3+ T cells and as geometric mean of the fluorescence intensity, respectively. The different TIL groups shown in each of FIGS. 38A-38D include: (1) unprocessed TILs (i.e., no squeeze processing) (1^{st} bar in each of the anti-CD3 antibody clones, “No Contact”); (2) TILs squeeze processed with no payload (i.e., squeezed but no mRNA delivered) (2^{nd} bar in each of the anti-CD3 antibody clones; “Empty”); (3) TILs squeezed with the mRNAs using constriction having a 3.5 μm diameter (3^{rd} bar in each of the anti-CD3 antibody clones; “10-3-70 SF”); and (4) TILs squeezed with the mRNAs using constriction having a 4 μm diameter (last bar in each of the anti-CD3 antibody clones; “10-4-70 ST”).

FIGS. 39A-39H provide comparison of mbIL-2 and mb-IL-12 in TILs that were first expanded via REP processing using different concentrations of anti-CD3 antibody (OKT3 clone) and then subsequently squeeze processed with mRNAs encoding the mbIL-2 or mbIL-12. FIGS. 39A and 39E show mbIL-2 expression as a percentage of total CD3+ T cells in squeeze processed TILs that were previously expanded in the presence of the anti-CD3 antibody at either 6 ng/mL or 30 ng/mL, respectively. FIGS. 39B and 39F show mbIL-2 expression as a geometric mean of the fluorescence intensity in squeeze processed TILs that were previously expanded in the presence of the anti-CD3 antibody at either 6 ng/mL or 30 ng/mL, respectively. FIGS. 39C and 39G show mbIL-12 expression as a percentage of total CD3+ T cells in squeeze processed TILs that were previously expanded in the presence of the anti-CD3 antibody at either 6 ng/mL or 30 ng/mL, respectively. FIGS. 39D and 39H show mbIL-2 expression as a geometric mean of the fluorescence intensity in squeeze processed TILs that were previously expanded in the presence of the anti-CD3 antibody at either 6 ng/mL or 30 ng/mL, respectively. For each of FIGS. 39A-39H, the different TIL groups included: (1) unprocessed TILs (i.e., no squeeze processing) (“No Contact”); (2) TILs squeeze processed with no payload (“Empty”); and (3) TILs squeeze processed with the mRNAs.

FIGS. 40A-40D provide comparison of CD62L expression on the TILs described in FIGS. 39A-39H. FIGS. 40A and 40B show CD62L expression (as percentage of total CD8+ T cells and as geometric mean fluorescence intensity among CD8+ T cells, respectively) in squeeze-processed TILs that were previously expanded in the presence of 6 ng/mL of anti-CD3 antibody (OKT3). FIGS. 40C and 40D show CD62L expression (as percentage of total CD8+ T cells and as geometric mean fluorescence intensity among CD8+ T cells, respectively) in squeeze-processed TILs that were previously expanded in the presence of 30 ng/mL of anti-CD3 antibody (OKT3).

FIGS. 41A-41D show the effect of constriction diameter and pressure during squeeze processing on mRNA delivery to TILs and subsequent expression of the encoded mbIL-2 and mbIL-12. FIGS. 41A and 41B show the expression of mbIL-2 as the percentage of total CD3+ cells and as geometric mean fluorescence intensity among the total CD3+ cells, respectively. FIGS. 41C and 41D show the expression of mbIL-12 as the percentage of total CD3+ cells and as geometric mean fluorescence intensity among the total CD3+ cells, respectively. The different TIL groups shown in each of FIGS. 41A-41D include: (1) unprocessed TILs (i.e., no squeeze processing) (1^{st} bar; circle), (2) TILs squeeze processed with no payload (i.e., squeezed but no mRNA delivered) (2^{nd} bar, square), (3) TILs squeezed with only mRNA encoding mbIL-2 (third bar, triangle), (4) TILs squeezed with only mRNA encoding mbIL-12 (fourth bar; inverted triangle), (5) TILs squeezed with both mRNA encoding mbIL-2 and mRNA encoding mbIL-12 at 45 psi (5^{th} bar; diamond), and (6) TILs squeeze with both mRNA encoding mbIL-2 and mRNA encoding mbIL-12 at 60 psi (last bar; open circle).

FIGS. 42A-42D show the effect of cell density on the delivery of mRNAs into TILs using squeeze processing. FIGS. 42A and 42B show the expression of mbIL-2 as the percentage of total CD3+ cells and as geometric mean fluorescence intensity among the total CD3+ cells, respectively. FIGS. 42C and 42D show the expression of mbIL-12 as the percentage of total CD3+ cells and as geometric mean fluorescence intensity among the total CD3+ cells, respectively. The different TIL groups shown in each of FIGS. 42A-42D include. (1) unprocessed TILs (i.e., no squeeze processing). (2) TILs squeeze processed with no payload (i.e., squeezed but no mRNA delivered), (3) TILs squeezed with both mRNA encoding mbIL-2 and mRNA encoding mbIL-12 at a cell density of 20×10⁶ cells/mL, and (4) TILs squeezed with both mRNA encoding mbIL-2 and mRNA encoding mbIL-12 at a cell density of 20×10⁶ cells/mL.

FIG. 43A provides representative flow cytometry plots showing CD127 expression (x-axis) on squeeze processed TILs at various time points (i.e., 0 hour, 4 hours, 24 hours, 48 hours, and 8 days) after squeeze processing. The different TIL groups shown include: (1) unprocessed TILs (i.e., no squeeze processing) (“No Contact”; 1^{st} column), (2) TILs squeeze processed with no payload (i.e., squeezed but no mRNA delivered) (“Empty”; 2^{nd} column), (3) TILs squeezed with only mRNA encoding mbIL-2 (middle column), (4) TILs squeezed with only mRNA encoding mbIL-12 (4^{th} column), and (5) TILs squeezed with both mRNA encoding mbIL-2 and mRNA encoding mbIL-12 (last column).

FIGS. 43B and 43C provide quantitative comparison squeeze processed TILs expressing certain central memory T cell-like phenotypic markers at various time points after squeeze processing (i.e., 0 hour, 4 hours, 24 hours, 48 hours, and 8 days). FIG. 43B shows the percentage of CD62L+ CD45RO+ CD8+ T cells that were positive for CD127 expression. FIG. 44B shows the percentage of total CD8+ T cells that were positive for the expression of CD62L, CD127, and CD45RO. The different TIL groups shown include: (1) unprocessed TILs (i.e., no squeeze processing) (“No Contact”, small circle), (2) TILs squeeze processed with no payload (i.e., squeezed but no mRNA delivered) (“Empty”, square), (3) TILs squeezed with only mRNA encoding mbIL-2 (triangle), (4) TILs squeezed with only mRNA encoding mbIL-12 (inverted triangle), and (5) TILs squeezed with both mRNA encoding mbIL-2 and mRNA encoding mbIL-12 (large circle).

FIGS. 44A-44E provide comparison of viability and mRNA delivery after squeeze processing under the following delivery parameters: 10-4.5-70 ST chip at 60 psi at room temperature. The TILs were squeeze delivered both mRNA encoding mbIL-2 and mRNA encoding mbIL-12 (right bar in each of FIGS. 44A-44E). Unprocessed TILs (i.e., no squeeze delivery) were used as control (left bar in each of FIGS. 44A-44E). FIG. 44A provides a comparison of the percent viability FIGS. 44B and 44C show mbIL-2 expression as percentage of total CD3+ T cells and as geometric mean of the fluorescence intensity, respectively. FIGS. 44D and 44E show mbIL-12 expression as percentage of total CD3+ T cells and as geometric mean of the fluorescence intensity, respectively.

FIGS. 45A-45D provide comparison of mbIL-2 and mbIL-12 expression in TILs after squeeze delivery of a single mRNA (encoding either mbIL-2 or mbIL-12) or two mRNAs (i.e., mRNA encoding mbIL-2 and mRNA encoding mbIL-12) using the following delivery parameters: 10-4.5-70 ST chip at 60 psi at room temperature. FIGS. 45A and 45B show mbIL-2 expression as percentage of total CD3+ T cells and as geometric mean of the fluorescence intensity, respectively. FIGS. 45C and 45D show mbIL-12 expression as percentage of total CD3+ T cells and as geometric mean of the fluorescence intensity, respectively. The different TIL groups shown include: (1) unprocessed TILs (i.e., no squeeze processing) (“No Contact”, circle; 1^{st} bar in each of the figures); (2) TILs squeeze processed with no payload (i.e., squeezed but no mRNA delivered) (“Empty”; square; 2^{nd} bar in each of the figures); (3) TILs squeezed with only mRNA encoding mbIL-2 (triangle; middle bar in each of the figures); (4) TILs squeezed with only mRNA encoding mbIL-12 (inverted triangle: fourth bar in each of the figures); and (5) TILs squeezed with both mRNA encoding mbIL-2 and mRNA encoding mbIL-12 (diamond; last bar).

FIGS. 46A and 46B show the effect of human serum albumin (HSA) concentration on both viability and recovery, respectively, post thaw of cryopreserved squeeze-processed TILs. As further described in Example 30, unprocessed TILs (i.e., no squeeze processing) (circle, darker line) and TILs squeeze processed with mRNA encoding mbIL-2 and mRNA encoding mbIL-12 (square; lighter line) were cryopreserved in a formulation comprising varying concentrations of HSA.

FIGS. 47A and 47B show percent viability of unprocessed TILs and squeeze-processed TILs, respectively, at various time points post-thaw. The TILs are the same as those described in FIGS. 46A and 46B—i.e., previously cryopreserved in a formulation comprising HSA at a concentration of 0% (circle), 1.25% (square), 2.5% (triangle), 5% (inverted triangle), or 10% (diamond).

FIGS. 48A-48D show mbIL-2 and mbIL-12 expression at various time points post-thaw. The TILs are the same as those described in FIGS. 47A and 47B FIGS. 48A and 48C show mbIL-2 expression as percentage of total CD3+ T cells and as geometric mean of the fluorescence intensity, respectively. FIGS. 48B and 48D show mbIL-12 expression as percentage of total CD3+ T cells and as geometric mean of the fluorescence intensity, respectively.

FIGS. 49A and 49B show the effect of DMSO concentration on both viability and recovery, respectively, post thaw of cryopreserved squeeze-processed TILs. As further described in Example 30, unprocessed TILs (i.e., no squeeze processing) (circle; darker line) and TILs squeeze processed with mRNA encoding mbIL-2 and mRNA encoding mbIL-12 (square; lighter line) were cryopreserved in a formulation comprising varying concentrations of DMSO.

FIGS. 50A and 50B show percent viability of unprocessed TILs and squeeze-processed TILs, respectively, at various time points post-thaw. The TILs are the same as those described in FIGS. 49A and 49B i.e., previously cryopreserved in a formulation comprising DMSO at a concentration of 0% (circle), 1.25% (square), 2.5% (triangle), or 5% (inverted triangle).

### DETAILED DESCRIPTION OF THE DISCLOSURE

In some aspects, provided are methods of modulating the activity and/or proliferative capacity of tumor-infiltrating lymphocytes (TILs), wherein the TILs are modified to increase expression of one or more co-stimulatory molecules and/or one or more cytokines. In some aspects, provided are methods of modulating the activity and/or proliferative capacity of tumor-infiltrating lymphocytes (TILs), wherein the TILs are prepared by a process comprising: a) passing a cell suspension comprising input TILs through a cell-deforming constriction, wherein a diameter of the constriction is a function of a diameter of the input TILs in the suspension, thereby causing perturbations of the input TILs large enough for one or more nucleic acids encoding one or more cytokines, one or more nucleic acids encoding one or more anti-apoptotic factors, and/or one or more nucleic acids encoding one or more co-stimulatory molecules to pass through to form perturbed input TILs; and b) incubating the perturbed input TILs with the one or more nucleic acids encoding one or more cytokines, one or more nucleic acids encoding one or more anti-apoptotic factors, and/or one or more nucleic acids encoding one or more co-stimulatory molecules to allow the one or more nucleic acids encoding one or more cytokines, one or more nucleic acids encoding one or more anti-apoptotic factors, and/or one or more nucleic acids encoding one or more co-stimulatory molecules to enter the perturbed input TILs; wherein the nucleic acids are expressed, thereby generating the TILs comprising the one or more cytokines, the one or more anti-apoptotic factors, and/or the one or more co-stimulatory molecules. In some aspects, provided herein is a method of modulating the activity and/or proliferative capacity of TILs, comprising intracellularly delivering one or more nucleic acids encoding one or more cytokines to the TILs in some aspects, provided herein is a method of modulating the activity and/or proliferative capacity of TILs, comprising intracellularly delivering one or more nucleic acids encoding one or more co-stimulatory molecules to the TILs. In some aspects, provided herein is a method of modulating the activity and/or proliferative capacity of TILs, comprising intracellularly delivering one or more nucleic acids encoding one or more anti-apoptotic factors to the TILs. In some aspects, provided herein is a method of modulating the activity and/or proliferative capacity of TILs, comprising intracellularly delivering (i) one or more nucleic acids encoding one or more cytokines, (ii) one or more nucleic acids encoding one or more anti-apoptotic factor, and/or (iii) one or more nucleic acids encoding one or more co-stimulatory molecules to the TILs. In some aspects, intracellularly delivering the one or more nucleic acids comprise the squeeze processing methods described herein.

Accordingly, in some aspects, provided herein is a method of modulating the activity and/or proliferative capacity of TILs, comprising passing a cell suspension comprising TILs through a cell-deforming constriction, thereby causing perturbations of the TILs such that (i) one or more nucleic acids encoding one or more cytokines, (ii) one or more nucleic acids encoding one or more anti-apoptotic factors. (iii) one or more nucleic acids encoding one or more co-stimulatory molecules, or (iv) any combination of (i) to (iii) enter the TILs through the perturbations when contacted with the TILs. In some aspects, such a method can further comprise contacting the TILs with the (i) one or more nucleic acids encoding one or more cytokines, (ii) one or more nucleic acids encoding one or more anti-apoptotic factors, (iii) one or more nucleic acids encoding one or more co-stimulatory molecules, or (iv) any combination of (i) to (iii). As further described and demonstrated herein, in some aspects, the one or more cytokines, one or more anti-apoptotic factors, and/or one or more co-stimulatory molecules can modulate the activity and/or proliferative capacity of the TILs.

In some aspects, provided are compositions comprising modified TILs, wherein the TILs are modified to increase expression of one or more of co-stimulatory molecules, one or more anti-apoptotic factors, and/or one or more cytokines. Accordingly, in some aspects, provided herein is a composition comprising modified TILs which exhibit increased expression of one or more co-stimulatory molecules as compared to reference TILs (e.g., corresponding TILs that have not been modified as described herein). In some aspects, provided herein is a composition comprising modified TILs which exhibit increased expression of one or more anti-apoptotic factors as compared to reference TILs (e.g., corresponding TILs that have not been modified as described herein). In some aspects, provided herein is a composition comprising modified TILs which exhibit increased expression of one or more cytokines as compared to reference TILs (e.g., corresponding TILs that have not been modified as described herein). In some aspects, provided herein is a composition comprising modified TILs which exhibit increased expression of both one or more cytokines and one or more co-stimulatory molecules as compared to reference TILs (e.g., corresponding TILs that have not been modified as described herein) In some aspects, provided herein is a composition comprising modified TILs which exhibit increased expression of each of one or more cytokines, one or more anti-apoptotic factors, and one or more co-stimulatory molecules as compared to reference TILs (e.g., corresponding TILs that have not been modified as described herein).

In some aspects, provided are compositions comprising modified TILs, wherein the modified TILs are prepared by a process comprising: a) passing a cell suspension comprising input TILs through a cell-deforming constriction, wherein a diameter of the constriction is a function of a diameter of the input TILs in the suspension, thereby causing perturbations of the input TILs large enough for one or more nucleic acids encoding one or more cytokines, one or more nucleic acids encoding one or more anti-apoptotic factors, and/or one or more nucleic acids encoding one or more co-stimulatory molecules to pass through to form perturbed input TILs; and b) incubating the perturbed input TILs with the one or more nucleic acids encoding one or more cytokines, one or more nucleic acids encoding one or more anti-apoptotic factors, and/or one or more nucleic acids encoding one or more co-stimulatory molecules to allow the one or more nucleic acids encoding one or more cytokines, one or more nucleic acids encoding one or more anti-apoptotic factors, and/or one or more nucleic acids encoding one or more co-stimulatory molecules to enter the perturbed input TILs; wherein the nucleic acids are expressed, thereby generating the modified TILs comprising the one or more cytokines and/or the one or more co-stimulatory molecules. Accordingly, in some aspects, the modified TILs described herein have been passed through a cell-deforming constriction, wherein the cell-deforming constriction deformed the TILs thereby causing perturbations of the TILs such that one or more nucleic acids encoding one or more cytokines had entered the TILs through the perturbations when contacted with the TILs. In some aspects, the modified TILs described herein have been passed through a cell-deforming constriction, wherein the cell-deforming constriction deformed the TILs thereby causing perturbations of the TILs such that one or more nucleic acids encoding one or more co-stimulatory molecules had entered the TILs through the perturbations when contacted with the TILs. In some aspects, the modified TILs described herein have been passed through a cell-deforming constriction, wherein the cell-deforming constriction deformed the TILs thereby causing perturbations of the TILs such that one or more nucleic acids encoding one or more anti-apoptotic factors had entered the TILs through the perturbations when contacted with the TILs. In some aspects, the modified TILs described herein have been passed through a cell-deforming constriction, wherein the cell-deforming constriction deformed the TILs thereby causing perturbations of the TILs such that both (i) one or more nucleic acids encoding one or more cytokines and (ii) one or more nucleic acids encoding one or more co-stimulatory molecules had entered the TILs through the perturbations when contacted with the TILs. In some aspects, the modified TILs described herein have been passed through a cell-deforming constriction, wherein the cell-deforming constriction deformed the TILs thereby causing perturbations of the TILs such that each of one or more nucleic acids encoding one or more cytokines, one or more nucleic acids encoding one or more anti-apoptotic factors, and one or more nucleic acids encoding one or more co-stimulatory molecules had entered the TILs through the perturbations when contacted with the TILs.

In some aspects, provided are methods of producing modified TILs comprising a chimeric membrane-bound cytokine, the method comprising introducing a nucleic acid encoding the chimeric membrane-bound cytokine to the TILs. In some aspects, provided are methods of producing modified TILs comprising a chimeric membrane-bound cytokine, comprising: a) passing a cell suspension comprising input TILs through a cell-deforming constriction, wherein a diameter of the constriction is a function of a diameter of the input TILs in the suspension, thereby causing perturbations of the input TILs large enough for a nucleic acid encoding the chimeric membrane-bound cytokine to pass through to form a perturbed input TILs; and b) incubating the perturbed input TILs with the nucleic acid encoding the chimeric membrane-bound cytokine to allow the nucleic acid to enter the perturbed input TILs where the nucleic acid encoding the chimeric membrane-bound cytokine is expressed, thereby generating the modified TILs comprising a chimeric membrane-bound cytokine.

The techniques and procedures described or referenced herein are generally well understood and commonly employed using conventional methodology by those skilled in the art, such as, for example, the widely utilized methodologies described in Molecular Cloning: A Laboratory Manual (Sambrook et al., 4^{th} ed., Cold Spring Harbor Laboratory Press, Cold Spring Harbor, N.Y., 2012); Current Protocols in Molecular Biology (F. M. Ausubel, et al. eds., 2003); the series Methods in Enzymology (Academic Press, Inc.), PCR 2: A Practical Approach (M. J. MacPherson, B. D. Hames and G. R. Taylor eds., 1995); Antibodies, A Laboratory Manual (Harlow and Lane, eds., 1988), Culture of Animal Cells: A Manual of Basic Technique and Specialized Applications (R. I. Freshney, 6^{th} ed., J. Wiley and Sons, 2010); Oligonucleotide Synthesis (M. J. Gait, ed., 1984), Methods in Molecular Biology, Humana Press, Cell Biology: A Laboratory Notebook (J. E. Cellis, ed., Academic Press, 1998), Introduction to Cell and Tissue Culture (J. P. Mather and P. E. Roberts, Plenum Press, 1998); Cell and Tissue Culture: Laboratory Procedures (A. Doyle, J. B. Griffiths, and D. G. Newell, eds., J. Wiley and Sons, 1993-8); Handbook of Experimental Immunology (D. M. Weir and C. C. Blackwell, eds., 1996); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biolog (Ausubel et al., eds., J. Wiley and Sons, 2002), Immunobiology (C. A. Janeway et al, 2004); Antibodies (P. Finch, 1997), Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane, Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J B. Lippincott Company, 2011).

### Definitions

For purposes of interpreting this specification, the following definitions will apply and whenever appropriate, terms used in the singular will also include the plural and vice versa. In the event that any definition set forth below conflicts with any document incorporated herein by reference, the definition set forth shall control.

As used herein, the singular form “a”, “an”, and “the” includes plural references unless indicated otherwise.

It is understood that aspects and aspects of the disclosure described herein include “comprising,” “consisting,” and “consisting essentially of” aspects and aspects.

The term “about” as used herein refers to the usual error range for the respective value readily known to the skilled person in this technical field Reference to “about” a value or parameter herein includes (and describes) aspects that are directed to that value or parameter per se.

As used herein, “treatment” is an approach for obtaining beneficial or desired clinical results. “Treatment” as used herein, covers any administration or application of a therapeutic for disease in a mammal, including a human. For purposes of this disclosure, beneficial or desired clinical results include, but are not limited to, any one or more of: alleviation of one or more symptoms, diminishment of extent of disease, preventing or delaying spread (e.g., metastasis, for example metastasis to the lung or to the lymph node) of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total). Also encompassed by “treatment” is a reduction of pathological consequence of a proliferative disease. The methods of the disclosure contemplate any one or more of these aspects of treatment.

As used herein, the term “prophylactic treatment” refers to treatment, wherein an individual is known or suspected to have or be at risk for having a disorder but has displayed no symptoms or minimal symptoms of the disorder. An individual undergoing prophylactic treatment can be treated prior to onset of symptoms. In some aspects, an individual can be treated if they have a precancerous lesion.

As used herein, by “combination therapy” is meant that a first agent be administered in conjunction with another agent. “In conjunction with” refers to administration of one treatment modality in addition to another treatment modality, such as administration of a composition of TILs as described herein in addition to administration of an immunoconjugate as described herein to the same individual. As such, “in conjunction with” refers to administration of one treatment modality before, during, or after delivery of the other treatment modality to the individual.

The term “simultaneous administration,” as used herein, means that a first therapy and second therapy in a combination therapy are administered with a time separation of no more than about 15 minutes, such as no more than about any of 10, 5, or 1 minutes. When the first and second therapies are administered simultaneously, the first and second therapies can be contained in the same composition (e.g., a composition comprising both a first and second therapy) or in separate compositions (e.g., a first therapy in one composition and a second therapy is contained in another composition).

As used herein, the term “sequential administration” means that the first therapy and second therapy in a combination therapy are administered with a time separation of more than about 15 minutes, such as more than about any of 20, 30, 40, 50, 60, or more minutes. Either the first therapy or the second therapy can be administered first. The first and second therapies are contained in separate compositions, which can be contained in the same or different packages or kits.

As used herein, the term “concurrent administration” means that the administration of the first therapy and that of a second therapy in a combination therapy overlap with each other.

In the context of cancer, the term “treating” includes any or all of killing cancer cells, inhibiting growth of cancer cells, inhibiting replication of cancer cells, lessening of overall tumor burden and ameliorating one or more symptoms associated with the disease.

The term “pore” as used herein refers to an opening, including without limitation, a hole, tear, cavity, aperture, break, gap, or perforation within a material. In some examples, (where indicated) the term refers to a pore within a surface of the present disclosure. In other examples, (where indicated) a pore can refer to a pore in a cell membrane.

The term “membrane” as used herein refers to a selective barrier or sheet containing pores. The term includes a pliable sheet-like structure that acts as a boundary or lining. In some examples, the term refers to a surface or filter containing pores. This term is distinct from the term “cell membrane”.

The term “filter” as used herein refers to a porous article that allows selective passage through the pores. In some examples the term refers to a surface or membrane containing pores.

The term “exogenous” when used in reference to an agent, such as an antigen or an adjuvant, with relation to a cell refers to an agent outside of the cell or an agent delivered into the cell from outside the cell. The cell can or can not have the agent already present, and can or can not produce the agent after the exogenous agent has been delivered.

The term “heterogeneous” as used herein refers to something which is mixed or not uniform in structure or composition. In some examples the term refers to pores having varied sizes, shapes or distributions within a given surface.

The term “homogeneous” as used herein refers to something which is consistent or uniform in structure or composition throughout. In some examples, the term refers to pores having consistent sizes, shapes, or distribution within a given surface.

The term “homologous” as used herein refers to a molecule which is derived from the same organism. In some examples, the term refers to a nucleic acid or protein which is normally found or expressed within the given organism.

The term “heterologous” as it relates to nucleic acid sequences such as coding sequences and control sequences, denotes sequences that are not normally joined together, and/or are not normally associated with a particular cell. Thus, a “heterologous” region of a nucleic acid construct or a vector is a segment of nucleic acid within or attached to another nucleic acid molecule that is not found in association with the other molecule in nature. For example, a heterologous region of a nucleic acid construct could include a coding sequence flanked by sequences not found in association with the coding sequence in nature. Another example of a heterologous coding sequence is a construct where the coding sequence itself is not found in nature (e.g., synthetic sequences having codons different from the native gene). Similarly, a cell transformed with a construct which is not normally present in the cell would be considered heterologous for purposes of this disclosure. Allelic variation or naturally occurring mutational events do not give rise to heterologous DNA, as used herein.

The term “heterologous” as it relates to amino acid sequences such as peptide sequences and polypeptide sequences, denotes sequences that are not normally joined together, and/or are not normally associated with a particular cell. Thus, a “heterologous” region of a peptide sequence is a segment of amino acids within or attached to another amino acid molecule that is not found in association with the other molecule in nature. For example, a heterologous region of a peptide construct could include the amino acid sequence of the peptide flanked by sequences not found in association with the amino acid sequence of the peptide in nature. Another example of a heterologous peptide sequence is a construct where the peptide sequence itself is not found in nature (e.g., synthetic sequences having amino acids different as coded from the native gene). Similarly, a cell transformed with a vector that expresses an amino acid construct which is not normally present in the cell would be considered heterologous for purposes of this disclosure. Allelic variation or naturally occurring mutational events do not give rise to heterologous peptides, as used herein.

As used herein, the term “inhibit” can refer to the act of blocking, reducing, eliminating, or otherwise antagonizing the presence, or an activity of, a particular target Inhibition can refer to partial inhibition or complete inhibition. For example, inhibiting an immune response can refer to any act leading to a blockade, reduction, elimination, or any other antagonism of an immune response. In other examples, inhibition of the expression of a nucleic acid can include, but not limited to reduction in the transcription of a nucleic acid, reduction of mRNA abundance (e.g., silencing mRNA transcription), degradation of mRNA, inhibition of mRNA translation, and so forth. In another example, inhibit can refer to the act of slowing or stopping growth; for example, retarding or preventing the growth of a tumor cell.

As used herein, the term “suppress” can refer to the act of decreasing, reducing, prohibiting, limiting, lessening, or otherwise diminishing the presence, or an activity of, a particular target Suppression can refer to partial suppression or complete suppression. For example, suppressing an immune response can refer to any act leading to decreasing, reducing, prohibiting, limiting, lessening, or otherwise diminishing an immune response. In other examples, suppression of the expression of a nucleic acid can include, but not limited to reduction in the transcription of a nucleic acid, reduction of mRNA abundance (e.g., silencing mRNA transcription), degradation of mRNA, inhibition of mRNA translation, and so forth.

As used herein, the term “enhance” can refer to the act of improving, boosting, heightening, or otherwise increasing the presence, or an activity of, a particular target. For example, enhancing an immune response can refer to any act leading to improving, boosting, heightening, or otherwise increasing an immune response. In one exemplary example, enhancing an immune response can refer to employing an antigen and/or adjuvant to improve, boost, heighten, or otherwise increase an immune response. In other examples, enhancing the expression of a nucleic acid can include, but not limited to increase in the transcription of a nucleic acid, increase in mRNA abundance (e.g., increasing mRNA transcription), decrease in degradation of mRNA, increase in mRNA translation, and so forth. As used herein to describe the modified TILs of the present disclosure, in some aspects, the term can refer to the one or more properties of the modified TILs that are improved or increased compared to corresponding non-modified TILs. Non-limiting examples of such properties are provided throughout the present disclosure. Accordingly, unless indicated otherwise, the modified TILs described herein can be further referred to herein as “enhanced TILs.”

As used herein, the term “modulate” can refer to the act of changing, altering, varying, or otherwise modifying the presence, or an activity of, a particular target. For example, modulating an immune response can refer to any act leading to changing, altering, varying, or otherwise modifying an immune response. In some examples, “modulate” refers to enhancing the presence or activity of a particular target. In some examples, “modulate” refers to suppressing the presence or activity of a particular target. In other examples, modulating the expression of a nucleic acid can include, but not limited to a change in the transcription of a nucleic acid, a change in mRNA abundance (e.g., increasing mRNA transcription), a corresponding change in degradation of mRNA, a change in mRNA translation, and so forth.

As used herein, the term “induce” can refer to the act of initiating, prompting, stimulating, establishing, or otherwise producing a result. For example, inducing an immune response can refer to any act leading to initiating, prompting, stimulating, establishing, or otherwise producing a desired immune response. In other examples, inducing the expression of a nucleic acid can include, but not limited to initiation of the transcription of a nucleic acid, initiation of mRNA translation, and so forth.

The term “polynucleotide” or “nucleic acid” as used herein refers to a polymeric form of nucleotides of any length, either ribonucleotides or deoxyribonucleotides. Thus, this term includes, but is not limited to, single-, double- or multi-stranded DNA or RNA, genomic DNA, cDNA, DNA-RNA hybrids, or a polymer comprising purine and pyrimidine bases, or other natural, chemically or biochemically modified, non-natural, or derivatized nucleotide bases. The backbone of the polynucleotide can comprise sugars and phosphate groups (as can typically be found in RNA or DNA), or modified or substituted sugar or phosphate groups. Alternatively, the backbone of the polynucleotide can comprise a polymer of synthetic subunits such as phosphoramidates and phosphorothioates, and thus can be an oligodeoxynucleoside phosphoramidate (P-NH2), a mixed phosphorothioate-phosphodiester oligomer, or a mixed phosphoramidate-phosphodiester oligomer. In addition, a double-stranded polynucleotide can be obtained from the single stranded polynucleotide product of chemical synthesis either by synthesizing the complementary strand and annealing the strands under appropriate conditions, or by synthesizing the complementary strand de novo using a DNA polymerase with an appropriate primer.

The terms “polypeptide” and “protein” are used interchangeably to refer to a polymer of amino acid residues, and are not limited to a minimum length. Such polymers of amino acid residues can contain natural or non-natural amino acid residues, and include, but are not limited to, peptides, oligopeptides, dimers, trimers, and multimers of amino acid residues Both full-length proteins and fragments thereof are encompassed by the definition. The terms also include post-expression modifications of the polypeptide, for example, glycosylation, sialylation, acetylation, phosphorylation, and the like. Furthermore, for purposes of the present disclosure, a “polypeptide” refers to a protein which includes modifications, such as deletions, additions, and substitutions (generally conservative in nature), to the native sequence, as long as the protein maintains the desired activity. These modifications can be deliberate, as through site-directed mutagenesis, or can be accidental, such as through mutations of hosts which produce the proteins or errors due to PCR amplification.

As used herein, by “pharmaceutically acceptable” or “pharmacologically compatible” is meant a material that is not biologically or otherwise undesirable, e.g., the material can be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug Administration.

As used herein, “microfluidic systems” refers to systems in which low volumes (e.g., mL, nL, pL, fL) of fluids are processed to achieve the discrete treatment of small volumes of liquids. Certain implementations described herein include multiplexing, automation, and high throughput screening. The fluids (e.g., a buffer, a solution, a payload-containing solution, or a cell suspension) can be moved, mixed, separated, or otherwise processed. In some aspects described herein, microfluidic systems are used to apply mechanical constriction to a cell suspended in a buffer, inducing perturbations in the cell (e.g., holes) that allow a payload or compound to enter the cytosol of the cell.

For any of the structural and functional characteristics described herein, methods of determining these characteristics are known in the art.

Methods of Enhancing Activity and/or Proliferation of Tumor Infiltrating Lymphocytes

As used herein, tumor infiltrating lymphocytes (TILs) can comprise any or all lymphocytic cell populations that have invaded the tumor tissue TILs have been described in a number of solid tumors, including breast cancer, and are emerging as an important biomarker in predicting the efficacy and outcome of treatment. In breast cancer, TILs can comprise primarily of cytotoxic (CD8+) and helper (CD4+) T cells, and a smaller proportion of B- and NK cells (Pruneri et al, Breast 2018 February; 37:207-214; Whitford et al., Eur J Cancer, 1992; 28(2-3):350-6). Large numbers of TILs can correlate with the presence of tertiary lymphoid structures in tumors, which additionally housed the follicular helper T cells (Tfh) responsible for lymphocyte generation (Gu-Trantien et al., J Clin Invest, 2013 July; 123(7):2873-92).

TILs are useful therapeutically due to their increased specificity to tumor antigens. For therapeutic purposes, TILs can be isolated from tumor tissue by one or more methods, including but not limited to enzymatic digestion. After isolation from the tumor, subsets of TILs can optionally be isolated to increase purity of sub-populations. Subsequently the TILs can be expanded in one or more stages (Dudley et al., J Immunother. 2003; 26(4): 332-342; Jin et al., J Immunother. 2012 April; 35(3): 283-292) In some examples, TILs can be expanded from thousands of cells to billions of cells. The expanded TILs can be infused into an individual to treat one or more tumors. In some aspects, the TILs are autologous to the individual. In some aspects, the TILs are allogeneic to the individual.

In some aspects, TILs can be isolated from carcinomas with mechanical dissociation, enzymatic disaggregation and/or density gradient centrifugation (Baldan et al., Br J Cancer 2015 Apr. 28; 112(9):1510-8; Tan and Lei, Methods Mol Biol. 2019; 1960:93-99).

Method of Enhancing Activity and/or Proliferation of Tumor Infiltrating Lymphocytes

in some aspects, provided are methods for modulating the activity and/or proliferation of tumor-infiltrating lymphocytes (TILs). More specifically, the TILs of the present disclosure have been modified such that they differ from corresponding naturally existing TILs. For instance, as described and demonstrated herein, the TILs have been modified to express certain engineered proteins (e.g., membrane-bound cytokines described herein), exhibit increased expression of one or more co-stimulatory molecules/cytokines, and/or exhibit other proteins that increase the persistence and/or tumor killing activity of the TILs. As is apparent from the present disclosure, in some aspects, because of such modifications, the TILs described herein are capable of having enhanced activity and/or proliferation as compared to corresponding TILs that have not been modified as described herein. While the present disclosure uses the expression “activity and/or proliferation,” such expression is not intended to be limited to any particular function of the TILs described herein. Accordingly, unless indicated otherwise, the term “activity,” as used herein, refers to the overall functional capabilities of the TILs. Non-limiting examples of such functional capabilities include: ability to kill tumor cells, ability to resist exhaustion, ability to persist long-term, or combinations thereof.

In some aspects, provided are methods for modulating the activity and/or proliferative capacity of tumor-infiltrating lymphocytes (TILs), wherein the TILs are modified to increase expression of one or more co-stimulatory molecules and/or one or more cytokines. In some aspects, provided are methods for modulating the activity and/or proliferative capacity of TILs, wherein the TILs are modified to increase expression of one or more of co-stimulatory molecules. In some aspects, provided herein are methods for modulating the activity and/or proliferative capacity of TILs, wherein the TILs are modified to increase expression of one or more engineered proteins/cytokines that are capable of enhancing the function of the TILs. In some aspects, provided herein are methods for modulating the activity and/or proliferative capacity of TILs, wherein the TILs are modified to express certain molecules (e.g., Bcl-2) that are capable of increasing the persistence and/or tumor killing activity of the TILs.

In some aspects, after the modification, the expression of one or more co-stimulatory molecules is increased compared to that of reference TILs (e.g., corresponding TILs that have not been modified as described herein). In some aspects, the co-stimulatory molecule is B7-H2 (ICOSL), B7-1 (CD80), B7-2 (CD86), CD70, LIGHT, HVEM, CD40, 4-1BBL, OX40L, TL1A, GITRL, CD30L, TIM4, SLAM, CD48, CD58, CD155, or CD112. In some aspects, the co-stimulatory molecule is CD86. In some aspects, the TILs are modified to increase expression of one or more of B7-H2 (ICOSL), B7-1 (CD80), B7-2 (CD86), CD70, LIGHT, HVEM, CD40, 4-1BBL, OX40L, TL1A, GITRL, CD30L, TIM4, SLAM, CD48, CD58, CD155, or CD112. In some aspects, the TILs are modified to increase expression of CD86. As further described herein, in some aspects, modifying the TILs to increase expression of one or more co-stimulatory molecules comprises intracellularly delivering one or more of the co-stimulatory molecules to the TILs such that the TILs have increased expression of the one or more co-stimulatory molecules. In some aspects, the one or more co-stimulatory molecules are delivered to the TILs using the squeeze processing methods described herein.

In some aspects, provided are methods for modulating the activity and/or proliferative capacity of TILs, wherein the method comprises modifying the TILs to comprise a nucleic acid encoding the co-stimulatory molecules in the TILs. In some aspects, the method comprises modifying the TILs to comprise one or more nucleic acids encoding one or more of: B7-H2 (ICOSL), B7-1 (CD80), B7-2 (CD86), CD70, LIGHT, HVEM, CD40, 4-1BBL, OX40L, TL1A, GITRL, CD30L, TIM4, SLAM, CD48, CD58, CD155, or CD112. In some aspects, the method comprises modifying the TILs to comprise one or more nucleic acids encoding CD86. In some aspects, the nucleic acid encoding the co-stimulatory molecules is an mRNA. In some aspects, the nucleic acid encoding CD86 is an mRNA. Accordingly, in some aspects, modifying the TILs to increase expression of one or more co-stimulatory molecules comprises intracellularly delivering one or more nucleic acids (e.g., mRNA) encoding one or more of the cytokines to the TILs. In some aspects, the one or more nucleic acids are delivered to the TILs using the squeeze processing methods described herein.

In some aspects, provided are methods for modulating the activity and/or proliferative capacity of tumor-infiltrating lymphocytes (TILs), wherein the TILs are modified to increase expression of one or more co-stimulatory molecules and/or one or more cytokines. In some aspects, the method comprises modifying the TILs to increase expression of one or more co-stimulatory molecules as compared to that of reference TILs (e.g., corresponding TILs that have not been modified as described herein). In some aspects, the method comprises modifying the TILs to increase expression of one or more cytokines as compared to that of reference TILs (e.g., corresponding TILs that have not been modified as described herein). In some aspects, the method comprises modifying the TILs to increase expression of both one or more cytokines and one or more co-stimulatory molecules as compared to reference TILs (e.g., corresponding TILs that have not been modified as described herein).

In some aspects, provided are methods for modulating the activity and/or proliferative capacity of TILs, wherein the TILs are modified to increase expression of one or more of cytokines. In some aspects, the cytokine is a type I cytokine. In some aspects, the cytokine is a type 2 cytokine. In some aspects, the cytokine comprises one or more of IL-2, IL-7, IL-15, IL-10, IL-12, IFN-α, or IL-21, or a functional variant thereof. As used herein, the term “functional variant” refers to a variant of a polypeptide or protein (e.g., cytokine) having substantial or significant sequence identity to the polypeptide or protein and retaining at least one of the biological activities of the polypeptide or protein. A functional variant of a polypeptide or protein can be prepared by means known in the art in view of the present disclosure. A functional variant can include one or more modifications to the amino acid sequence of the polypeptide or protein. In some aspects, the modifications change one or more physicochemical properties of the polypeptide or protein, for example, by improving the thermal stability of the polypeptide or protein, altering the substrate specificity, changing the optimal pH, reduce immunogenicity, and the like. In some aspects, the modifications alter one or more of the biological activities of the polypeptide or protein, so long as they do not destroy or abolish all of the biological activities of the polypeptide or protein.

In some aspects, the cytokine is IL-2 or a functional variant thereof, IL-7 or a functional variant thereof, IL-15 or a functional variant thereof, and/or IL-12 or a functional variant thereof. In some aspects, the cytokine is IL-2 or a functional variant thereof. In some aspects, the cytokine is IL-12 or a functional variant thereof. In some aspects, the cytokine is IL-7 or a functional variant thereof. In some aspects, the cytokine is IL-15 or a functional variant thereof. As further described herein, in some aspects, modifying the TILs to increase expression of one or more cytokines comprises intracellularly delivering one or more nucleic acids (e.g., mRNA) encoding one or more of the cytokines to the TILs. In some aspects, the one or more nucleic acids are delivered to the TILs using the squeeze processing methods described herein.

As described earlier, in some aspects, provided herein are methods of modulating the activity and/or proliferative capacity of TILs, wherein the method comprises modifying the TILs such that the TILs express certain engineered proteins. In some aspects, the modified TILs exhibit increased expression of the engineered proteins as compared to corresponding TILs that have not been modified. As demonstrated herein, in some aspects, the engineered protein comprises a membrane-bound cytokine. In some aspects, the membrane-bound cytokine is a cytokine that normally is not membrane-bound but has been engineered to be membrane-bound. Such membrane-bound cytokine is also referred to herein as “chimeric membrane-bound cytokine.” Unless indicated otherwise, the terms “membrane-bound cytokine” and “chimeric membrane-bound cytokine” are used interchangeably.

In some aspects, provided are methods for modulating the activity and/or proliferative capacity of TILs, wherein the method comprises modifying the TILs to comprise a nucleic acid encoding a chimeric membrane-bound cytokine. As it will be apparent to those skilled in the arts, such modified TILs will translate the nucleic acid and subsequently express the encoded chimeric membrane-bound cytokine Therefore, unless indicated otherwise, where a TIL is modified to comprise a nucleic acid encoding a protein (e.g., chimeric membrane-bound cytokine), the TIL will subsequently express the encoded protein. In some aspects, the method comprises modifying the TILs to comprise one or more nucleic acids encoding one or more of: chimeric membrane-bound cytokine in the TILs, wherein the cytokine is IL-2, IL-7, IL-15, IL-10, IL-12, IFN-α, or IL-21. In some aspects, the method comprises modifying the TILs to comprise one or more nucleic acids encoding chimeric membrane-bound cytokine in the TILs, wherein the cytokine is IL-2, IL-7, IL-15, and/or IL-12. In some aspects, the method comprises modifying the TILs to comprise one or more nucleic acids encoding a membrane-bound IL-2 or a functional variant thereof. In some aspects, the method comprises modifying the TILs to comprise one or more nucleic acids encoding a membrane-bound IL-12 or a variant thereof. In some aspects, the method comprises modifying the TILs to comprise one or more nucleic acids encoding a membrane bound IL-7 or a functional variant thereof. In some aspects, the method comprises modifying the TILs to comprise one or more nucleic acids encoding a membrane-bound IL-15. In some aspects, the nucleic acid encoding the chimeric membrane-bound cytokine is an mRNA encoding the chimeric membrane-bound cytokine. In some aspects, the one or more mRNAs encoding chimeric membrane-bound cytokines are mRNAs encoding IL-2, IL-7, IL-15, and/or IL-12. More specifically, in some aspects, the mRNA encodes a membrane-bound IL-2. In some aspects, the mRNA encodes a membrane-bound IL-12 In some aspects, the mRNA encodes a membrane-bound IL-7. In some aspects, the mRNA encodes a membrane-bound IL-15.

In some aspects, the TIL is modified to increase expression of one or more cytokines. In some aspects, the cytokine is IL-10, IL-15, IL-12, IL-2, IFN-α, IFN-γ, IL-21, IL-7, or a functional variant thereof. In some aspects, the cytokine is IFN-α or a functional variant thereof. In some aspects, the cytokine is a variant cytokine (such as a modified cytokine), such as a chimeric membrane-bound cytokine. In some aspects, the TIL is modified to increase expression of one or more chimeric membrane-bound cytokines (such as membrane bound IL-10, IL-15, IL-12, IL-2, IFN-α, IFN-γ, IL-21, or IL-7).

In some aspects, the cytokine is modified, and the modified cytokine is a fusion protein comprising the cytokine and a transmembrane domain. In some aspects, the cytokine is joined to the transmembrane domain by a peptide linker. In some aspects, the transmembrane domain is a transferrin receptor protein 1 (TFRC) or a tumor necrosis factor (e.g., FasL) transmembrane domain. In some aspects, the transmembrane domain comprises the amino acid sequence of SEQ ID NO: 11 or SEQ ID NO: 12. In some aspects, the peptide linker is a G₄S (SEQ ID NO: 5) linker or an EAAAK (SEQ ID NO 6) linker. In aspects, the G₄S (SEQ ID NO: 5) linker comprises any one of 2, 3, 4, 5, 6, 7, 8, 9, or 10 repeats of G₄S sequence. In some aspects, the EAAAK (SEQ ID NO 6) linker comprises any one of 2, 3, 4, 5, 6, 7, 8, 9, or 10 repeats of EAAAK sequence. In some aspects, the peptide linker is (G₄S)₃ (SEQ ID NO: 3) or (EAAAK)₃ (SEQ ID NO: 4). In some aspects, the chimeric membrane-bound cytokine comprises the amino acid sequence of any one of SEQ ID NOs: 7-10 and 13. In some aspects, the TIL is modified to comprise a nucleic acid that results in increased expression and/or secretion of the one or more cytokines. In some aspects, the nucleic acid encoding the chimeric membrane-bound cytokine comprises the nucleotide sequence of SEQ ID NO: 1 or 2. In some aspects, the chimeric membrane-bound cytokine enhances the half-life of the cytokine in an individual compared to a non-membrane-bound cytokine. In some aspects, the half-life of the chimeric membrane-bound cytokine is increased by about any one of: 10%, 25%, 50%, 75%, 100%, 2-fold, 3-fold, 4-fold, 5-fold, 10-fold, 25-fold, 50-fold, 100-fold, 200-fold, or 500-fold or more compared to a non-membrane-bound cytokine. In some aspects, the membrane-bound cytokine in the modified TIL prolongs the spatial association of the cytokine with an antigen presented by an antigen-presenting cell, by about any one of: 1, 2, 3, 4, 6, 8, 12, 16, 20, 24, 28, 32, 36, 48, 72, 96 or more hours, compared to a TIL comprising a non-membrane-bound cytokine. In some aspects, a TIL comprising membrane-bound cytokine exhibits a local cytokine concentration that is higher by about any one of: 10%, 25%, 50%, 75%, 100%, 2-fold, 3-fold, 4-fold, 5-fold, 10-fold, 25-fold, 50-fold, 100-fold, 200-fold, or 500-fold or more compared to a corresponding TIL comprising a non-membrane-bound cytokine. In some aspects, the membrane-bound cytokine is a membrane-bound chemokine.

In some aspects, there is provided a composition for enhancing the activity of a TIL, the composition comprising a chimeric membrane-bound cytokine in the TIL. In some aspects, the cytokine is modified, and the modified cytokine is a fusion protein comprising the cytokine and a transmembrane domain. In some aspects, the cytokine is joined to the transmembrane domain by a peptide linker. In some aspects, the transmembrane domain is a transferrin receptor protein 1 (TFRC) or a tumor necrosis factor (e.g., FasL) transmembrane domain. In some aspects, the transmembrane domain comprises the amino acid sequence of SEQ ID NO. 11 or SEQ ID NO: 12. In some aspects, the peptide linker is a G₄S (SEQ ID NO: 5) linker or an EAAAK (SEQ ID NO. 6) linker. In aspects, the G₄S (SEQ ID NO: 5) linker comprises any one of 2, 3, 4, 5, 6, 7, 8, 9, or 10 repeats of G₄S sequence. In some aspects, the EAAAK (SEQ ID NO 6) linker comprises any one of 2, 3, 4, 5, 6, 7, 8, 9, or 10 repeats of EAAAK sequence. In some aspects, the peptide linker is (G₄S)₃ (SEQ ID NO: 3) or (EAAAK)₃ (SEQ ID NO: 4). In some aspects, the chimeric membrane-bound cytokine comprises the amino acid sequence of any one of SEQ ID NOs 7-10 and 13 In some aspects, the TIL comprises a nucleic acid that results in increased expression and/or secretion of the one or more cytokines. In some aspects, the nucleic acid encoding the chimeric membrane-bound cytokine comprises the nucleotide sequence of SEQ ID NO: 1 or 2. In some aspects, the TIL comprises an mRNA that results in increased expression and/or secretion of the one or more cytokines. In some aspects, the chimeric membrane-bound cytokine enhances the half-life of the cytokine in an individual compared to a non-membrane-bound cytokine. In some aspects, the half-life of the chimeric membrane-bound cytokine is increased by about any one of: 10%, 25%, 50%, 75%, 100%, 2-fold, 3-fold, 4-fold, 5-fold, 10-fold, 25-fold, 50-fold, 100-fold, 200-fold, or 500-fold or more compared to a non-membrane-bound cytokine. In some aspects, the membrane-bound cytokine prolongs the spatial association of the cytokine with the antigens presented by the TIL introduced with the chimeric membrane-bound cytokine, by about any one of: 1, 2, 3, 4, 6, 8, 12, 16, 20, 24, 28, 32, 36, 48, 72, 96 or more hours, compared to a corresponding TIL comprising a non-membrane-bound cytokine. In some aspects, a TIL comprising the membrane-bound cytokine exhibits a local cytokine concentration that is higher by about any one of: 10%, 25%, 50%, 75%, 100%, 2-fold, 3-fold, 4-fold, 5-fold, 10-fold, 25-fold, 50-fold, 100-fold, 200-fold, or 500-fold or more compared to a corresponding TIL comprising a non-membrane-bound cytokine. In some aspects, the membrane-bound cytokine is a membrane-bound chemokine.

In some aspects, provided herein is a method for modulating the activity and/or proliferative capacity of TILs, wherein the TILs are modified to comprise a nucleic acid encoding a protein that is capable of increasing the persistence of the TILs (e.g., when administered to a subject in need thereof). In some aspects, provided herein is a method of modulating the activity and/or proliferative capacity of TILs, wherein the TILs are modified to comprise a nucleic acid encoding a protein that is capable of increasing the tumor killing activity of the TILs. In some aspects, provided herein is a method for modulating the activity and/or proliferative capacity of TILs, wherein the TILs are modified to comprise a nucleic acid encoding a protein that is capable of both increasing the persistence and tumor killing activity of the TILs.

In some aspects, provided are methods for modulating the activity and/or proliferative capacity of tumor-infiltrating lymphocytes (TILs), wherein the TILs are modified by a process comprising: a) passing a cell suspension comprising input TILs through a cell-deforming constriction, wherein a diameter of the constriction is a function of a diameter of the input TILs in the suspension, thereby causing perturbations of the input TILs large enough for one or more nucleic acids encoding one or more cytokines (e.g., chimeric membrane-bound cytokines), one or more nucleic acids encoding a protein that increases the persistence and/or tumor killing activity (e.g., anti-apoptotic factor, e.g., Bcl-2), and/or one or more nucleic acids encoding one or more co-stimulatory molecules to pass through to form perturbed input TILs; and b) incubating the perturbed input TILs with the one or more nucleic acids encoding one or more cytokines (e.g., chimeric membrane-bound cytokines), one or more nucleic acids encoding a protein that increases the persistence and/or tumor killing activity, and/or one or more nucleic acids encoding one or more co-stimulatory molecules to allow the one or more nucleic acids encoding one or more cytokines (e.g., chimeric membrane-bound cytokines), one or more nucleic acids encoding a protein that increases the persistence and/or tumor killing activity, and/or one or more nucleic acids encoding one or more co-stimulatory molecules to enter the perturbed input TILs; wherein the nucleic acids are expressed, thereby generating the modified TILs comprising the one or more cytokines, proteins that increase persistence and/or tumor killing activity, and/or the one or more co-stimulatory molecules.

Accordingly, in some aspects, a method of modulating the activity and/or proliferative capacity of TILs, comprises passing a cell suspension comprising the TILs through a cell-deforming constriction, thereby causing perturbations of the TILs such that one or more cytokines (e.g. one or more nucleic acids encoding one or more cytokines) enter the TILs through the perturbations when contacted with the TILs. In some aspects, a method of modulating the activity and/or proliferative capacity of TILs, comprises passing a cell suspension comprising the TILs through a cell-deforming constriction, thereby causing perturbations of the TILs such that one or more co-stimulatory molecules (e.g., one or more nucleic acids encoding one or more co-stimulatory molecules) enter the TILs through the perturbations when contacted with the TILs. In some aspects, a method of modulating the activity and/or proliferative capacity of TILs, comprises passing a cell suspension comprising the TILs through a cell-deforming constriction, thereby causing perturbations of the TILs such that one or more proteins that enhance persistence and/or tumor killing activity (e.g., anti-apoptotic factors, e.g., Bcl-2) (e.g., one or more nucleic acids encoding the one or more proteins) enter the TILs through the perturbations when contacted with the TILs. In some aspects, a method of modulating the activity and/or proliferative capacity of TILs, comprises passing a cell suspension comprising the TILs through a cell-deforming constriction, thereby causing perturbations of the TILs such that one or more co-stimulatory molecules (e.g., one or more nucleic acids encoding one or more co-stimulatory molecules) and one or more cytokines (e.g., one or more nucleic acids encoding one or more cytokines) enter the TILs through the perturbations when contacted with the TILs. In some aspects, a method of modulating the activity and/or proliferative capacity of TILs, comprises passing a cell suspension comprising the TILs through a cell-deforming constriction, thereby causing perturbations of the TILs such that one or more co-stimulatory molecules (e.g., one or more nucleic acids encoding one or more co-stimulatory molecules), one or more proteins that enhance persistence and/or tumor killing activity (e.g., anti-apoptotic factors, e.g., Bcl-2) (e.g., one or more nucleic acids encoding one or more proteins that enhance persistence and/or tumor killing activity), and one or more cytokines (e.g., one or more nucleic acids encoding one or more cytokines) enter the TILs through the perturbations when contacted with the TILs. In some aspects, the nucleic acids encoding the cytokines, the nucleic acids encoding the proteins that enhance persistence and/or tumor killing activity (e.g., anti-apoptotic factors, e.g., Bcl-2), and/or one or the nucleic acids encoding the co-stimulatory molecule are mRNAs. In some aspects, the cytokine is a membrane-bound cytokine, and the nucleic acid encoding the chimeric membrane-bound cytokine is an mRNA encoding the chimeric membrane-bound cytokine.

In some aspects, provided are methods for modulating the activity and/or proliferative capacity of tumor-infiltrating lymphocytes (TILs), wherein the TILs are modified by a process comprising: a) passing a cell suspension comprising input TILs through a cell-deforming constriction, wherein a diameter of the constriction is a function of a diameter of the input TILs in the suspension, thereby causing perturbations of the input TILs large enough for the one or more nucleic acids encoding one or more cytokines (e.g., chimeric membrane-bound cytokines), one or more nucleic acids encoding one or more proteins that enhance persistence and/or tumor killing activity (e.g., anti-apoptotic factors, e.g., Bcl-2), and/or the one or more co-stimulatory molecules to pass through to form perturbed input TILs; and b) incubating the perturbed input TILs with the one or more nucleic acids encoding one or more cytokines, one or more nucleic acids encoding one or more proteins that enhance persistence and/or tumor killing activity (e.g., anti-apoptotic factors, e.g., Bcl-2), and/or the one or more co-stimulatory molecules to enter the perturbed input TILs; wherein the nucleic acids are expressed thereby generating the modified TILs comprising the one or more cytokines, one or more proteins that enhance persistence and/or tumor killing activity, and/or the one or more co-stimulatory molecules. In some aspects, the nucleic acids encoding the one or more cytokines are mRNAs. In some aspects, wherein the cytokine is a membrane-bound cytokine, the nucleic acid encoding the chimeric membrane-bound cytokine is an mRNA encoding the chimeric membrane-bound cytokine.

In some aspects, provided are methods for modulating the activity and/or proliferative capacity of tumor-infiltrating lymphocytes (TILs), wherein the method comprises incubating the TILs with the nucleic acid encoding the cytokine (e.g., chimeric membrane-bound cytokine) before, during and/or after passing the cell suspension through the cell-deforming constriction. In some aspects, the method comprises incubating the TILs with the nucleic acid encoding the cytokine (e.g., chimeric membrane-bound cytokine) before the TILs (or cell suspension comprising the TILs) are passed through the cell-deforming constriction. In some aspects, the method comprises incubating the TILs with the nucleic acid encoding the cytokine (e.g., chimeric membrane-bound cytokine) during the passing of the TILs (or cell suspension comprising the TILs) through the cell-deforming constriction. In some aspects, the method comprises incubating the TILs with the nucleic acid encoding the cytokine (e.g., chimeric membrane-bound cytokine) after the TILs (or cell suspension comprising the TILs) have passed through the cell-deforming constriction. In some aspects, the method comprises incubating the TILs with the nucleic acid encoding the cytokine (e.g., chimeric membrane-bound cytokine) before, during, and after the passing of the TILs (or cell suspension comprising the TILs) through the cell-deforming constriction. As used herein, the term “incubating” comprises bringing at least a first component (e.g., nucleic acids encoding cytokine) and a second component (e.g., TILs) together, such that the first component and the second component are in closer physical proximity and can therefore interact (i.e., in contact).

In some aspects, the method comprises incubating the TILs with a nucleic acid encoding a co-stimulatory molecule before, during, and/or after passing the TILs (or cell suspension comprising the TILs) through the cell-deforming constriction. In some aspects, the method comprises incubating the TILs with the nucleic acid encoding the co-stimulatory molecule before the TILs (or cell suspension comprising the TILs) are passed through the cell-deforming constriction. In some aspects, the method comprises incubating the TILs with the nucleic acid encoding the co-stimulatory molecule during the passing of the TILs (or cell suspension comprising the TILs) through the cell-deforming constriction. In some aspects, the method comprises incubating the TILs with the nucleic acid encoding the co-stimulatory molecules after the TILs (or cell suspension comprising the TILs) have passed through the cell-deforming constriction. In some aspects, the method comprises incubating the TILs with the nucleic acid encoding the co-stimulatory molecules before, during, and after the passing of the TILs (or cell suspension comprising the TILs) through the cell-deforming constriction.

In some aspects, the method comprises incubating the TILs with a nucleic acid encoding a protein that enhances the persistence and/or tumor killing activity before, during, and/or after passing the TILs (or cell suspension comprising the TILs) through the cell-deforming constriction. In some aspects, the method comprises incubating the TILs with the nucleic acid before the TILs (or cell suspension comprising the TILs) are passed through the cell-deforming constriction. In some aspects, the method comprises incubating the TILs with the nucleic acid during the passing of the TILs (or cell suspension comprising the TILs) through the cell-deforming constriction. In some aspects, the method comprises incubating the TILs with the nucleic acid after the TILs (or cell suspension comprising the TILs) have passed through the cell-deforming constriction. In some aspects, the method comprises incubating the TILs with the nucleic acid before, during, and after the passing of the TILs (or cell suspension comprising the TILs) through the cell-deforming constriction.

In some aspects, the method comprises incubating the TILs with the nucleic acid encoding the cytokine (e.g., chimeric membrane-bound cytokine), the nucleic acid encoding the protein that enhances persistence and/or tumor killing activity, and/or the co-stimulatory molecule before, during and/or after passing the cell suspension through the cell-deforming constriction. In some aspects, the method comprises incubating the TILs with the nucleic acid encoding the cytokine (e.g., chimeric membrane-bound cytokine), the nucleic acid encoding the protein that enhances persistence and/or tumor killing activity, and the nucleic acid encoding the co-stimulatory molecule before, during and/or after passing the cell suspension through the cell-deforming constriction. In some aspects, the method comprises incubating the TILs with the nucleic acid encoding the cytokine (e.g., chimeric membrane-bound cytokine), the nucleic acid encoding the protein that enhances persistence and/or tumor killing activity, and the nucleic acid encoding the one or more co-stimulatory molecules before, during and/or after passing the cell suspension through the cell-deforming constriction. In some aspects, the nucleic acid encoding the cytokine (e.g., chimeric membrane-bound cytokine) is an mRNA encoding the cytokine (e.g., chimeric membrane-bound cytokine). In some aspects, the nucleic acid encoding the co-stimulatory molecule is a mRNA. In some aspects, the nucleic acid encoding the protein that enhances persistence and/or tumor killing activity.

In some aspects, the method comprises incubating the TILs with the nucleic acid encoding the cytokine (e.g., membrane-bound cytokine) and the nucleic acid encoding the co-stimulatory molecule before the TILs (or cell suspension comprising the TILs) are passed through the cell-deforming constriction. In some aspects, the method comprises incubating the TILs with the nucleic acid encoding the cytokine (e.g., membrane-bound cytokine) and the nucleic acid encoding the co-stimulatory molecule during the passing of the TILs (or cell suspension comprising the TILs) through the cell-deforming constriction. In some aspects, the method comprises incubating the TILs with the nucleic acid encoding the cytokine (e.g., membrane-bound cytokine) and the nucleic acid encoding the co-stimulatory molecules after the TILs (or cell suspension comprising the TILs) have passed through the cell-deforming constriction. In some aspects, the method comprises incubating the TILs with the nucleic acid encoding the cytokine (e.g., membrane-bound cytokine) and the nucleic acid encoding the co-stimulatory molecules before, during, and after the passing of the TILs (or cell suspension comprising the TILs) through the cell-deforming constriction.

In some aspects, provided are methods for modulating the activity and/or proliferative capacity of tumor-infiltrating lymphocytes (TILs), wherein the method comprises incubating the TILs with the nucleic acid encoding the chimeric membrane-bound cytokine before passing the cell suspension through the cell-deforming constriction. In some aspects, the method comprises incubating the TILs with the nucleic acid encoding the chimeric membrane-bound cytokine and the co-stimulatory molecule before passing the cell suspension through the cell-deforming constriction. In some aspects, the method comprises incubating the TILs with the nucleic acid encoding the chimeric membrane-bound cytokine and the nucleic acid encoding the co-stimulatory molecule before passing the cell suspension through the cell-deforming constriction. In some aspects, the method comprises incubating the TILs with the nucleic acid encoding the chimeric membrane-bound cytokine and the nucleic acid encoding the one or more co-stimulatory molecules before passing the cell suspension through the cell-deforming constriction. In some aspects, the nucleic acid encoding the chimeric membrane-bound cytokine is an mRNA encoding the chimeric membrane-bound cytokine.

In some aspects according to any one of the methods described herein, the TILs are mammalian TILs. In some aspects, the TILs are monkey, mouse, dog, cat, horse, rat, sheep, goat, pig, or rabbit TILs. In some aspects, the TILs are primate TILs. In some aspects, the TILs are human TILs.

In some aspects, for any of the methods provided herein (e.g., those provided above), the method can further comprise an expansion phase during which TILs are rapidly expanded. In some aspects, the methods provided herein comprises a first expansion phase and a second expansion phase Where the methods comprise two expansion phases, the first expansion phase is also referred to herein as “pre-REP” and the second expansion phase is also referred to herein as “REP.” In some aspects, the first expansion phase occurs prior to the modifying step (i.e., wherein the TILs are modified to comprise the one or more nucleic acids encoding a cytokine and/or one or more nucleic acids encoding a co-stimulatory molecule). In some aspects, the second expansion phase occurs prior to the modifying step. In some aspects, both the first expansion phase and the second expansion phase occurs prior to the modifying step.

In some aspects, the expansion phase comprises culturing the TILs with one or more agents, wherein the agents are capable of expanding the TILs such that the number of TILs is increased as compared to the number of the TILs prior to the expansion. In some aspects, the agent that is capable of expanding the TILs comprise a cytokine. In some aspects, the cytokine comprises IL-15. In some aspects, the cytokine does not comprise IL-2. In some aspects, the agent that is capable of expanding the TILs comprise an antibody. In some aspects, the antibody comprises an anti-CD3 antibody. In some aspects, the anti-CD3 antibody is not OKT3 In some aspects, the agent that is capable of expanding the TILs comprise a feeder cell. Non-limiting example of feeder cells that can be used include irradiated PBMCs. As used herein, the term “feeder cell” refers to cells that are co-cultured with the TILs in order to adjust to or promote an environment required for the culture of the TILs that are to be expanded. In some aspects, the expansion phase comprises culturing the TILs with a cytokine, wherein the cytokine does not comprise IL-2. In some aspects, the expansion phase comprises culturing the TILs with a cytokine and an antibody, wherein the cytokine is not IL-2 and the antibody is not OKT3. In some aspects, the expansion phase comprises culturing the TILs with a cytokine, antibody, and feeder cells, wherein the cytokine is not IL-2 and the antibody is not OKT3.

In some aspects, the expansion phase is for at least about 1 day, at least about 2 days, at least about 3 days, at least about 4 days, at least about 5 days, at least about 6 days, at least about 7 days, at least about 8 days, at least about 9 days, at least about 10 days, at least about 1 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, or at least about 30 days. In some aspects, the expansion phase is between about 1 day to about 10 days in duration. In some aspects, the expansion phase is between about 3 days to about 11 days in duration. In some aspects, the expansion phase is between about 7 days to about 14 days in duration. In some aspects, the expansion phase is between about 7 days to about 12 days in duration. In some aspects, the expansion phase is between about 7 days to about 15 days in duration. In some aspects, the expansion phase is between about 7 days to about 16 days in duration. In some aspects, the expansion phase is between about 14 days to about 30 days.

In some aspects, a method of enhancing the activity and/or proliferation of TILs provided herein comprises a first expansion phase and a second expansion phase. In some aspects, the first expansion phase comprises culturing TILs in the presence of a cytokine, wherein the cytokine is not IL-2. In some aspects, the first expansion phase comprises culturing TILs in the presence of a cytokine, wherein the cytokine comprises IL-15. In some aspects, the second expansion phase comprises culturing TILs in the presence of a cytokine and an antibody, wherein the cytokine is not IL-2 and the antibody is not OKT3. In some aspects, the second expansion phase comprises culturing TILs in the presence of a cytokine, an antibody, and feeder cells, wherein the cytokine is not IL-2 and the antibody is not OKT3 In some aspects, the second expansion phase comprises culturing TILs in the presence of a cytokine and an antibody, wherein the cytokine comprises IL-15 and the antibody is not OKT3. The second expansion phase is also referred to herein as “REP.” Accordingly, as used herein, “post-REP TILs” refer to TILs that have undergone the second expansion phase.

In some aspects, the first expansion phase is for at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, or at least about 20 days. In some aspects, the first expansion phase is between about 1 day to about 10 days in duration. In some aspects, the first expansion phase is between about 3 days to about 11 days in duration. In some aspects, the first expansion phase is between about 7 days to about 14 days.

In some aspects, the second expansion phase is for at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, or at least about 20 days. In some aspects, the second expansion phase is between about 7 days to about 16 days in duration. In some aspects, the second expansion phase is between about 7 days to about 15 days in duration. In some aspects, the second expansion phase is between about 7 days to about 14 days in duration. In some aspects, the second expansion phase is between about 7 days to about 13 days in duration. In some aspects, the second expansion phase is between about 7 days to about 12 days in duration. In some aspects, the second expansion phase is between about 7 days to about 11 days in duration.

Methods of rapidly expanding TILs are known in the art. For instance, as first described by the Rosenberg group, the “rapid expansion protocol” uses OKT3 (an anti-CD3 antibody), high dose IL-2, and irradiated allogenic feeder cells. See Dudley et al., J Immunother 26:332-342 (2003), which is incorporated herein by reference in its entirety. As described and demonstrated herein, the expansion methods described herein (e.g., provided above) differ from such methods known in the art that high dose of IL-2 is not required. Accordingly, compared to expansion methods in the art, the methods provided herein are associated with less toxicity and do not require the pre-conditioning methods often used when high dose of IL-2 is used. Additionally, as is apparent from the present disclosure, the expansion methods provided herein (e.g., provided above) do not involve the use of OKT3 (also known as muromonab-CD3 or ORTHOCLONE OKT3®).

Accordingly, in some aspects, provided herein is a method of enhancing the activity and/or proliferation of TILs comprising: (a) an expansion phase and (b) a modifying phase, wherein the expansion phase comprises culturing TILs in the presence of a cytokine which is not IL-2, and wherein the modifying phase comprises passing the TILs from the expansion phase through a cell-deforming constriction, thereby causing perturbations of the TILs such that a nucleic acid encoding a cytokine and/or a nucleic acid encoding a co-stimulatory molecule enters the TILs through the perturbations when contacted with the TILs. In some aspects, provided herein is a method of enhancing the activity and/or proliferation of TILs comprising: (a) an expansion phase and (b) a modifying phase, wherein the expansion phase comprises culturing TILs in the presence of a cytokine which is not IL-2 and an antibody which is not OKT3, and wherein the modifying phase comprises passing the TILs from the expansion phase through a cell-deforming constriction, thereby causing perturbations of the TILs such that a nucleic acid encoding a cytokine and/or a nucleic acid encoding a co-stimulatory molecule enters the TILs through the perturbations when contacted with the TILs. In some aspects, provided herein is a method of enhancing the activity and/or proliferation of TILs comprising: (a) a first expansion phase, (b) a second expansion phase, and (c) a modifying phase, wherein the first expansion phase comprises culturing TILs in the presence of a cytokine which is not IL-2 (e.g., IL-15), wherein the second expansion phase comprises culturing TILs from (a) in the presence of a cytokine which is not IL-2 and an anti-CD3 antibody which is not OKT3, and wherein the modifying phase comprises passing the TILs from (b) through a cell-deforming constriction, thereby causing perturbations of the TILs such that a nucleic acid encoding a cytokine and/or a nucleic acid encoding a co-stimulatory molecule enters the TILs through the perturbations when contacted with the TILs.

Compositions of Modified TILs with Enhanced Activity and/or Proliferation

In some aspects, provided are compositions comprising modified TILs with enhanced activity and/or proliferative capacity.

In some aspects, provided are compositions comprising modified TILs, wherein the TILs are modified to increase expression of certain engineered proteins (e.g., membrane-bound cytokines), co-stimulatory molecules, cytokines, and/or proteins that are capable of enhancing the persistence and/or tumor killing activity of the TILs (e.g., Bcl-2).

In some aspects, provided are compositions comprising modified TILs, wherein the TILs are modified to increase expression of one or more co-stimulatory molecules, one or more proteins that enhance persistence and/or tumor killing activity (e.g., anti-apoptotic factors, e.g., Bcl-2), and/or one or more cytokines. In some aspects, provided are compositions comprising modified TILs, wherein the TILs are modified to increase expression of one or more of co-stimulatory molecules. In some aspects, the co-stimulatory molecule is B7-H2 (ICOSL), B7-1 (CD80), B7-2 (CD86), CD70, LIGHT, HVEM, CD40, 4-1BBL, OX40L, TL1A, GITRL, CD30L, TIM4, SLAM, CD48, CD58, CD155, or CD112. In some aspects, the co-stimulatory molecule is CD86. In some aspects, the TILs are modified to increase expression of one or more of B7-H2 (ICOSL), B7-1 (CD80), B7-2 (CD86), CD70, LIGHT, HVEM, CD40, 4-1BBL, OX40L, TL1A, GITRL, CD30L, TIM4, SLAM, CD48, CD58, CD155, or CD112. In some aspects, the TILs are modified to increase expression of CD86.

In some aspects, provided are compositions comprising modified TILs, wherein the TILs are modified to express a nucleic acid encoding the co-stimulatory molecules in the TILs. In some aspects, the TILs are modified to express one or more nucleic acids encoding one or more of: B7-H2 (ICOSL), B7-1 (CD80), B7-2 (CD86), CD70, LIGHT, HVEM, CD40, 4-1BBL, OX40L, TL1A, GITRL, CD30L, TIM4, SLAM, CD48, CD58, CD155, or CD112. In some aspects, the TILs are modified to express one or more nucleic acids encoding CD86. In some aspects, one or more of the nucleic acid is mRNA. As described herein, in some aspects, the TILs can be modified to express the nucleic acid using a squeeze processing method provided herein.

In some aspects, provided are compositions comprising modified TILs, wherein the TILs are modified to increase expression of one or more co-stimulatory molecules and/or one or more cytokines. In some aspects, provided are compositions comprising modified TILs, wherein the TILs are modified to increase expression of one or more of cytokines. In some aspects, the cytokine is a type I cytokine. In some aspects, the cytokine is a type 2 cytokine. In some aspects, the cytokine comprises one or more of: IL-2, IL-7, IL-15, IL-10, IL-12, IFN-α, or IL-21, or a functional variant thereof in some aspects, the cytokine is IL-2 or a functional variant thereof, Il-7 or a functional variant thereof, IL-15 or a functional variant thereof, and/or IL-12 or a functional variant thereof. In some aspects, the cytokine is IL-2 or a functional variant thereof. In some aspects, the cytokine is IL-12 or a functional variant thereof. In some aspects, the cytokine is IL-7 or a functional variant thereof. In some aspects, the cytokine is IL-15 or a functional variant thereof.

In some aspects, provided herein are compositions comprising modified TILs that have been modified to express certain engineered proteins. In some aspects, provided are compositions comprising modified TILs, wherein the TILs are modified to express a nucleic acid encoding a chimeric membrane-bound cytokine in the TILs. In some aspects, the TILs are modified to express one or more nucleic acids encoding one or more of chimeric membrane-bound cytokine in the TILs, wherein the cytokine is IL-2, IL-7, IL-15, IL-10, IL-12, IFN-α, or IL-21. In some aspects, the TILs are modified to express one or more nucleic acids encoding chimeric membrane-bound cytokine in the TILs, wherein the cytokine is IL-2 and/or IL-12. In some aspects, the method comprises modifying the TILs to express a membrane-bound IL-2 or a functional variant thereof. In some aspects, the method comprises modifying the TILs to express a membrane-bound IL-12 or a variant thereof. In some aspects, the method comprises modifying the TILs to express a membrane bound IL-7 or a functional variant thereof. In some aspects, one or more of the nucleic acid is mRNA. In some aspects, the method comprises modifying the TILs to express a membrane bound IL-S or a functional variant thereof. In some aspects, one or more of the nucleic acid is mRNA.

In some aspects, the TIL are modified to increase expression of one or more cytokines. In some aspects, the cytokine is IL-10, IL-15, IL-12, IL-2, IFN-α, IFN-γ, IL-21, IL-7, or a functional variant thereof. In some aspects, the cytokine is IFN-α or a functional variant thereof. In some aspects, the cytokine is a variant cytokine (such as a modified cytokine), such as a chimeric membrane-bound cytokine. In some aspects, the TIL is modified to increase expression of one or more chimeric membrane-bound cytokines (such as membrane bound IL-10, IL-15, IL-12, IL-2, IFN-α, IFN-γ, IL-21, or IL-7).

In some aspects, the cytokine is modified, and the modified cytokine is a fusion protein comprising the cytokine and a transmembrane domain. In some aspects, the cytokine is joined to the transmembrane domain by a peptide linker. In some aspects, the transmembrane domain is a transferrin receptor protein 1 (TFRC) or a tumor necrosis factor (e.g., FasL) transmembrane domain. In some aspects, the transmembrane domain comprises the amino acid sequence of SEQ ID NO: 11 or SEQ ID NO. 12. In some aspects, the peptide linker is a G₄S (SEQ ID NO. 5) linker or an EAAAK (SEQ ID NO: 6) linker. In aspects, the G₄S (SEQ ID NO: 5) linker comprises any one of 2, 3, 4, 5, 6, 7, 8, 9, or 10 repeats of G₄S sequence. In some aspects, the EAAAK (SEQ ID NO: 6) linker comprises any one of 2, 3, 4, 5, 6, 7, 8, 9, or 10 repeats of EAAAK sequence. In some aspects, the peptide linker is (G₄S)₃ (SEQ ID NO: 3) or (EAAAK)₃ (SEQ ID NO: 4). In some aspects, the chimeric membrane-bound cytokine comprises the amino acid sequence of any one of SEQ ID NOs 7-10 and 13. In some aspects, the TIL is modified to comprise a nucleic acid that results in increased expression and/or secretion of the one or more cytokines. In some aspects, the nucleic acid encoding the chimeric membrane-bound cytokine comprises the nucleotide sequence of SEQ ID NO 1 or 2. In some aspects, the TIL is modified to comprise an mRNA that results in increased expression and/or secretion of the one or more cytokines. In some aspects, the cytokine is a Signal 3 effector in stimulating T cell activation. In some aspects, the chimeric membrane-bound cytokine enhances the half-life of the cytokine in an individual compared to a non-membrane-bound cytokine. In some aspects, the half-life of the chimeric membrane-bound cytokine is increased by about any one of: 10%, 25%, 50%, 75%, 100%, 2-fold, 3-fold, 4-fold, 5-fold, 10-fold, 25-fold, 50-fold, 100-fold, 200-fold, or 500-fold or more compared to a non-membrane-bound cytokine. In some aspects, the membrane-bound cytokine in the modified TIL prolongs the spatial association of the cytokine with an antigen presented by an antigen-presenting cell, by about any one of: 1, 2, 3, 4, 6, 8, 12, 16, 20, 24, 28, 32, 36, 48, 72, 96 or more hours, compared to a TIL comprising a non-membrane-bound cytokine. In some aspects, a TIL comprising membrane-bound cytokine exhibits a local cytokine concentration that is higher by about any one of 10%, 25%, 50%, 75%, 100%, 2-fold, 3-fold, 4-fold, 5-fold, 10-fold, 25-fold, 50-fold, 100-fold, 200-fold, or 500-fold or more compared to a corresponding TIL comprising a non-membrane-bound cytokine. In some aspects, the membrane-bound cytokine is a membrane-bound chemokine.

In some aspects, there is provided a composition for enhancing the activity of a TIL, the composition comprising a chimeric membrane-bound cytokine in the TIL. In some aspects, the cytokine is modified, and the modified cytokine is a fusion protein comprising the cytokine and a transmembrane domain. In some aspects, the cytokine is joined to the transmembrane domain by a peptide linker. In some aspects, the transmembrane domain is a transferrin receptor protein 1 (TFRC) or a tumor necrosis factor (e.g., FasL) transmembrane domain. In some aspects, the transmembrane domain comprises the amino acid sequence of SEQ ID NO: 11 or SEQ ID NO: 12. In some aspects, the peptide linker is a G₄S (SEQ ID NO. 5) linker or an EAAAK (SEQ ID NO: 6) linker. In aspects, the G₄S (SEQ ID NO: 5) linker comprises any one of 2, 3, 4, 5, 6, 7, 8, 9, or 10 repeats of G₄S sequence. In some aspects, the EAAAK (SEQ ID NO: 6) linker comprises any one of 2, 3, 4, 5, 6, 7, 8, 9, or 10 repeats of EAAAK sequence. In some aspects, the peptide linker is (G₄S)₃ (SEQ ID NO. 3) or (EAAAK)₃ (SEQ ID NO 4). In some aspects, the chimeric membrane-bound cytokine comprises the amino acid sequence of any one of SEQ ID NOs: 7-10 and 13. In some aspects, the TIL comprises a nucleic acid that results in increased expression and/or secretion of the one or more cytokines. In some aspects, the nucleic acid encoding the chimeric membrane-bound cytokine comprises the nucleotide sequence of SEQ ID NO: 1 or 2. In some aspects, the cytokine is a Signal 3 effector in stimulating T cell activation. In some aspects, the chimeric membrane-bound cytokine enhances the half-life of the cytokine in an individual compared to a non-membrane-bound cytokine. In some aspects, the half-life of the chimeric membrane-bound cytokine is increased by about any one of: 10%, 25%, 50%, 75%, 100%, 2-fold, 3-fold, 4-fold, 5-fold, 10-fold, 25-fold, 50-fold, 100-fold, 200-fold, or 500-fold or more compared to a non-membrane-bound cytokine. In some aspects, the membrane-bound cytokine prolongs the spatial association of the cytokine with the antigens presented by the TIL introduced with the chimeric membrane-bound cytokine, by about any one of: 1, 2, 3, 4, 6, 8, 12, 16, 20, 24, 28, 32, 36, 48, 72, 96 or more hours, compared to a corresponding TIL comprising a non-membrane-bound cytokine. In some aspects, a TIL comprising the membrane-bound cytokine exhibits a local cytokine concentration that is higher by about any one of: 10%, 25%, 50%, 75%, 100%, 2-fold, 3-fold, 4-fold, 5-fold, 10-fold, 25-fold, 50-fold, 100-fold, 200-fold, or 500-fold or more compared to a corresponding TIL comprising a non-membrane-bound cytokine. In some aspects, the membrane-bound cytokine is a membrane-bound chemokine.

In some aspects, compositions provided herein comprise modified TILs that have been modified to express proteins that are capable of increasing the persistence of the TILs. In some aspects, a composition comprises modified TILs that have been modified to express proteins that are capable of increasing the tumor killing activity of the TILs. In some aspects, a composition comprises modifying TILs that have been modified to express proteins that are capable of increasing both the persistence and tumor killing activity of the TILs.

In some aspects, provided are compositions comprising modified TILs, wherein the TILs are modified by a process comprising: a) passing a cell suspension comprising input TILs through a cell-deforming constriction, wherein a diameter of the constriction is a function of a diameter of the input TILs in the suspension, thereby causing perturbations of the input TILs large enough for one or more nucleic acids encoding one or more cytokines, one or more nucleic acids encoding one or more proteins that enhance persistence and/or tumor killing activity (e.g., anti-apoptotic factors, e.g., Bcl-2), and/or one or more nucleic acids encoding one or more co-stimulatory molecules to pass through to form perturbed input TILs; and b) incubating the perturbed input TILs with the one or more nucleic acids encoding one or more cytokines, the one or more nucleic acids encoding the one or more proteins that enhance persistence and/or tumor killing activity (e.g., anti-apoptotic factors, e.g., Bcl-2), and/or one or more nucleic acids encoding one or more co-stimulatory molecules to allow the one or more nucleic acids encoding one or more cytokines, the one or more nucleic acids encoding the one or more proteins that enhance persistence and/or tumor killing activity (e.g., anti-apoptotic factors, e.g., Bcl-2), and/or one or more nucleic acids encoding one or more co-stimulatory molecules to enter the perturbed input TILs; wherein the nucleic acids are expressed, thereby generating the modified TILs comprising the one or more cytokines, the one or more proteins that enhance persistence and/or tumor killing activity (e.g., anti-apoptotic factors, e.g., Bcl-2), and/or the one or more co-stimulatory molecules. In some aspects, the nucleic acids encoding the cytokines, the one or more proteins that enhance persistence and/or tumor killing activity (e.g., anti-apoptotic factors, e.g., Bcl-2), and/or one or the nucleic acids encoding the co-stimulatory molecule are mRNAs. In some aspects, wherein the cytokine is a membrane-bound cytokine, the nucleic acid encoding the chimeric membrane-bound cytokine is an mRNA encoding the chimeric membrane-bound cytokine.

In some aspects, provided are compositions comprising modified TILs, wherein the TILs are modified by a process comprising: a) passing a cell suspension comprising input TILs through a cell-deforming constriction, wherein a diameter of the constriction is a function of a diameter of the input TILs in the suspension, thereby causing perturbations of the input TILs large enough for the one or more nucleic acids encoding one or more cytokines, one or more nucleic acids encoding one or more proteins that enhance persistence and/or tumor killing activity (e.g., anti-apoptotic factors, e.g., Bcl-2), and/or the one or more co-stimulatory molecules to pass through to form perturbed input TILs; and b) incubating the perturbed input TILs with the one or more nucleic acids encoding one or more cytokines, one or more nucleic acids encoding one or more proteins that enhance persistence and/or tumor killing activity (e.g., anti-apoptotic factors, e.g., Bcl-2), and/or the one or more co-stimulatory molecules to enter the perturbed input TILs; wherein the nucleic acids are expressed thereby generating the modified TILs comprising the one or more cytokines, the one or more proteins that enhance persistence and/or tumor killing activity (e.g., anti-apoptotic factors, e.g., Bcl-2), and/or the one or more co-stimulatory molecules. In some aspects, the nucleic acids encoding the one or more cytokines are mRNAs. In some aspects, wherein the cytokine is a membrane-bound cytokine, the nucleic acid encoding the chimeric membrane-bound cytokine is an mRNA encoding the chimeric membrane-bound cytokine.

In some aspects, provided are compositions comprising modified TILs, wherein the TILs are modified in a process comprising incubating the TILs with the nucleic acid encoding the chimeric membrane-bound cytokine before, during and/or after passing the cell suspension through the cell-deforming constriction. In some aspects, the TILs are modified in a process comprising incubating the TILs with the nucleic acid encoding the chimeric membrane-bound cytokine and the co-stimulatory molecule before, during and/or after passing the cell suspension through the cell-deforming constriction. In some aspects, the TILs are modified in a process comprising incubating the TILs with the nucleic acid encoding the chimeric membrane-bound cytokine and the nucleic acid encoding the co-stimulatory molecule before, during and/or after passing the cell suspension through the cell-deforming constriction. In some aspects, the TILs are modified in a process comprising incubating the TILs with the nucleic acid encoding the chimeric membrane-bound cytokine and the nucleic acid encoding the one or more co-stimulatory molecules before, during and/or after passing the cell suspension through the cell-deforming constriction. In some aspects, the nucleic acid encoding the chimeric membrane-bound cytokine is an mRNA encoding the chimeric membrane-bound cytokine.

In some aspects, provided are compositions comprising modified TILs, wherein the TILs are modified in a process comprising incubating the TILs with the nucleic acid encoding the chimeric membrane-bound cytokine before passing the cell suspension through the cell-deforming constriction. In some aspects, the TILs are modified in a process comprising incubating the TILs with the nucleic acid encoding the chimeric membrane-bound cytokine and the co-stimulatory molecule before passing the cell suspension through the cell-deforming constriction. In some aspects, the TILs are modified in a process comprising incubating the TILs with the nucleic acid encoding the chimeric membrane-bound cytokine and the nucleic acid encoding the co-stimulatory molecule before passing the cell suspension through the cell-deforming constriction. In some aspects, the TILs are modified in a process comprising incubating the TILs with the nucleic acid encoding the chimeric membrane-bound cytokine and the nucleic acid encoding the one or more co-stimulatory molecules before passing the cell suspension through the cell-deforming constriction. In some aspects, the nucleic acid encoding the chimeric membrane-bound cytokine is an mRNA encoding the chimeric membrane-bound cytokine.

In some aspects according to any one of the compositions described herein, the TILs are mammalian TILs. In some aspects, the TILs are monkey, mouse, dog, cat, horse, rat, sheep, goat, pig, or rabbit TILs. In some aspects, the TILs are primate TILs. In some aspects, the TILs are human TILs.

### Methods of Treatment, Compositions for Use as a Medicine, and Use of Pharmaceutical Composition in Manufacture of a Medicament

In some aspects, there is provided a method of modulating (e.g., increasing or decreasing) an immune response in an individual, comprising administering modified TILs which exhibit increased expression of one or more cytokines, one or more proteins that enhance persistence and/or tumor killing activity (e.g., anti-apoptotic factors, e.g., Bcl-2), and/or one or more co-stimulatory molecules. For instance, in some aspects, present disclosure provides a method of modulating an immune response in an individual in need thereof, comprising administering to the subject a modified TIL which exhibits increased expression of one or more cytokines (e.g., membrane-bound cytokines). In some aspects, present disclosure provides a method of modulating an immune response in an individual in need thereof, comprising administering to the subject a modified TIL which exhibits increased expression of one or more proteins that enhance persistence and/or tumor killing activity of the TILs (e.g., anti-apoptotic factors, e.g., Bcl-2). In some aspects, present disclosure provides a method of modulating an immune response in an individual in need thereof, comprising administering to the subject a modified TIL which exhibits increased expression of one or more co-stimulatory molecules. In some aspects, provided herein is a method of modulating an immune response in an individual in need thereof, comprising administering to the subject a modified TIL which exhibits increased expression of both (i) one or more cytokines (e.g., membrane-bound cytokines) and (ii) one or more co-stimulatory molecules. In some aspects, the modified TILs are prepared according to any one of the methods described herein. In some aspects, provided herein is a method of modulating an immune response in an individual in need thereof, comprising administering to the subject a modified TIL which exhibits increased expression of both (i) one or more cytokines (e.g., membrane-bound cytokines). (ii) one or more co-stimulatory molecules, and (iii) one or more proteins that enhance persistence and/or tumor killing activity (e.g., anti-apoptotic factors, e.g., Bcl-2). In some aspects, the modified TILs are prepared according to any one of the methods described herein. In some aspects, there is provided a method of modulating an immune response in an individual, comprising administering any one of the compositions comprising modified TILs described herein.

As is apparent from the present disclosure, in some aspects, modulating an immune response comprises enhancing or increasing an immune response as compared to the immune response in a reference subject (e.g., corresponding individual who did not receive the modified TILs described herein). In some aspects, modulating an immune response comprises altering the type of immune response (e.g., from a Th2 to a Th1; or from an antibody response to a CD8 T cell mediated response). As described below, such ability to modulate immune responses in an individual can be useful in treating a wide range of diseases and disorders.

In some aspects, there is provided a method of treating a cancer, an infectious disease, or a viral-associated disease in an individual, comprising administering modified TILs prepared according to any one of the methods described herein. In some aspects, there is provided a method of treating a cancer, an infectious disease, or a viral-associated disease in an individual, comprising administering any one of the compositions comprising modified TILs described herein. Accordingly, some aspects of the present disclosure related to a method of treating a cancer in a subject in need thereof, comprising administering to the subject any of the modified TILs described herein. In some aspects, provided herein is a method of treating an infectious disease in a subject in need thereof, comprising administering to the subject any of the modified TILs described herein. In some aspects, provided herein is a method of treating a viral-associated disease in a subject in need thereof, comprising administering to the subject any of the modified TILs described herein. Non-limiting examples of cancers, infectious diseases, and viral-associated diseases that can be treated with the present disclosure are provided elsewhere herein.

In some aspects, the method of treating provided herein comprises multiple administrations of a therapeutic agent. Accordingly, in some aspects, a treatment method provided herein comprises multiple administration of the modified TILs or composition comprising modified TILs. In some aspects, the modified TILs (or composition comprising the modified TILs) are administered to the subject at least about two times, at least about three times, at least about four times, at least about five times, at least about six times, at least about seven times, at least about eight times, at least about nine times, or at least about 10 times or more.

As is apparent from the present disclosure, the modified TILs (or composition comprising the modified TILs) can be administered to the subject using any suitable routes of administration Non-limiting examples of such administration routes include: intratumorally, orally, pulmonarily, intranasally, parenterally (intravenously, intra-arterially, intramuscularly, intraperitoneally, or subcutaneously), rectally, intralymphatically, intrathecally, periocularly or topically. In some aspects, the modified TILs or composition comprising modified TILs are administered intravenously or intratumorally. In some aspects, the individual is a mammal. In some aspects, the individual is a monkey, mouse, dog, cat, horse, rat, sheep, goat, pig, or rabbit. In some aspects, the individual is a primate. In some aspects, the individual is human. In some aspects, the modified TILs or the composition comprising modified TILs are administered prior to, concurrently with, or following administration of another therapy. In some aspects, the another therapy is checkpoint therapy.

In some aspects, there is provided a pharmaceutical composition for stimulating an immune response in an individual, wherein the composition comprises an effective amount of any one of the compositions comprising modified TILs described herein. In some aspects, there is provided a pharmaceutical composition for use as a medicine, wherein the pharmaceutical composition comprises an effective amount of any one of the compositions comprising modified TILs described herein.

In some aspects, there is provided a pharmaceutical composition for treating a cancer, an infectious disease, or a viral-associated disease in an individual, wherein the pharmaceutical composition comprises an effective amount of any one of the compositions comprising modified TILs described herein.

In some aspects, provided are uses of a pharmaceutical composition in the manufacture of a medicament for stimulating an immune response in an individual, wherein the pharmaceutical composition comprises an effective amount of any one of the compositions comprising modified TILs described herein.

In some aspects, provided are uses of a pharmaceutical composition in the manufacture of a medicament for treating a cancer, an infectious disease, or a viral-associated disease in an individual, wherein the pharmaceutical composition comprises an effective amount of composition of any one of the compositions comprising modified TILs described herein.

In some aspects, there is provided a composition for use as a medicine, wherein the composition comprises an effective amount of modified TILs comprising a chimeric membrane-bound cytokine. In some aspects, provided are compositions for treating a cancer, an infectious disease, or a viral-associated disease with a composition in an individual, wherein the composition comprises an effective amount of TILs comprising a chimeric membrane-bound cytokine. In some aspects, provided are methods of treating a cancer, an infectious disease, or a viral-associated disease in an individual, comprising administering a composition comprising an effective amount of TILs comprising a chimeric membrane-bound cytokine to the individual. In some aspects, there is provided a composition for use as a medicine, wherein the composition comprises an effective amount of modified TILs, wherein the TILs have been modified to express a protein that enhances the persistence and/or tumor killing activity of the TILs (e.g., Bcl-2) In some aspects, provided are compositions for treating a cancer, an infectious disease, or a viral-associated disease with a composition in an individual, wherein the composition comprises an effective amount of modified TILs, wherein the TILs have been modified to express a protein that enhances the persistence and/or tumor killing activity of the TILs (e.g., Bcl-2). In some aspects, provided are methods of treating a cancer, an infectious disease, or a viral-associated disease in an individual, comprising administering a composition comprising an effective amount of modified TILs, wherein the TILs have been modified to express a protein that enhances the persistence and/or tumor killing activity of the TILs (e.g., Bcl-2).

In some aspects, provided are uses of a composition comprising an effective amount of TILs comprising a chimeric membrane-bound cytokine in the manufacture of a medicament for stimulating an immune response in an individual and/or treating a cancer, an infectious disease, or a viral-associated disease in an individual. In some aspects, provided are uses of a composition comprising an effective amount of modified TILs that express a protein that enhances persistence and/or tumor killing activity in the manufacture of a medicament for stimulating an immune response in an individual and/or treating a cancer, an infectious disease, or a viral-associated disease in an individual.

In some aspects, according to any one of the pharmaceutical compositions, compositions for use, or uses described herein, the composition is formulated for multiple administrations. In some aspects, the composition is suitable for administration to a subject using any of the administration routes described herein. In some aspects, the composition is administered intravenously or intratumorally. In some aspects, the individual is a mammal. In some aspects, the individual is a monkey, mouse, dog, cat, horse, rat, sheep, goat, pig, or rabbit. In some aspects, the individual is a primate. In some aspects, the individual is human. In some aspects, the composition is formulated for administration prior to, concurrently with, or following administration of another therapy. In some aspects, the another therapy is checkpoint therapy.

In some aspects, according to any one of the methods, pharmaceutical compositions, compositions for use, or uses described herein, the TILs are modified by a process comprising: a) passing a cell suspension comprising input TILs through a cell-deforming constriction, wherein a diameter of the constriction is a function of a diameter of the input TILs in the suspension, thereby causing perturbations of the input TILs large enough for one or more nucleic acids encoding one or more cytokines, one or more nucleic acids encoding one or more proteins that enhance persistence and/or tumor killing activity (e.g., anti-apoptotic factors, e.g., Bcl-2), and/or one or more nucleic acids encoding one or more co-stimulatory molecules to pass through to form perturbed input TILs; and b) incubating the perturbed input TILs with the one or more nucleic acids encoding one or more cytokines, one or more nucleic acids encoding one or more proteins that enhance persistence and/or tumor killing activity (e.g., anti-apoptotic factors, e.g., Bcl-2), and/or one or more nucleic acids encoding one or more co-stimulatory molecules to allow the one or more nucleic acids encoding one or more cytokines, one or more nucleic acids encoding one or more proteins that enhance persistence and/or tumor killing activity (e.g., anti-apoptotic factors, e.g., Bcl-2), and/or one or more nucleic acids encoding one or more co-stimulatory molecules to enter the perturbed input TILs; wherein the nucleic acids are expressed, thereby generating the modified TILs comprising the one or more cytokines and/or the one or more co-stimulatory molecules. In some aspects, the one or more nucleic acids encoding the cytokines, one or more nucleic acids encoding the protein that enhance persistence and/or tumor killing activity (e.g., anti-apoptotic factors, e.g., Bcl-2), and/or one or the nucleic acids encoding the co-stimulatory molecule are mRNAs. In some aspects, wherein the cytokine is a membrane-bound cytokine, the nucleic acid encoding the chimeric membrane-bound cytokine is an mRNA encoding the chimeric membrane-bound cytokine.

In some aspects according to any one of the methods, pharmaceutical compositions, compositions for use, or uses described herein, the TILs are modified by a process comprising: a) passing a cell suspension comprising input TILs through a cell-deforming constriction, wherein a diameter of the constriction is a function of a diameter of the input TILs in the suspension, thereby causing perturbations of the input TILs large enough for the one or more nucleic acids encoding one or more cytokines, the one or more proteins that enhance persistence and/or tumor killing activity (e.g., anti-apoptotic factors, e.g., Bcl-2), and/or the one or more co-stimulatory molecules to pass through to form perturbed input TILs; and b) incubating the perturbed input TILs with the one or more nucleic acids encoding one or more cytokines, the one or more proteins that enhance persistence and/or tumor killing activity (e.g., anti-apoptotic factors, e.g., Bcl-2), and/or the one or more co-stimulatory molecules to enter the perturbed input TILs; wherein the nucleic acids are expressed thereby generating the modified TILs comprising the one or more cytokines, the one or more proteins that enhance persistence and/or tumor killing activity (e.g., anti-apoptotic factors, e.g., Bcl-2), and/or the one or more co-stimulatory molecules. In some aspects, the nucleic acids encoding the one or more cytokines are mRNAs. In some aspects, wherein the cytokine is a membrane-bound cytokine, the nucleic acid encoding the chimeric membrane-bound cytokine is an mRNA encoding the chimeric membrane-bound cytokine.

### Modulation of Activity and Proliferative Capacity of TILs

As is apparent from the present disclosure, modified TILs described herein exhibit one or more improved properties compared to corresponding TILs that have not been modified as described herein. In some aspects, according to any one of the methods, pharmaceutical compositions, compositions for use, or uses described herein, the modified TILs exhibit increased in vivo persistence compared to corresponding TILs that are not modified. Not to be bound by any one theory, in some aspects, in addition to or in lieu of the expression of one or more proteins that enhance persistence and/or tumor killing activity (e.g., anti-apoptotic factors, e.g., Bcl-2), the increased expression of one or more cytokines (e.g., membrane-bound cytokines) and/or increased expression of one or more co-stimulatory molecules can enhance the in vivo persistence of the TILs. Accordingly, in some aspects, the modified TILs expressing membrane-bound IL-2, IL-7, IL-15, and/or IL-12 exhibit increased in vivo persistence compared to corresponding TILs not expressing membrane-bound IL-2, IL-7, IL-15, and/or IL-12. In some aspects, the modified TILs having increased expression of one or more co-stimulatory molecules exhibit increased in vivo persistence compared to corresponding TILs that have not been modified to exhibit increased expression of the one or more co-stimulatory molecules. In some aspects, the increased in vivo persistence is due to an increase in the survival of the modified TILs. In some aspects, the increased in vivo persistence is due an increased proliferative capacity of the modified TILs. In some aspects, the increased in vivo persistence is due to both an increase in survival and increase in proliferative capacity.

In some aspects, according to any one of the methods, pharmaceutical compositions, compositions for use, or uses described herein, the modified TILs of the present disclosure (i.e., having increased expression of one or more cytokines and/or increased expression of one or more co-stimulatory molecules) exhibit increased in vivo circulation time compared to corresponding TILs that are not modified. In some aspects, the modified TILs expressing membrane-bound IL-2, IL-7, IL-15, and/or IL-12 exhibit increased in vivo circulation time compared to corresponding TILs not expressing membrane-bound IL-2, IL-7, IL-15, and/or IL-12. In some aspects, the modified TILs having increased expression of one or more co-stimulatory molecules exhibit increased in vivo circulation as compared to corresponding TILs that have not been modified to exhibit increased expression of the one or more co-stimulatory molecules. In some aspects, the modified TILs having increased expression of one or more proteins that enhance persistence and/or tumor killing activity (e.g., anti-apoptotic factors, e.g., Bcl-2) exhibit increased in vivo circulation as compared to corresponding TILs that have not been modified.

Generally, the long-term survival and/or persistence of cells (e.g., immune cells) require the addition of exogenous cytokines (e.g., IL-2, IL-7, and/or IL-15). As used herein, the term “exogenous cytokines” refer to cytokines that are provided to the TILs (i.e., not expressed by the TLs in a membrane-bound form) such that the cytokines can act on the TILs. As described herein, in some aspects, the exogenous cytokine comprises exogenous IL-2. In some aspects, the exogenous cytokine does not comprise exogenous IL-2 In some aspects, the exogenous cytokine does not comprise exogenous IL-15. In some aspects, the exogenous cytokine comprises exogenous IL-15 As demonstrated herein, in some aspects according to any one of the methods, pharmaceutical compositions, compositions for use, or uses described herein, the modified TILs of the present disclosure (e.g., having increased expression of one or more cytokines, increased expression of one or more proteins that enhance persistence and/or tumor killing activity (e.g., anti-apoptotic factors, e.g., Bcl-2), and/or increased expression of one or more co-stimulatory molecules) exhibit increased in vivo persistence in the absence of exogenous cytokines compared to corresponding TILs that are not modified. In some aspects, the modified TILs expressing membrane-bound IL-2, IL-7, IL-15, and/or IL-12 exhibit increased in vivo persistence in the absence of exogenous cytokines compared to corresponding TILs not expressing membrane-bound cytokine (e.g., membrane-bound IL-2, IL-7, IL-15, and/or IL-12) In some aspects, the modified TLs having increased expression of one or more co-stimulatory molecules exhibit increased in vivo persistence in the absence of exogenous cytokines as compared to corresponding TILs that have not been modified to exhibit increased expression of the one or more co-stimulatory molecules. In some aspects, the modified TILs expressing membrane-bound cytokine (e.g., IL-2, IL-7, IL-15, and/or IL-12) exhibit increased expression of markers of T cell stemness. Exemplary markers that are traditionally used to identify T_{SCM} cells are known and provided elsewhere in the present disclosure. In some aspects, the modified TILs having increased expression of one or more co-stimulatory molecules exhibit increased expression of markers of T cell stemness in the absence of exogenous cytokines as compared to corresponding TILs that have not been modified as such. Accordingly, in some aspects, modified TILs described herein more closely resemble T_{SCM} cells (in function and/or phenotype) as compared to corresponding TILs that have not been modified as such.

In some aspects according to any one of the methods, pharmaceutical compositions, compositions for use, or uses described herein, the modified TILs exhibit increased in vivo circulation time in the absence of exogenous cytokines compared to corresponding TILs that are not modified. In some aspects, the modified TILs expressing membrane-bound cytokines (e.g., membrane-bound IL-2, IL-7, IL-15, and/or IL-12) exhibit increased in vivo circulation time in the absence of exogenous cytokines compared to corresponding TILs not expressing membrane-bound cytokines (e.g., membrane-bound IL-2, IL-7, IL-15, and/or IL-12). In some aspects, the modified TILs having increased expression of one or more co-stimulatory molecules exhibit increased in vivo circulation time in the absence of exogenous cytokines as compared to corresponding TILs that have not been modified to exhibit increased expression of the one or more co-stimulatory molecules. In some aspects, the modified TILs having increased expression of one or more proteins that are capable of enhancing persistence and/or tumor killing activity exhibit increased in vivo circulation time in the absence of exogenous cytokines as compared to corresponding TILs that have not been modified as such.

In some aspects, according to any one of the methods, pharmaceutical compositions, compositions for use, or uses described herein, the modified TILs exhibit increased in vivo persistence wherein TILs were cultured in the absence of exogenous cytokines for any one of 1, 2, 3, 4, 5, 6, 7, 8, 9, or 10 days, compared to corresponding TILs that are not modified. In some aspects, the TILs are cultured in the absence of exogenous cytokines for at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15, at least about 16 days, at least about 17 days, at least about 18 days, at least about 19 days, or at least about 20 days. In some aspects, the modified TILs are cultured in the absence of exogenous cytokines for between about 1 day to about 20 days. In some aspects, the modified TILs are cultured in the absence of exogenous cytokines for between about 1 day to about 10 days in some aspects, the modified TILs are cultured in the absence of exogenous cytokines for between about 3 days to about 11 days. In some aspects, the modified TILs are cultured in the absence of exogenous cytokines for between about 7 days to about 14 days. In some aspects, after the culturing, the modified TILs exhibit increased in vivo persistence as compared to corresponding TILs that have not been modified.

In some aspects, modified TILs described herein have been cultured in the absence of exogenous cytokines for at least about 1 day, wherein after the culturing, the modified T cells exhibit increased persistence compared to corresponding TILs that have not been modified as described herein. In some aspects, the modified TILs have been cultured in the absence of exogenous cytokines for at least about 2 days, wherein after the culturing, the modified T cells exhibit increased persistence compared to corresponding TILs that have not been modified as described herein. In some aspects, the modified TILs have been cultured in the absence of exogenous cytokines for at least about 3 days, wherein after the culturing, the modified T cells exhibit increased persistence compared to corresponding TILs that have not been modified as described herein. In some aspects, the modified TILs have been cultured in the absence of exogenous cytokines for at least about 4 days, wherein after the culturing, the modified T cells exhibit increased persistence compared to corresponding TILs that have not been modified as described herein. In some aspects, the modified TILs have been cultured in the absence of exogenous cytokines for at least about 5 days, wherein after the culturing, the modified T cells exhibit increased persistence compared to corresponding TILs that have not been modified as described herein. In some aspects, the modified TILs have been cultured in the absence of exogenous cytokines for at least about 6 days, wherein after the culturing, the modified T cells exhibit increased persistence compared to corresponding TILs that have not been modified as described herein. In some aspects, the modified TILs have been cultured in the absence of exogenous cytokines for at least about 7 days, wherein after the culturing, the modified T cells exhibit increased persistence compared to corresponding TILs that have not been modified as described herein. In some aspects, the modified TILs have been cultured in the absence of exogenous cytokines for at least about 8 days, wherein after the culturing, the modified T cells exhibit increased persistence compared to corresponding TILs that have not been modified as described herein. In some aspects, the modified TILs have been cultured in the absence of exogenous cytokines for at least about 9 days, wherein after the culturing, the modified T cells exhibit increased persistence compared to corresponding TILs that have not been modified as described herein. In some aspects, the modified TILs have been cultured in the absence of exogenous cytokines for at least about 10 days, wherein after the culturing, the modified T cells exhibit increased persistence compared to corresponding TILs that have not been modified as described herein. In some aspects, the modified TILs have been cultured in the absence of exogenous cytokines for at least about 11 days, wherein after the culturing, the modified T cells exhibit increased persistence compared to corresponding TILs that have not been modified as described herein. In some aspects, the modified TILs have been cultured in the absence of exogenous cytokines for at least about 12 days, wherein after the culturing, the modified T cells exhibit increased persistence compared to corresponding TILs that have not been modified as described herein. In some aspects, the modified TILs have been cultured in the absence of exogenous cytokines for at least about 13 days, wherein after the culturing, the modified T cells exhibit increased persistence compared to corresponding TILs that have not been modified as described herein. In some aspects, the modified TILs have been cultured in the absence of exogenous cytokines for at least about 14 days, wherein after the culturing, the modified T cells exhibit increased persistence compared to corresponding TILs that have not been modified as described herein. In some aspects, the modified TILs have been cultured in the absence of exogenous cytokines for at least about 15 days, wherein after the culturing, the modified T cells exhibit increased persistence compared to corresponding TILs that have not been modified as described herein. In some aspects, the modified TILs have been cultured in the absence of exogenous cytokines for at least about 16 days, wherein after the culturing, the modified T cells exhibit increased persistence compared to corresponding TILs that have not been modified as described herein. In some aspects, the modified TILs have been cultured in the absence of exogenous cytokines for at least about 17 days, wherein after the culturing, the modified T cells exhibit increased persistence compared to corresponding TILs that have not been modified as described herein. In some aspects, the modified TILs have been cultured in the absence of exogenous cytokines for at least about 18 days, wherein after the culturing, the modified T cells exhibit increased persistence compared to corresponding TILs that have not been modified as described herein. In some aspects, the modified TILs have been cultured in the absence of exogenous cytokines for at least about 19 days, wherein after the culturing, the modified T cells exhibit increased persistence compared to corresponding TILs that have not been modified as described herein. In some aspects, the modified TILs have been cultured in the absence of exogenous cytokines for at least about 20 days, wherein after the culturing, the modified T cells exhibit increased persistence compared to corresponding TILs that have not been modified as described herein. In some aspects, the modified TILs expressing membrane-bound cytokines (e.g., membrane-bound IL-2, IL-7, IL-15, and/or IL-12) exhibit increased in vivo persistence wherein TILs were cultured in the absence of exogenous cytokines for any one of 1, 2, 3, 4, 5, 6, 7, 8, 9, or 10 days, compared to corresponding TILs not expressing membrane-bound cytokines (e.g., membrane-bound IL-2, IL-7, IL-15, and/or IL-12). In some aspects, the modified TILs expressing membrane-bound cytokines (e.g., membrane-bound IL-2, IL-7, IL-15, and/or IL-12) are cultured in the absence of exogenous cytokines for at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15, at least about 16 days, at least about 17 days, at least about 18 days, at least about 19 days, or at least about 20 days. In some aspects, the modified TILs expressing membrane-bound cytokines (e.g., membrane-bound IL-2, IL-7, IL-15, and/or IL-12) are cultured in the absence of exogenous cytokines for between about 1 day to about 20 days. In some aspects, the modified TILs expressing membrane-bound cytokines (e.g., membrane-bound IL-2, IL-7, IL-15, and/or IL-12) are cultured in the absence of exogenous cytokines for between about 1 day to about 10 days. In some aspects, the modified TILs expressing membrane-bound cytokines (e.g., membrane-bound IL-2, IL-7, IL-15, and/or IL-12) are cultured in the absence of exogenous cytokines for between about 3 days to about 11 days. In some aspects, the modified TILs expressing membrane-bound cytokines (e.g., membrane-bound IL-2, IL-7, IL-15, and/or IL-12) are cultured in the absence of exogenous cytokines for between about 7 days to about 14 days. In some aspects, after the culturing, the modified TILs exhibit increased in vivo persistence as compared to corresponding TILs that have not been modified (e.g., does not express membrane-bound cytokines, e.g., membrane-bound IL-2, IL-7, IL-15, and/or IL-12).

In some aspects according to any one of the methods, pharmaceutical compositions, compositions for use, or uses described herein, the modified TILs exhibit increased in vivo circulation time in the absence of exogenous cytokines wherein TILs were cultured in the absence of exogenous cytokines for any one of 1, 2, 3, 4, 5, 6, 7, 8, 9, or 10 days, compared to corresponding TILs that are not modified. In some aspects, the TILs are cultured in the absence of exogenous cytokines for at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15, at least about 16 days, at least about 17 days, at least about 18 days, at least about 19 days, or at least about 20 days, wherein after the culturing, the modified TILs exhibit increased in vivo circulation time as compared to corresponding TILs that have not been modified. In some aspects, the modified TILs expressing membrane-bound cytokines (e.g., membrane-bound IL-2, IL-7, IL-15, and/or IL-12) exhibit increased in vivo circulation time wherein TILs were cultured in the absence of exogenous cytokines for any one of 1, 2, 3, 4, 5, 6, 7, 8, 9, or 10 days, compared to corresponding TILs not expressing membrane-bound cytokines (e.g., membrane-bound IL-2, IL-7, IL-15, and/or IL-12) In some aspects, the modified TILs expressing membrane-bound cytokines (e.g., membrane-bound IL-2, IL-7, IL-15, and/or IL-12) are cultured in the absence of exogenous cytokines for at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15, at least about 16 days, at least about 17 days, at least about 18 days, at least about 19 days, or at least about 20 days, wherein after the culturing, the modified TILs exhibit increased in vivo persistence as compared to corresponding TILs that have not been modified (e.g., does not express membrane-bound cytokines, e.g., membrane-bound IL-2, IL-7, IL-15, and/or IL-12).

In some aspects according to any one of the methods, pharmaceutical compositions, compositions for use, or uses described herein, the modified TILs have increased expression of one or more markers of central memory T-cells (such as but not limited to CD62L) compared to corresponding TILs that are not modified. As used herein, the term “central memory T cells” or “T_{CM} cells” refer to memory T cells that express at least CD45RO, CCR7, CD62L, and/or CD127. Accordingly, in some aspects, the methods of modifying TILs described herein (e.g., using squeeze processing to deliver one or more nucleic acids encoding a cytokine and/or one or more nucleic acids encoding a co-stimulatory molecule to TILs) are useful for increasing the expression of one or more central memory T cell markers on the TILs. In some aspects, the modified TILs expressing membrane-bound cytokines (e.g., membrane-bound IL-2, IL-7, IL-15, and/or IL-12) have increased expression of one or more markers of central memory T-cells compared to corresponding TILs not expressing membrane-bound cytokines (e.g., membrane-bound IL-2, IL-7, IL-15 and/or IL-12. In some aspects, the modified TILs expressing one or more proteins that enhance persistence and/or tumor killing activity (e.g., anti-apoptotic factors, e.g., Bcl-2) have increased expression of one or more markers of central memory T-cells compared to corresponding TILs that do not express the one or more proteins.

In some aspects according to any one of the methods, pharmaceutical compositions, compositions for use, or uses described herein, the modified TILs have increased expression of one or more markers of central memory T-cells when cultured in the absence of exogenous cytokines, compared to corresponding TILs that are not modified. In some aspects, the modified TILs have increased expression of one or more markers of central memory T-cells when cultured in the absence of exogenous cytokines for any one of 1, 2, 3, 4, 5, 6, 7, 8, 9, or 10 days, compared to corresponding TILs that are not modified. In some aspects, the TILs are cultured in the absence of exogenous cytokines for at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15, at least about 16 days, at least about 17 days, at least about 18 days, at least about 19 days, or at least about 20 days, wherein after the culturing, the modified TILs exhibit increased expression of one or more markers of central memory T cells, as compared to corresponding TILs that have not been modified. In some aspects, the modified TILs expressing membrane-bound cytokines (e.g., membrane-bound IL-2, IL-7, IL-15 and/or IL-12) have increased expression of one or more markers of central memory T-cells when cultured in the absence of exogenous cytokines, compared to corresponding TILs not expressing membrane-bound cytokines (e.g., membrane-bound IL-2, IL-7, IL-15, and/or IL-12). In some aspects, the modified TILs expressing membrane-bound cytokines (e.g., membrane-bound IL-2, IL-7, IL-15, and/or IL-12) are cultured in the absence of exogenous cytokines for at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15, at least about 16 days, at least about 17 days, at least about 18 days, at least about 19 days, or at least about 20 days, wherein after the culturing, the modified TILs exhibit increased expression of one or more central memory T cell markers as compared to corresponding TILs that have not been modified (e.g., does not express membrane-bound IL-2, IL-7, IL-15, and/or IL-12). In some aspects, the exogenous cytokine comprises exogenous IL-2. In some aspects, the exogenous cytokine does not comprise exogenous IL-2. In some aspects, the exogenous cytokine does not comprise exogenous IL-15. In some aspects, the exogenous cytokine comprises exogenous IL-15.

In some aspects according to any one of the methods, pharmaceutical compositions, compositions for use, or uses described above, the expression of one or more markers of central memory T-cells in the modified TILs is increased by about any one of: 10%, 25%, 50%, 75%, 100%, 1.2-fold, 1.5-fold, 1.8-fold, 2-fold, 3-fold, 4-fold, 5-fold, 8-fold, or more than 10-fold as compared to corresponding TILs that are not modified.

In some aspects according to any one of the methods, pharmaceutical compositions, compositions for use, or uses described herein, the modified TILs have increased expression of one or more markers of T-cell self-renewal (also referred to herein as markers for “stem cell-like memory T cells”) (such as but not limited to TCF1) compared to corresponding TILs that are not modified. As used herein, the term “stem cell-like memory T cells,” “T memory stein cells,” or “T_{SCM} cells” refer to memory T cells that express CD95, CD45RA, CCR7, and CD62L and are endowed with the stem cell-like ability to self-renew and the multipotent capacity to reconstitute the entire spectrum of memory and effector subsets. Accordingly, in some aspects, the methods of modifying TILs described herein (e.g., using squeeze processing to deliver one or more nucleic acids encoding a cytokine and/or one or more nucleic acids encoding a co-stimulatory molecule to TILs) are useful for increasing the expression of one or more T_{SCM} cell markers on the TILs. In some aspects, the modified TILs expressing membrane-bound cytokines (e.g., membrane-bound IL-2, IL-7, IL-15, and/or IL-12) have increased expression of one or more markers of T-cell self-renewal compared to corresponding TILs not expressing membrane-bound cytokines (e.g., membrane-bound IL-2, IL-7, IL-15, and/or IL-12). In some aspects, the modified TILs expressing one or more proteins that enhance persistence and/or tumor killing activity (e.g., anti-apoptotic factors, e.g., Bcl-2) have increased expression of one or more markers of T-cell self-renewal compared to corresponding TILs that do not express the one or more proteins.

In some aspects, according to any one of the methods, pharmaceutical compositions, compositions for use, or uses described herein, the modified TILs have increased expression of one or more markers of T-cell self-renewal when cultured in the absence of exogenous cytokines, compared to corresponding TILs that are not modified. In some aspects, the modified TILs have increased expression of one or more markers of T-cell self-renewal when cultured in the absence of exogenous cytokines for any one of 1, 2, 3, 4, 5, 6, 7, 8, 9, or 10 days, compared to corresponding TILs that are not modified. In some aspects, the TILs are cultured in the absence of exogenous cytokines for at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15, at least about 16 days, at least about 17 days, at least about 18 days, at least about 19 days, or at least about 20 days, wherein after the culturing, the modified TILs exhibit increased expression of one or more T_{SCM} cell markers as compared to corresponding TILs that have not been modified. In some aspects, the modified TILs expressing membrane-bound cytokines (e.g., membrane-bound IL-2, IL-7, IL-15, and/or IL-12) have increased expression of one or more markers of T-cell self-renewal when cultured in the absence of exogenous cytokines, compared to corresponding TILs not expressing membrane-bound cytokines (e.g., membrane-bound IL-2, IL-7, IL-15, and/or IL-12). In some aspects, the modified TILs expressing membrane-bound cytokines (e.g., membrane-bound IL-2, IL-7, IL-15, and/or IL-12) are cultured in the absence of exogenous cytokines for at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15, at least about 16 days, at least about 17 days, at least about 18 days, at least about 19 days, or at least about 20 days, wherein after the culturing, the modified TILs exhibit increased expression of one or more T_{SCM} cell markers as compared to corresponding TILs that have not been modified (e.g., does not express membrane-bound cytokines, e.g., IL-2, IL-7, IL-15, and/or IL-12) In some aspects, the exogenous cytokine comprises exogenous IL-2. In some aspects, the exogenous cytokine does not comprise exogenous IL-2. In some aspects, the exogenous cytokine does not comprise exogenous IL-15. In some aspects, the exogenous cytokine comprises exogenous IL-15.

In some aspects according to any one of the methods, pharmaceutical compositions, compositions for use, or uses described above, the expression of one or more markers of T-cell self-renewal in the modified TILs is increased by about any one of: 10%, 25%, 50%, 75%, 100%, 1.2-fold, 1.5-fold, 1.8-fold, 2-fold, 3-fold, 4-fold, 5-fold, 8-fold, or more than 10-fold as compared to corresponding TILs that are not modified.

In some aspects according to any one of the methods, pharmaceutical compositions, compositions for use, or uses described herein, the modified TILs have reduced expression of one or more markers of T-cell exhaustion compared to corresponding TILs that are not modified. Non-limiting examples of exhaustion markers include. PD-1, CD39, TIM-3, TIGIT, and/or LAG-3. Accordingly, in some aspects, the methods of modifying TILs described herein (e.g., using squeeze processing to deliver one or more nucleic acids encoding a cytokine, one or more nucleic acids encoding a protein that enhances persistence and/or tumor killing activity, and/or one or more nucleic acids encoding a co-stimulatory molecule to TILs) are useful for reducing exhaustion in the TILs, e.g., as evidenced by reduced expression of one or more exhaustion markers. In some aspects, the modified TILs expressing membrane-bound cytokines (e.g., membrane-bound IL-2, IL-7, IL-15, and/or IL-12) have reduced expression of one or more markers of T-cell exhaustion compared to corresponding TILs not expressing membrane-bound cytokines (e.g., membrane-bound IL-2, IL-7, IL-15, and/or IL-12). In some aspects, the modified TILs provided herein which express increased expression of a co-stimulatory molecule have reduced expression of one or more markers of T-cell exhaustion as compared to corresponding TILs that do not express increased expression of the co-stimulatory molecule. In some aspects, the modified TILs described herein which express a protein that increase the persistence and/or tumor killing activity have reduced expression of one or more markers of T-cell exhaustion as compared to corresponding TILs that do not express the protein.

In some aspects according to any one of the methods, pharmaceutical compositions, compositions for use, or uses described herein, the modified TILs have reduced expression of one or more markers of T-cell exhaustion when cultured in the absence of exogenous cytokines, compared to corresponding TILs that are not modified. In some aspects, the modified TILs have reduced expression of one or more markers of T-cell exhaustion when cultured in the absence of exogenous cytokines for any one of 1, 2, 3, 4, 5, 6, 7, 8, 9, or 10 days, compared to corresponding TILs that are not modified. In some aspects, the TILs are cultured in the absence of exogenous cytokines for at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15, at least about 16 days, at least about 17 days, at least about 18 days, at least about 19 days, or at least about 20 days, wherein after the culturing, the modified TILs exhibit reduced expression of one or more markers of exhaustion, as compared to corresponding TILs that have not been modified. In some aspects, the modified TILs expressing membrane-bound cytokines (e.g., membrane-bound IL-2, IL-7, IL-15, and/or IL-12) have reduced expression of one or more markers of T-cell exhaustion when cultured in the absence of exogenous cytokines, compared to corresponding TILs not expressing membrane-bound cytokines (e.g., membrane-bound IL-2, IL-7, IL-S, and/or IL-12). In some aspects, the modified TILs expressing membrane-bound cytokines (e.g., membrane-bound IL-2, IL-7, IL-15, and/or IL-12) are cultured in the absence of exogenous cytokines for at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15, at least about 16 days, at least about 17 days, at least about 18 days, at least about 19 days, or at least about 20 days, wherein after the culturing, the modified TILs exhibit reduced expression of one or more markers of exhaustion, as compared to corresponding TILs that have not been modified (e.g., does not express membrane-bound cytokines, e.g., IL-2, IL-7, IL-15, and/or IL-12). In some aspects, the exogenous cytokine comprises exogenous IL-2. In some aspects, the exogenous cytokine comprises exogenous IL-2 In some aspects, the exogenous cytokine does not comprise exogenous IL-15. In some aspects, the exogenous cytokine comprises exogenous IL-15.

In some aspects according to any one of the methods, pharmaceutical compositions, compositions for use, or uses described above, the expression of one or more markers of T cell exhaustion in the modified TILs is reduced by about any one of: 10%, 25%, 50%, 75%, 100%, 1.2-fold, 1.5-fold, 1.8-fold, 2-fold, 3-fold, 4-fold, 5-fold, 8-fold, or more than 10-fold as compared to corresponding TILs that are not modified.

In some aspects according to any one of the methods, pharmaceutical compositions, compositions for use, or uses described herein, the modified TILs have increased expression of one or more of T-bet, EOMES, TCF1, CD127, CD103, CD45RO, CCR5, and CD62L compared to corresponding TILs that are not modified. In some aspects, the modified TILs expressing membrane-bound cytokines (e.g., membrane-bound IL-2, IL-7, IL-15, and/or IL-12) have increased expression of one or more of T-bet, EOMES, TCF1, CD127, CD103, CD45RO, CCR5, and CD62L compared to corresponding TILs not expressing membrane-bound cytokines (e.g., membrane-bound IL-2, IL-7, IL-15, and/or IL-12). In some aspects, modified TILs that have increased expression of a co-stimulatory molecule have increased expression of one or more of T-bet, EOMES, TCF1, CD127, CD103, CD45RO, CCR5, and CD62L as compared to corresponding TILs that do not have the increased expression. In some aspects, modified TILs that express a protein that increase the persistence and/or tumor killing activity have increased expression of one or more of T-bet, EOMES, TCF1, CD127, CD103, CD45RO, CCR5, and CD62L as compared to corresponding TILs that do not express the protein.

In some aspects according to any one of the methods, pharmaceutical compositions, compositions for use, or uses described herein, the modified TILs have increased expression of one or more of T-bet, EOMES, TCF1, CD127, CD103, CD45RO, CCR5, and CD62L when cultured in the absence of exogenous cytokines, compared to corresponding TILs that are not modified. In some aspects, the modified TILs have increased expression of one or more of T-bet, EOMES, TCF1, CD127, CD103, CD45RO, CCR5, and CD62L when cultured in the absence of exogenous cytokines for any one of 1, 2, 3, 4, 5, 6, 7, 8, 9, or 10 days, compared to corresponding TILs that are not modified. In some aspects, the TILs are cultured in the absence of exogenous cytokines for at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15, at least about 16 days, at least about 17 days, at least about 18 days, at least about 19 days, or at least about 20 days, wherein after the culturing, the modified TILs exhibit increased expression of one or more of T-bet, EOMES, TCF1, CD127, CD103, CD45RO, CCR5, and CD62L as compared to corresponding TILs that have not been modified. In some aspects, the modified TILs expressing membrane-bound cytokines (e.g., membrane-bound IL-2, IL-7, IL-15, and/or IL-12) have increased expression of one or more of T-bet, EOMES, TCF1, CD127, CD103, CD45RO, CCR5, and CD62L when cultured in the absence of exogenous cytokines, compared to corresponding TILs not expressing membrane-bound cytokines (e.g., membrane-bound IL-2, IL-7, IL-15, and/or IL-12). In some aspects, the modified TILs expressing membrane-bound cytokines (e.g., membrane-bound IL-2, IL-7, IL-15, and/or IL-12) are cultured in the absence of exogenous cytokines for at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15, at least about 16 days, at least about 17 days, at least about 18 days, at least about 19 days, or at least about 20 days, wherein after the culturing, the modified TILs exhibit increased expression of one or more of T-bet, EOMES, TCF1, CD127, CD103, CD45RO, CCR5, and CD62L, as compared to corresponding TILs that have not been modified (e.g., does not express membrane-bound cytokines, e.g., IL-2, IL-7, IL-15, and/or IL-12). In some aspects, the exogenous cytokine comprises exogenous IL-2. In some aspects, the exogenous cytokine does not comprise exogenous IL-2 In some aspects, the exogenous cytokine does not comprise exogenous IL-15. In some aspects, the exogenous cytokine comprises exogenous IL-15.

In some aspects according to any one of the methods, pharmaceutical compositions, compositions for use, or uses described above, the expression of one or more of T-bet, EOMES, TCF1, CD127, CD103, CD45RO, CCR5, and CD62L in the modified TILs is increased by about any one of: 10%, 25%, 50%, 75%, 100%, 1.2-fold, 1.5-fold, 1.8-fold, 2-fold, 3-fold, 4-fold, 5-fold, 8-fold, or more than 10-fold as compared to corresponding TILs that are not modified.

In some aspects, according to any one of the methods, pharmaceutical compositions, compositions for use, or uses described herein, the modified TILs have decreased expression of one or more of T-bet, EOMES, CD39 and CD69 compared to corresponding TILs that are not modified. In some aspects, the modified TILs expressing membrane-bound cytokines (e.g., membrane-bound IL-2, IL-7, IL-15, and/or IL-12) have decreased expression of one or more of T-bet, EOMES, CD39 and CD69 compared to corresponding TILs not expressing membrane-bound cytokines (e.g., membrane-bound IL-2, IL-7, and/or IL-12).

In some aspects, according to any one of the methods, pharmaceutical compositions, compositions for use, or uses described herein, the modified TILs have decreased expression of one or more of T-bet, EOMES, CD39 and CD69 when cultured in the absence of exogenous cytokines, compared to corresponding TILs that are not modified. In some aspects, the modified TILs have decreased expression of one or more of T-bet, EOMES, CD39 and CD69 when cultured in the absence of exogenous cytokines for any one of 1, 2, 3, 4, 5, 6, 7, 8, 9, or 10 days, compared to corresponding TILs that are not modified. In some aspects, the TILs are cultured in the absence of exogenous cytokines for at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15, at least about 16 days, at least about 17 days, at least about 18 days, at least about 19 days, or at least about 20 days, wherein after the culturing, the modified TILs exhibit increased expression of one or more of T-bet, EOMES, CD39 and CD69 as compared to corresponding TILs that have not been modified. In some aspects, the modified TILs expressing membrane-bound cytokines (e.g., membrane-bound IL-2, IL-7, IL-15, and/or IL-12) have decreased expression of one or more of T-bet, EOMES, CD39 and CD69 when cultured in the absence of exogenous cytokines, compared to corresponding TILs not expressing membrane-bound cytokines (e.g., membrane-bound IL-2, IL-7, IL-15, and/or IL-12). In some aspects, the modified TILs expressing membrane-bound cytokines (e.g., membrane-bound IL-2, IL-7, IL-15, and/or IL-12) are cultured in the absence of exogenous cytokines for at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15, at least about 16 days, at least about 17 days, at least about 18 days, at least about 19 days, or at least about 20 days, wherein after the culturing, the modified TILs exhibit increased expression of one or more of T-bet, EOMES, CD39 and CD69, as compared to corresponding TILs that have not been modified (e.g., does not express membrane-bound cytokines, e.g., IL-?, IL-7, IL-15, and/or IL-12). In some aspects, the exogenous cytokine comprises exogenous IL-2. In some aspects, the exogenous cytokine does not comprise exogenous IL-2. In some aspects, the exogenous cytokine does not comprise exogenous IL-15 In some aspects, the exogenous cytokine comprises exogenous IL-15.

In some aspects according to any one of the methods, pharmaceutical compositions, compositions for use, or uses described above, the expression of one or more of T-bet, EOMES, CD39 and CD69 in the modified TILs is decreased by about any one of 10%, 25%, 50%, 75%, 100%, 1.2-fold, 1.5-fold, 1.8-fold, 2-fold, 3-fold, 4-fold, 5-fold, 8-fold, or more than 10-fold as compared to corresponding TILs that are not modified.

In some aspects, according to any one of the methods, pharmaceutical compositions, compositions for use, or uses described herein, the modified TILs exhibit increased proliferation compared to corresponding TILs that are not modified. In some aspects, the modified TILs expressing membrane-bound cytokines (e.g., membrane-bound IL-2, IL-7, IL-15, and/or IL-12) exhibit increased proliferation, compared to corresponding TILs not expressing membrane-bound cytokines (e.g., membrane-bound IL-2, IL-7, IL-15, and/or IL-12). In some aspects, the modified TILs of the present disclosure that have increased expression of a co-stimulatory molecule exhibit increased proliferation compared to corresponding TILs that do not have increased expression of the co-stimulatory molecule. In some aspects, the modified TILs of the present disclose that express a protein that enhances the persistence and/or tumor killing activity exhibit increased proliferation as compared to corresponding TILs that do not express the protein.

In some aspects according to any one of the methods, pharmaceutical compositions, compositions for use, or uses described herein, the modified TILs exhibit increased proliferation when cultured in media not comprising exogenous cytokines (such as but not limited to exogenous IL-2), as compared to corresponding TILs that are not modified. In some aspects, the modified TILs exhibit increased proliferation when cultured in media not comprising exogenous cytokines (such as but not limited to exogenous IL-2) for any one of 1, 2, 3, 4, 5, 6, 7, 8, 9, or 10 days, as compared to corresponding TILs that are not modified. In some aspects, the TILs are cultured in the absence of exogenous cytokines for at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15, at least about 16 days, at least about 17 days, at least about 18 days, at least about 19 days, or at least about 20 days, wherein after the culturing, the modified TILs exhibit increased proliferation as compared to corresponding TILs that have not been modified. In some aspects, the modified TILs expressing membrane-bound cytokines (e.g., membrane-bound IL-2, IL-7, IL-15, and/or IL-12) exhibit increased proliferation when cultured in media not comprising exogenous cytokines (such as but not limited to exogenous IL-2), compared to corresponding TILs not expressing membrane-bound cytokines (e.g., membrane-bound IL-2, IL-7, IL-15, and/or IL-12) In some aspects, the modified TILs expressing membrane-bound IL-2, IL-7, IL-15, and/or IL-12 are cultured in the absence of exogenous cytokines for at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15, at least about 16 days, at least about 17 days, at least about 18 days, at least about 19 days, or at least about 20 days, wherein after the culturing, the modified TILs exhibit increased proliferation, as compared to corresponding TILs that have not been modified (e.g., does not express membrane-bound cytokines, e.g., membrane-bound IL-2, IL-7, IL-15, and/or IL-12). In some aspects, the exogenous cytokine comprises exogenous IL-2. In some aspects, the exogenous cytokine does not comprise exogenous IL-2. In some aspects, the exogenous cytokine does not comprise IL-15. In some aspects, the exogenous cytokine comprises exogenous IL-15.

In some aspects, according to any one of the methods, pharmaceutical compositions, compositions for use, or uses described above, the proliferation rate in the modified TILs is increased by about any one of: 10%, 25%, 50%, 75%, 100%, 1.2-fold, 1.5-fold, 1.8-fold, 2-fold, 3-fold, 4-fold, 5-fold, 8-fold, or more than 10-fold as compared to corresponding TILs that are not modified.

In some aspects, according to the methods, pharmaceutical compositions, compositions for use, or uses described herein, the TILs comprising the chimeric membrane-bound IL-2, IL-7, IL-15, and/or IL-12 are prepared by a) passing a cell suspension comprising input TILs through a cell-deforming constriction, wherein a diameter of the constriction is a function of a diameter of the input TILs in the suspension, thereby causing perturbations of the input TILs large enough for a nucleic acid encoding the chimeric membrane-bound IL-2, IL-7, IL-15, and/or IL-12 to pass through to form a perturbed input TILs; and b) incubating the perturbed input TILs with the nucleic acid encoding the chimeric membrane-bound IL-2, IL-7, IL-15, and/or IL-12 to allow the nucleic acid to enter the perturbed input TILs where the nucleic acid encoding the chimeric membrane-bound IL-2, IL-7, IL-15, and/or IL-12 is expressed; thereby generating TILs comprising chimeric membrane-bound IL-2, IL-7, IL-15, and/or IL-12.

### Constriction-Mediated Delivery

As described and demonstrated herein, method of modifying TILs provided herein comprises passing the TILs (e.g., cell suspension comprising the TILs) through a cell-deforming constriction, which causes perturbations in the TILs such that one or more nucleic acids encoding a cytokine and/or one or more nucleic acids encoding a co-stimulatory molecule enter the TILs through the perturbations when contacted with the TILs. As used herein, a “perturbation” refers to any opening in the cell membrane (e.g., of a TIL) that is not present under normal steady state conditions (e.g., no deformation force applied to the cells). Perturbation can comprise a hole, tear, cavity, aperture, pore, break, gap, perforation, or combinations thereof. As described herein, as a cell passes through a constriction (such as those of the present disclosure), it experiences various forces due to the constraining physical environment, including but not limited to mechanical deforming forces and/or shear forces that causes perturbations in the cell membrane.

In some aspects, the width of the constriction is about 10% to about 99% of the mean diameter of the input TILs. In some aspects, the width of the constriction is any one of about 10% to about 90%, about 10% to about 80%, about 10% to about 70%, about 20% to about 60%, about 40% to about 60%, about 30% to about 45%, about 50% to about 99%, about 50% to about 90%, about 50% to about 80%, about 50% to about 70%, about 60% to about 90%, about 60% to about 80%, or about 60% to about 70% of the mean diameter of the input TILs. In some aspects, the width of the constriction is about 5 μm to about 12 μm, about 6 μm to about 12 μm, about 8 μm to about 11 μm, about 9 μm to about 11 μm, about 9.5 μm to about 10.5 μm, about 8 μm to about 15 μm, about 10 μm to about 15 μm, or about 12 μm to about 15 μm. In some aspects, the width of the constriction is about 10 μm. In some aspects, the width of the constriction is about 3 μm to about 6 μm. In some aspects, the width of the constriction is about 4.2 μm to about 6 μm. In some aspects, the width of the constriction is about 4.2 μm to about 4.8 μm. In some aspects, the width of the constriction is about 3 μm to about 5 μm. In some aspects, the width of the constriction is about 3 μm to about 3.5 μm. In some aspects, the width of the constriction is about 3.5 μm to about 4 μm. In some aspects, the width of the constriction is about 4 μm to about 4.5 μm. In some aspects, the width of the constriction is about 3.2 μm to about 3.8 μm. In some aspects, the width of the constriction is about 3.8 μm to about 4.3 μm. In some aspects, the width of the constriction is about or less than any one of 2 μm, 2.5 μm, 3 μm, 3.5 μm, 4 μm, 4.5 μm, 5 μm, 5.5 μm, 6 μm, 6.5 μm, 7 μm, 7.5 μm, 8 μm, 8.5 μm, 9 μm, 9.5 μm, 10 μm, 10.5 μm, 11 μm, 11.5 μm, 12 μm, 12.5 μm, 13 μm, 13.5 μm, 14 μm, 14.5 μm or 15 μm. In some aspects, the width of the constriction is about or less than any one of 3.0 μm, 3.1 μm, 3.2 μm, 3.3 μm, 3.4 μm, 3.5 μm, 3.6 μm, 3.7 μm, 3.8 μm, 3.9 μm, 4.0 μm, 4.1 μm, 4.2 μm, 4.3 μm, 4.4 μm, 4.5 μm, 4.6 μm, 4.7 μm, 4.8 μm, 4.9 μm, or 5.0 μm. In some aspects, the width of the constriction is about 3.5 μm. In some aspects, the width of the constriction is about 4 μm. In some aspects, the width of the constriction is about 4.5 μm. In some aspects, the cell suspension comprising the input TILs are passed through multiple constrictions wherein the multiple constrictions are arranged in series and/or in parallel. In some aspects, the multiple constrictions are arranged in series. In some aspects, the multiple constrictions are arranged in parallel. In some aspects, the TILs can be passed through at least about 2, at least about 3, at least about 4, at least about 5, at least about 6, at least about 7, at least about 8, at least about 9, at least about 10, at least about 20, at least about 30, at least about 40, at least about 50, at least about 75, at least about 100, at least about 150, at least about 200, at least about 250, at least about 300, at least about 350, at least about 400, at least about 450, at least about 500, at least about 550, at least about 600, at least about 650, at least about 700, at least about 750, at least about 800, at least about 850, at least about 900, at least about 950, at least about 1,000 or more separate constrictions. In some aspects, the cells described herein are passed through more than about 1,000 separate constrictions. In some aspects, each of the constrictions are the same (e.g., has the same length, width, and/or depth) In some aspects, one or more of the constrictions are different.

Constrictions Used in Generating Compositions of TILs Comprising Co-Stimulatory Molecule and/or Cytokine

In some aspects, the disclosure provides compositions of TILs comprising a co-stimulatory molecule or cytokine. More specifically, as described herein, the TILs described herein have been modified such that they differ from naturally existing (i.e., non-modified) TILs. For instance, in some aspects, the modified TILs described herein exhibit increased expression of one or more cytokines (e.g., membrane-bound cytokines) in some aspects, the modified TILs described herein exhibit increased expression of one or more co-stimulatory molecules. In some aspects, the modified TILs described herein exhibit expression of bone one or more cytokines and one or more co-stimulatory molecules. As further described elsewhere in the present disclosure, the increased expression of one or more cytokines and/or one or more co-stimulatory molecules can improve one or more properties of the TILs. As also described and demonstrated herein, to increase the expression of one or more cytokines and/or one or more co-stimulatory molecules, the TILs can be modified to comprise a nucleic acid encoding a cytokine and/or a nucleic acid encoding a co-stimulatory molecule. In some aspects, the nucleic acids encoding co-stimulatory molecule and/or cytokine are delivered to the TILs intracellularly.

In some aspects, the nucleic acids are introduced into the TILs by passing the cell through a constriction such that transient pores are introduced to the membrane of the cell thereby allowing the nucleic acids to enter the cell Examples of constriction-based delivery of compounds into a cell are provided by WO 2013/059343, WO 2015/023982, WO 2016/070136, WO2017041050, WO2017008063, WO 2017/192785, WO 2017/192786, WO 2019/178005, WO 2019/178006, WO 2020/072833, WO2020154696, and WO2020176789, each of which is incorporated herein by reference in its entirety.

In some aspects, the nucleic acids are delivered into the TILs to produce the TILs of the disclosure by passing a cell suspension comprising the TILs through a constriction, wherein the constriction deforms the cells thereby causing a perturbation of the cells such that the nucleic acids enters the cells, wherein the nucleic acids are expressed. In some aspects, the constriction is contained within a microfluidic channel. In some aspects, multiple constrictions can be placed in parallel and/or in series within the microfluidic channel.

In some aspects, the constriction within the microfluidic channel includes an entrance portion, a center point, and an exit portion. In some aspects, the length, depth, and width of the constriction within the microfluidic channel can vary. In some aspects, the width of the constriction within the microfluidic channel is a function of the diameter of the TILs. Methods to determine the diameter of TILs are known in the art; for example, high-content imaging, cell counters or flow cytometry.

In some aspects, the length, depth, and/or width of the constriction can vary in some aspects, by modulating (e.g., increasing or decreasing) the length, depth, and/or width of the constriction, the delivery efficiency of a nucleic acid (e.g., encoding a cytokine, a protein that enhances persistence and/or tumor killing activity, and/or co-stimulatory molecule) can be regulated.

In some aspects of the constriction-based delivery of nucleic acids encoding co-stimulatory molecules or cytokines to TILs, the width of the constriction is about 2 μm to about 15 μm. In some aspects, the width of the constriction is about 3 μm to about 10 μm. In some aspects, the width of the constriction is about 3 μm to about 6 μm. In some aspects, the width of the constriction is about 4.2 μm to about 6 μm. In some aspects, the width of the constriction is about 4.2 μm to about 4.8 μm. In some aspects, the width of the constriction is about 3 μm to about 5 μm. In some aspects, the width of the constriction is about 3 μm to about 3.5 μm. In some aspects, the width of the constriction is about 3.5 μm to about 4 μm. In some aspects, the width of the constriction is about 4 μm to about 4.5 μm. In some aspects, the width of the constriction is about 3.2 μm to about 3.8 μm. In some aspects, the width of the constriction is about 3.8 μm to about 4.3 μm. In some aspects, the width of the constriction is about or less than any one of 2 μm, 25 μm, 3 μm, 3.5 μm, 4 μm, 4.5 μm, 5 μm, 55 μm, 6 μm, 65 μm, 7 μm, 7.5 μm, 8 μm, 8.5 μm, 9 μm, 9.5 μm, 10 μm, 10.5 μm, 11 μm, 11.5 μm, 12 μm, 12.5 μm, 13 μm, 13.5 μm, 14 μm, 14.5 μm or 15 μm. In some aspects, the width of the constriction is about or less than any one of 3.0 μm, 3.1 μm, 3.2 μm, 3.3 μm, 3.4 μm, 3.5 μm, 3.6 μm, 3.7 μm, 3.8 μm, 3.9 μm, 4.0 μm, 4.1 μm, 4.2 μm, 4.3 μm, 4.4 μm, 4.5 μm, 4.6 μm, 4.7 μm, 4.8 μm, 4.9 μm, or 5.0 μm. In some aspects, the width of the constriction is about 3.5 μm. In some aspects, the width of the constriction is about 4 μm. In some aspects, the width of the constriction is about 4.5 μm.

In some aspects of the constriction-based delivery of nucleic acids encoding co-stimulatory molecules or cytokines to TILs, the width of the constriction is about 3 μm to about 20 μm. In some aspects, the width of the constriction is about 5 μm to about 15 μm. In some aspects, the width of the constriction is about 8 μm to about 12 μm. In some aspects, the width of the constriction is about 9 μm to about 1 μm. In some aspects, the width of the constriction is about 9.5 μm to about 10.5 μm. In some aspects, the width of the constriction is about 7 μm to about 9 μm in some aspects, the width of the constriction is about 8 μm to about 10 μm. In some aspects, the width of the constriction is about 9 μm to about 11 μm. In some aspects, the width of the constriction is about 10 μm to about 12 μm. In some aspects, the width of the constriction is about 11 μm to about 13 μm. In some aspects, the width of the constriction is about 5 μm to about 12 μm, about 6 μm to about 12 μm, about 8 μm to about 11 μm, about 9 μm to about 11 μm, about 9.5 μm to about 10.5 μm, about 8 pin to about 15 μm, about 10 μm to about 15 μm, or about 12 μm to about 15 μm. In some aspects, the width of the constriction is about 9.7 μm to about 10.3 μm. In some aspects, the width of the constriction is about or less than any one of 3 μm, 3.5 μm, 4 μm, 4.5 μm, 5 μm, 5.5 μm, 6 μm, 6.5 μm, 7 μm, 7.5 μm, 8 μm, 8.5 μm, 9 μm, 9.5 μm, 10 μm, 10.5 μm, 11 μm, 11.5 μm, 12 μm, 12.5 μm, 13 μm, 13.5 μm, 14 μm, 14.5 μm, 15 μm, 15.5 μm, 16 μm, 16.5 μm, 17 μm, 17.5 μm, or 18 μm. In some aspects, the width of the constriction is about or less than any one of 9.0 μm, 9.1 μm, 9.2 μm, 9.3 μm, 9.4 μm, 9.5 μm, 9.6 μm, 9.7 μm, 9.8 μm, 99 pin, 100 μm, 10.1 μm, 10.2 μm, 10.3 μm, 10.4 μm, 10.5 μm, 10.6 μm, 10.7 μm, 10.8 μm, 10.9 μm, or 11.0 μm. In some aspects, the width of the constriction is about 10.0 μm.

In some aspects of the disclosure, the composition comprises a plurality of TILs. In sone aspects, the width of the constriction is about 10% to about 99% of the mean diameter of a subpopulation of TILs having the smallest diameter within the population of TILs. In some aspects, the width of the constriction is any one of about 10% to about 90%, about 10% to about 80%, about 10% to about 70%, about 20% to about 60%, about 40% to about 60%, about 30% to about 45%, about 50% to about 99%, about 50% to about 90%, about 50% to about 80%, about 50% to about 70%, about 60% to about 90%, about 60% to about 80%, or about 60% to about 70% of the mean diameter of a subpopulation of TILs having the smallest diameter within the population of TILs. In some aspects, the width of the constriction is any one of about 10% to about 20%, about 20% to about 30%, about 30% to about 40%, about 40% to about 50%, about 50% to about 60%, about 60% to about 70%, about 70% to about 80%, about 80% to about 90%, or about 90% to about 99% of the mean diameter of a subpopulation of TILs having the smallest diameter within the population of TILs. In some aspects, the width of the constriction is any one of about 10%, 15%, 20%, 25%, 30%, 35%, 40%, 45%, 50%, 55%, 60%, 65%, 70%, 75%, 80%, 85%, 90%, 95%, or 99% of the mean diameter of a subpopulation of TILs having the smallest diameter within the population of TILs.

In some aspects of the disclosure, the composition comprises a plurality of TILs. In some aspects, the width of the constriction is about 10% to about 99% of the mean diameter of a subpopulation of TILs having the largest diameter within the population of TILs. In some aspects, the width of the constriction is any one of about 10% to about 90%, about 10% to about 80%, about 10% to about 70%, about 20% to about 60%, about 40% to about 60%, about 30% to about 45%, about 15% to about 30%, about 15% to about 20%, about 20% to about 25%, about 25% to about 30%, about 20% to about 30%, about 30% to about 70%, or about 30% to about 60% of the mean diameter of a subpopulation of TILs having the largest diameter within the population of TILs. In some aspects, the width of the constriction is any one of about 5% to about 10%, about 10% to about 20%, about 20% to about 30%, about 30% to about 40%, about 40% to about 50%, about 50% to about 60%, about 60% to about 70%, about 70% to about 80%, about 80% to about 90%, or about 90% to about 99% of the mean diameter of a subpopulation of TILs having the largest diameter within the population of TILs. In some aspects, the width of the constriction is any one of about 5%, 10%, 15%, 20%, 25%, 30%, 35%, 40%, 45%, 50%, 55%, 60%, 65%, 70%, 75%, 80%, 85%, 90%, 95%, or 99% of the mean diameter of a subpopulation of TILs having the largest diameter within the population of TILs.

In some aspects, the length of the constriction is up to about 1000 μm. In some aspects, the length of the constriction is Lip to about 950 μm in some aspects, the length of the constriction is up to about 900 μm. In some aspects, the length of the constriction is up to about 850 μm. In some aspects, the length of the constriction is up to about 800 μm. In some aspects, the length of the constriction is up to about 750 μm. In some aspects, the length of the constriction is up to about 700 μm. In some aspects, the length of the constriction is up to about 650 μm. In some aspects, the length of the constriction is up to about 600 μm in some aspects, the length of the constriction is up to about 550 μm. In some aspects, the length of the constriction is up to about 500 μm. In some aspects, the length of the constriction is up to about 450 μm. In some aspects, the length of the constriction is up to about 400 μm. In some aspects, the length of the constriction is up to about 350 μm. In some aspects, the length of the constriction is up to about 300 μm. In some aspects, the length of the constriction is up to about 250 μm. In some aspects, the length of the constriction is up to about 200 μm. In some aspects, the length of the constriction is Lip to about 150 μm in some aspects, the length of the constriction is up to 100 μm. In some aspects, the length of the constriction is up to about 50 μm. In some aspects, the length is about 1 μm, about 5 μm, 10 μm, about 20 μm, about 30 μm, about 40 μm, about 50 μm, about 60 pin, about 70 μm, about 80 μm, about 90 μm, or about 100 μm. In some aspects, the length of the constriction is less than 1 μm. In some aspects, the length of the constriction is less than about 1 μm, less than about 5 μm, less than about 10 μm, less than about 20 μm, less than about 30 μm, less than about 40 μm, less than about 50 μm, less than about 60 μm, less than about 70 μm, less than about 80 μm, less than about 90 μm, or less than about 100 μm. In some aspects, the constriction has a length of about 10 μm. In some aspects, the constriction has a length of about 30 pin. In some aspects, the constriction has a length of about 70 μm. In some aspects, the constriction has a length of about 0 μm. For example, in some aspects, a microfluidic device (e.g., chip) useful for the present disclosure comprises a constriction that resembles two points of a diamond coming together, such that the length of the constriction is about 0 μm.

In some aspects, the depth of the constriction is at least about 1 μm. In some aspects, the depth of the constriction is at least about 1 μm, at least about 2 μm, at least about 3 μm, at least about 4 μm, at least about 5 μm, at least about 10 μm, at least about 20 μm, at least about 30 μm, at least about 40 μm, at least about 50 μm, at least about 60 μm, at least about 70 μm, at least about 80 μm, at least about 90 μm, at least about 100 μm, at least about 110 μm, or at least about 120 μm. In some aspects, the depth is between about 5 μm to about 90 μm. In some aspects, the depth is about 5 μm, about 10 μm, about 15 μm, about 20 μm, about 30 μm, about 40 μm, about 50 μm, about 60 pin, about 70 μm, about 80 μm, or about 90 μm. In some aspects, the depth of the constriction is about 70 μm. In some aspects, the depth of the constriction is about 20 μm.

In some aspects, a cell-deforming constriction that can be used in modifying the TILs (e.g., to increase the expression of one or more cytokines, one or more proteins that enhance persistence and/or tumor killing activity (e.g., anti-apoptotic factors, e.g., Bcl-2), and/or one or more co-stimulatory molecules) has a length of about 10 μm, a width of about 4 μm, and a depth of about 70 μm. In some aspects, the cell-deforming constriction has a length of about 10 μm, a width of about 4.5 μm, and a depth of about 70 μm.

A number of parameters can influence the delivery of a compound to TILs for stimulating an immune response by the methods described herein. In some aspects, the cell suspension is contacted with the compound before, concurrently, or after passing through the constriction. The TILs can pass through the constriction suspended in a solution that includes the compound to deliver, although the compound can be added to the cell suspension after the TILs pass through the constriction. In some aspects, the compound to be delivered is coated on the constriction.

Examples of parameters that can influence the delivery of the compound into the TILs include, but are not limited to, the dimensions of the constriction, the entrance angle of the constriction, the surface properties of the constrictions (e.g., roughness, chemical modification, hydrophilic, hydrophobic, etc.), the operating flow speeds (e.g., cell transit time through the constriction), the cell concentration, the concentration of the compound in the cell suspension, buffer in the cell suspension, and the amount of time that the TILs recover or incubate after passing through the constrictions can affect the passage of the delivered compound into the TILs Additional parameters influencing the delivery of the compound into the TILs can include the velocity of the TILs in the constriction, the shear rate in the constriction, the viscosity of the cell suspension, the velocity component that is perpendicular to flow velocity, and time in the constriction. In addition, multiple chips comprising channels in series and/or in parallel can impact delivery to TILs Multiple chips in parallel can be useful to enhance throughput. Such parameters can be designed to control delivery of the compound (e.g., one or more nucleic acids encoding a cytokine, one or more nucleic acids encoding a protein that enhances persistence and/or tumor killing activity, and/or one or more nucleic acids encoding one or more co-stimulatory molecule).

In some aspects, the cell concentration ranges from about 10 to at least about 10¹² cells/mL or any concentration or range of concentrations therebetween. In some aspects, the cell density is about 1×10³ cells/mL, about 1×10⁴ cells/mL, about 1×10⁵ cells/mL, about 1×10⁶ cells/mL, about 2×10⁶ cells/mL, about 3×10⁶ cells/mL, about 4×10⁶ cells/mL, about 5×10⁶ cells/mL, about 6×10⁶ cells/mL, about 7×10⁶ cells/mL, about 8×10⁶ cells/mL, about 9×10⁶ cells/mL, about 1×10⁷ cells/mL, about 2×10⁷ cells/mL, about 3×10⁷ cells/mL, about 4×10⁷ cells/mL, about 5×10⁷ cells/mL, about 6×10⁷ cells/mL, about 7×10⁷ cells/mL, about 8×10⁷ cells/mL, about 9×10⁷ cells/mL, about 1×10⁸ cells/mL, about 1.1×10⁸ cells/mL, about 1.2×10⁸ cells/mL, about 1.3×10⁸ cells/mL, about 1.4×10⁸ cells/mL, about 1.5×10⁸ cells/mL, about 2.0×10⁸ cells/mL, about 3.0×10⁸ cells/mL, about 4.0×10⁸ cells/mL, about 5.0×10⁸ cells/mL, about 6.0×10⁸ cells/mL, about 7.0×10⁸ cells/mL, about 8.0×10⁸ cells/mL, about 9.0×10⁸ cells/mL, about 1.0×10⁹ cells/mL, about 2.0×10⁹ cells/mL, about 3.0×10⁹ cells/mL, about 4×10⁹ cells/mL, or about 5×10⁹ cells/mL or more. In some aspects, the cell density is between about 6×10⁷ cells/mL and about 1.2×10⁸ cells/mL.

In some aspects, delivery compound concentrations can range from about 10 ng/mL to about 1 g/mL or any concentration or range of concentrations therebetween. In some aspects, delivery compound concentrations can range from about 1 pM to at least about 2 M or any concentration or range of concentrations therebetween.

In some aspects, the concentration of co-stimulatory molecules incubated with the TILs is between about 0.01 μM and about 10 mM. For example, in some aspects, the concentration of co-stimulatory molecules incubated with the TILs is any of less than about 0.01 μM, about 0.1 μM, about 1 μM, about 10 μM, about 100 μM, about 1 mM or about 10 mM. In some aspects, the concentration of co-stimulatory molecules incubated with the TILs is greater than about 10 mM. In some aspects, the concentration of co-stimulatory molecules incubated with the TILs is any of between about 0.01 μM and about 0.1 μM, between about 0.1 μM and about 1 μM, between about 1 μM and about 10 μM, between about 10 μM and about 100 μM, between about 100 μM and about 1 mM, or between 1 mM and about 10 mM. In some aspects, the concentration of co-stimulatory molecules incubated with the TILs is between about 0.1 μM and about 1 mM. In some aspects, the concentration of co-stimulatory molecules incubated with the TILs is between about 0.1 μM and about 10 μM. In some aspects, the concentration of co-stimulatory molecules thereof incubated with the TILs is 1 μM.

In some aspects, the concentration of nucleic acids encoding the cytokines and/or the co-stimulatory molecules incubated with TILs is between about 1 nM and about 1 mM. In some aspects, the concentration of nucleic acids encoding the cytokines and/or the co-stimulatory molecules incubated with TILs is less than about 0.1 nM, about 1 nM, about 0.01 μM, about 0.1 μM, about 1 μM, about 10 μM, about 100 μM, about 1 mM or about 10 mM. In some aspects, the concentration of nucleic acids encoding the cytokines and/or the co-stimulatory molecules incubated with TILs is greater than about 10 mM. In some aspects, the concentration of nucleic acids encoding the cytokines and/or the co-stimulatory molecules incubated with TILs is any of between about 0.1 nM to about 1 nM, about 1 nM to about 10 nM, about 10 nM to about 100 nM, about 0.1 μM and about 1 μM, between about 1 μM and about 10 μM, between about 10 μM and about 100 μM, between about 100 μM and about 1 mM, or between 1 mM and about 10 mM. In some aspects, the concentration of nucleic acids encoding the cytokines and/or the co-stimulatory molecules incubated with TILs is between about 10 nM and about 100 nM. In some aspects, the concentration of nucleic acids encoding the cytokines and/or the co-stimulatory molecules incubated with TILs is between about 1 nM and about 10 nM. In some aspects, the concentration of nucleic acids encoding the cytokines and/or the co-stimulatory molecules incubated with TILs is about 50 nM. In some aspects, the nucleic acid is an mRNA.

In some aspects, the TILs comprise the nucleic acids encoding the cytokines and/or the co-stimulatory molecules at a concentration between about 1 nM and about 1 mM. In some aspects, the TILs comprises nucleic acids encoding the cytokines and/or the co-stimulatory molecules at a concentration of any of less than about 0.1 nM, about 1 nM, about 0.01 μM, about 0.1 μM, about 1 μM, about 10 μM, about 100 μM, about 1 mM or about 10 mM. In some aspects, the TILs comprise the nucleic acids encoding the cytokines and/or the co-stimulatory molecules at a concentration of greater than about 10 mM. In some aspects, the TILs comprise the nucleic acid encoding the cytokines and/or the co-stimulatory molecules at a concentration of any of between about 0.1 nM to about 1 nM, about 1 nM to about 10 nM, about 10 nM to about 100 nM, about 0.1 μM and about 1 μM, between about 1 μM and about 10 μM, between about 10 μM and about 100 μM, between about 100 μM and about 1 mM, or between 1 mM and about 10 mM. In some aspects, the TILs comprise the nucleic acid encoding the cytokines and/or the co-stimulatory molecules at a concentration between about 10 nM and about 100 nM. In some aspects, the TILs comprise the nucleic acid encoding the cytokines and/or the co-stimulatory molecules at a concentration between about 1 nM and about 10 nM. In some aspects, the TILs comprise the nucleic acid encoding the cytokines and/or the co-stimulatory molecules at a concentration of about 50 nM. In some aspects, the nucleic acid is an mRNA.

Various methods can be utilized to drive the cells through the constrictions. For example, pressure can be applied by a pump on the entrance side (e.g., gas cylinder, or compressor), a vacuum can be applied by a vacuum pump on the exit side, capillary action can be applied through a tube, and/or the system can be gravity fed. Displacement based flow systems can also be used (e.g., syringe pump, peristaltic pump, manual syringe or pipette, pistons, etc.). In some aspects, the cells are passed through the constrictions by positive pressure. In some aspects, the cells are passed through the constrictions by constant pressure or variable pressure. In some aspects, pressure is applied using a syringe in some aspects, pressure is applied using a pump. In some aspects, the pump is a peristaltic pump or a diaphragm pump. In some aspects, pressure is applied using a vacuum. In some aspects, the cells are passed through the constrictions by g-force. In some aspects, the cells are passed through the constrictions by capillary pressure.

In some aspects, fluid flow directs the cells through the constrictions. In some aspects, the fluid flow is turbulent flow prior to the cells passing through the constriction. Turbulent flow is a fluid flow in which the velocity at a given point varies erratically in magnitude and direction. In some aspects, the fluid flow through the constriction is laminar flow. Laminar flow involves uninterrupted flow in a fluid near a solid boundary in which the direction of flow at every point remains constant. In some aspects, the fluid flow is turbulent flow after the cells pass through the constriction. The velocity at which the cells pass through the constrictions can be varied. In some aspects, the cells pass through the constrictions at a uniform cell speed. In some aspects, the cells pass through the constrictions at a fluctuating cell speed.

In some aspects, the pressure is about 1 psi, about 2 psi, about 3 psi, about 4 psi, about 5 psi, about 6 psi, about 7 psi, about 8 psi, about 9 psi, about 10 psi, about 15 psi, about 20 psi, about 25 psi, about 30 psi, about 35 psi, about 40 psi, about 50 psi, about 55 psi, about 60 psi, about 65 psi, about 70 psi, about 75 psi, about 80 psi, about 85 psi, about 90 psi, about 95 psi, about 100 psi, about 105 psi, about 110 psi, about 120 psi, about 130 psi, about 140 psi, about 150 psi, about 160 psi, about 170 psi, about 180 psi, about 190 psi, or about 200 psi or more. In some aspects, the pressure is between about 30 psi and about 110 psi. In some aspects, the pressure is about 30 psi. In some aspects, the pressure is about 60 psi.

### Systems and Kits

In some aspects, the disclosure provides a system comprising one or more of the constriction, an TIL suspension, nucleic acids encoding the cytokines and/or the co-stimulator molecules for use in the methods disclosed herein. The system can include any aspect described for the methods disclosed above, including microfluidic channels or a surface having pores to provide cell-deforming constrictions, cell suspensions, cell perturbations, delivery parameters, compounds, and/or applications etc. In some aspect, the cell-deforming constrictions are sized for delivery to TILs. In some aspects, the delivery parameters, such as operating flow speeds, cell and compound concentration, velocity of the cell in the constriction, and the composition of the cell suspension (e.g., osmolarity, salt concentration, serum content, cell concentration, pH, etc.) are optimized for enhancing the activity and/or proliferative capacity of TILs.

Also provided are kits or articles of manufacture for use in treating individuals with a cancer or an infection. In some aspects, the kit comprises a TIL comprising intracellularly nucleic acids encoding the cytokines and/or the co-stimulatory molecules. In some aspects, the kit comprises one or more of the constriction, a TIL suspension, nucleic acids encoding the cytokines, protein that enhances persistence and/or tumor killing activity, and/or the co-stimulatory molecules for use in generating modified TILs for use in treating an individual with cancer or infection. In some aspects, the kits comprise the compositions described herein (e.g. a microfluidic channel or surface containing pores, cell suspensions, and/or compounds) in suitable packaging. Suitable packaging materials are known in the art, and include, for example, vials (such as sealed vials), vessels, ampules, bottles, jars, flexible packaging (e.g., sealed Mylar or plastic bags), and the like. These articles of manufacture can further be sterilized and/or sealed.

The disclosure also provides kits comprising components of the methods described herein and can further comprise instructions for performing said methods treat an individual in need thereof and/or instructions for introducing cytokines and/or the co-stimulatory molecules into a TIL. The kits described herein can further include other materials, including other buffers, diluents, filters, needles, syringes, and package inserts with instructions for performing any methods described herein; e.g., instructions for treating an individual in need thereof or instructions for modifying TILs to contain intracellularly cytokines, proteins that enhance persistence and/or tumor killing activity (e.g., anti-apoptotic factors, e.g., Bcl-2), and/or the co-stimulatory molecules.

### Exemplary Embodiments

Embodiment 1. A method of modulating the activity, persistence, and/or proliferative capacity of tumor-infiltrating lymphocytes (TILs), wherein the TILs are modified to increase expression of one or more co-stimulatory molecules, anti-apoptotic factors, and/or one or more cytokines.

Embodiment 2. A method of modulating the activity and/or proliferative capacity of tumor-infiltrating lymphocytes (TILs), wherein the TILs are modified to increase expression of one or more of co-stimulatory molecules.

Embodiment 3. The method of embodiment I or 2, wherein the co-stimulatory molecule is B7-H2 (ICOSL), B7-1 (CD80), B7-2 (CD86), CD70, LIGHT, HVEM, CD40, 4-1BBL, OX40L, TL1A, GITRL, CD30L, TIM4, SLAM, CD48, CD58, CD155, or CD112.

Embodiment 4. The method of embodiment 1 or 2, wherein the co-stimulatory molecule is CD86.

Embodiment 5. A method of modulating the phenotype and/or proliferative capacity of TILs, wherein the TILs are modified to increase expression of one or more cytokines.

Embodiment 6. The method of any one of embodiments 1 and 3-5, wherein the TILs are modified to comprise a chimeric membrane-bound cytokine.

Embodiment 7. The method of embodiment 6, wherein the chimeric membrane-bound cytokine is a fusion protein comprising the cytokine and a transmembrane domain.

Embodiment 8. The method of embodiment 7, wherein the cytokine is joined to the transmembrane domain by a peptide linker.

Embodiment 9. The method of embodiment 8 wherein the peptide linker is (G4S)3 (SEQ ID NO: 3) or (EAAAK)3 (SEQ ID NO: 4).

Embodiment 10. The method of any one of embodiments 1 and 3-9, wherein the cytokine is a Type I cytokine.

Embodiment 11. The method of any one of embodiments I and 3-10, wherein the cytokine is IL-15, IL-12, IL-2, IL-7, IFN α, IFN β, or IL-21 or functional variant thereof.

Embodiment 12. The method of embodiment 11, wherein the cytokine is IL-2 or a functional variant thereof, IL-7 or a functional variant thereof, IL-15 or a functional variant thereof, and/or IL-12 or a functional variant thereof.

Embodiment 13. The method of any one of embodiments 7-12, wherein the chimeric membrane-bound cytokine comprises the amino acid sequence of any one of SEQ ID NOs 7-10 and 13.

Embodiment 14. The method of any one of embodiments 1-13, wherein the modified TILs comprise increased expression of one or more cytokines and/or one or more of co-stimulatory molecules, wherein the TILs are prepared by a process comprising:
a) passing a cell suspension comprising input TILs through a cell-deforming constriction, wherein a diameter of the constriction is a function of a diameter of the input TILs in the suspension, thereby causing perturbations of the input TILs large enough for one or more nucleic acids encoding one or more cytokines and/or one or more nucleic acids encoding one or more co-stimulatory molecules to pass through to form perturbed input TILs; and
b) incubating the perturbed input TILs with the one or more nucleic acids encoding one or more cytokines and/or one or more nucleic acids encoding one or more co-stimulatory molecules to allow the one or more nucleic acids encoding one or more cytokines and/or one or more nucleic acids encoding one or more co-stimulatory molecules to enter the perturbed input TILs; wherein the nucleic acids are expressed, thereby generating the modified TILs comprising the one or more cytokines and/or the one or more co-stimulatory molecules.

Embodiment 15. The method of any one of embodiments 1-13, wherein the modified TILs comprises increased expression of one or more cytokines and/or one or more of co-stimulatory molecules, wherein the TILs are prepared by a process comprising:
a) passing a cell suspension comprising input TILs through a cell-deforming constriction, wherein a diameter of the constriction is a function of a diameter of the input TILs in the suspension, thereby causing perturbations of the input TILs large enough for the one or more nucleic acids encoding one or more cytokines and/or the one or more co-stimulatory molecules to pass through to form perturbed input TILs; and
b) incubating the perturbed input TILs with the one or more nucleic acids encoding one or more cytokines and/or the one or more co-stimulatory molecules to enter the perturbed input TILs; wherein the nucleic acids are expressed thereby generating the modified TILs comprising the one or more cytokines and/or the one or more co-stimulatory molecules.

Embodiment 16. The method of embodiment 14 or 15, wherein the method comprises
(a) incubating the TILs with the nucleic acid encoding the chimeric membrane-bound cytokine before, during and/or after passing the cell suspension through the cell-deforming constriction
(b) incubating the TILs with the nucleic acid encoding the chimeric membrane-bound cytokine and the co-stimulatory molecule before, during and/or after passing the cell suspension through the cell-deforming constriction;
(c) incubating the TILs with the nucleic acid encoding the chimeric membrane-bound cytokine and the nucleic acid encoding the co-stimulatory molecule before, during and/or after passing the cell suspension through the cell-deforming constriction; or
(d) incubating the TILs with the nucleic acid encoding the chimeric membrane-bound cytokine and the nucleic acid encoding the one or more co-stimulatory molecules before, during and/or after passing the cell suspension through the cell-deforming constriction.

Embodiment 17. The method of embodiment 14 or 15, wherein the method comprises
(a) incubating the TILs with the nucleic acid encoding the chimeric membrane-bound cytokine before passing the cell suspension through the cell-deforming constriction
(b) incubating the TILs with the nucleic acid encoding the chimeric membrane-bound cytokine and the co-stimulatory molecule before passing the cell suspension through the cell-deforming constriction;
(c) incubating the TILs with the nucleic acid encoding the chimeric membrane-bound cytokine and the nucleic acid encoding the co-stimulatory molecule before passing the cell suspension through the cell-deforming constriction; or
(d) incubating the TILs with the nucleic acid encoding the chimeric membrane-bound cytokine and the nucleic acid encoding the one or more co-stimulatory molecules before passing the cell suspension through the cell-deforming constriction.

Embodiment 18. The method of any one of embodiments 14-17, wherein one or more of the nucleic acids is mRNA.

Embodiment 19. The method of any one of embodiments 1-18, wherein the modified TILs have increased expression of one or more of T-bet, EOMES, TCF1. CD127, CD103, CD45RO, CCR5, and CD62L compared to corresponding TILs that are not modified.

Embodiment 20. The method of any one of embodiments 1-18, wherein the modified TILs have increased expression of one or more of T-bet, EOMES, TCF1, CD127, CD103, CD45RO, CCR5, and CD62L when cultured in the absence of exogenous cytokines, as compared to corresponding TILs that are not modified.

Aspect 21. The method of embodiment 19 or 20, wherein the expression of one or more of T-bet, EOMES, TCF1, CD127, CD103, CD45RO, CCR5, and CD62L in the modified TILs is increased by more than about 1.2-fold, 1.5-fold, 1.8-fold, 2-fold, 3-fold, 4-fold, 5-fold, 8-fold, or more than 10-fold as compared to corresponding TILs that are not modified.

Embodiment 22. The method of any one of embodiments 1-21, wherein the modified TILs exhibit increased proliferation compared to corresponding TILs that are not modified.

Embodiment 23. The method of any one of embodiments 1-21, wherein the modified TILs exhibit increased proliferation when cultured in media not comprising exogenous cytokines, as compared to corresponding TILs that are not modified.

Embodiment 24. The method of any one of embodiments 20-23, wherein the exogenous cytokine is IL-2 and/or IL-12, optionally wherein the exogenous cytokine is IL-2.

Embodiment 25. A composition comprising modified TILs, wherein the TILs are modified to increase expression of one or more of co-stimulatory molecules and/or one or more cytokines.

Embodiment 26. A composition comprising modified TILs, wherein the TILs are modified to increase expression of one or more of co-stimulatory molecules.

Embodiment 27. The composition of embodiment 25 or 26, wherein the co-stimulatory molecule is B7-H2 (ICOSL), B7-1 (CD80), B7-2 (CD86), CD70, LIGHT, HVEM, CD40, 4-1BBL, OX40L, TL1A, GITRL, CD30L, TIM4, SLAM, CD48, CD58, CD155, or CD112.

Embodiment 28. The composition of embodiment 27, wherein the co-stimulatory molecule is CD86.

Embodiment 29. A composition comprising modified TILs, wherein the TILs are modified to increase expression of one or more cytokines.

Embodiment 30. The composition of any one of embodiments 25 and 27-29, wherein the TILs are modified to comprise a chimeric membrane-bound cytokine.

Embodiment 31. The composition of embodiment 30, wherein the chimeric membrane-bound cytokine is a fusion protein comprising the cytokine and a transmembrane domain.

Embodiment 32. The composition of embodiment 31, wherein the cytokine is joined to the transmembrane domain by a peptide linker.

Embodiment 33. The composition of embodiment 32, wherein the peptide linker is (G4S)3 (SEQ ID NO: 3) or (EAAAK)3 (SEQ ID NO: 4).

Embodiment 34. The composition of any one of embodiments 25 and 27-33, wherein the cytokine is a Type I cytokine.

Embodiment 35. The composition of any one of embodiments 25 and 27-34, wherein the cytokine is IL-15, IL-12, IL-2, IL-7, IFN α, IFN β, or IL-21 or functional variant thereof.

Embodiment 36. The method of embodiment 35, wherein the cytokine is IL-2 or a functional variant thereof, IL-7 or a functional variant thereof. IL-15 or a functional variant thereof, and/or IL-12 or a functional variant thereof.

Embodiment 37. The composition of any one of embodiments 30-36, wherein the chimeric membrane-bound cytokine comprises the amino acid sequence of any one of SEQ ID NOs: 7-10 and 13.

Embodiment 38. The composition of any one of embodiments 25-37, wherein the modified TILs comprise increased expression of one or more cytokines and/or one or more of co-stimulatory molecules, wherein the modified TILs are prepared by a process comprising:
a) passing a cell suspension comprising input TILs through a cell-deforming constriction, wherein a diameter of the constriction is a function of a diameter of the input TILs in the suspension, thereby causing perturbations of the input TILs large enough for one or more nucleic acids encoding one or more cytokines and/or one or more nucleic acids encoding one or more co-stimulatory molecules to pass through to form perturbed input TILs; and
b) incubating the perturbed input TILs with the one or more nucleic acids encoding one or more cytokines and/or one or more nucleic acids encoding one or more co-stimulatory molecules to allow the one or more nucleic acids encoding one or more cytokines and/or one or more nucleic acids encoding one or more co-stimulatory molecules to enter the perturbed input TILs; wherein the nucleic acids are expressed, thereby generating the modified TILs comprising the one or more cytokines and/or the one or more co-stimulatory molecules.

Embodiment 39. The composition of any one of embodiments 25-37, wherein the TILs comprises increased expression of one or more cytokines and/or one or more of co-stimulatory molecules, wherein the TILs are prepared by a process comprising:
a) passing a cell suspension comprising input TILs through a cell-deforming constriction, wherein a diameter of the constriction is a function of a diameter of the input TILs in the suspension, thereby causing perturbations of the input TILs large enough for the one or more nucleic acids encoding one or more cytokines and/or the one or more co-stimulatory molecules to pass through to form perturbed input TILs; and
b) incubating the perturbed input TILs with the one or more nucleic acids encoding one or more cytokines and/or the one or more co-stimulatory molecules to enter the perturbed input TILs, wherein the nucleic acids are expressed thereby generating the modified TILs comprising the one or more cytokines and/or the one or more co-stimulatory molecules.

Embodiment 40. The composition of embodiment 38 or 39, wherein the process of preparing the modified TILs comprises:
(a) incubating the TILs with the nucleic acid encoding the chimeric membrane-bound cytokine before, during and/or after passing the cell suspension through the cell-deforming constriction
(b) incubating the TILs with the nucleic acid encoding the chimeric membrane-bound cytokine and the co-stimulatory molecule before, during and/or after passing the cell suspension through the cell-deforming constriction;
(c) incubating the TILs with the nucleic acid encoding the chimeric membrane-bound cytokine and the nucleic acid encoding the co-stimulatory molecule before, during and/or after passing the cell suspension through the cell-deforming constriction; or
(d) incubating the TILs with the nucleic acid encoding the chimeric membrane-bound cytokine and the nucleic acid encoding the one or more co-stimulatory molecules before, during and/or after passing the cell suspension through the cell-deforming constriction.

Embodiment 41. The composition of any one of embodiment 38-39, wherein the process of preparing the modified TILs comprises:
(a) incubating the TILs with the nucleic acid encoding the chimeric membrane-bound cytokine before passing the cell suspension through the cell-deforming constriction
(b) incubating the TILs with the nucleic acid encoding the chimeric membrane-bound cytokine and the co-stimulatory molecule before passing the cell suspension through the cell-deforming constriction;
(c) incubating the TILs with the nucleic acid encoding the chimeric membrane-bound cytokine and the nucleic acid encoding the co-stimulatory molecule before passing the cell suspension through the cell-deforming constriction; or
(d) incubating the TILs with the nucleic acid encoding the chimeric membrane-bound cytokine and the nucleic acid encoding the one or more co-stimulatory molecules before passing the cell suspension through the cell-deforming constriction.

Embodiment 42. The composition of any one of embodiments 38-41, wherein one or more of the nucleic acids is mRNA.

Embodiment 43. The composition of any one of embodiments 2542, wherein the modified TILs have increased expression of one or more of T-bet, EOMES, TCF1, CD127, CD103, CD45RO, CCR5, and CD62L compared to corresponding TILs that are not modified.

Embodiment 44. The composition of any one of embodiments 25-43, wherein the modified TILs have increased expression of one or more of T-bet, EOMES, TCF1, CD127, CD103, CD45RO, CCR5, and CD62L when cultured in the absence of exogenous cytokines, compared to corresponding TILs that are not modified.

Embodiment 45. The composition of embodiment 43 or 44, wherein the expression of one or more of T-bet, EOMES, TCF1, CD127, CD103, CD45RO, CCR5, and CD62L in modified TILs is increased by more than about 1.2-fold, 1.5-fold, 1.8-fold, 2-fold, 3-fold, 4-fold, 5-fold, 8-fold, or more than 10-fold compared to the corresponding TILs that are not modified.

Embodiment 46. The composition of any one of embodiments 25-45, wherein the modified TILs exhibit increased proliferation compared to corresponding TILs that are not modified.

Embodiment 47. The composition of any one of embodiments 25-45, wherein the modified TILs exhibit increased proliferation when cultured in media not comprising exogenous cytokines, as compared to corresponding TILs that are not modified.

Embodiment 48. The composition of any one of embodiments 4447, wherein the exogenous cytokine is IL-2 and/or IL-12; optionally wherein the exogenous cytokine is IL-2.

Embodiment 49. A method of modulating an immune response in an individual, comprising administering the modified TILs to an individual, wherein the modified TILs are prepared according to the method of any one of embodiments 1-24.

Embodiment 50. A method of modulating an immune response in an individual, comprising administering the composition of any one of embodiments 25-48.

Embodiment 51. A method for treating a cancer, an infectious disease, or a viral-associated disease in an individual, comprising administering the modified TILs to an individual, wherein the modified TILs are prepared according to the method of any one of embodiments 1-24.

Embodiment 52. A method for treating a cancer, an infectious disease, or a viral-associated disease in an individual, comprising administering the composition of any one of embodiments 2548.

Embodiment 53. The method of any one of embodiments 49-52, wherein the method comprises multiple administration of the modified TILs, or multiple administration of the composition.

Embodiment 54. The method of any one of embodiments 49-53, wherein the modified TILs is administered intravenously or intratumorally.

Embodiment 55. The method of any one of embodiments 49-54, wherein the individual is a human.

Embodiment 56. The method of any one of embodiments 49-55, wherein the modified TILs are administered prior to, concurrently with, or following administration of another therapy.

Embodiment 57. A pharmaceutical composition for stimulating an immune response in an individual, wherein the composition comprises an effective amount of composition of any one of embodiments 25-48.

Embodiment 58. A pharmaceutical composition for use as a medicine, wherein the pharmaceutical composition comprises an effective amount of composition of any one of embodiments 25-48.

Embodiment 59. A pharmaceutical composition for treating a cancer, an infectious disease, or a viral-associated disease in an individual, wherein the pharmaceutical composition comprises an effective amount of composition of any one of embodiments 25-48.

Embodiment 60. The pharmaceutical composition of embodiment 58 or 59, wherein the composition comprising modified TILs is administered prior to, concurrently with, or following administration of another therapy.

Embodiment 61. The method of any one of embodiments 1-24 and 49-56 or the composition of any one of embodiments 25-48 and 57-60, wherein the width of the constriction is about 10% to about 99% of the mean diameter of the input TILs.

Embodiment 62. The method of any one of embodiments 1-24 and 49-56 or the composition of any one of embodiments 25-48, wherein the width of the constriction is about 3.5 μm to about 4.2 μm, or about 3.5 μm to about 4.8 pin, or about 3.5 μm to about 6 μm, or about 5 μm to about 12 μm, or about 12 μm to about 15 μm, or about 6 μm to about 12 μm, or about 8 μm to about 11 μm, or about 9 μm to about 11 μm.

Embodiment 63. The method of any one of embodiments 1-24 and 49-56 or the composition of any one of embodiments 25-48 and 57-60, wherein the width of the constriction is about 3 μm to about 5 μm.

Embodiment 64. The method of any one of embodiments 1-24 and 49-56 or the composition of any one of embodiments 25-48 and 57-60, wherein the width of the constriction is about 4 μm.

Embodiment 65. The method of any one of embodiments 1-24 and 49-56 or the composition of any one of embodiments 25-48 and 57-60, wherein the cell suspension comprising the plurality of input TILs are passed through multiple constrictions wherein the multiple constrictions are arranged in series and/or in parallel.

Embodiment 66. A kit for use in the method of any one of embodiments 1-24, 49-56 and 61-65.

Embodiment 67. A kit comprising the composition of any one of embodiments 25-48 and 57-65.

Embodiment 68. The kit of embodiment 66 or 67, wherein the kit further comprises one or more of buffers, diluents, filters, needles, syringes, or package inserts with instructions for administering the composition to an individual.

Embodiment 69. A method of producing TILs comprising a chimeric membrane-bound cytokine, the method comprising introducing a nucleic acid encoding the chimeric membrane-bound cytokine to the TILs.

Embodiment 70. The method of embodiment 69, wherein the TILs comprising the chimeric membrane-bound cytokine are prepared by:
a) passing a cell suspension comprising input TILs through a cell-deforming constriction, wherein a diameter of the constriction is a function of a diameter of the input TILs in the suspension, thereby causing perturbations of the input TILs large enough for a nucleic acid encoding the chimeric membrane-bound cytokine to pass through to form a perturbed input TILs; and
b) incubating the perturbed input TILs with the nucleic acid encoding the chimeric membrane-bound cytokine to allow the nucleic acid to enter the perturbed input TILs where the nucleic acid encoding the chimeric membrane-bound cytokine is expressed; thereby generating TILs comprising a chimeric membrane-bound cytokine.

Embodiment 71. The method of embodiment 70, wherein the method comprises incubating the TILs with the nucleic acid encoding the chimeric membrane-bound cytokine thereof before, during and/or after passing the cell suspension through the cell-deforming constriction.

Embodiment 72. The method of embodiment 70, wherein the method comprises incubating the TILs with the nucleic acid encoding the chimeric membrane-bound cytokine before passing the cell suspension through the cell-deforming constriction.

Embodiment 73. The method of any one of embodiments 69-72, wherein the nucleic acid encoding the chimeric membrane-bound cytokine is an mRNA encoding the chimeric membrane-bound cytokine.

Embodiment 74. The method of any one of embodiments 70-73, wherein the width of the constriction is about 10% to about 99% of the mean diameter of the input TILs.

Embodiment 75. The method of any one of embodiments 70-74, wherein the width of the constriction is about 5 μm to about 12 μm, or about 6 μm to about 12 μm, or about 8 μm to about 11 μm, or about 9 μm to about 11 μm, or about 12 μm to about 15 μm.

Embodiment 76. The method of any one of embodiments 70-75, wherein the width of the constriction is about 10 μm.

Embodiment 77. The method of any one of embodiments 70-76, wherein the width of the constriction is about 8 μm.

Embodiment 78. The method of any one of embodiments 70-77, wherein the cell suspension comprising the plurality of input TILs are passed through multiple constrictions wherein the multiple constrictions are arranged in series and/or in parallel.

Embodiment 79. Use of a pharmaceutical composition in the manufacture of a medicament for stimulating an immune response in an individual, wherein the pharmaceutical composition comprises an effective amount of composition of any one of embodiments 25-48.

Embodiment 80. Use of a pharmaceutical composition in the manufacture of a medicament for treating a cancer, an infectious disease, or a viral-associated disease in an individual, wherein the pharmaceutical composition comprises an effective amount of composition of any one of embodiments 25-48.

Embodiment 81. The use of embodiment 79 or 80, wherein the pharmaceutical composition is formulated for multiple administration.

Embodiment 82. The use of any one of embodiments 79-81, wherein the pharmaceutical composition is administered intravenously or intratumorally.

Embodiment 83. The use of any one of embodiments 79-82, wherein the individual is a human.

### EXAMPLES

Those skilled in the art will recognize that several aspects are possible within the scope and spirit of this disclosure. The disclosure will now be described in greater detail by reference to the following non-limiting examples. The following examples further illustrate the disclosure but, of course, should not be construed as in any way limiting its scope.

### Example 1: Tumor Infiltrating Lymphocytes (TILs) Express Membrane Bound Cytokines Over 48 Hours Subsequent to Constriction Mediated Delivery mRNAs Encoding Membrane-Bound Cytokines

This example shows the expression of membrane-bound IL-2 (e.g., mbIL-2) and/or membrane-bound IL-12 (e.g., mbIL-12) in tumor infiltrating lymphocytes (TILs) over 48 hours, subsequent to constriction mediated delivery of mbIL-2 and/or mbIL-12 mRNA.

### Methods

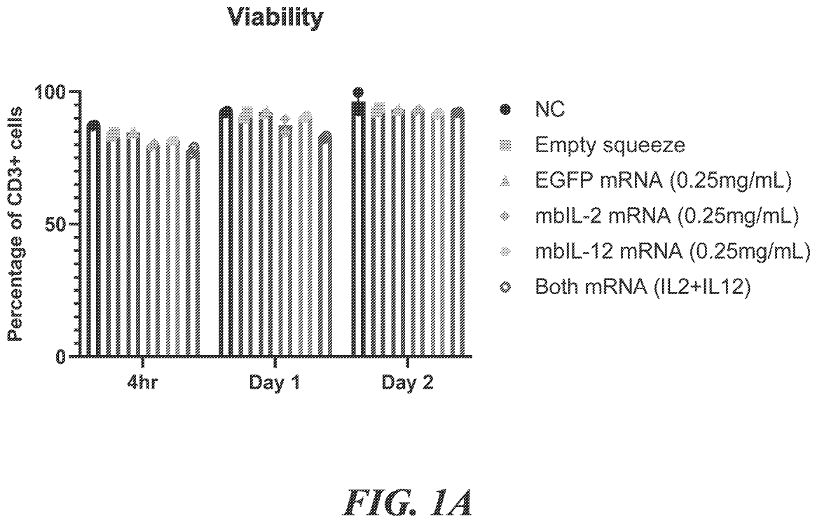

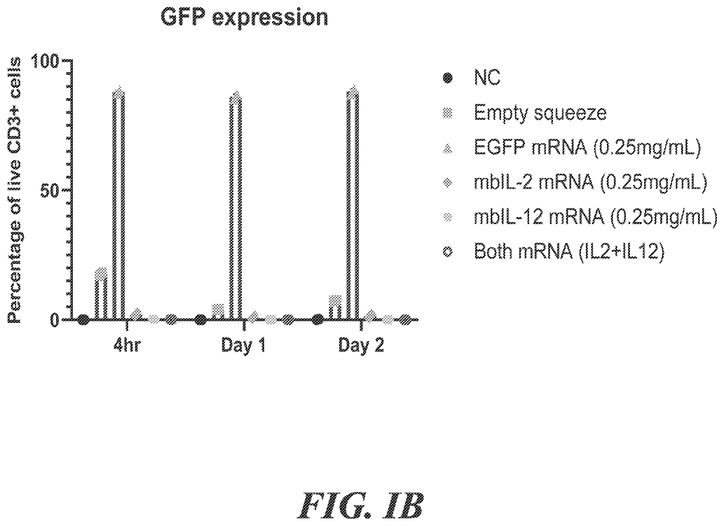

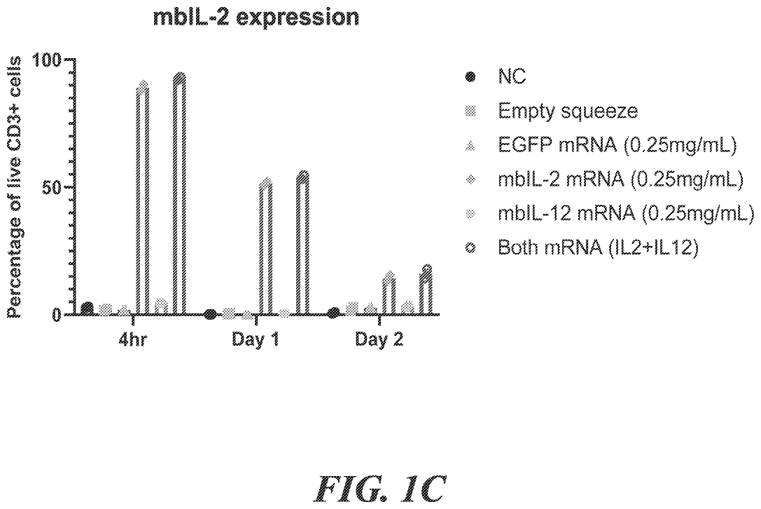

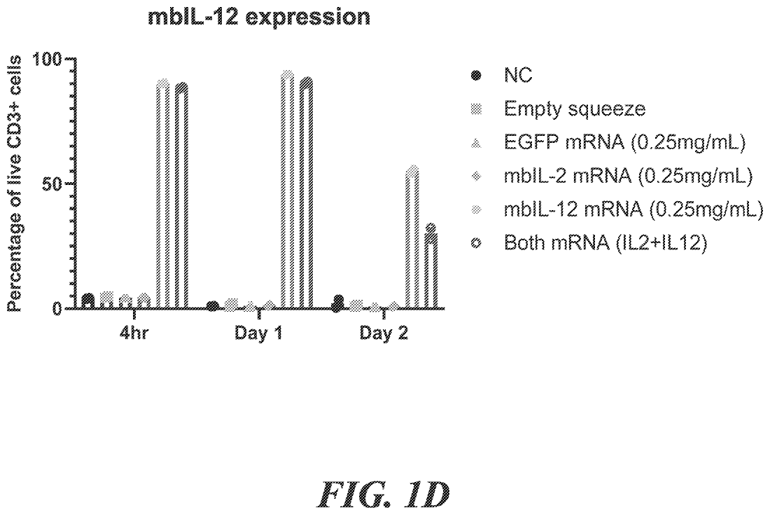

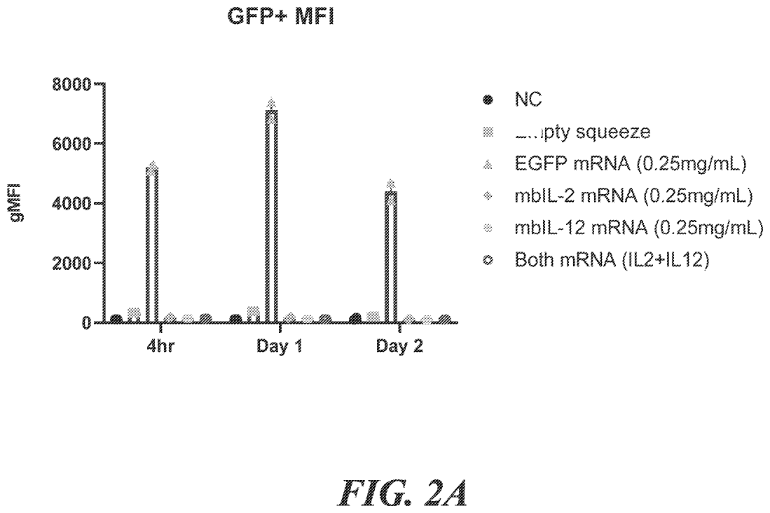

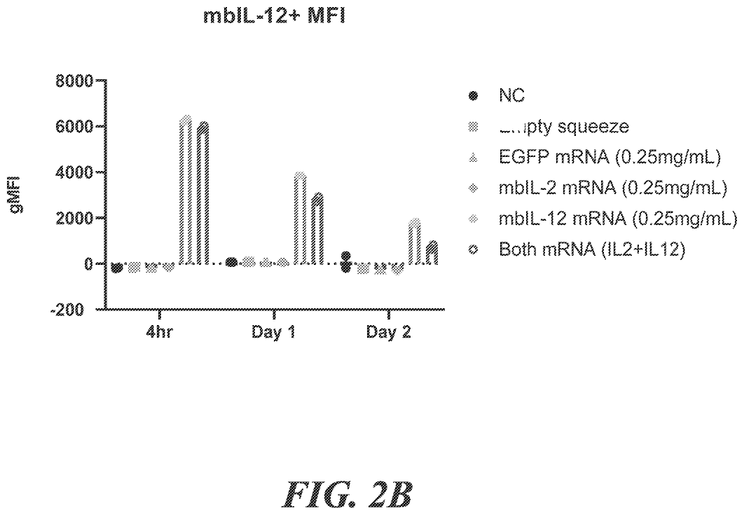

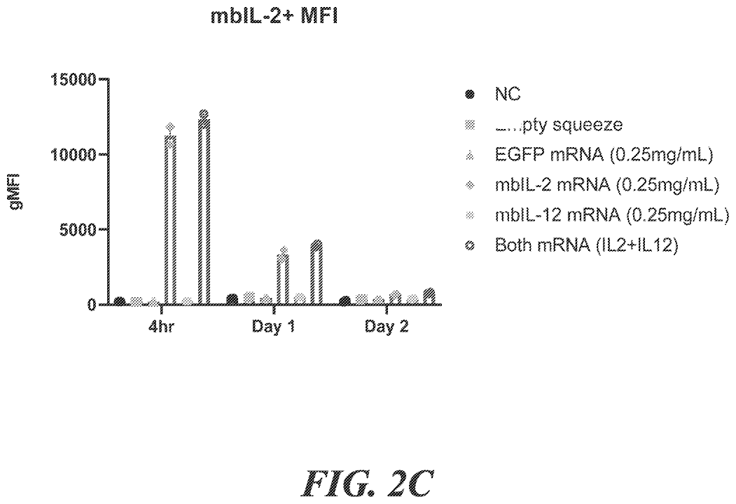

TILs were thawed and cultured in media comprising a high concentration of IL-2 (3000 IU/mL) for 24 hours. Following culture, mRNA encoding mbIL-2 (0.25 mg/mL), mRNA encoding mbIL-12 (0.25 mg/mL), or mRNAs encoding mbIL-2 and mbIL-12 (0.25 mg/mL each; total 0.5 mg/mL) were delivered to the TILs by constriction mediated delivery (“squeezing”). Specifically, TILs were squeeze-processed at 30 psi using a ST-10-040-70 chip (10 um length, 4 um width, 70 um depth) in the presence of the mRNAs as described Constriction mediated delivery was likewise performed with empty payload and GFP mRNA to serve as controls for downstream analysis. The TILs comprising the squeeze-delivered mRNAs were resuspended in culture media comprising a high concentration of IL-2 (3000 IU/mL). Fluorescence-activated cell sorting (FACS) was used to determine the expression of the membrane bound cytokines at 4 hours, 24 hours, and 48 hours following resuspension. In particular, cellular viability, percentage of cells expressing GFP mbIL-2 and/or mbIL-12, as well as mean fluorescence intensities (e.g., MFI) of GFP expression, mbIL-2 expression, and mbIL-12 expression, were evaluated (FIGS. 1A-ID and FIGS. 2A-2C). Table 1 provides an outline of the FACS protocols used to evaluate the TILs.

TABLE 1
Flow chart corresponding to the assays from Example 1.

| | Emission | | | | Antibody | Final |
|---|---|---|---|---|---|---|
| | (nm) | Channel | Fluorochromes | Target | Clone | Dilution |
| Violet | 440/50 | VL-1 | Pacific Blue | CD3 | SK7 | 1:200 |
| (405 nm) | | | | | | |
| Blue | 530/30 | BL-1 | GFP | mRNA | N/A | Delivery |
| (488 nm) | | | | Expression | | at 0.25 |
| | | | | | | mg/mL |
| Yellow | 585/16 | YL-1 | PE | mbIL12 | 20C2 | 1:200 |
| (561 nm) | | | | | | |
| Red | 670/14 | RL-1 | APC | mbIL2 | MQ1- | 1:200 |
| (638 nm) | | | | | 17H12 | |
| | 780/60 | RL-3 | Live/Dead near | Viability | | 1:1000 |
| | | | IR | | | |

### Results

As shown in FIG. 1A, viability was consistently between 80-90% at all measured time points post-squeezing (4 hours, Day 1, and Day 2). The viability of TILs increases overtime in high IL-2 culture media, as shown in the comparison of viability of TILs at 4 hours versus Day 2.

The GFP mRNA positive control indicated that mRNAs were effectively delivered and expressed in TILs using the constricted mediated delivery method (FIG. 1B and FIG. 2A). TILs successfully expressed mbIL-2 and mbIL-12, and both mbIL-2 and mbIL-12 simultaneously, when mRNA was delivered via a constriction mediated delivery method as indicated by both percentage of cells expression the cytokine, and MFI expression (see, e.g., FIGS. 1C-1) and FIGS. 2B-2C).

### Example 2: TILs Express Membrane Bound Cytokines Over 7 Days when Cultured with Varying IL-2 Concentrations Upon Constriction Mediated Delivery of mRNAs Encoding Membrane Bound Cytokines

This example shows the expression of membrane-bound IL-2 (e.g., mbIL-2) or membrane-bound IL-12 (e.g., mbIL-12) over 7 days when culture in the presence of varying IL-2 concentrations following constriction mediated delivery of mRNAs encoding mbIL-2 and mbIL-12 mRNA.

### Methods

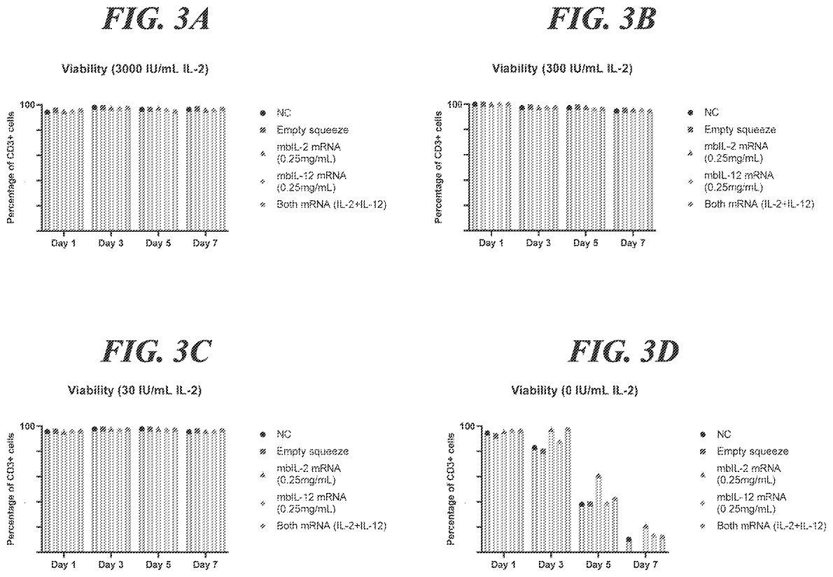

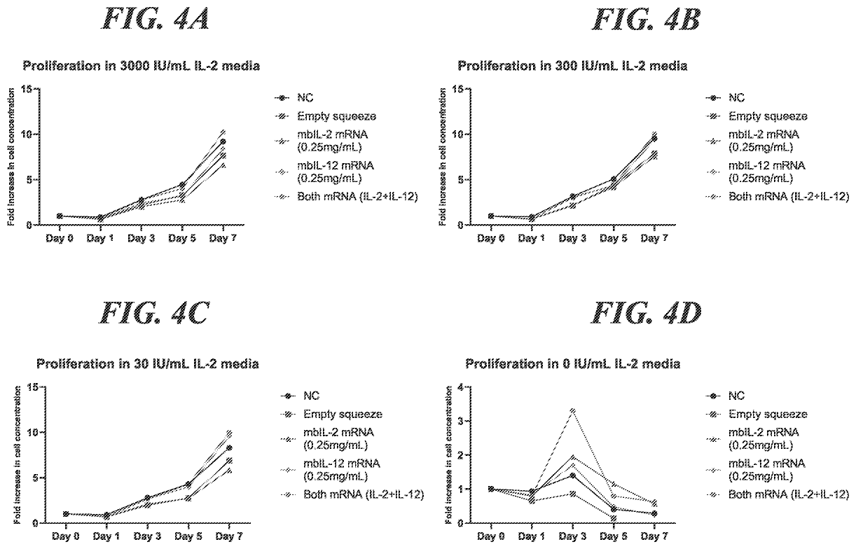

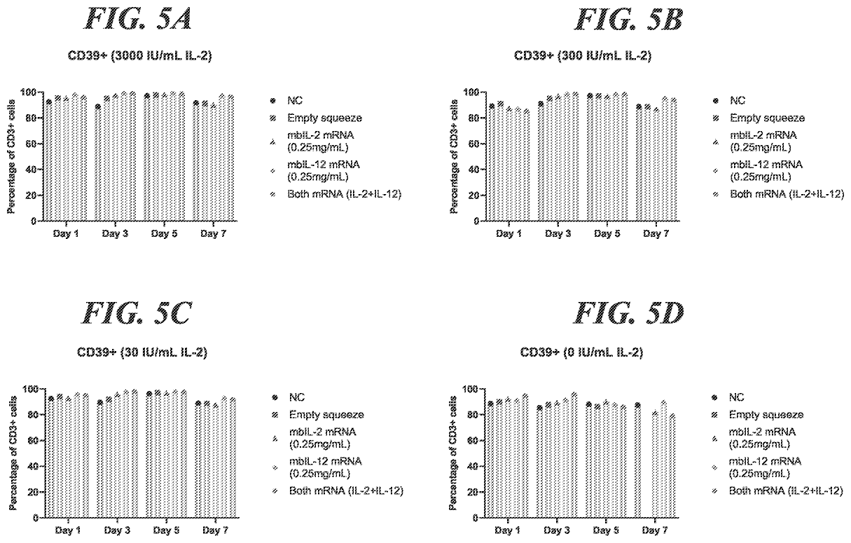

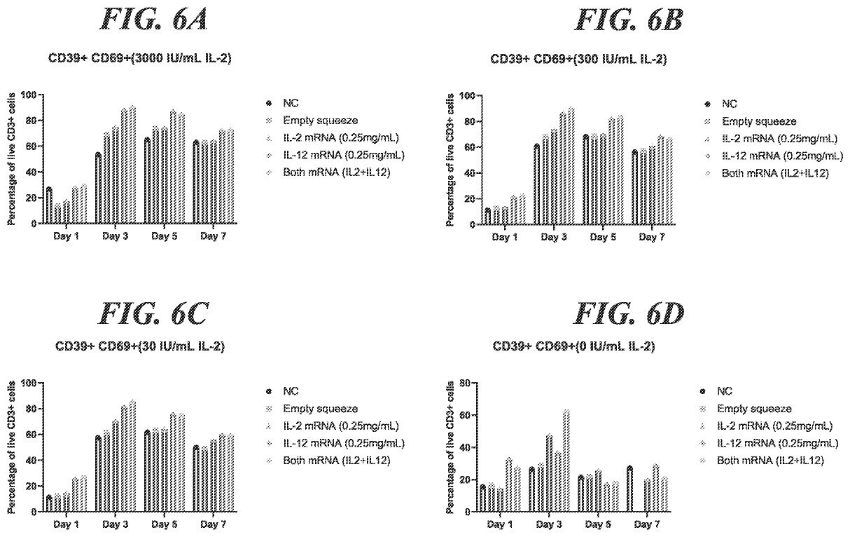

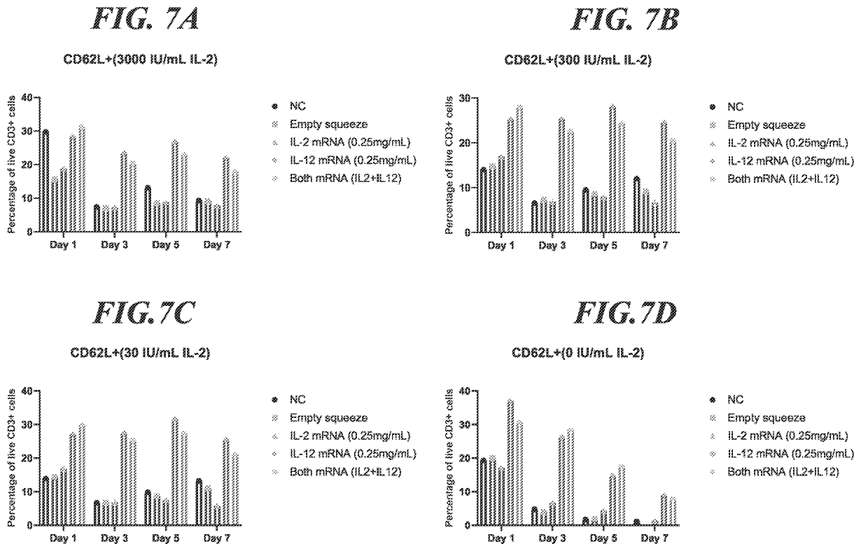

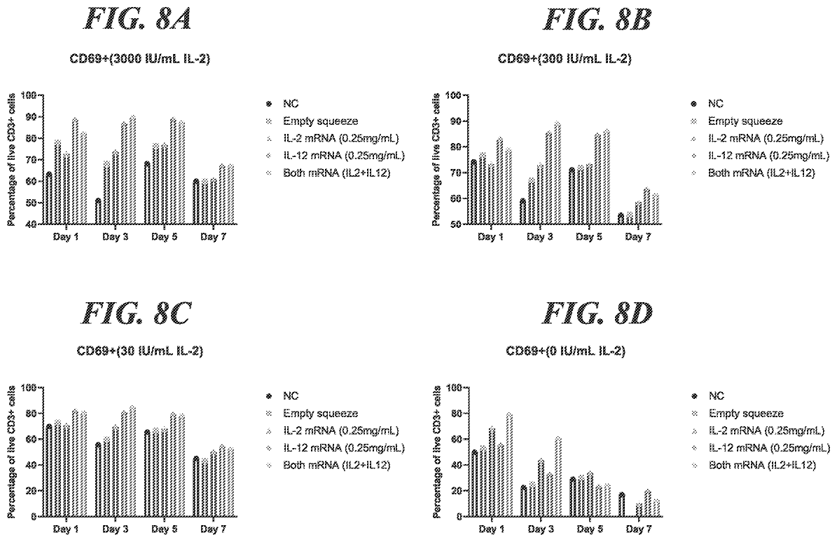

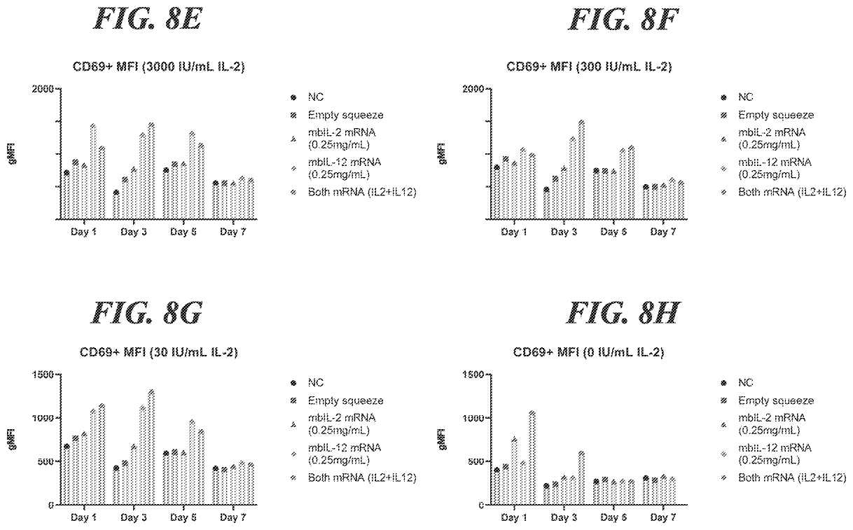

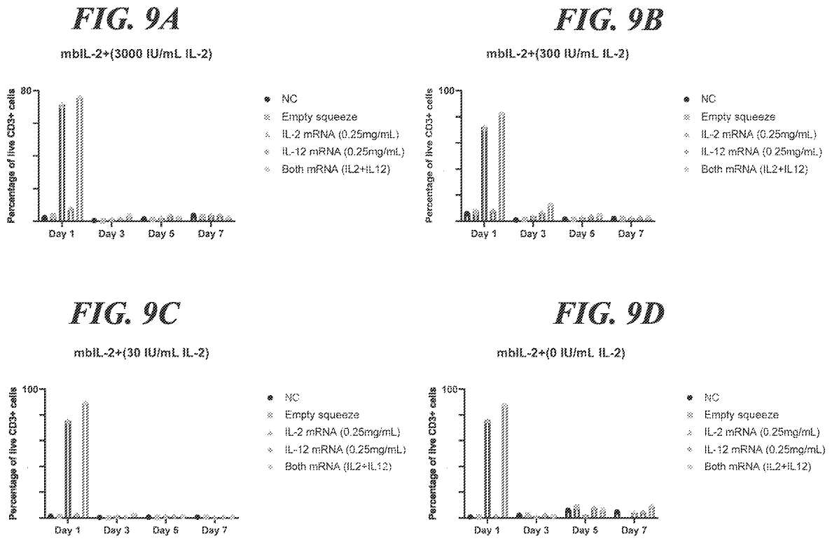

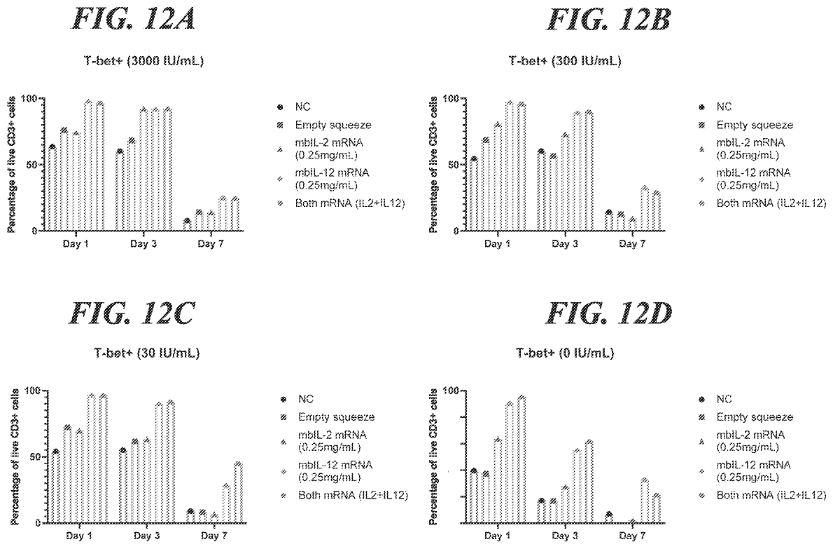

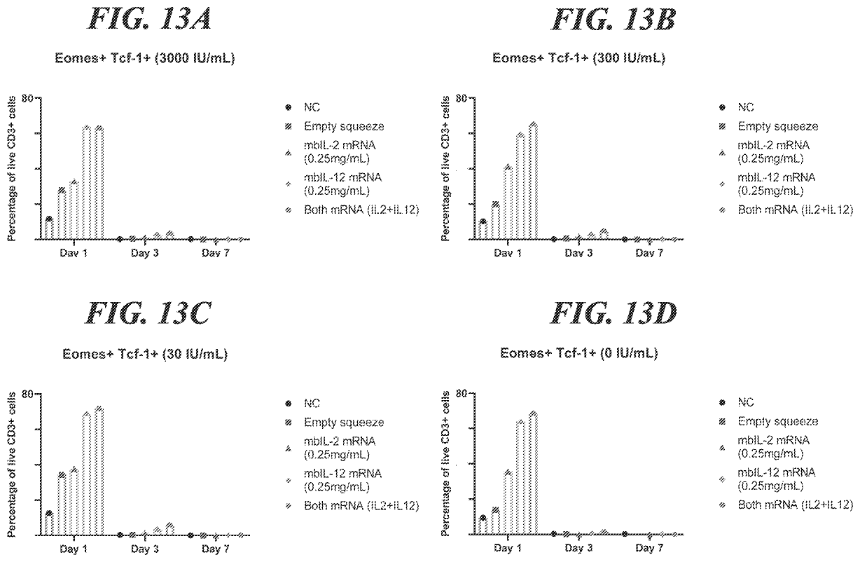

TILs were thawed and cultured in media comprising a high concentration of IL-2 (3000 IU/mL) for 24 hours. Following culture, mRNA encoding mbIL-2 (0.25 mg/mL), mbIL-12 (0.25 mg/mL), or both mbIL-2 and mbIL-12 (0.25 mg/mL) were delivered to the TILs by constriction mediated delivery. TILs squeeze-processed at 30 psi using a ST-10-040-70 chip (10 um length, 4 um width, 70 un depth) in the presence of the mRNAs as described. The TILs comprising the squeeze-delivered mRNAs were resuspended in culture media comprising the following concentrations of IL-2 for 7 total days: 3000 IU/mL, 300 IU/mL, 30 IU/mL, and 0 IU/mL. Fluorescence-activated cell sorting (FACS) was used to determine the expression of cytokines at 1 day, 3 days, and 7 days following resuspension. In particular, cellular viability, proliferation, percentage of cells expressing cytokines, and the mean fluorescence intensities (e.g., MFI) of the expression of mbIL-2 and mbIL-12 were evaluated. Viability of TILs following squeeze-delivery of mRNAs and incubation in the presence of IL-2 is shown in FIGS. 3A-3D Proliferation of TILs Viability of TILs following squeeze-delivery of mRNAs and incubation in the presence of IL-2 is shown in FIGS. 4A-4D. Expression of CD39 on TILs following squeeze-delivery of mRNAs and incubation in the presence of IL-2 is shown in FIGS. 5A-5D. Co-expression of CD39 and CD69 in TILs following squeeze-delivery of mRNAs and incubation in the presence of IL-2 is shown in FIGS. 6A-6D. Expression of CD62L on TILs following squeeze-delivery of mRNAs and incubation in the presence of IL-2 is shown in FIGS. 7A-7D. Expression of CD69 on TILs following squeeze-delivery of mRNAs and incubation in the presence of IL-2 is shown in FIGS. 8A-8H. FIGS. 9A-9D, 10A-10D and 11A-11D show the percentage of TILs expressing mbIL-2, or mbIL-12, or both mbIL-2 and mbIL-12, respectively. The percentage of TILs expressing T-bet following squeeze-delivery of mRNAs and incubation in the presence of IL-2 is shown in FIGS. 12A-12D. The percentage of TILs expressing Eomes and TCF-1 following squeeze-delivery of mRNAs and incubation in the presence of IL-2 is shown in FIGS. 13A-13D. Each assay was performed for each concentration of IL-2 in the culture media.

TABLE 2
Flow chart corresponding to the cell
surface stain assays from Example 2.

| | Emission | | | | Final |
|---|---|---|---|---|---|
| | (nm) | Channel | Fluorochromes | Target | Dilution |
| Violet | 440/50 | VL-1 | Pacific Blue | IL-12 | 1:200 |
| (405 nm) | 603/28 | VL-3 | BV605 | CD62L | 1:200 |
| Blue | 530/30 | BL-1 | FITC | CD39 | 1:200 |
| (488 nm) | | | | | |
| Yellow | 585/16 | YL-1 | PE | CD3 | 1:200 |
| (561 nm) | 780/60 | YL-4 | PE-Cy7 | CD69 | 1:200 |
| Red | 670/14 | RL-1 | APC | IL-2 | 1:200 |
| (638 nm) | 780/60 | RL-3 | NIR | Live/Dead | 1:1000 |

TABLE 3
Flow chart corresponding to the nuclear
stain assays from Example 2.

| | Emission | | | | Final |
|---|---|---|---|---|---|
| | (nm) | Channel | Fluorochromes | Target | Dilution |
| Violet | 440/50 | VL-1 | BV421 | T-bet | 1:200 |
| (405 nm) | | | | | |
| Blue | 530/30 | BL-1 | FITC | Eomes | 1:200 |
| (488 nm) | 695/40 | BL-3 | PerCP/Cy5.5 | CD3 | 1:200 |
| Yellow | 585/16 | YL-1 | PE | TCF-1 | 1:200 |
| (561 nm) | | | | | |
| Red | 780/60 | RL-3 | NIR | Live/Dead | 1:1000 |
| (638 nm) | | | | | |

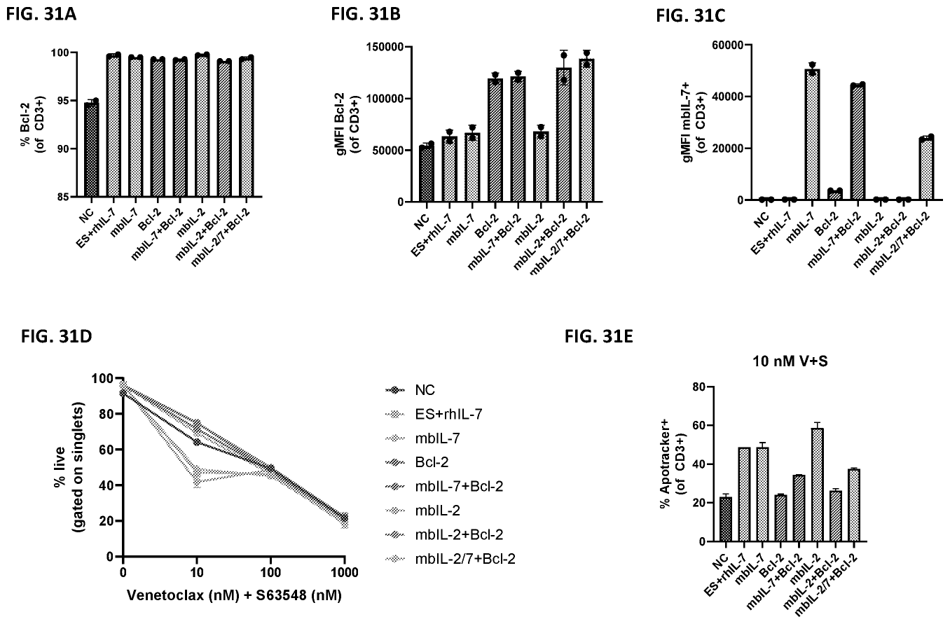

As shown in FIGS. 3A-3C, viability was consistently greater than about 85% for all cells tested in 3000 IU/mL, 300 IU/mL, and 30 IU/mL IL-2 culture media for all time points. The viability of TILs in media lacking IL-2 was greater than about 85% until day 3 (FIG. 31)). Proliferation increased for all TILs in media with 3000 IU/mL, 300 IU/mL, and 30 IU/mL IL-2 (FIGS. 4A-4C), and proliferation of TILs cultured in media lacking IL-2 was similar to that of corresponding TILs cultured in 3000 IU/mL IL-2 media up to day 3.

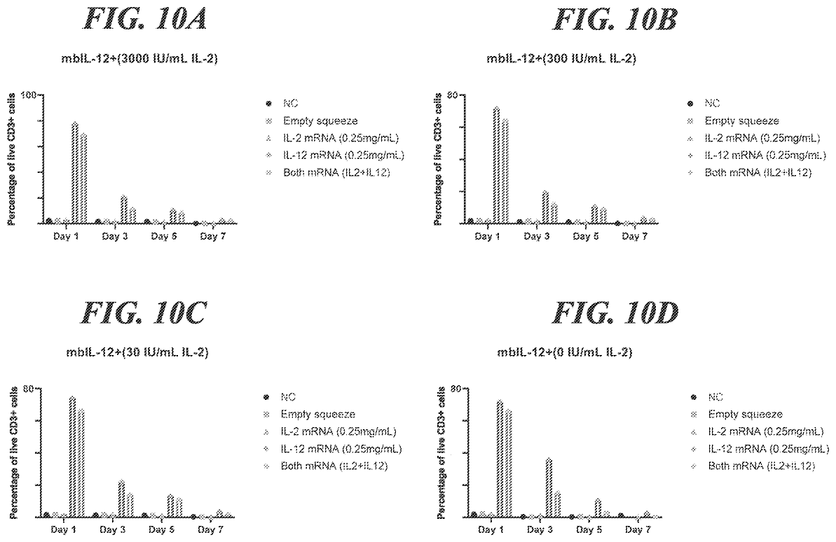

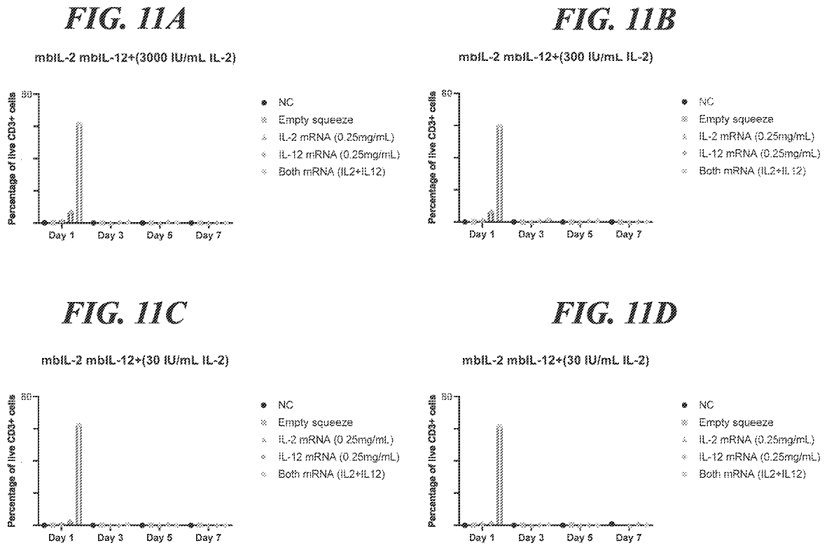

TILs successfully expressed mbIL-2, mbIL-12, or both mbIL-2 and mbIL-12 simultaneously upon squeeze mediated deliver of mRNA as indicated by percentage of cells expressing the respective cytokines (see, e.g., FIGS. 9A-9D, 10A-10D, and 11A-11D). As shown in FIGS. 9A-9D, expression of mbIL-2 was reduced over 2 days after squeeze delivery under each IL-2 media concentration. The mbIL-2 expression was highest on Day 1 following mRNA delivers, and negligible by Day 3 (FIGS. 9A-9D). Expression of mbIL-12 was similarly reduced over 2 days after squeeze-delivery at each IL-2 media concentration during incubation following squeeze-delivery of mRNAs (FIGS. 10A-10D) As shown in FIGS. 10A-10D, mbIL-12 expression was highest on Day 1 following squeeze delivery of mRNA and gradually decreased over time. Similarly, co-expression of mb-IL-2 and mbIL-12 was reduced over 2 days after squeeze-delivery at each IL-2 media concentration during incubation, following squeeze-delivery of the respective mRNAs (FIG. 11A-11D).

The phenotypes of TILs following squeeze delivery of mRNAs followed by incubation with IL-2 was also evaluated. FIGS. 5A-5D illustrate that expression of CD39 was similar across all TILs, regardless of IL-2 culture conditions. Expression of CD62L (central memory T-cells) was highest on Day 1 upon delivery of a combination of mbIL-2 and mbIL-12 mRNA delivery followed by incubation with IL-2 in the culture media (FIGS. 7A-7)). Expression of CD69 was similar on Day 1 and Day 7 in all TILs cultured in IL-2 media, but decreased by Day 3 (FIGS. 8A-8H). As shown in FIGS. 12A-12D, T-bet expression was enhanced with greater concentrations of IL-2 in the culture media in TILs expressing mbIL-2, mbIL-12, or both mbIL-2 and mbIL-12. TILs showed higher expression of Eomes and TCF-1 at Day 3 following squeeze mediated mRNA delivery (FIGS. 13A-13D).

### Example 3: TILs Express Membrane Bound Cytokines Over 3 Days when Cultured with Varying IL-2 Concentrations Upon Constriction Mediated Delivery of mRNAs Encoding mbIL-2, mbIL-12, and CD86

This example shows expression of mbIL-2 and mbIL-12 by TILs upon squeeze-mediated deliver of mRNA encoding mbIL-2 and mbIL-12 or CD86 and their impact on TIL phenotypes.

### Methods

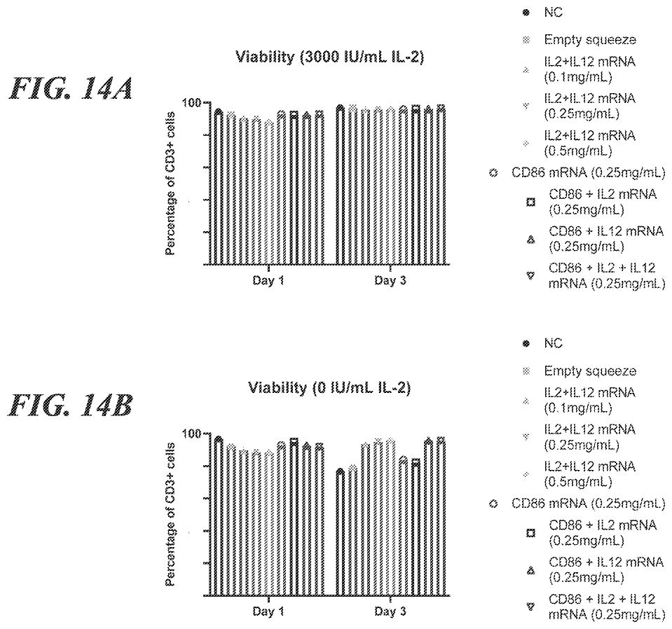

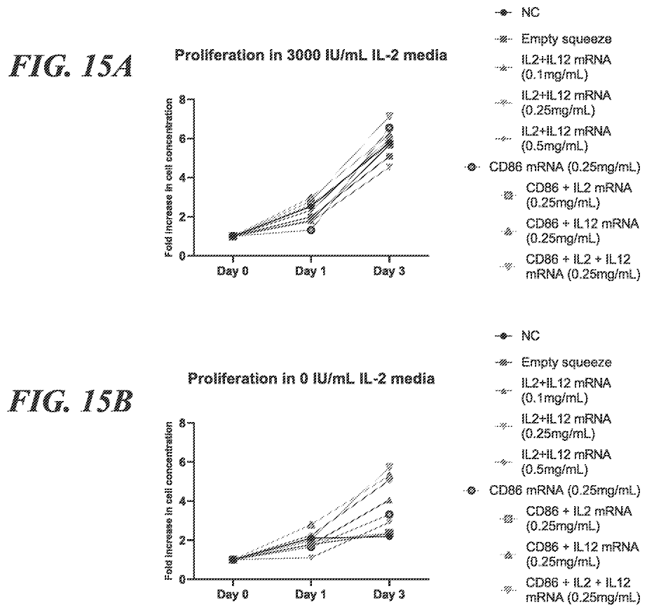

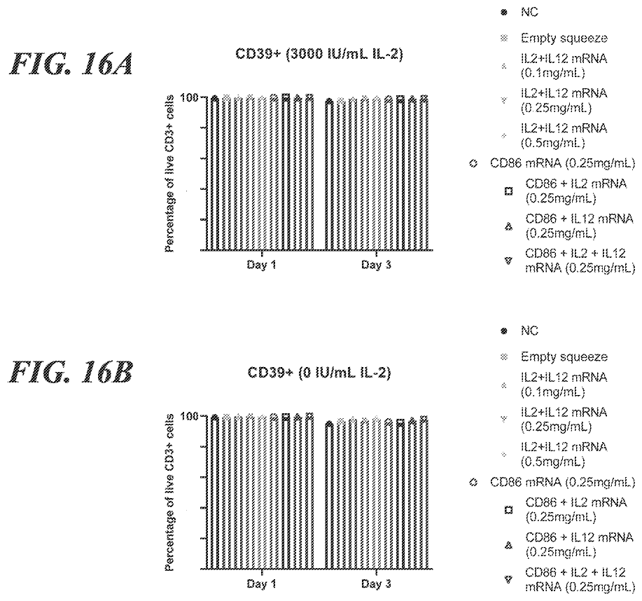

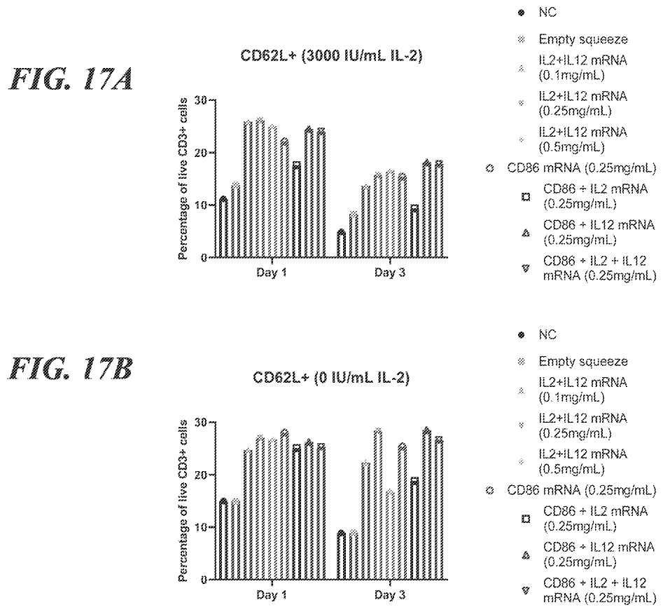

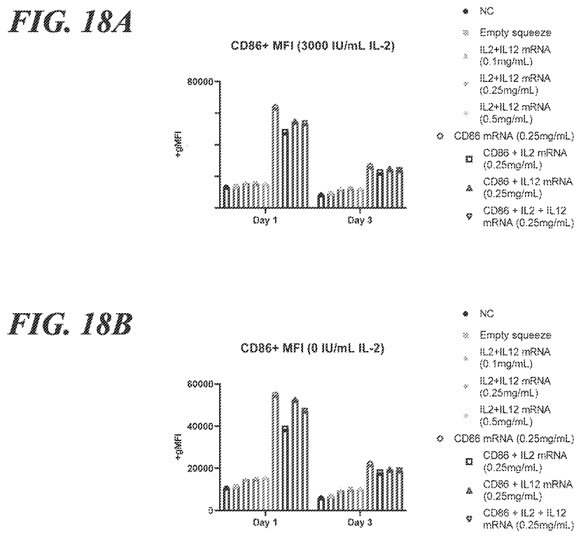

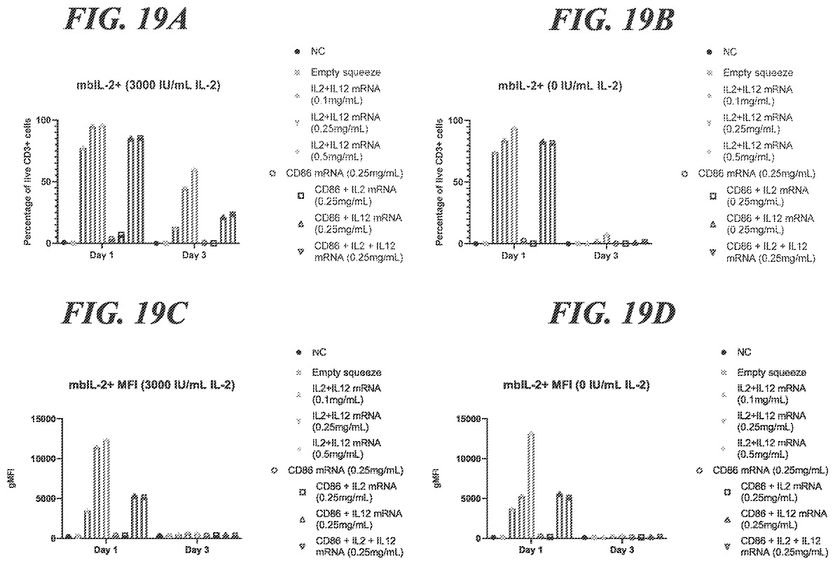

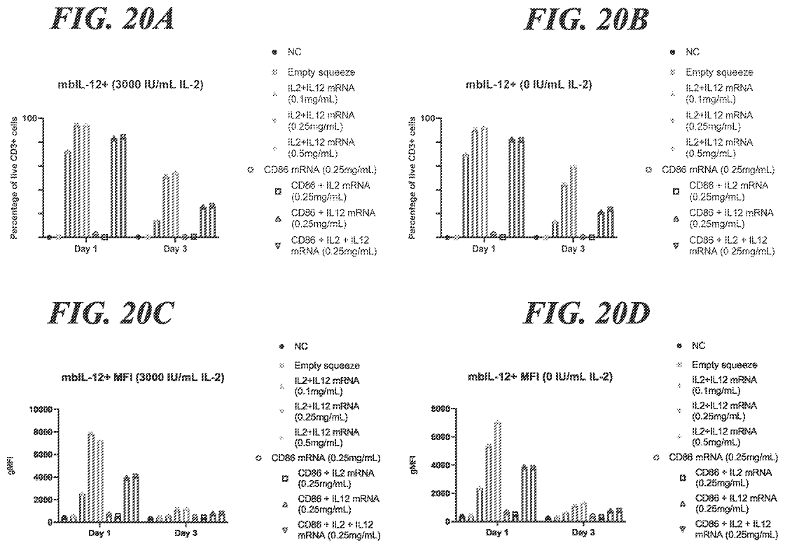

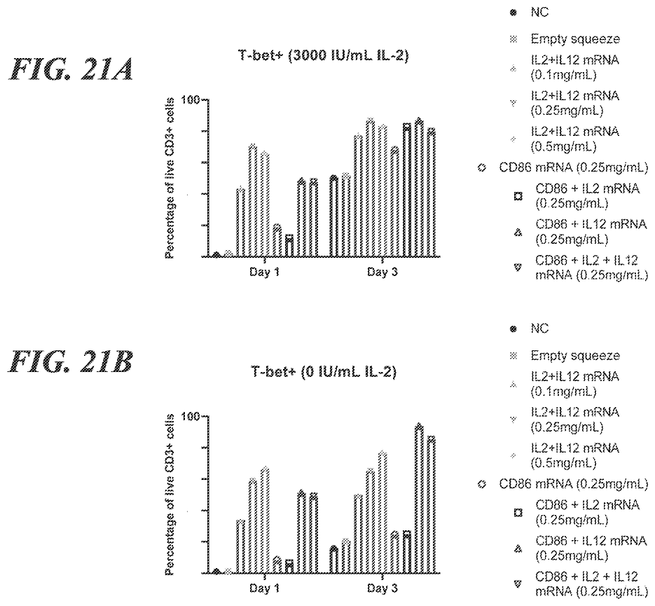

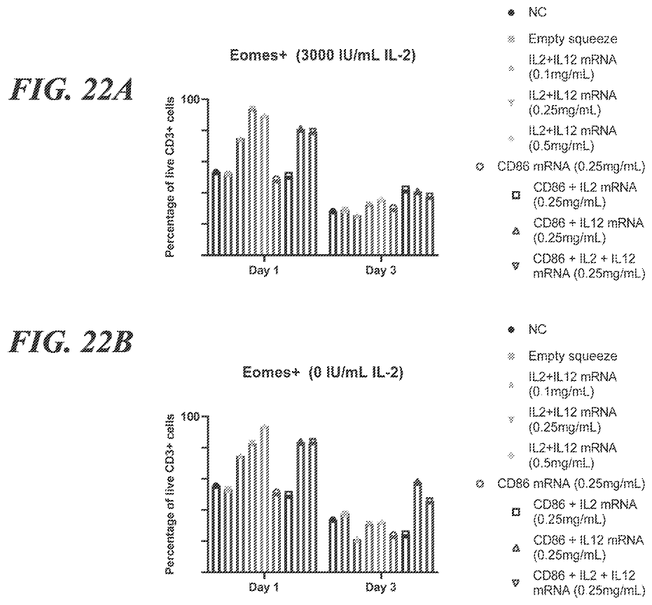

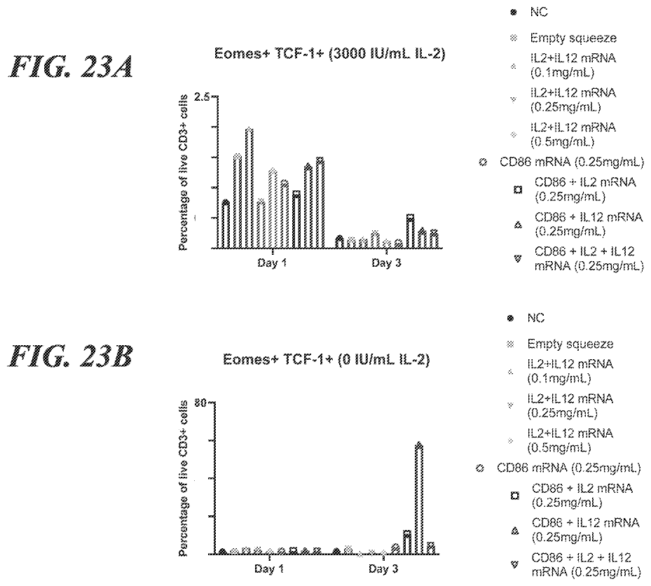

TILs were thawed and cultured in media comprising a high concentration of IL-2 (3000 IU/mL) for 24 hours Following culture, mRNAs encoding mbIL-2 and mbIL-12 (0.1 mg/mL), mbIL-2 and mbIL-12 (0.25 mg/mL), mbIL-2 and mbIL-12 (0.5 mg/mL), CD86 and mbIL-2 (0.25 mg/mL), CD86 and mbIL-12 (0.25 mg/mL), or CD86 and mbIL-2 and mbIL-12 (0.25 mg/mL each) were delivered to the TILs by squeeze-mediated processing at 30 psi using a ST-10-040-70 chip (10 um length, 4 um width, 70 um depth). The TILs comprising the constriction mediated delivered mRNAs were resuspended in culture media comprising 3000 IU/mL IL-2 or lacking IL-2 for 3 days. Fluorescence-activated cell sorting (FACS) was used to determine the expression of cytokines at 1 day and 3 days following resuspension. Viability of TILs following squeeze-delivery of mRNAs and incubation in the presence of IL-2 is shown in FIGS. 14A and 14B. Proliferation of TILs Viability of TILs following squeeze-delivery of mRNAs and incubation in the presence of IL-2 is shown in FIGS. 15A and 15B. Expression of CD39 on TILs following squeeze-delivery of mRNAs and incubation in the presence of IL-2 is shown in FIGS. 16A and 16B. Expression of CD62L on TILs following squeeze-delivery of mRNAs and incubation in the presence of IL-2 is shown in FIGS. 17A and 17B. FIGS. 18A and 18B show the mean fluorescent intensities of CD86. The percentage of TILs expressing mbIL-2 following squeeze-delivery of mRNAs and incubation in the presence of IL-2 is shown in FIGS. 19A-19D. The percentage of TILs expressing mbIL-12 following squeeze-delivery of mRNAs and incubation in the presence of IL-2 is shown in FIGS. 20A-20D. The percentage of TILs expressing T-bet following squeeze-delivery of mRNAs and incubation in the presence of IL-2 is shown in FIGS. 21A and 21B. The percentage of TILs expressing Eomes following squeeze-delivery of mRNAs and incubation in the presence of IL-2 is shown in FIGS. 22A and 22B. The percentage of TILs expressing Eomes+TCF-1 following squeeze-delivery of mRNAs and incubation in the presence of IL-2 is shown in FIGS. 23A and 23B.

Tables 4 and 5 provide an outline of the FACS protocols used to evaluate the surface and nuclear components of the TILs.

TABLE 4
Flow chart corresponding to the cell
surface stain assays from Example 3.

| | Emission | | | | Final |
|---|---|---|---|---|---|
| | (nm) | Channel | Fluorochromes | Target | Dilution |
| Violet | 440/50 | VL-1 | Pacific Blue | IL-12 | 1:200 |
| (405 nm) | 603/28 | VL-3 | BV605 | CD62L | 1:200 |
| Blue | 530/30 | BL-1 | FITC | CD39 | 1:200 |
| (488 nm) | | | | | |
| Yellow | 585/16 | YL-1 | PE | CD3 | 1:200 |
| (561 nm) | 780/60 | YL-4 | PE-Cy7 | CD86 | 1:200 |
| Red | 720/30 | RL-1 | APC | IL-2 | 1:200 |
| (638 nm) | 780/60 | RL-3 | NIR | Live/Dead | 1:1000 |

TABLE 5
Flow chart corresponding to the nuclear
stain assays from Example 3.

| | Emission | | | | Final |
|---|---|---|---|---|---|
| | (nm) | Channel | Fluorochromes | Target | Dilution |
| Violet | 440/50 | VL-1 | BV421 | T-bet | 1:200 |
| (405 nm) | | | | | |
| Blue | 695/40 | BL-3 | PerCP/Cy5.5 | CD3 | 1:200 |
| (488 nm) | | | | | |
| Yellow | 585/16 | YL-1 | PE | TCF-1 | 1:200 |
| (561 nm) | | | | | |
| | 670/14 | RL-1 | APC | Eomes | 1:200 |
| Red | 780/60 | RL-3 | NIR | Live/Dead | 1:1000 |
| (638 nm) | | | | | |

### Results

As shown in FIGS. 14A-14B, viability was consistently greater than about 85% in all replicates (e.g., from Day 1 and Day 3) for all cells tested in 3000 IU/mL. The viability of TILs in media lacking IL-2 was greater than about 85% until Day 3 (FIG. 14B). TILs in which mRNA encoding mbIL-2 and mbIL-12 or CD86 mRNA show increased viability in 0 IU/mL IL-2 media. Proliferation increased for TILs in which mRNA encoding mbIL-2 and mbIL-12, and/or CD86 mRNA were delivered using squeeze-mediated delivery (FIGS. 15A-15B).

TILs successfully expressed mbIL-2 and mbIL-12, and CD86, when mRNA was delivered via squeeze-mediated processing as indicated by both percentage of cells expressing the cytokines, and MFI expression (see, e.g., FIGS. 19A-19D and 20A-20D). As shown in FIGS. 19A-19D and 20A-20D, a higher concentration of mRNA encoding mbIL-2 and/or mbIL-12 mRNA (at 0.5 mg/mL) via squeezed-mediated processing showed comparable percentages of cells expressing the respective cytokines, and showed an overall increase in mbIL-2 and/or mbIL-12 expression, respectively, over 3 Days relative to squeeze-mediated processing of 0.25 mg/mL of mRNA encoding mbIL-2 and/or mbIL-12.

The phenotype of TILs receiving mRNAs by squeeze-mediated processing was also evaluated. FIGS. 16A-16B illustrate that CD39 was similar across all TILs, regardless of IL-2 culture conditions. CD62L (central memory T-cells) expression was improved in the presence of mbIL-12 mRNA (FIGS. 17A-17B). CD86 MFI was similarly improved in the presence of mbIL-12 mRNA in TILs (FIGS. 18A-18B). Eomes+ and TCF-1+ expression was enhanced in TILs that received mRNAs with greater concentrations of IL-2 in the culture media (FIGS. 23A-23B).

### Example 4: mbIL-2 and mbIL-12 Engineered TIL Expression Kinetics and Proliferation

To assess the expression kinetics of certain payloads described herein and to determine whether membrane-bound (mb) IL-2 and LL-12 can support viability and proliferation of TILs in the absence of exogenous cytokine support, post-REP TILs were loaded with mRNA encoding mbIL-2 and/or mbIL-12 using the squeeze processing methods provided herein. The specific methods used are provided below

### Squeeze Processing

Three 50M (i.e., 50×10⁶) cell vials were thawed and diluted with FACs buffer. Cells were centrifuged at 400 rcf at room temperature then washed and resuspended to 3-10M/mL in AIM V media containing 3000 IU/mL recombinant human (rh) IL-2. Cells were transferred to a T75 flask and cultured at 37° C. To allow the cells to recover from thaw, cells were cultured for 24 hr.

The cells were prepared for squeeze-processing. The cells were counted, filtered through a 40 um cell strainer, washed, and the concentration was adjusted to 20-50M/mL by adding XVivo-15. The following buffer solutions were prepared for each sample as shown in Table 6:

TABLE 6
Squeeze Processing Cell Suspension Solutions

| | |
|---|---|
| Group A | 100 μL XVivo-15 + 100 μL 2X TILs |
| Group B | 100 μL XVivo-15 + 100 μL 2X TILs |
| Group C | 100 μL mRNA mix (74.23 μL RPMI + 25 μL |
| | mbIL-2 mRNA + 0.77 μL NaCl) + 100 μL |
| | 2X TILs |
| Group D | 100 μL mRNA mix (48.46 μL RPMI + 25 μL |
| | mbIL-2 mRNA + 25 μL mbIL-12 mRNA + |
| | 1.54 μL NaCl) + 100 μL 2X TILs |

As shown above in Table 6, Group A was used as a control (i.e., not contacted with any payload and not squeeze processed) Group B was squeeze processed without any payload at room temperature using a microfluidic constriction (70 μm depth, 4.5 m width, and 10 μm length) at 30 psi. Groups C-D were squeeze processed at room temperature with the payloads shown above, i.e., Group C: mbIL-2 mRNA; Group D: mbIL-2 mRNA+mbIL-12 mRNA. Groups C-D were squeeze processed using the same conditions as in Group B (i.e., microfluidic constriction: 70 μm depth, 4.5 μm width, and 10 μm length; at 30 psi).

Afterwards, the cells from each of Groups A-D were separately transferred into their own 5 ml conical containing 1 mL RPMI+10% FBS. Cells from all groups were centrifuged separately, at 400 rcf for 4 minutes at room temperature. The supernatant was aspirated, and the cell populations were separately resuspended in 1 mL of RPMI+10% FBS media and cultured in technical duplicate with or without 3000 IU/mL rhIL-2 for 72 hours at 37° C.

### FACS Analysis

At 4, 24, 48 and 72 hrs, 200 ul of cells were aspirated and centrifuged at 400 rcf for 4 minutes at room temperature. The supernatant was discarded, and the cells were resuspended in 50 μL of L/D (1:200 dilution in FACs buffer) and FC block (1:50 dilution in FACs buffer). Cells were incubated at room temperature in the dark for 10 minutes. The cells were spun down at room temperature at 400 ref for 4 minutes. The cells were then incubated at 4° C. for 20 minutes with fluorescent anti-human antibodies targeting CD3, CD4, CD8, CD45RO, CD62L, IL-2, and IL-12p70. After incubation 150 μL of FACs isolation buffer were added to each sample and the samples were spun at 400 ref for 4 minutes. The cells were spun down at room temperature at 400 rcf for 4 minutes. The supernatant was discarded, and the cells resuspended with 200 μL of FACs isolation buffer. The cells were then analyzed using an Attune Flow cytometer.

### Results

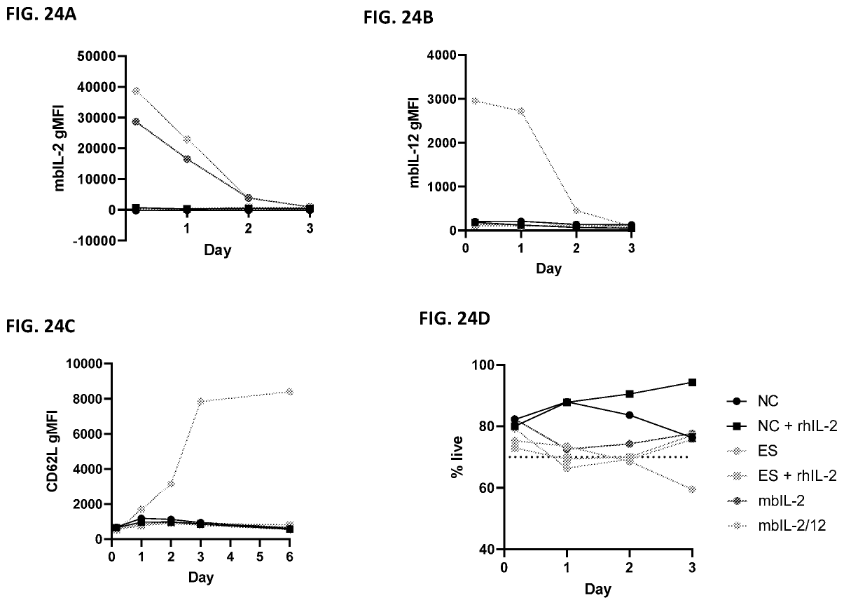

As shown in FIG. 24A-E, squeeze processing induced high expression of the delivered mRNA. All mRNAs showed expression above background for at least 48 hr (FIG. 24A-B). TILs exhibited a mbIL-12-dependent increase in CD62L expression over time, which was sustained for 6 days, beyond the window of expression of mbIL-12 (FIG. 24C). TILs proliferated and remained at least 70% viable for at least 72 hr after squeeze processing (FIG. 24D-E).

Collectively, the results provided herein demonstrate that the squeeze processing methods provided herein can be used to produce TILs that upregulate CD62L, retain high viability, and proliferate for 3 days without requiring exogenous cytokine support.

### Example 5: mbIL-2 and mbIL-12 Engineered TIL Adoptive Transfer in NSG Mice

To determine whether membrane-bound (mb) IL-2 and IL-12 can enhance TIL persistence in vivo in a NSG mouse, post-REP TILs were loaded with mRNA encoding mbIL-2 and/or mbIL-12 using the squeeze processing methods provided herein. The specific methods used are provided below.

### Squeeze Processing

Three 50M cell vials were thawed and diluted with FACs buffer. Cells were centrifuged at 400 rcf at room temperature then washed and resuspended to 3-IOM/mL in AlM V media containing 3000 IU/mL recombinant human (rh) IL-2. Cells were transferred to a G-Rex 6 well plate and cultured at 37° C. To allow the cells to recover from thaw, cells were cultured for 48 hr.

The cells were prepared for squeeze-processing. The cells were counted, filtered through a 40-um cell strainer, washed, and the concentration was adjusted to 80M/mL by adding XVivo-15. The following buffer solutions were prepared for each sample as shown in Table 7:

TABLE 7
Squeeze Processing Cell Suspension Solutions

| | |
|---|---|
| Group A | 400 μL XVivo-15 + 400 μL 2X TILs |
| Group B | 400 μL mRNA mix (296.92 μL XVivo-15 + |
| | 100 μL mbIL-2 mRNA + 3.08 μL NaCl) + |
| | 400 μL 2X TILs |
| Group C | 400 μL mRNA mix (193.84 μL XVivo15 + |
| | 100 μL mbIL-2 mRNA + 100 μL mbIL-12 |
| | mRNA + 6.16 μL NaCl) + 400 μL 2X TILs |

As shown above in Table 7, Group A was used as a control and was squeeze processed without any payload at room temperature using a microfluidic constriction (70 μm depth, 4.5 μm width, and 10 μm length) at 30 psi Groups B-C were squeeze processed using the same conditions as in Group B (i.e., microfluidic constriction: 70 μm depth, 4.5 μm width, and 10 μm length; at 30 psi) at room temperature with the payloads shown above using, i.e., Group B: mbIL-2 mRNA; Group C: mbIL-2 mRNA+mbIL-12 mRNA.

Afterwards, the cells from each of Groups A-C were separately transferred into their own 5 ml conical containing 1 mL RPMI+10% FBS. Cells from all groups were centrifuged separately, at 400 rcf for 4 minutes at room temperature. The supernatant was aspirated, and the cell populations were separately resuspended in either cold Dilution buffer or pre-warmed RPMI+10% FBS media. Cell suspended in Dilution buffer were suspended at 30M/mL and 100 uL per mouse was injected intravenously into the tail vein. Cells suspended in RPMI+10% FBS were cultured in vitro for 72 hours at 37° C.

Mice received no exogenous cytokine support. At 1-day post-transfer, 50-200 uL of peripheral blood was collected from mice via cheek bleed. At 5 days post-transfer, mice were sacrificed, and a terminal cardiac bleed and spleen harvest were performed. Blood was collected into EDTA-containing tubes and processed via Ficoll gradient to collect PBMCs. Spleen was collected into Eppendorf tubes containing FACs buffer, processed through a 70-um cell filter, and underwent RBS lysis via ACK buffer. In parallel with in vitro control cells, ex vivo cells were resuspended in 200 uL of FACs buffer in a 96-well plate and processed for FACS analysis as described herein.

### FACS Analysis

At day 1 and day 5, 200 ul of cells were centrifuged at 400 rcf for 4 minutes at room temperature. The supernatant was discarded, and the cells were resuspended in 50 μL of LD (1:200 dilution in FAC's buffer) and FC block (1:50 dilution in FACs buffer). Cells were incubated at room temperature in the dark for 10 minutes. The cells were spun down at room temperature at 400 rcf for 4 minutes. The cells were then incubated at 4° C. for 20 minutes with fluorescent anti-human antibodies targeting CD3, CD4, CD8, CD45RO, CD62L, IL-2, and IL-12p70 After incubation 150 μL of FACs isolation buffer were added to each sample and the samples were spun at 400 ref for 4 minutes. The cells were spun down at room temperature at 400 rcf for 4 minutes. The supernatant was discarded, and the cells resuspended with 200 μL of FACs isolation buffer. The cells were then analyzed using an Attune Flow cytometer.

### Results

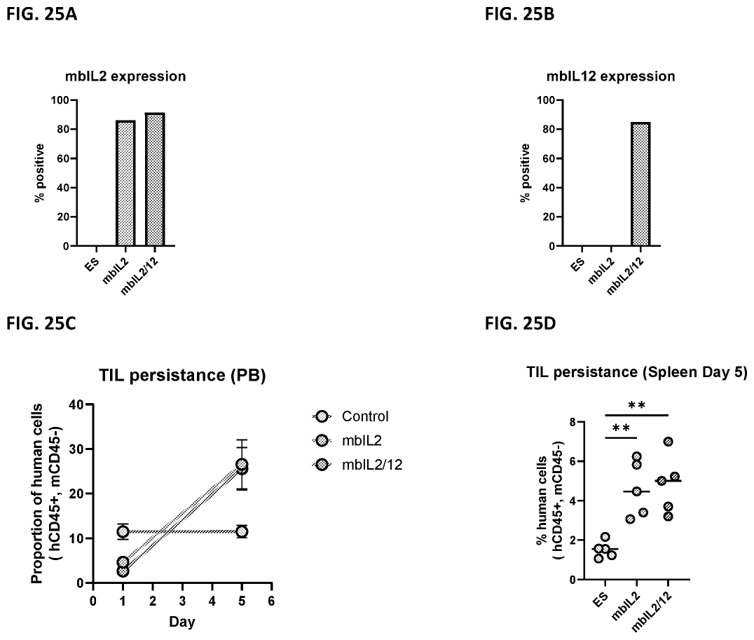

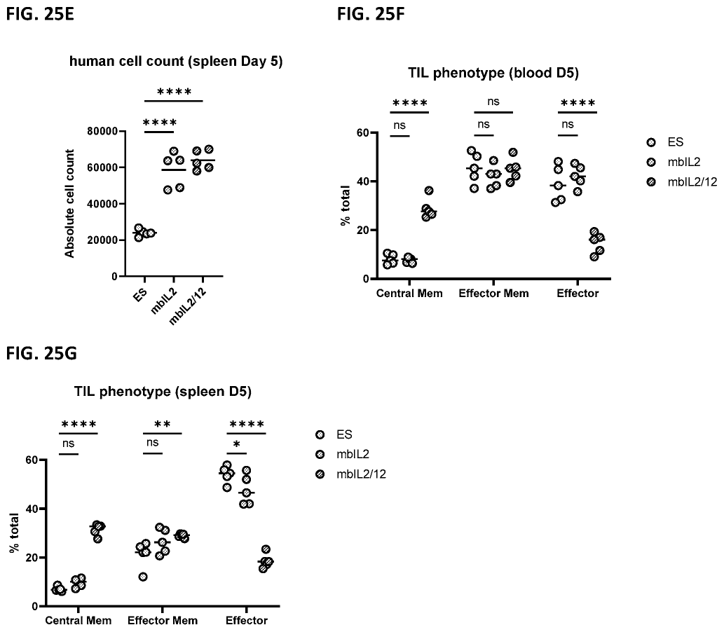

As shown in FIG. 25A-B, squeeze processing induced high expression of the delivered mRNA at day 1. Both mbIL-2 TILs and mbIL-2/12 TILs exhibited enhanced persistence compared to control squeezed TILs on day 5. Enhanced persistence was observed in the blood (FIG. 25C) and spleen, as percentage (FIG. 25D) and absolute cell number (FIG. 25E). mbIL-2/12 TILs also upregulated CD62L, a marker of central memory T cells, in vivo, and these cells were present both in blood (FIG. 25F) and spleen (FIG. 25G).

Collectively, the results provided herein demonstrate that the squeeze processing methods provided herein can be used to produce TILs with enhanced in vivo persistence in both blood and spleen compared to control TILs without cytokine support, and this enhanced persistence lasts through at least 5 days. mbIL-2/12 TILs also exhibited upregulation of CD62L, a marker of central memory, in both blood and spleen.

### Example 6: IFN-γ Response in mbIL-2 and mbIL-2/12 TILs Stimulated with Anti-CD3 Antibody

To assess if TILs membrane-bound (nib) IL-2 and/or mbIL-12 produce IFN-γ in response to T cell receptor (TCR) stimulation via an anti-CD3 agonist antibody (i.e., Signal 1), fresh post-REP TILs were loaded with mRNA encoding mbIL-2 and/or mbIL-12 using the squeeze processing methods provided herein. The specific methods used are provided below.

### Squeeze Processing

Fresh post-REP TILs were collected from a 6-well G-Rex plate, centrifuged at 400 ref at room temperature, and then washed and resuspended to 3-10M/mL in AIM V media without rhIL-2 to remove rhIL-2-dependent induction of pSTAT5 prior to squeeze processing. Cells were transferred to a T25 flask and cultured at 37° C. for 24 hr. Then, the cells were then prepared for squeeze-processing. The cells were counted, filtered through a 40-um cell strainer, washed, and the concentration was adjusted to 30M/mL by adding XVivo-15. The following buffer solutions were prepared for each sample as shown in Table 9:

TABLE 9
Squeeze Processing Cell Suspension Solutions

| | |
|---|---|
| Group A | 100 μL XVivo-15 + 100 μL 2X TILs |
| Group B | 100 μL XVivo-15 + 100 μL 2X TILs |
| Group C | 100 μL mRNA mix (74.23 μL RPMI + 25 μL mbIL-2 |
| | mRNA + 0.77 μL NaCl) + 100 μL 2X TILs |
| Group D | 100 μL mRNA mix (48.46 μL RPMI + 25 μL mbIL-2 |
| | mRNA + 25 μL mbIL-12 mRNA + 1.54 μL NaCl) + |
| | 100 μL 2X TILs |

As shown above in Table 9, Group A was used as a control (i.e., not contacted with any payload and not squeeze processed) Group B was squeeze processed without any payload at room temperature using a microfluidic constriction (70 μm depth, 4.5 μm width, and 10 μm length) at 30 psi. Groups C-D were squeeze processed at room temperature with the payloads shown above, i.e., Group C: mbIL-2 mRNA; Group D: mbIL-2 mRNA+mbIL-12 mRNA. Groups C-D were squeeze processed using the same conditions as in Group B (i.e., microfluidic constriction 70 μm depth, 4.5 μm width, and 10 m length; at 30 psi).

Afterwards, the cells from each of Group A-D were separately transferred into their own 5 ml conical containing 1 mL RPMI+10% FBS. Cells from all groups were centrifuged separately, at 400 rcf for 4 minutes at room temperature. The supernatant was aspirated, and the cell populations were separately resuspended in 1 mL of RPMI+10% FBS media and cultured in technical duplicate with or without 3000 IU/mIL rhIL-2, 125 ng/mL rhIL-12, and/or 30 ng/mL soluble α-CD3 antibody at 37° C. After 4-6 hours of incubation to allow for mRNA translation and transport, protein transport was inhibited by addition of a 1:1000 final dilution each of Monensin and Brefeldin A. Cells were incubated for a total of 24 hours post-squeeze processing.

### FACS Analysis

At 24 hours, 200 ul of cells were aspirated and centrifuged at 400 ref for 4 minutes at room temperature. The supernatant was discarded, and the cells were resuspended in 50 piL of L/D (1:200 dilution in FACs buffer) and FC block (1:50 dilution in FACs buffer). Cells were incubated at room temperature in the dark for 10 minutes. The cells were spun down at room temperature at 400 rcf for 4 minutes. The cells were then incubated at 4° C. for 20 minutes with 50 μL of fluorescent anti-human antibodies targeting CD3, CD4, CD8, IL-2, and IL-12p70 After incubation 150 μL of FACs isolation buffer were added to each sample and the samples were spun at 400 rcf for 4 minutes. The cells were resuspended in 200 L pre-warmed Fixation buffer and incubated at 37° C. for 15 minutes. After incubation the cells were spun down at room temperature at 400 rcf for 4 minutes. The supernatant was discarded, and the cells were resuspended in 200 μL pre-chilled Permeabilization buffer and incubated at −20° C. for 1 hr After incubation the cells were spun down at room temperature at 1000 rcf for 4 minutes. The supernatant was discarded, and the cells were incubated at room temperature for 30 minutes with 50 μL of fluorescent anti-human antibody targeting IFN-γ. After incubation 150 μL of FACs isolation buffer were added to each sample and the cells were spun down at room temperature at 1000 rcf for 4 minutes. The supernatant was discarded, and the cells resuspended with 200 μL of FACs isolation buffer. The cells were then analyzed using an Attune Flow cytometer.

### Results

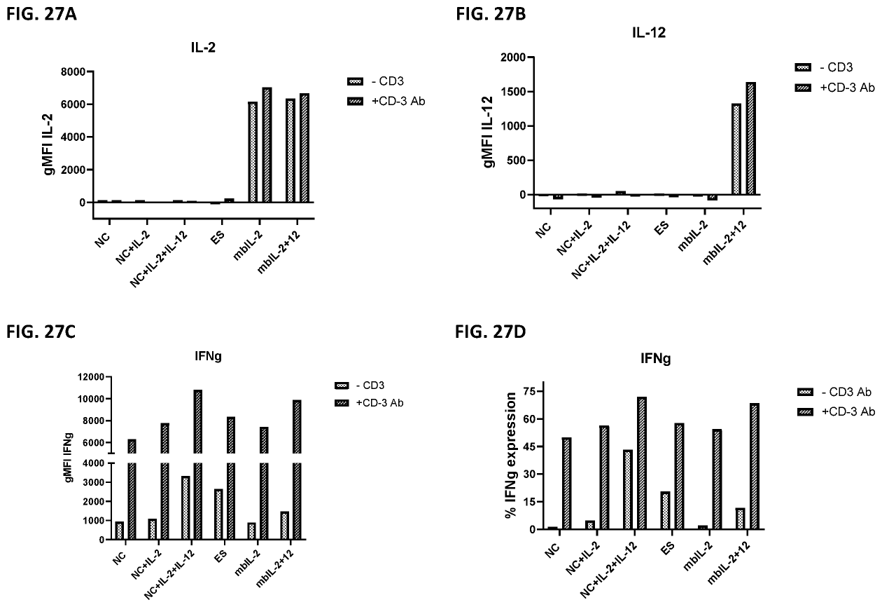

As shown in FIG. 27A-B, squeeze processing induced high expression of the delivered mRNA In the absence of Signal 1, a rh- and mbIL-12-dependent release of IFN-γ was observed (FIG. 27C-D). In the presence of Signal 1, at least 50% of all TIL groups produced IFN-γ, and production was further increased in groups with rh- or mbIL-12 (FIG. 27C-D).

Collectively, the results provided herein demonstrate that the squeeze processing methods provided herein can be used to produce TILs that express membrane-bound IL-12 capable of triggering IFN-γ production, and this production can synergize with Signal 1 stimulation.

### Example 8: PSTAT5 Expression in mbIL-2 and mbIL-7 TILs Starved of rhIL-2 Prior to Squeeze Processing

To assess if membrane-bound (mb) IL-7 can induce phosphorylation of STAT5 downstream of endogenous IL-7R signaling and to compare IL-7-dependent pSTAT5 induction to IL-2-dependent induction, post-REP TILs cultured in the absence of recombinant human (rh) IL-2 for 24 hr were loaded with mRNA encoding mbIL-2 and/or mbIL-7 using the squeeze processing methods provided herein. The specific methods used are provided below.

### Squeeze Processing

Fresh post-REP TILs were collected from a 6-well G-Rex plate, centrifuged at 400 rcf at room temperature, and then washed and resuspended to 3-10M/mL in AIM V media without rhIL-2 to remove rhIL-2-dependent induction of pSTAT5 prior to squeeze processing. Cells were transferred to a T25 flask and cultured at 37° C. for 24 hr. Then, the cells were then prepared for squeeze-processing. The cells were counted, filtered through a 40-um cell strainer, washed, and the concentration was adjusted to 25M/mL by adding XVivo-15. The following buffer solutions were prepared for each sample as shown in Table 10.

TABLE 10
Squeeze Processing Cell Suspension Solutions

| | |
|---|---|
| Group A | 100 μL XVivo-15 + 100 μL 2X TILs |
| Group B | 100 μL XVivo-15 + 100 μL 2X TILs |
| Group C | 100 μL mRNA mix (74.23 μL RPMI + 25 μL mbIL-2 |
| | mRNA + 0.77 μL NaCl) + 100 μL 2X TILs |
| Group D | 100 μL mRNA mix (26.09 μL RPMI + 71.7 μL mbIL-7 |
| | mRNA + 2.21 μL NaCl) + 100 μL 2X TILs |
| Group E | 100 μL mRNA mix (0.22 μL RPMI + 25 μL mbIL-2 |
| | mRNA + 71.7 μL mbIL-7 mRNA + 2.98 μL NaCl) + |
| | 100 μL 2X TILs |

As shown above in Table 10, Group A was used as a control (i.e., not contacted with any payload and not squeeze processed). Group B was squeeze processed without any payload at room temperature using a microfluidic constriction (70 μm depth, 4.5 μm width, and 10 μm length) at 30 psi. Groups C-E were squeeze processed at room temperature with the payloads shown above, i.e., Group C: 500 μg/mL mbIL-2 mRNA; Group D: 350 μg/mL mbIL-7 mRNA; Group E: 500 μg/mL mbIL-2 mRNA+350 μg/mL mbIL-7 mRNA. Groups C-E were squeeze processed using the same conditions as in Group B (i.e., microfluidic constriction. 10 μm depth, 4.5 μm width, and 10 μm length; at 30 psi).

Afterwards, the cells from each of Group A-E were separately transferred into their own 5 ml conical containing 1 mL RPMI+10% FBS. Cells from all groups were centrifuged separately, at 400 rcf for 4 minutes at room temperature. The supernatant was aspirated, and the cell populations were separately resuspended in 1 mL of RPMI+10% FBS media and cultured in technical duplicate with or without 3000 IU/mL rhIL-2 for 72 hours at 37° C.

### FACS Analysis

At 4, 24, 48 and 72 hrs, 200 ul of cells were aspirated and centrifuged at 400 rcf for 4 minutes at room temperature. The supernatant was discarded, and the cells were resuspended in 50 μL of L/D (1:200 dilution in FACs buffer), FC block (1:50 dilution in FACs buffer). Cells were incubated at room temperature in the dark for 10 minutes. The cells were spun down at room temperature at 400 rcf for 4 minutes. The cells were then incubated at 4° C. for 20 minutes with 50 μL of fluorescent ani-human antibodies targeting CD3, CD4, CD8, IL-2, and IL-7R (CD127) and a biotinylated anti-human antibody targeting IL-7. After incubation 150 μL of FACs isolation buffer were added to each sample and the samples were spun at 400 rcf for 4 minutes. The cells were incubated at 4° C. for 20 minutes with 50 μL of fluorescent streptavidin. After incubation 150 μL of FACs isolation buffer were added to each sample and the samples were spun at 400 rcf for 4 minutes. The cells were resuspended in 200 μL pre-warmed Fixation buffer and incubated at 37° C. for 15 minutes. After incubation the cells were spun down at room temperature at 400 rcf for 4 minutes. The supernatant was discarded, and the cells were resuspended in 200 μL pre-chilled Permeabilization buffer and incubated at −20° C. for 1 hr. After incubation the cells were spun down at room temperature at 1000 rcf for 4 minutes. The supernatant was discarded, and the cells were incubated at room temperature for 30 minutes with 50 μL of fluorescent anti-human antibody targeting pSTAT5 (Tyr694). After incubation 150 μL of FACs isolation buffer were added to each sample and the cells were spun down at room temperature at 1000 rcf for 4 minutes. The supernatant was discarded, and the cells resuspended with 200 μL of FACs isolation buffer. The cells were then analyzed using an Attune Flow cytometer

### Results

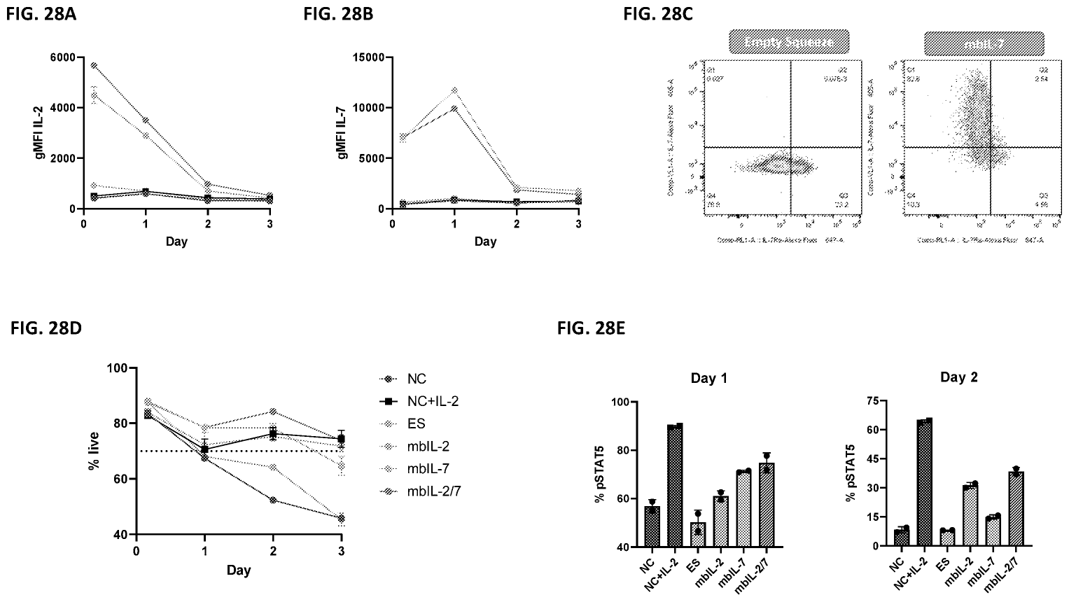

As shown in FIG. 28A-B, squeeze processing induced high expression of the delivered mRNA. All mRNAs showed expression above background for at least 48 hr, with mbIL-2 expression peaking within the first 4 hours of expression, whereas mbIL-7 expression peaks at 24 hours (FIG. 28A-B). mbIL-7 TILs expressing IL-7R (i.e., double positive cells) were absent, suggesting mbIL-7 binds its endogenous receptor and blocks the IL-7R antibody epitope (FIG. 28C). Whereas mbIL-2 supported TIL viability above 70% for 3 days, mbIL-7 did not, although day 3 viability of mbIL-7 TILs was 65% (FIG. 28D). TILs receiving either rhIL-2, mbIL-2 mRNA, or mbIL-7 mRNA had increased pSTAT5 signal over control TILs for the first 48 hours after cell processing (FIG. 28E). Although mbIL-7 TILs exhibited higher pSTAT5 signal than mbIL-2 TILs at 24 hours post-cell processing, mbIL-2 TILs exhibited higher pSTAT5 signal at 48 hours post-cell processing (FIG. 28E). Co-squeezed mbIL-2/7 TILs exhibited the most robust pSTAT5 expression over time (FIG. 28E).

Collectively, the results provided herein demonstrate that the squeeze processing methods provided herein can be used to produce TILs that express membrane-bound IL-2 and membrane bound IL-7, each capable of triggering endogenous signaling to phosphorylate STATS. TILs co-squeezed with both mbIL-2 and mb-IL-7 exhibited the highest viability and most pSTAT5 over the first 48 hours post-cell processing.

### Example 9: Engineered mbIL-2, mbIL-7, and/or mbIL-12 TIL:Matched Tumor Co-Culture Killing Assay and IFN-γ Release

To determine whether membrane-bound (mb) IL-2, IL-7, and/or and IL-12 can enhance TIL-mediated killing of autologous tumor cells in the absence of exogenous cytokine support, post-REP TILs were loaded with mRNA encoding mbIL-2, mbIL-7, and/or mbIL-12 using the squeeze processing methods provided herein, and co-cultured with matched donor tumor cells. The specific methods used are provided below.

### Squeeze Processing

The cells were prepared for squeeze-processing. Fresh TILs on day 14 of REP were counted, filtered through a 40-um cell strainer, washed, and the concentration was adjusted to 35M/mL by adding XVivo-15. The following buffer solutions were prepared for each sample as shown in Table 11:

TABLE 11
Squeeze Processing Cell Suspension Solutions

| | |
|---|---|
| Group A | 300 μL XVivo-15 + 300 μL 2X TILs |
| Group B | 100 μL XVivo-15 + 100 μL 2X TILs |
| Group C | 100 μL mRNA mix (87.12 μL RPMI + 12.5 μL mbIL-2 |
| | mRNA + 0.385 μL NaCl) + 100 μL 2X TILs |
| Group D | 100 μL mRNA mix (74.23 μL RPMI + 12.5 μL mbIL-2 |
| | mRNA + 12.5 μL mbIL-12 mRNA + 0.77 μL NaCl) + |
| | 100 μL 2X TILs |
| Group E | 100 μL mRNA mix (60.66 μL RPMI + 12.5 μL mbIL-2 |
| | mRNA + 12.5 μL mbIL-12 mRNA + 13.2 μL mbIL-7 |
| | mRNA + 1.18 μL NaCl) + 100 μL 2X TILs |

As shown above in Table 11, Group A was used as a control (i.e., not contacted with any payload and not squeeze processed). Group 13 was squeeze processed without any payload at room temperature using a microfluidic constriction (70 μm depth, 4.5 m width, and 10 μm length) at 30 psi. Groups C-E were squeeze processed at room temperature with the payloads shown above, i.e., Group C: mbIL-2 mRNA; Group D: mbIL-2 mRNA+mbIL-12 mRNA; Group E mbIL-2 mRNA+mbIL-7 mRNA+mbIL-12 mRNA. Groups C-E were squeeze processed using the same conditions as in Group B (i.e., microfluidic constriction: 70 μm depth, 4.5 μm width, and 10 μm length; at 30 psi).

Afterwards, the cells from each of Groups A-E were separately transferred into their own 5 ml conical containing 1 mL RPMI+10% FBS. Cells from all groups were centrifuged separately, at 400 rcf for 4 minutes at room temperature. The supernatant was aspirated, and the cell populations were separately resuspended in 1 mL of AIM V media.

In parallel, adherent tumor cells isolated from the initial resected tumor mass and cultured in high-glucose DMEM+10% FBS were collected, counted, stained with 1 μM Cell-Trace Far Red dye, washed, and plated at 1E4 cells per well in a 96 well flat-bottom tissue culture-treated plate. To allow cells to adhere, tumor cells were incubated for at least 1 hour at 37° C.

Squeeze processed TILs were added to the autologous tumor cells at a 5:1 ratio (i.e., 5E4 TILs per well containing 1E4 tumor cells), along with a 1:3000 final dilution of a fluorescent Caspase-3/7 dye. Designated control wells received 3000 IU/mL rhIL-2 and/or 125 ng/mL rhIL-12. The co-culture plate was incubated at 37° C. in an IncuCyte S3 for 24 hours, where images were collected every 30 minutes using the following settings: Phase, Red—300 ms, Green—300 ms, 10× Objective, 3 images/well.

### FACS Analysis and ELISA Analysis

At 24 hours, the cells were spun down at room temperature at 400 rcf for 4 minutes Supernatant was collected for ELISA analysis, described below. For FACS analysis, both adherent and in-suspension cells were collected and centrifuged at 400 rcf for 4 minutes at room temperature. The supernatant was discarded, and the cells were resuspended in 50 μL of L/D (1:200 dilution in FACs buffer), FC block (1:50 dilution in FACs buffer), and Apotracker Dye (1:200 dilution in FACs buffer). Cells were incubated at room temperature in the dark for 10 minutes. The cells were spun down at room temperature at 400 rcf for 4 minutes. The cells were then incubated at 4° C. for 20 minutes with fluorescent anti-human antibodies targeting CD3, CD4, CD8, CD45RO, CD62L, IL-2, and IL-12p70, and a biotinylated anti-human antibody targeting IL-7. After incubation 150 μL of FACs isolation buffer were added to each sample and the samples were spun at 400 rcf for 4 minutes. The cells were then incubated at 4° C. for 10 minutes with fluorescent streptavidin. After incubation 150 μL of FACs isolation buffer were added to each sample and the samples were spun at 400 rcf for 4 minutes. The supernatant was discarded, and the cells resuspended with 200 μL of FACs isolation buffer. The cells were then analyzed using an Attune Flow cytometer.

For ELISA analysis, cell supernatant was kept cold, diluted 1:3 with FACs buffer, and added to wells pre-coated with an anti-human IFN-γ antibody. In parallel, a standard curve of IFN-γ was added to pre-coated wells. Samples were incubated at room temperature for 2 hours, washed, and IFN-γ conjugate was added to each well. Samples were incubated at room temperature for 2 hours, washed, and Substrate Solution was added to each well. Samples were incubated at room temperature for 30 minutes in the dark, after which Stop Solution was added to each well. OD₄₅₀ was measured and supernatant IFN-γ concentrations were calculated by extrapolating values from the standard curve.

### Results

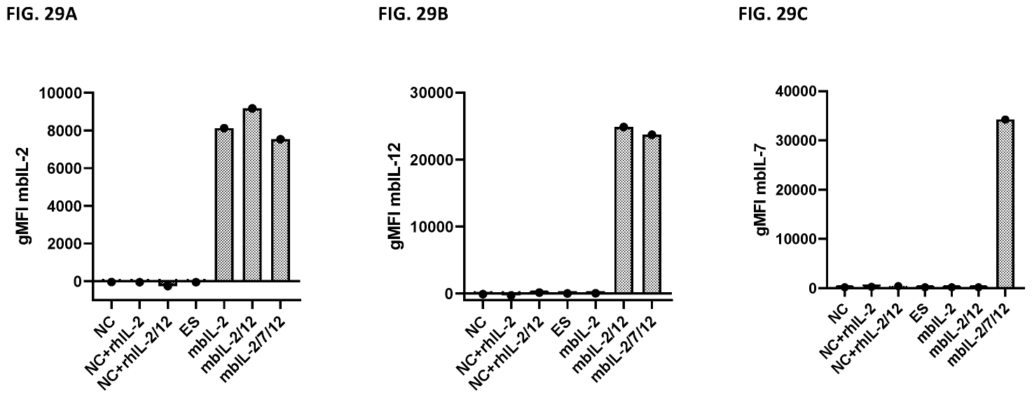

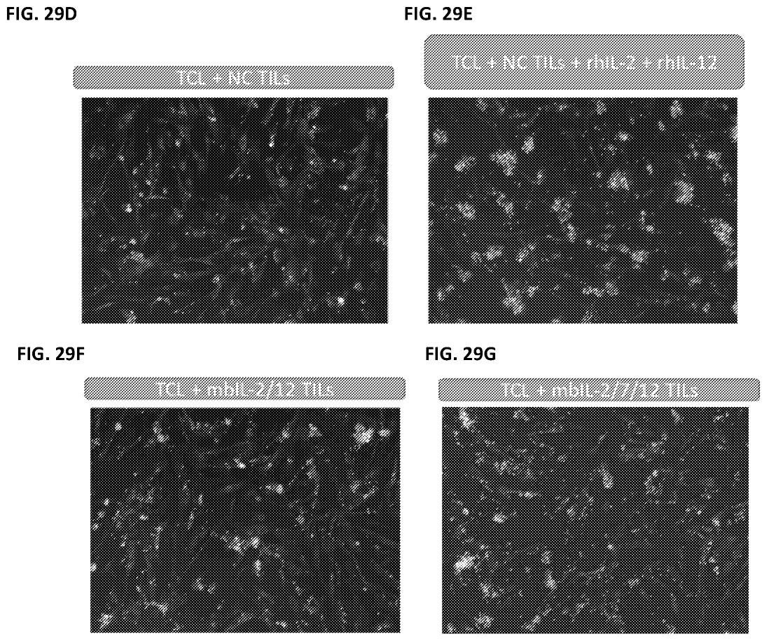

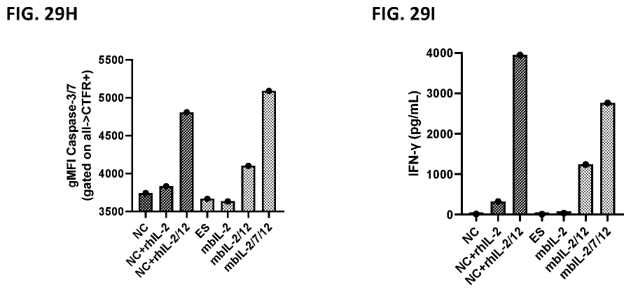

As shown in FIG. 29A-C, squeeze processing induced high expression of the delivered mRNA. All mRNAs showed expression above background at 24 hours (FIG. 29A-C). Representative co-culture images at 24 hours suggest that while control TILs without exogenous cytokine support induce cell killing as measured by fluorescent Caspase-3/7 dye, addition of rhIL-2 and rhIL-12, mbIL-2/12, and/or mbIL-2/7/12 greatly improves cell killing (FIG. 29D-29G). Additionally, quantification of Caspase-3/7 dye intensity of Cell-Trace Far Red-dyed tumor cells via flow cytometry (FIG. 29H) and IFN-γ release via ELISA (FIG. 29I) indicate mbIL-2/12 TILs have improved tumor killing and cytokine release over control TILs without cytokine support, and that adding mbIL-7 mRNA during squeeze processing further improves function, as 2/7/12 TILs are functionally equivalent in vitro to TILs receiving cytokine support (FIG. 29H-29I).

Collectively, the results provided herein demonstrate that the squeeze processing methods provided herein can be used to produce TILs that kill autologous tumor cells in vitro without requiring exogenous cytokine support, and the extent of tumor killing by mbIL-2/7/12 TILs is equivalent to that of TILs cultured in rhIL-2 and rhIL-12.

### Example 10: pSTAT5 Expression in mbIL-2/7/TILs Starved of rhIL-2 Prior to Squeeze Processing

To assess if membrane-bound (mb) IL-7 can induce phosphorylation of STATS downstream of endogenous receptor signaling and to assess whether pSTAT5 is agnostic to if the phosphorylation originates from IL-7R vs. IL-2R, post-REP TILs cultured in the absence of recombinant human (rh) IL-2 for 24 hr were loaded with mRNA encoding mbIL-2 and/or mbIL-7, using the squeeze processing methods provided herein. The specific methods used are provided below.

### Squeeze Processing

Fresh post-REP TILs were collected from a 6-well G-Rex plate, centrifuged at 400 ref at room temperature, and then washed and resuspended to 3-10M/mL in AIM V media without rhIL-2 to remove rhIL-2-dependent induction of pSTAT5 prior to squeeze processing. Cells were transferred to a T25 flask and cultured at 37° C. for 24 hr. Then, the cells were then prepared for squeeze-processing. The cells were counted, filtered through a 40-um cell strainer, washed, and the concentration was adjusted to 150M/mL by adding XVivo-15. The following buffer solutions were prepared for each sample as shown in Table 12:

TABLE 12
Squeeze Processing Cell Suspension Solutions

| | |
|---|---|
| Group A | 100 μL XVivo-15 + 100 μL 2X TILs |
| Group B | 100 μL XVivo-15 + 100 μL 2X TILs |
| Group C | 100 μL mRNA mix (87.12 μL RPMI + 12.5 μL mbIL-2 |
| | mRNA + 0.385 μL NaCl) + 100 μL 2X TILs |
| Group D | 100 μL mRNA mix (74.23 μL RPMI + 25 μL mbIL-2 |
| | mRNA + 0.77 μL NaCl) + 100 μL 2X TILs |
| Group E | 100 μL mRNA mix (87.12 μL RPMI + 12.5 μL mbIL-7 |
| | mRNA + 0.385 μL NaCl) + 100 μL 2X TILs |
| Group F | 100 μL mRNA mix (74.23 μL RPMI + 12.5 μL mbIL-2 |
| | mRNA + 12.5 μL mbIL-7 mRNA + 0.77 μL NaCl) + |
| | 100 μL 2X TILs |

As shown above in Table 12, Group A was used as a control (i.e., not contacted with any payload and not squeeze processed). Group 13 was squeeze processed without any payload at room temperature using a microfluidic constriction (70 μm depth, 4.5 m width, and 10 μm length) at 30 psi. Groups C-H were squeeze processed at room temperature with the payloads shown above, i.e., Group C: mbIL-2 mRNA; Group D: (2×) mbIL-2 mRNA; Group E: mbIL-7 mRNA, Group F mbIL-2 mRNA+mbIL-7 mRNA; Groups C-F were squeeze processed using the same conditions as in Group B (i.e., microfluidic constriction: 70 μm depth, 4.5 μm width, and 10 μm length; at 30 psi).

Afterwards, the cells from each of Group A-F were separately transferred into their own 5 ml conical containing 1 mL RPMI+10% FBS. Cells from all groups were centrifuged separately, at 400 rcf for 4 minutes at room temperature. The supernatant was aspirated, and the cell populations were separately resuspended in 1 mL of AIM V media and cultured in technical duplicate with or without 3000 IU/mIL rhIL-2, 125 ng/mL rhIL-7, and/or 125 ng/mL rhIL-21 at 37° C.

### FACS Analysis

At day 1, day 2, and day 5, 200 ul of cells were aspirated and centrifuged at 400 rcf for 4 minutes at room temperature. The supernatant was discarded, and the cells were resuspended in 50 μL of L/D (1:200 dilution in FACs buffer) and FC block (1:50 dilution in FACs buffer). Cells were incubated at room temperature in the dark for 10 minutes. The cells were spun down at room temperature at 400 rcf for 4 minutes. The cells were then incubated at 4° C. for 20 minutes with 50 μL of fluorescent anti-human antibodies targeting CD3, CD4, CD8, IL-2, and IL-21, and a biotinylated anti-human antibody targeting IL-7. After incubation 150 μL of FACs isolation buffer were added to each sample and the samples were spun at 400 rcf for 4 minutes. The cells were then incubated at 4° C. for 10 minutes with 50 μL of fluorescent streptavidin. After incubation 150 μL of FACs isolation buffer were added to each sample and the samples were spun at 400 rcf for 4 minutes. The cells were resuspended in 200 μL pre-warmed Fixation buffer and incubated at 37° C. for 15 minutes. After incubation the cells were spun down at room temperature at 400 rcf for 4 minutes. The supernatant was discarded, and the cells were resuspended in 200 μL pre-chilled Permeabilization buffer and incubated at −20° C. for 1 hr. After incubation the cells were spun down at room temperature at 1000 rcf for 4 minutes. The supernatant was discarded, and the cells were incubated at room temperature for 30 minutes with 50 μL of fluorescent anti-human antibody targeting pSTAT5 (Tyr694). After incubation 150 μL of FACs isolation buffer were added to each sample and the cells were spun down at room temperature at 1000 rcf for 4 minutes. The supernatant was discarded, and the cells resuspended with 200 μL of FACs isolation buffer. The cells were then analyzed using an Attune Flow cytometer.

### Results

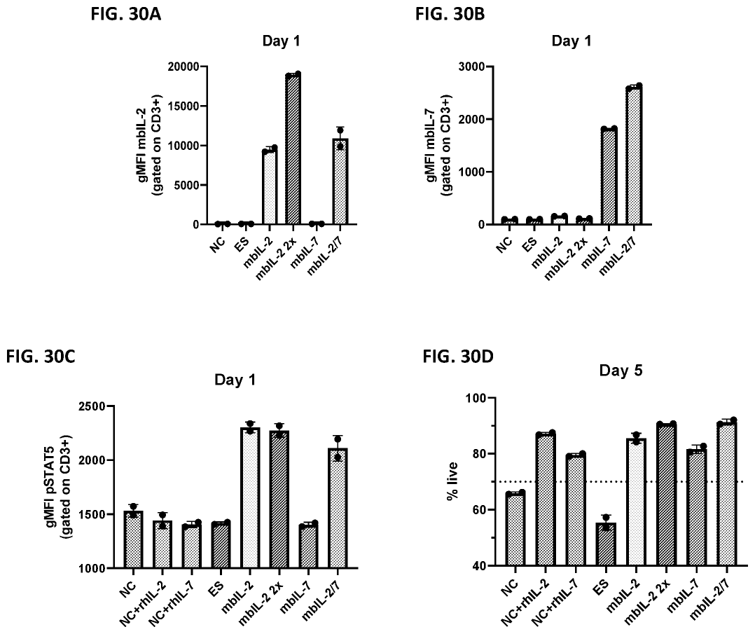

As shown in FIG. 30A-B, squeeze processing induced high expression of the delivered mbIL-2 and mbIL-7 mRNAs at day 1. A dose-dependent increase in mbIL-2 expression was observed (FIG. 30A) mbIL-2 induced similar pSTAT5 signal regardless of mRNA concentration, indicating 250 μg/mL mbIL-2 mRNA is sufficient to drive maximal pSTAT5 at day 1 (FIG. 30C). Whereas control TILs dropped below 70% viability by day 5, mbIL-2 and mbIL-7, each supported TIL viability above 70% through day 5 (FIG. 30D), suggesting each membrane-bound cytokine is sufficient to drive TIL survival.

Collectively, the results provided herein demonstrate that the squeeze processing methods provided herein can be used to produce TILs that express mbIL-2 and mbIL-7, each capable of supporting cell survival and proliferation.

### Example 11: mbIL-2 mRNA, mbIL-7 mRNA, and Bcl-2 mRNA Engineered TIL Resistance to Intrinsic Apoptosis

To determine the expression profile of Bcl-2 mRNA in TILs and to assess if 13-2 mRNA and/or membrane bound (mb) IL-2 mRNA or mbIL-7 mRNA, post-REP TILs were loaded with mRNA encoding mbIL-2, mbIL-7, and/or Bcl-2 using the squeeze processing methods provided herein and incubated in the presence of inducers of intrinsic cell apoptosis. The specific methods used are provided below.

### Squeeze Processing

Fresh post-REP TILs were collected from a 6-well G-Rex plate, centrifuged at 400 rcf at room temperature, and then washed and resuspended to 3-10M/mL in AIM V and transferred to a T25 flask and cultured at 37° C. for 24 hr. The cells were then prepared for squeeze-processing. The cells were counted, filtered through a 40-um cell strainer, washed, and the concentration was adjusted to 60M/mL by adding XVivo-15. The following buffer solutions were prepared for each sample as shown in Table 13.

TABLE 13
Squeeze Processing Cell Suspension Solutions

| | |
|---|---|
| Group A | 100 μL XVivo-15 + 100 μL 2X TILs |
| Group B | 100 μL XVivo-15 + 100 μL 2X TILs |
| Group C | 100 μL mRNA mix (87.12 μL RPMI + 12.5 μL mbIL-7 |
| | mRNA + 0.385 μL NaCl) + 100 μL 2X TILs |
| Group D | 100 μL mRNA mix (87.12 μL RPMI + 12.5 μL Bcl-2 |
| | mRNA + 0.385 μL NaCl) + 100 μL 2X TILs |
| Group E | 100 μL mRNA mix (74.23 μL RPMI + 12.5 μL mbIL-7 |
| | mRNA + 12.5 μL Bcl-2 mRNA + 0.77 μL NaCl) + |
| | 100 μL 2X TILs |
| Group F | 100 μL mRNA mix (87.12 μL RPMI + 12.5 μL mbIL-2 |
| | mRNA + 0.385 μL NaCl) + 100 μL 2X TILs |
| Group G | 100 μL mRNA mix (74.23 μL RPMI + 12.5 μL mbIL-2 |
| | mRNA + 12.5 μL Bcl-2 mRNA + 0.77 μL NaCl) + |
| | 100 μL 2X TILs |
| Group H | 100 μL mRNA mix (61.35 μL RPMI + 12.5 μL mbIL-2 |
| | mRNA + 12.5 μL mbIL-7 mRNA + 12.5 μL Bcl-2 |
| | mRNA + 1.16 μL NaCl) + 100 μL 2X TILs |

As shown above in Table 13, Group A was used as a control (i.e., not contacted with any payload and not squeeze processed). Group 13 was squeeze processed without any payload at room temperature using a microfluidic constriction (70 μm depth, 4.5 un width, and 10 μm length) at 30 psi. Groups C-H were squeeze processed at room temperature with the payloads shown above, i.e., Group C: mbIL-7 mRNA; Group D: Bcl-2 mRNA; Group E: mbIL-7 mRNA+Bcl-2 mRNA; Group F: mbIL-2 mRNA; Group G: mbIL-2 mRNA+Bcl-2 mRNA; Group H: mbIL-2 mRNA+mbIL-7 mRNA+Bcl-2 mRNA. Groups C-H were squeeze processed using the same conditions as in Group B (i.e., microfluidic constriction: 70 μm depth, 4.5 μm width, and 10 μm length; at 30 psi).

Afterwards, the cells from each of Group A-H were separately transferred into their own 5 ml conical containing 1 mL RPMI+10% FBS. Cells from all groups were centrifuged separately, at 400 rcf for 4 minutes at room temperature. The supernatant was aspirated, and the cell populations were separately resuspended in 1 mL of AIM V media and cultured in technical duplicate with or without 125 ng/mL rhIL-7, and/or 10 nM, 100 nM, or 1000 nM each of Venetoclax and S63548 at 37° C.

### FACS Analysis

At 24 hours, 200 ul of cells were aspirated and centrifuged at 400 ref for 4 minutes at room temperature. The supernatant was discarded, and the cells were resuspended in 50 NL of L/D (1.200 dilution in FACs buffer), FC block (1:50 dilution in FACs buffer), and Apotracker Green (1:300 dilution in FACs buffer). Cells were incubated at room temperature in the dark for 10 minutes. The cells were spun down at room temperature at 400 rcf for 4 minutes. The cells were then incubated at 4° C. for 20 minutes with 50 μL of fluorescent anti-human antibodies targeting CD3, CD8, and IL-7R, and a biotinylated anti-human antibody targeting IL-7. After incubation 150 μL of FACs isolation buffer were added to each sample and the samples were spun at 400 rcf for 4 minutes. The cells were then incubated at 4° C. for 10 minutes with 50 μL of fluorescent streptavidin. After incubation 150 μL of FACs isolation buffer were added to each sample and the samples were spun at 400 rcf for 4 minutes. The cells were resuspended in 200 μL pre-warned Fixation buffer and incubated at 37° C. for 15 minutes. After incubation the cells were spun down at room temperature at 400 rcf for 4 minutes. The supernatant was discarded, and the cells were resuspended in 200 μL pre-chilled Permeabilization buffer and incubated at −20° C. for 1 hr. After incubation the cells were spun down at room temperature at 1000 rcf for 4 minutes. The supernatant was discarded, and the cells were incubated at room temperature for 30 minutes with 50 μL of fluorescent anti-human antibody targeting Bcl-2. After incubation 150 μL of FACs isolation buffer were added to each sample and the cells were spun down at room temperature at 1000 rcf for 4 minutes. The supernatant was discarded, and the cells resuspended with 200 μL of FACs isolation buffer. The cells were then analyzed using an Attune Flow cytometer.

### Results

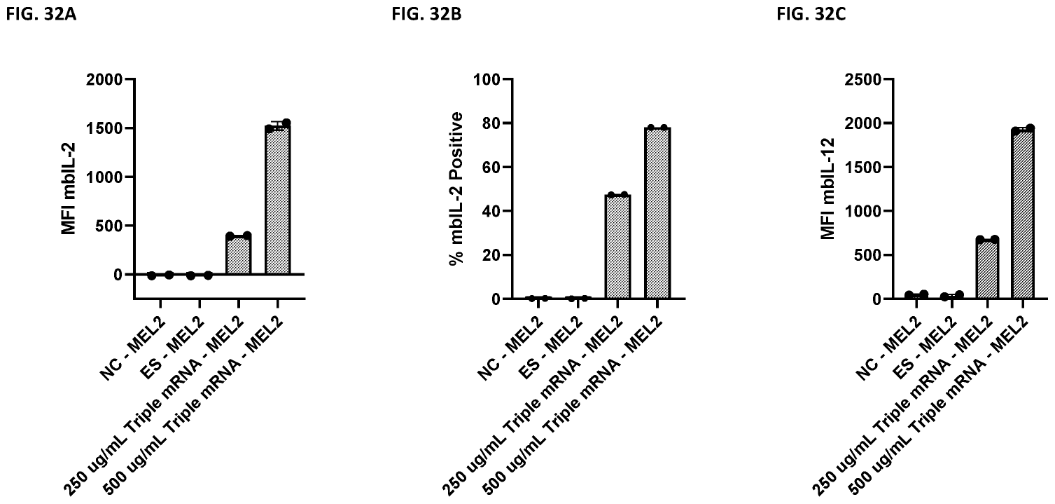

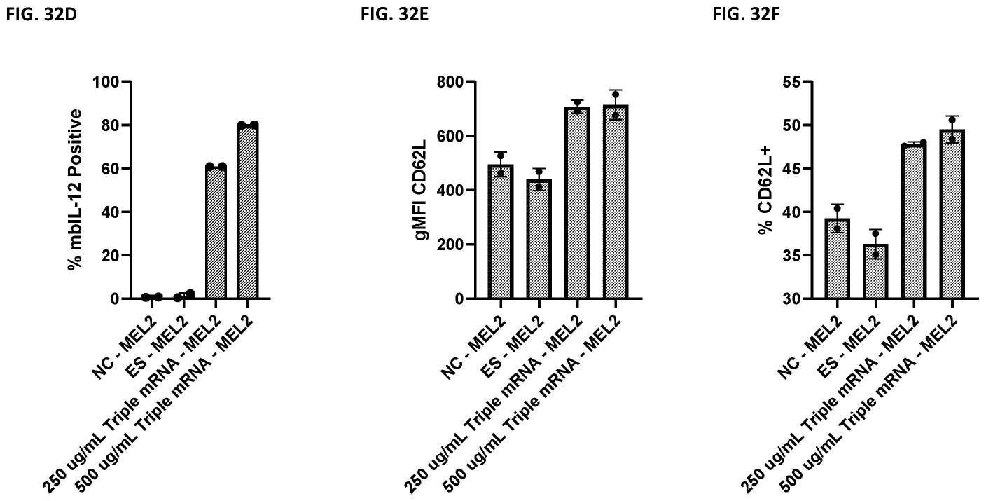

As shown in FIG. 31A-C, squeeze processing induced high expression of the delivered mbIL-7 and Bcl-2 mRNAs. Baseline Bcl-2 expression was 95%, but delivery of Bcl-2 mRNA further increased Bcl-2 expression (FIG. 31A-B). mbIL-2 expression was not determined in this experiment (data not shown). Bcl-2 inhibitor Venetoclax (V) and Mcl-2 inhibitor S63548 (S) induced apoptosis as measured by Apotracker dye, and the strongest dynamic range of cell killing was at 10 nM (FIG. 31D). In the presence of 10 nM V+S, Bcl-2 mRNA is protective against apoptosis, however, neither mbIL-2 mRNA alone nor mbIL-7 mRNA alone was protective (FIG. 32I).

Collectively, the results provided herein demonstrate that the squeeze processing methods provided herein can be used to produce TILs that express Bcl-2 above baseline at 24 hours, and this elevated expression of Bcl-2 is protective against induction of intrinsic apoptosis.

### Example 12: mbIL-2, mbIL-12, and CD86 mRNA Engineered TIL Expression

To assess the expression kinetics of certain payloads described herein and to determine whether membrane-bound (mb) IL-2 and IL-12 and CD86 mRNA are expressed by TILs, post-REP TILs were loaded with mRNA encoding mbIL-2, mbIL-12, or CD86 mRNAs using the squeeze processing methods provided herein. The specific methods used are provided below.

### Squeeze Processing

The cells were prepared for squeeze-processing. Fresh TILs on day 14 of REP were counted, filtered through a 40-urn cell strainer, washed, and the concentration was adjusted to 35M/mL by adding RPMI. The following buffer solutions were prepared for each sample as shown in Table 14:

TABLE 14
Squeeze Processing Cell Suspension Solutions

| | |
|---|---|
| Group A | 100 μL RPMI + 100 μL 2X TILs |
| Group B | 100 μL RPMI + 100 μL 2X TILs |
| Group C | 100 μL mRNA mix (51.34 μL RPMI + 10 μL Dextran + |
| | 12.5 μL mbIL-2 mRNA + 12.5 μL mbIL-12 mRNA + 12.5 |
| | μL CD86 mRNA + 1.16 μL NaCl) + 100 μL 2X TILs |
| Group D | 100 μL mRNA mix (12.69 μL RPMI + 10 μL Dextran + |
| | 25 μL mbIL-2 mRNA + 25 μL mbIL-12 mRNA + 25 μL |
| | CD86 mRNA + 2.31 μL NaCl) + 100 μL 2X TILs |

As shown above in Table 14, Group A was used as a control (i.e., not contacted with any payload and not squeeze processed). Group B was squeeze processed without any payload at room temperature using a microfluidic constriction (70 μm depth, 4.0 μm width, and 10 μm length) at 30 psi. Groups C-D were squeeze processed at room temperature with the payloads shown above, i.e., Group C: Dextran+mbIL-2 mRNA 4+mbIL-12 mRNA+CD86 mRNA; Group D: Dextran+(2×) mbIL-2 mRNA+(2×) mbIL-12 mRNA+(2×) CD86 mRNA. Groups C-D were squeeze processed using the same conditions as in Group 13 (i.e., microfluidic constriction: 70 μm depth, 4.0 μm width, and 10 μm length; at 30 psi).

Afterwards, the cells from each of Group A-D were separately transferred into their own 5 ml conical containing 1 mL RPMI+10% FBS. Cells from all groups were centrifuged separately, at 400 rcf for 4 minutes at room temperature. The supernatant was aspirated, and the cell populations were separately resuspended in 1 mL of RPMI+10% FBS media and cultured in technical duplicate for 24 hours at 37° C.

### FACS Analysis

At 24 hours, 200 ul of cells were aspirated and centrifuged at 400 rcf for 4 minutes at room temperature. The supernatant was discarded, and the cells were resuspended in 50 μL of L/D (1:200 dilution in FACs buffer) and FC block (1:50 dilution in FACs buffer) Cells were incubated at room temperature in the dark for 10 minutes. The cells were spun down at room temperature at 400 rcf for 4 minutes. The cells were then incubated at 4 C for 20 minutes with fluorescent anti-human antibodies targeting CD3, CD4, CD8, CD45RA, CD62L, CD86, IL-2, and IL-12p70. After incubation 150 μL of FACs isolation buffer were added to each sample and the samples were spun at 400 rcf for 4 minutes. The cells were spun down at room temperature at 400 rcf for 4 minutes. The supernatant was discarded, and the cells resuspended with 200 μL of FACs isolation buffer. The cells were then analyzed using an Attune Flow cytometer.

### Results

As shown in FIG. 32A-F, squeeze processing induced high expression of the delivered mRNA. All mRNAs showed expression above background at 24 hr (FIG. 32A-D). TILs exhibited a mbIL-12-dependent increase in CD62L expression (FIG. 32E-F).

Collectively, the results provided herein demonstrate that the squeeze processing methods provided herein can be used to produce TILs that upregulate CD62L in a mbIL-12-dependent manner after 24 hours of culture.

### Example 13: CD25 Occupancy in mbIL-2 mRNA Engineered TILs

To determine whether membrane-bound (mb) IL-2 bind endogenous IL-2Rα, also known as CD25, expressed on TILs, post-REP TILs were loaded with mRNA encoding mbIL-2 using the squeeze processing methods provided herein, and CD25 binding was determined by using two different anti-human CD25 antibodies, one expected to compete with mbIL-2 for binding, and one that binds an epitope outside of the binding region of IL-2. The specific methods used are provided below.

### Squeeze Processing

One 50M cell vial was thawed and diluted with FACs buffer per donor. Cells were centrifuged at 400 rcf at room temperature then washed and resuspended to 3-10M/mL in AIM V media containing 3000 IU/mL recombinant human (rh) IL-2. Cells were transferred to a T75 flask and cultured at 37° C. To allow the cells to recover from thaw, cells were cultured for 24 hr.

The cells were prepared for squeeze-processing. A control vial of PBMCs was thawed and diluted with FACs buffer. All cells were counted, filtered through a 40-um cell strainer, washed, and the concentration was adjusted to 60-70M/mL by adding RPMI. The following buffer solutions were prepared for each sample as shown in Table 15:

TABLE 15
Squeeze Processing Cell Suspension Solutions

| | |
|---|---|
| Group A - TIL Donor 1 | 100 μL RPMI + 100 μL 2X TILs |
| Group B - TIL Donor 1 | 100 μL mRNA mix (77.12 μL RPMI + 10 μL |
| | Dextran + 12.5 μL mbIL-2 mRNA + 0.385 |
| | μL NaCl) + 100 μL 2X TILs |
| Group C - TIL Donor 2 | 100 μL RPMI + 100 μL 2X TILs |
| Group D - TIL Donor 2 | 100 μL mRNA mix (77.12 μL RPMI + 10 μL |
| | Dextran + 12.5 μL mbIL-2 mRNA + 0.385 |
| | μL NaCl) + 100 μL 2X TILs |
| Group E - TIL Donor 3 | 100 μL RPMI + 100 μL 2X TILs |
| Group F - TIL Donor 3 | 100 μL mRNA mix (77.12 μL RPMI + 10 μL |
| | Dextran + 12.5 μL mbIL-2 mRNA + 0.385 |
| | μL NaCl) + 100 μL 2X TILs |
| Group G - PBMC Donor | 100 μL RPMI + 100 μL 2X TILs |
| Group H - PBMC Donor | 100 μL mRNA mix (77.12 μL RPMI + 10 μL |
| | Dextran + 12.5 μL mbIL-2 mRNA + 0.385 |
| | μL NaCl) + 100 μL 2X TILs |

As shown above in Table 15, Groups A, C, E, and G were squeeze processed without any payload at room temperature using the following microfluidic constrictions: Group A: 70 μm depth, 4.0 μm width, and 10 μm length at 30 psi, Group C: 70 μm depth, 4.0 μm width, and 10 μm length at 60 psi, Group E: 70 μm depth, 6.0 μm width, and 10 μm length at 60 psi, and Group G: 70 μm depth, 3.5 μm width, and 10 μm length at 60 psi. Groups B, D, F, and H were squeeze processed at room temperature with the payloads, constrictions, and psi shown above, i.e., Group B: 70 μm depth, 4.0 μm width, and 10 μm length at 30 psi, Group D: 70 μm depth, 4.0 μm width, and 10 μm length at 60 psi, Group F: 70 μm depth, 6.0 μm width, and 10 μm length at 60 psi, and Group H: 70 μm depth, 3.5 μm width, and 10 μm length at 60 psi.

Afterwards, the cells from each of Group A-H were separately transferred into their own 5 ml conical containing 1 mL RPMI+10% FBS. Cells from all groups were centrifuged separately, at 400 rcf for 4 minutes at room temperature. The supernatant was aspirated, and the cell populations were separately resuspended in 1 mL of RPMI+10% FBS media and cultured in technical duplicate for 24 hours at 37° C.

### FACS Analysis

At 4, 24, 48, and 72 hours, 200 ul of cells were aspirated and centrifuged at 400 rcf for 4 minutes at room temperature. The supernatant was discarded, and the cells were resuspended in 50 μL of LID (1:200 dilution in FACs buffer) and FC block (1:50 dilution in FACs buffer). Cells were incubated at room temperature in the dark for 10 minutes. The cells were spun down at room temperature at 400 ref for 4 minutes. The cells were then incubated at 4 C for 20 minutes with fluorescent anti-human antibodies targeting CD3, CD4, CD8, IL-2, CD25 (clone BC96 and/or clone M-A251). After incubation 150 μL of FACs isolation buffer were added to each sample and the samples were spun at 400 ref for 4 minutes. The cells were spun down at room temperature at 400 rcf for 4 minutes. The supernatant was discarded, and the cells resuspended with 200 μL of FACs isolation buffer. The cells were then analyzed using an Attune Flow cytometer.

### Results

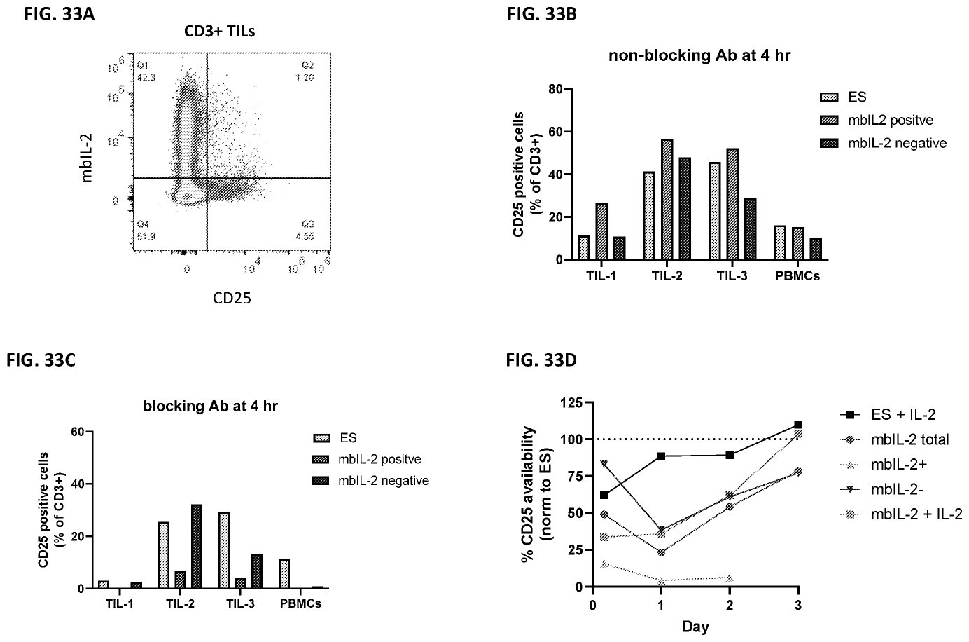

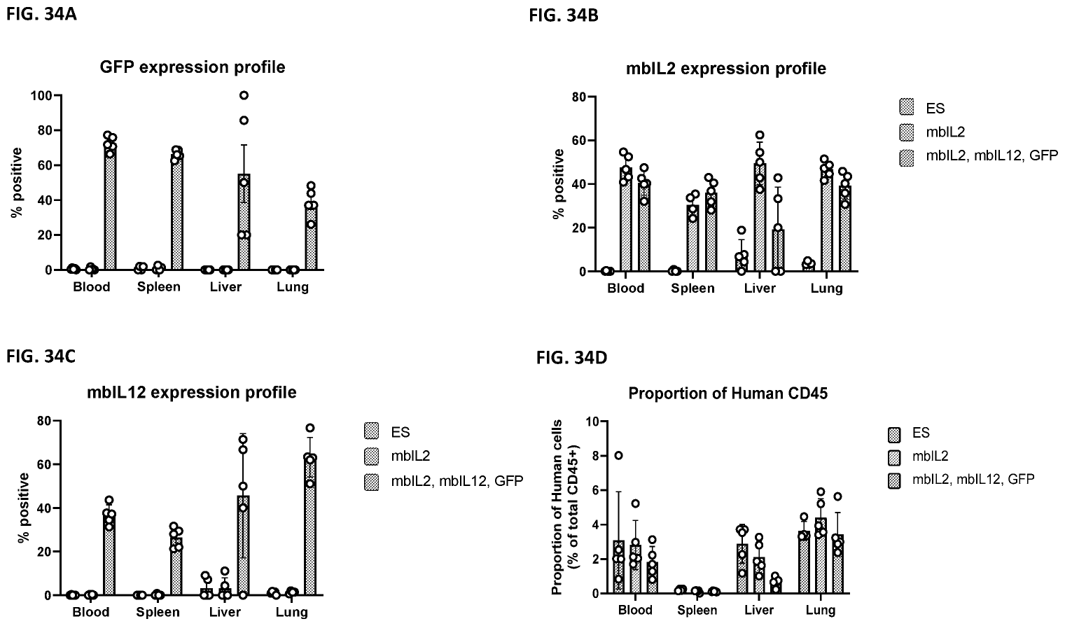

As shown in FIG. 33A, squeeze processing induced high expression of the delivered mRNA. CD25 expression as detected by a non-blocking CD25 antibody ranged from 4-60% across three TIL donors (FIG. 33B). However, CD25 expression was greatly decreased in mbIL-2 positive cells, as shown by a lack of double positive CD25⁺ mIL-2⁺ cells (FIG. 34A; Q2), suggesting mbIL-2 is bound to CD25. CD25 expression was decreased across all donors in mbIL-2 positive cells, including PBMCs, but not in control TILs or TILs not expressing mbIL-2 (FIG. 33C). When normalized to CD25 expression in donor-matched control TILs, CD25 is blocked throughout the expression window of mbIL-2 (FIG. 33D). CD25 expression returns to 75% by day 3, suggesting 25% of CD25 remains occupied by mbIL-2 on day 3 (FIG. 33D).

Collectively, the results provided herein demonstrate that the squeeze processing methods provided herein can be used to produce TILs expressing mbIL-2 capable of binding endogenous IL-2Rα, also known as CD25. The vast majority of CD25 is bound by mbIL-2 throughout the expression window of mbIL-2 and raises the possibility that mbIL-2 is expressed on the TIL surface on day 3 but is bound to CD25 and therefore not detected by anti-IL-2 antibody.

### Example 14: eGFP, mbIL-2, and mbIL-12 Engineered TIL Adoptive Transfer in NSG Mice

To determine the distribution of squeeze engineered TILs within peripheral blood and organs of an NSG mouse after 24 hours of persistence, post-REP TILs were loaded with mRNA encoding GFP, mbIL-2, and/or mbIL-12 using the squeeze processing methods provided herein and adoptively transferred into NSG mice. The specific methods used are provided below.

### Squeeze Processing

Two 50M cell vials were thawed and diluted with FACs buffer. Cells were centrifuged at 400 rcf at room temperature then washed and resuspended to 3-10M/mL in AIM V media containing 3000 IUI/mL recombinant human (rh) IL-2. Cells were transferred to a G-Rex 6 well plate and cultured at 37° C. To allow the cells to recover from thaw and to expand the cells sufficiently, cells were cultured for 24 days.

The cells were prepared for squeeze-processing. The cells were counted, filtered through a 40-um cell strainer, washed, and the concentration was adjusted to 175M/mL by adding RPMI. The following buffer solutions were prepared for each sample as shown in Table 16:

TABLE 16
Squeeze Processing Cell Suspension Solutions

| | |
|---|---|
| Group A | 125 μL RPMI + 125 μL 2X TILs |
| Group B | 125 μL mRNA mix (92.79 μL RPMI + 31.25 μL mbIL-2 |
| | mRNA + 0.96 μL NaCl) + 125 μL 2X TILs |
| Group C | 125 μL mRNA mix (28.36 μL RPMI + 31.25 μL eGFP |
| | mRNA + 31.25 μL mbIL-2 mRNA + 31.25 μL mbIL-12 |
| | mRNA + 2.89 μL NaCl) + 125 μL 2X TILs |

As shown above in Table 16, Group A was used as a control and was squeeze processed without any payload at room temperature using a microfluidic constriction (70 μm depth, 4.0 μm width, and 10 μm length) at 30 psi. Groups B-C were squeeze processed using the same conditions as in Group B (i.e., microfluidic constriction: 70 μm depth, 4.0 μm width, and 10 μm length; at 30 psi) at room temperature with the payloads shown above, i.e., Group B: mbIL-2 mRNA; Group C: eGFP mRNA+mbIL-2 mRNA+mbIL-12 mRNA.

Afterwards, the cells from each of Groups A-C were separately transferred into their own 5 ml conical containing 1 mL RPMI+10% FBS. Cells from all groups were centrifuged separately, at 400 rcf for 4 minutes at room temperature. The supernatant was aspirated, and the cell populations were separately resuspended in either cold Dilution buffer or pre-warmed AIM V media. Cell suspended in Dilution buffer were suspended at 20M/mL and 100 uL per mouse was injected intravenously into the tail vein. Cells suspended in AIM V were cultured in vitro for 24 hours at 37° C.

Mice received no exogenous cytokine support. At 1-day post-transfer, mice were sacrificed, and a terminal cardiac bleed and spleen, lung, and liver harvest were performed. Blood was collected into EDTA-containing tubes and processed via Ficoll gradient to collect PBMCs. Spleen was collected into Eppendorf tubes containing FACs buffer, processed through a 70-um cell filter, and underwent RBS lysis via ACK buffer. Lung and liver were collected into Eppendorf tubes containing FACs buffer, digested with Collagenase A and DNase 1, processed through a 70-um cell filter, and underwent RBS lysis via ACK buffer. In parallel with in vitro control cells, ex vivo cells were resuspended in 200 μL of FACs buffer in a 96-well plate and processed for FACS analysis as described herein.

### FACS Analysis

At day 1, 200 ul of cells were centrifuged at 400 rcf for 4 minutes at room temperature. The supernatant was discarded, and the cells were resuspended in 50 μL of L/D (1:200 dilution in FACs buffer) and FC block (1:50 dilution in FACs buffer). Cells were incubated at room temperature in the dark for 10 minutes. The cells were spun down at room temperature at 400 rcf for 4 minutes. The cells were then incubated at 4 C for 20 minutes with fluorescent anti-human antibodies targeting CD45, CD3, CD4, CD8, CD25, IL-2, and IL-12p70, and a fluorescent anti-mouse antibody targeting CD45. After incubation 150 μL of FACs isolation buffer were added to each sample and the samples were spun at 400 rcf for 4 minutes. The cells were spun down at room temperature at 400 rcf for 4 minutes. The supernatant was discarded, and the cells resuspended with 200 μL of FACs isolation buffer. The cells were then analyzed using an Attune Flow cytometer.

### Results

As shown in FIG. 34A-C, squeeze processing induced high expression of the delivered mRNAs at day 1. TILs are not trapped in a particular organ at 1-day post-transfer and are equally distributed regardless of delivered mRNA (FIG. 34D). Of the organs collected, the lung contained the highest percentage of TILs (FIG. 34D).

Collectively, the results provided herein demonstrate that the squeeze processing methods provided herein can be used to produce TILs with payload expression in vivo in blood, spleen, liver, and lung.

### Example 15: mbIL-2 and mbIL-12 Engineered TIL Expanded T Cell Marker Flow Panel

To determine if post-REP TILs express T cell markers relevant to the tumor microenvironment and to determine if exogenous recombinant human (rh) cytokines induce similar phenotypic changes to matched membrane bound (mb) cytokines, post-REP TILs were loaded with mRNA encoding mbIL-2 and/or mbIL-12 using the squeeze processing methods provided herein. The specific methods used are provided below.

### Squeeze Processing

Fresh TILs on day 19 of REP were prepared for squeeze-processing. The cells were counted, filtered through a 40-um cell strainer, washed, and the concentration was adjusted to 20-50M/mL by adding XVivo-15. The following buffer solutions were prepared for each sample as shown in Table 17:

TABLE 17
Squeeze Processing Cell Suspension Solutions

| | |
|---|---|
| Group A | 100 μL XVivo-15 + 100 μL 2X TILs |
| Group B | 100 μL XVivo-15 + 100 μL 2X TILs |
| Group C | 100 μL mRNA mix (74.23 μL RPMI + 25 μL mbIL-2 |
| | mRNA + 0.77 μL NaCl) + 100 μL 2X TILs |
| Group D | 100 μL mRNA mix (48.46 μL RPMI + 25 μL mbIL-2 |
| | mRNA + 25 μL mbIL-12 mRNA + 1.54 μL NaCl) + |
| | 100 μL 2X TILs |

As shown above in Table 17, Group A was used as a control (i.e., not contacted with any payload and not squeeze processed). Group 13 was squeeze processed without any payload at room temperature using a microfluidic constriction (70 μm depth, 4.5 m width, and 10 μm length) at 30 psi. Groups C-D were squeeze processed at room temperature with the payloads shown above, i.e., Group C: mbIL-2 mRNA; Group D: mbIL-2 mRNA+mbIL-12 mRNA. Groups C-D were squeeze processed using the same conditions as in Group B (i.e., microfluidic constriction: 70 μm depth, 4.5 μm width, and 10 μm length; at 30 psi).

Afterwards, the cells from each of Groups A-D were separately transferred into their own 5 ml conical containing 1 mL RPMI+10% FBS. Cells from all groups were centrifuged separately, at 400 rcf for 4 minutes at room temperature. The supernatant was aspirated, and the cell populations were separately resuspended in 1 mL of RPMI+10% FBS media and cultured in technical duplicate with or without 3000 IU/mL rhIL-2 and/or 125 ng/mL rhIL-12 at 37° C.

### FACS Analysis

At 24 hours, 200 ul of cells were aspirated and centrifuged at 400 rcf for 4 minutes at room temperature. The supernatant was discarded, and the cells were resuspended in 50 μL of L/D (1:200 dilution in FACs buffer) and FC block (1:50 dilution in FACs buffer). Cells were incubated at room temperature in the dark for 10 minutes. The cells were spun down at room temperature at 400 rcf for 4 minutes. The cells were then incubated at 4 C for 20 minutes with fluorescent anti-human antibodies targeting CD3, CD4, CD8, CD39 CD45RO, CD62L, CD69, IL-2, and IL-12p70. After incubation 150 μL of FACs isolation buffer were added to each sample and the samples were spun at 400 rcf for 4 minutes. The cells were spun down at room temperature at 400 rcf for 4 minutes. The cells were resuspended in 200 μL pre-warmed Fixation buffer and incubated at 37° C. for 15 minutes. After incubation the cells were spun down at room temperature at 400 rcf for 4 minutes. The supernatant was discarded, and the cells were resuspended in 200 μL pre-chilled Permeabilization buffer and incubated at −20° C. for 1 hr. After incubation the cells were spun down at room temperature at 1000 rcf for 4 minutes. The supernatant was discarded, and the cells were incubated at room temperature for 30 minutes with 50 μL of fluorescent anti-human antibody targeting Ki-67. After incubation 150 μL of FACs isolation buffer were added to each sample and the cells were spun down at room temperature at 1000 rcf for 4 minutes. The supernatant was discarded, and the cells resuspended with 200 μL of FACs isolation buffer. The cells were then analyzed using an Attune Flow cytometer.

### Results

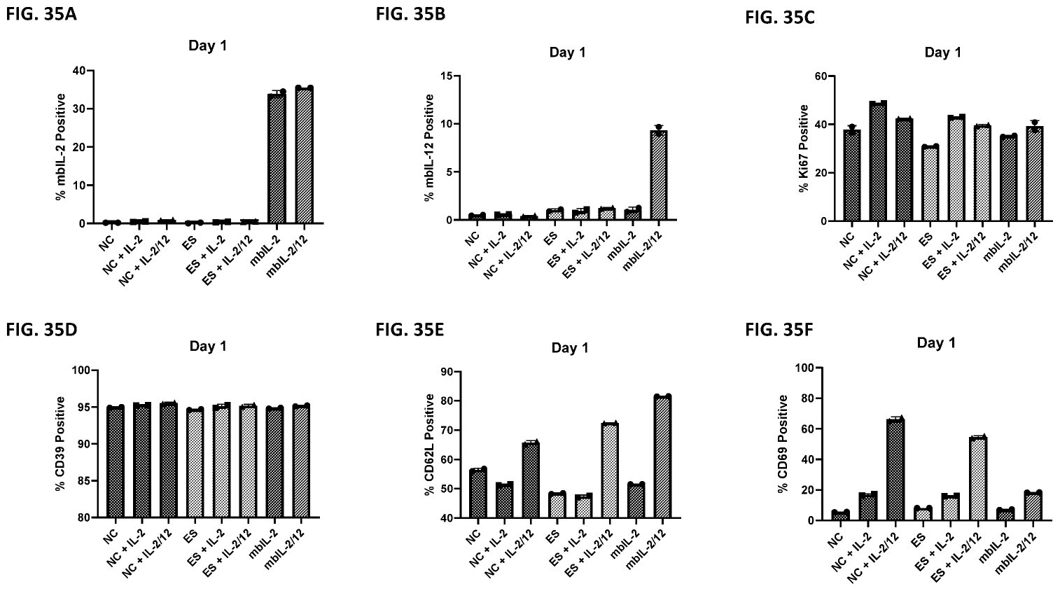

As shown in FIG. 35A-B, squeeze processing induced expression of the delivered mRNA. All mRNAs showed expression above background for at 24 hours (FIG. 35A-B). Around 40% of all TILs were proliferating regardless of culture condition, as measured by Ki-67 expression (FIG. 35C). Most TILs at baseline were CD39 positive and signal was not impacted by rh- or mb-IL-2 or -12 (FIG. 35D). Lastly, rh- and mb-IL-12 induced CD62L and CD69 upregulation (FIG. 35E-35F).

Collectively, the results provided herein demonstrate that the squeeze processing methods provided herein can be used to produce TILs expressing membrane-bound cytokines that induce the same phenotypic changes as their respective recombinant exogenous cytokines, such as IL-12-dependent induction of both CD62L, a marker of memory T cells, and CD69, a marker of resident memory T cells.

### Example 16: Proliferative Capacity of CD62L Positive mbIL-2 and mbIL-12 Engineered TILs

To assess if the increase in CD62L positive TILs in mbIL-12-engineered TILs is from conversion of previously CD62L negative TILs or from a higher proliferative capacity of baseline CD62L positive TILs, post-REP TILs were isolated based on baseline CD62L expression, dyed, and loaded with mRNA encoding mbIL-2 and/or mbIL-12 using the squeeze processing methods provided herein. The specific methods used are provided below.

### Squeeze Processing

Fresh TILs from two donors on day 14 of REP were prepared for squeeze-processing Donors were processed in parallel throughout the experiment. TILs were centrifuged at 400 rcf for 4 minutes at room temperature. The supernatant was aspirated, and the cell populations were separately resuspended in 1 mL FACs buffer Cells were incubated at 4 C for 20 minutes with fluorescent anti-human antibody targeting CD62L. After incubation 150 μL of FACs isolation buffer were added to each sample and the samples were spun at 400 rcf for 4 minutes. Cells were then isolated using magnetic beads that select based on fluorescence as a proxy for CD62L expression. Cell fractions were incubated for 20 minutes at 37 C with 1 μM final cytoplasmic dyes; CD62L positive cells were incubated with dye that excites/emits at 405/450 nm and CD62L negative cells were incubated with dye that excites/emits at 633/635 nm. The cells were then pooled, counted, filtered through a 40-um cell strainer, washed, and the concentration was adjusted to 50-80M/mL by adding XVivo-15. The following buffer solutions were prepared for each sample as shown in Table 18.

TABLE 18
Squeeze Processing Cell Suspension Solutions

| | |
|---|---|
| Group A, E | 100 μL XVivo-15 + 100 μL 2X TILs |
| Group B, F | 100 μL XVivo-15 + 100 μL 2X TILs |
| Group C, G | 100 μL mRNA mix (74.23 μL RPMI + 25 μL mbIL-2 |
| | mRNA + 0.77 μL NaCl) + 100 μL 2X TILs |
| Group D, H | 100 μL mRNA mix (48.46 μL RPMI + 25 μL mbIL-2 |
| | mRNA + 25 μL mbIL-12 mRNA + 1.54 μL NaCl) + |
| | 100 μL 2X TILs |

As shown above in Table 18, Groups A and E were used as controls (i.e., not contacted with any payload and not squeeze processed). Groups B and F were squeeze processed without any payload at room temperature using microfluidic constrictions (70 μm depth, 4.5 μm width, and 10 μm length) at 30 psi. Groups C-D were squeeze processed at room temperature with the payloads shown above, i.e., Group C: mbIL-2 mRNA, Group D mbIL-2 mRNA+mbIL-12 mRNA. Groups C-D were squeeze processed using the same conditions as in Group B (i.e., microfluidic constriction 70 μm depth, 4.5 μm width, and 10 μm length; at 30 psi).

Afterwards, the cells from each of Groups A-H were separately transferred into their own 5 ml conical containing 1 mL RPMI+10% FBS. Cells from all groups were centrifuged separately, at 400 rcf for 4 minutes at room temperature. The supernatant was aspirated, and the cell populations were separately resuspended in 2 mL of RPMI+10% FBS media and cultured in technical duplicate with or without 3000 IU/m L rhIL-2 and/or 125 ng/mL rhIL-12 at 37° C.

### FACS Analysis

At days 1, 4, and 6, 200 ul of cells were aspirated and centrifuged at 400 rcf for 4 minutes at room temperature. The supernatant was discarded, and the cells were resuspended in 50 μL of LA) (1.200 dilution in FACs buffer) and FC block (1:50 dilution in FACs buffer). Cells were incubated at room temperature in the dark for 10 minutes. The cells were spun down at room temperature at 400 ref for 4 minutes. The cells were then incubated at 4 C for 20 minutes with fluorescent anti-human antibodies targeting CD3, CD4, CD8, CD45RO, CD62L, IL-2, and IL-12p70. After incubation 150 μL of FACs isolation buffer were added to each sample and the samples were spun at 400 rcf for 4 minutes. The supernatant was discarded, and the cells resuspended with 200 μL of FACs isolation buffer. The cells were then analyzed using an Attune Flow cytometer.

### Results

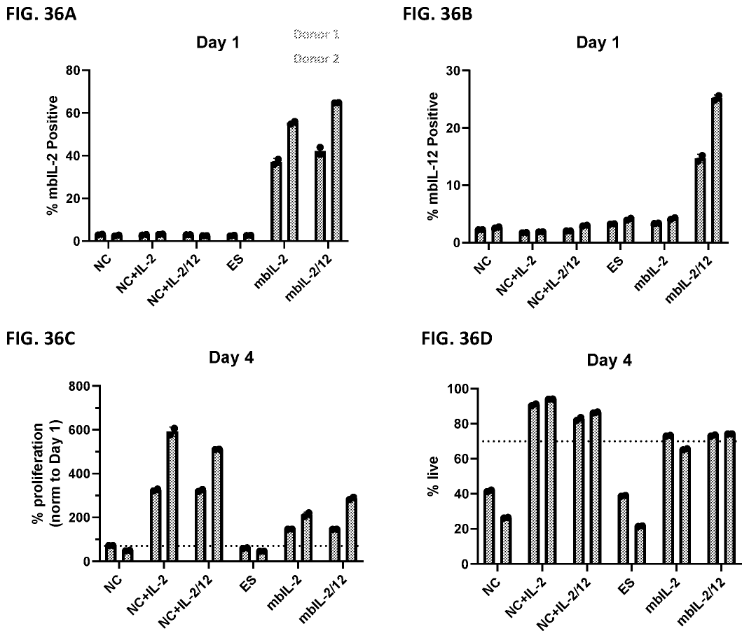

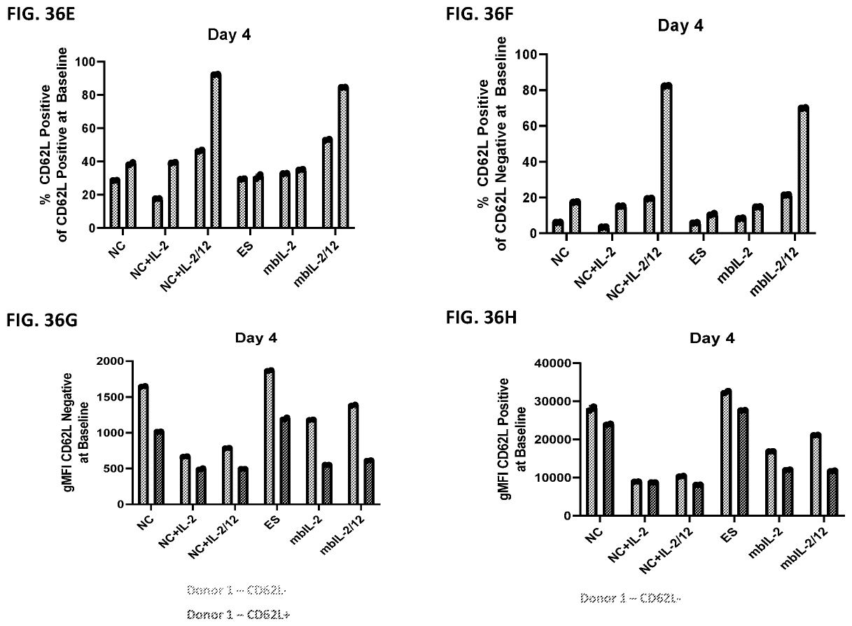

As shown in FIG. 36A-36B, squeeze processing induced expression of the delivered mRNA. All mRNAs showed expression above background at 24 hr (FIG. 36A-36B). mbIL-2 and/or mbIL-12 engineered TILs proliferated and maintained viability for 4 days post-squeeze processing, whereas control TILs without exogenous cytokines did not proliferate and exhibited less than 40% viability at the same timepoint (FIG. 36C-36D). A single application of CD62L selection enriched for 65% CD62L positive cells in the positive fraction, and 88% CD162L negative cells in the negative fraction (data not shown). While expression varied across donors, CD62L positive cells were enriched in both baseline CD62L positive and baseline CD62L negative fractions when cultured in rhIL-12 and/or engineered to express mbLL-12 (FIG. 36E-36F). CD62L positive cells also exhibited a higher proliferative capacity regardless of culture condition and/or baseline CD62L status (FIG. 36G-36H).

Collectively, the results provided herein demonstrate that the squeeze processing methods provided herein can be used to produce TILs that upregulate CD62L either through exogenous or membrane-bound mbIL-12 exposure, and that the emergence of CD62L positive TILs is both via conversion from CD62L negative cells to CD162L positive cells and from the superior proliferative capacity of CD62L positive cells.

### Example 17: mbIL-7 and Bcl-2 Engineered TILs Resistance to rhIL-7 “Sink” Effect

To assess if membrane bound (mb) IL-7 and/or Bcl-2-engineered TILs have increased resistance to limited rhIL-7 availability in vitro (i.e. mimicking the competition for serum IL-7 TILs are subjected to upon non-lymphodepleted human infusion), post-REP TILs were loaded with mRNA encoding mbIL-7 and/or Bcl-2 using the squeeze processing methods provided herein, and cultured in decreasing concentrations of recombinant human (rh) IL-7. The specific methods used are provided below.

### Squeeze Processing

Three 50M cell vials were thawed and diluted with FACs buffer. Cells were centrifuged at 400 rcf at room temperature then washed and resuspended to 3-10M/mL in AIM V media containing 3000 IU/mL recombinant human (rh) IL-2. Cells were transferred to a T75 flask and cultured at 37° C. To allow the cells to recover from thaw, cells were cultured for 72 hr.

Cells were prepared for squeeze-processing. Cells were collected, centrifuged at 400 rcf at room temperature, and then washed, resuspended to 3-10M/mL in AIM V media without rhIL-2 to remove rhIL-2-dependent support prior to squeeze processing, transferred to a T25 flask, and cultured at 37° C. for 24 hours. The cells were then counted, filtered through a 40-um cell strainer, washed, and the concentration was adjusted to 50M/mL by adding XVivo-15. The following buffer solutions were prepared for each sample as shown in Table 19:

TABLE 19
Squeeze Processing Cell Suspension Solutions

| | |
|---|---|
| Group A | 100 μL XVivo-15 + 100 μL 2X TILs |
| Group B | 100 μL XVivo-15 + 100 μL 2X TILs |
| Group C | 100 μL mRNA mix (87.12 μL RPMI + 12.5 μL mbIL-7 |
| | mRNA + 0.385 μL NaCl) + 100 μL 2X TILs |
| Group D | 100 μL mRNA mix (87.12 μL RPMI + 12.5 μL Bcl-2 |
| | mRNA + 0.385 μL NaCl) + 100 μL 2X TILs |
| Group E | 100 μL mRNA mix (74.23 μL RPMI + 12.5 μL mbIL-7 |
| | mRNA + 12.5 μL Bcl-2 mRNA + 0.77 μL NaCl) + |
| | 100 μL 2X TILs |

As shown above in Table 19, Group A was used as a control (i.e., not contacted with any payload and not squeeze processed). Group 13 was squeeze processed without any payload at room temperature using microfluidic constrictions (70 μm depth, 4.5 m width, and 10 μm length) at 30 psi. Groups C-E were squeeze processed at room temperature with the payloads shown above, i.e., Group C: mbIL-7 mRNA; Group D: Bcl-2 mRNA; Group E: mbIL-7 mRNA+Bcl-2 mRNA Groups C-E were squeeze processed using the same conditions as in Group B (i.e., microfluidic constriction: 70 μm depth, 4.5 un width, and 10 μm length; at 30 psi).

Afterwards, the cells from each of Group A-E were separately transferred into their own 5 ml conical containing 1 mL RPMI+10% F3S. Cells from all groups were centrifuged separately, at 400 rcf for 4 minutes at room temperature. The supernatant was aspirated, and the cell populations were separately resuspended in 1 mL of AIM V media and cultured in technical duplicate with or without 10 ng/mL, 1 ng/mL, 0.1 ng/mL, or 0.01 ng/mL rhIL-7 at 37° C.

### FACS Analysis

At days 1 and 5, 200 ul of cells were aspirated and centrifuged at 400 rcf for 4 minutes at room temperature. The supernatant was discarded, and the cells were resuspended in 50 μL of L/D (1:200 dilution in FACs buffer) and FC block (1:50 dilution in FACs buffer). Cells were incubated at room temperature in the dark for 10 minutes. The cells were spun down at room temperature at 400 rcf for 4 minutes. The cells were then incubated at 4 C for 20 minutes with 50 μL of a fluorescent anti-human antibody targeting CD3 and a biotinylated anti-human antibody targeting IL-7. After incubation 150 μL of FACs isolation buffer were added to each sample and the samples were spun at 400 rcf for 4 minutes. The cells were incubated at 4 C for 20 minutes with 50 μL of fluorescent streptavidin. After incubation 150 μL of FACs isolation buffer were added to each sample and the samples were spun at 400 rcf for 4 minutes. The cells were resuspended in 200 μL pre-warmed Fixation buffer and incubated at 37° C. for 15 minutes. After incubation the cells were spun down at room temperature at 400 rcf for 4 minutes. The supernatant was discarded, and the cells were resuspended in 200 μL pre-chilled Permeabilization buffer and incubated at −20° C. for 1 hr. After incubation the cells were spun down at room temperature at 1000 rcf for 4 minutes. The supernatant was discarded, and the cells were incubated at room temperature for 30 minutes with 50 μL of fluorescent anti-human antibody targeting Bcl-2. After incubation 150 μL of FACs isolation buffer were added to each sample and the cells were spun down at room temperature at 1000 rcf for 4 minutes. The supernatant was discarded, and the cells resuspended with 200 μL of FACs isolation buffer. The cells were then analyzed using an Attune Flow cytometer.

### Results

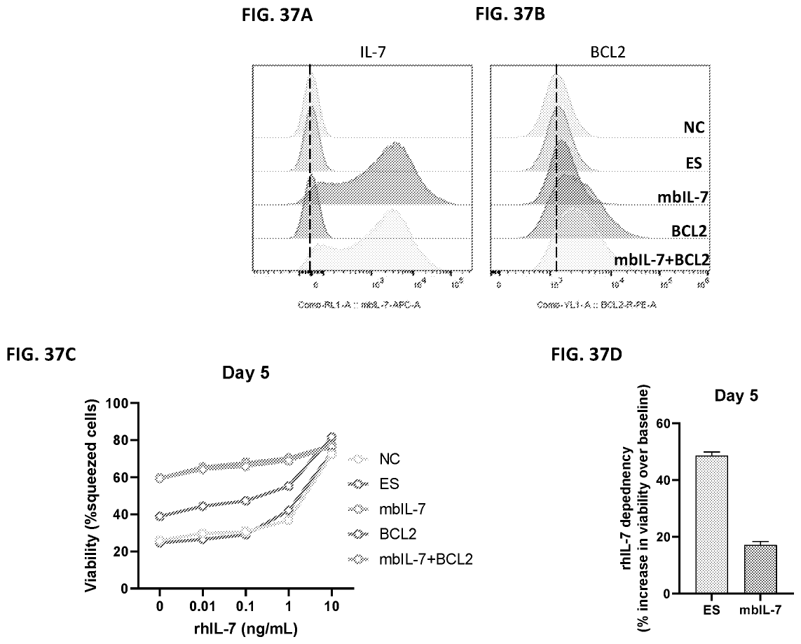

As shown in FIG. 37A-B, squeeze processing induced expression of the delivered mRNA All mRNAs showed expression above background at 24 hr (FIG. 37A-B). On day 5 of culture without rhIL-7 support, control TILs were 25% viable, whereas Bcl-2-engineered TILs were 40% viable, and mbIL-7 and/or co-engineered mbIL-2 and Bcl-2 TILs were 60% viable (FIG. 37C). Control TILs required 10 ng/mL rhIL-7 to attain viability equal to mbIL-7-engineered TILs (FIG. 37C-D), indicating that the modified TILs provided herein (squeeze processed with mRNA encoding membrane-bound IL-7) have improved ability to survive which is less dependent on exogenous cytokines such as IL-7.

Collectively, the results provided herein demonstrate that the squeeze processing methods provided herein can be used to produce TILs that express mbIL-7 and/or Bcl-2, that these engineered TILs have improved survival in rhIL-7-limiting conditions over control TILs, and that 250 μg/mL engineered mbIL-7 TILs provide the equivalent of approximately 10 ng/mL support.

### Example 18: Comparison of Microfluidic Chips and Anti-CD3 Antibody Clones

As described herein, TILs are often expanded to increase their number prior to their use in adoptive transfer studies Such expansion phase can involve the use of anti-CD3 antibodies to activate and induce the TILs to proliferate. Whether the specific anti-CD3 antibody clone used in the expansion phase was next assessed. Briefly, melanoma TILs were collected from REP cultures that contained different anti-CD3 clones (OKT3, SK7, HIT3a, or UCHT1). Following cell harvest, the post-REP TILs were resuspended at 40 million live cells/mL cell density as 2× concentration. The cells were mixed with equal volume of 2× mRNA solution and squeezed through various chip types (10-3.5-70 SF, 10-4-70 ST) and pressure (30 psi) at room temperature. Final concentration of cells was 20 million live cells/mL. Final mRNA concentration for squeeze was 250 mg/mL for each of mbIL-2 and mbIL-12 mRNA. Following the squeeze processing, cells were quenched in CTS OpTmizer media and spun immediately. Squeezed TILs were incubated for 4 hours in a 37° C. incubator in the presence of recombinant IL-2 (500 IU/mL). The squeezed TILs were assessed at 4 hours post squeeze by flow cytometry for viability, cell composition, and surface levels of mbIL-2 and mbIL-12.

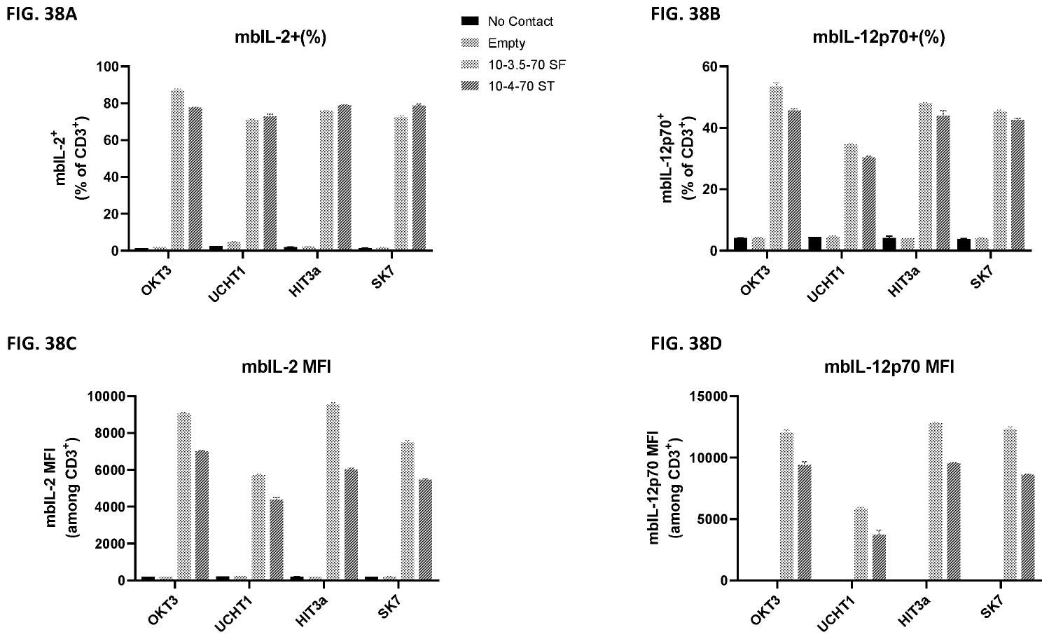

As shown in FIGS. 38A-38D, differences in mbIL-2 and mbIL-12 expression were observed using the different anti-CD3 antibody clones. Generally, higher expression (both as percentage of total cells and mean fluorescence intensity (MFI)) was observed with the OKT3 and HIT3a clones. Additionally, at least at the MFI level, noticeable differences were observed between the two microfluidic chips used. For both mbIL-2 and mbIL-12, the MFI was noticeably higher with the “10-3.5-70 SF” microfluidic chip. This was true for all the anti-CD3 antibody clones tested.

The above results demonstrate that the particular anti-CD3 antibody clone used during the REP expansion phase could have an effect on the delivery efficiency of the mRNAs to the TILs using squeeze processing. Additionally, the above data also suggests that, at least at the MFI level, the particular microfluidic chip used could have an effect.

### Example 19: Comparison of Anti-CD3 Antibody Concentration

As described herein, in some aspects, prior to modifying the TILs, the TILs can undergo a first expansion phase (pre-REP) and a second expansion phase (REP). Whether the concentration of the anti-CD3 antibody during the first expansion phase has an effect in producing the modified TILs described herein, the following methods were used. Briefly, lung TILs underwent a pre-REP process in the absence or presence of 6 or 30 ng/mL anti-CD3 antibody (OKT3 clone). Then, the TILs were collected and further expanded in a REP process. Afterwards, the cells were resuspended at 40 million live cells/mL cell density as 2× concentration. The cells were mixed with equal volume of 2× mRNA solution and squeezed through 10-4-70 ST at 60 psi at room temperature. Final concentration of cells was 20 million live cells/mL. Final mRNA concentration for squeeze was 250 mg/mL for each of mbIL-2 and mbIL-12 mRNA. Following squeezing, cells were quenched in CTS OpTmizer media and spun immediately. Squeezed TILs were incubated for 4 hours in a 37° C. incubator in the presence of recombinant IL-2 (500 IU/mL). The squeezed TILs were assessed at 4, 24, and 48 hours post squeeze by flow cytometry for viability, cell composition, surface levels of mbIL-2 and mbIL-12.

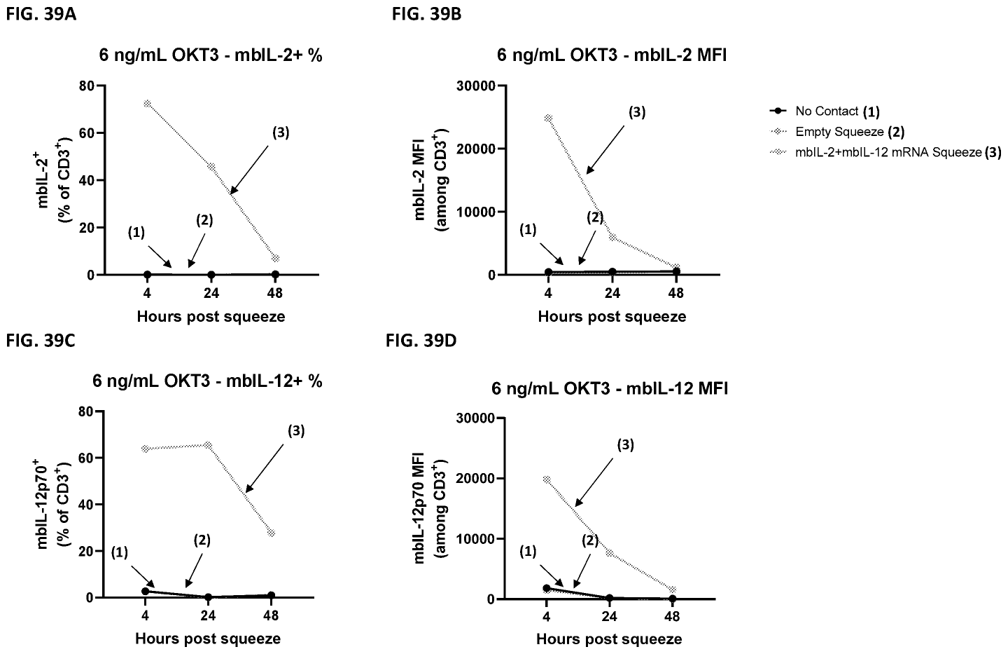

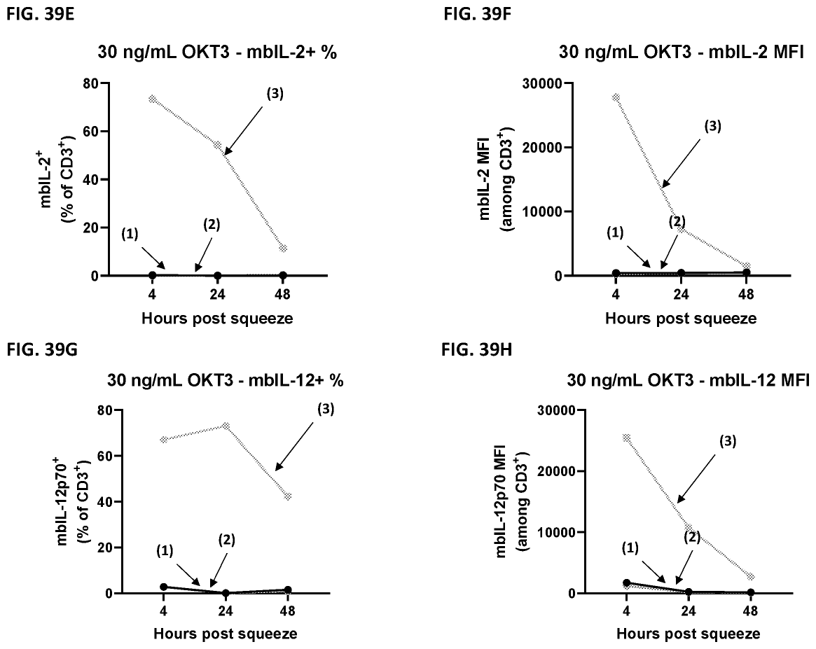

As shown in FIGS. 39A-39H, the concentration of the anti-CD3 antibody during the pre-REP process had minimal impact on the translation kinetics of mbIL-2 and mbIL-12 expression on the squeeze processed TILs. mbIL-2 and mbIL-12 had highest expression at 4 hours post squeeze processing, and subsequently decreased with time. The decrease in expression was much quicker for mbIL-2 as compared to mbIL-12. By about 48 hours post squeeze processing, there was minimal mbIL-2 and mbIL-12 expression observed in the TILs. Additionally, in agreement with some of the earlier Examples provided in the present disclosure, the modified TILs (i.e., squeeze processed with mRNA encoding mbIL-2 and mRNA encoding mbIL-12) upregulated CD62L expression with time, as compared to both the unprocessed TILs and TILs squeeze processed with no mRNA (i.e., empty squeeze).

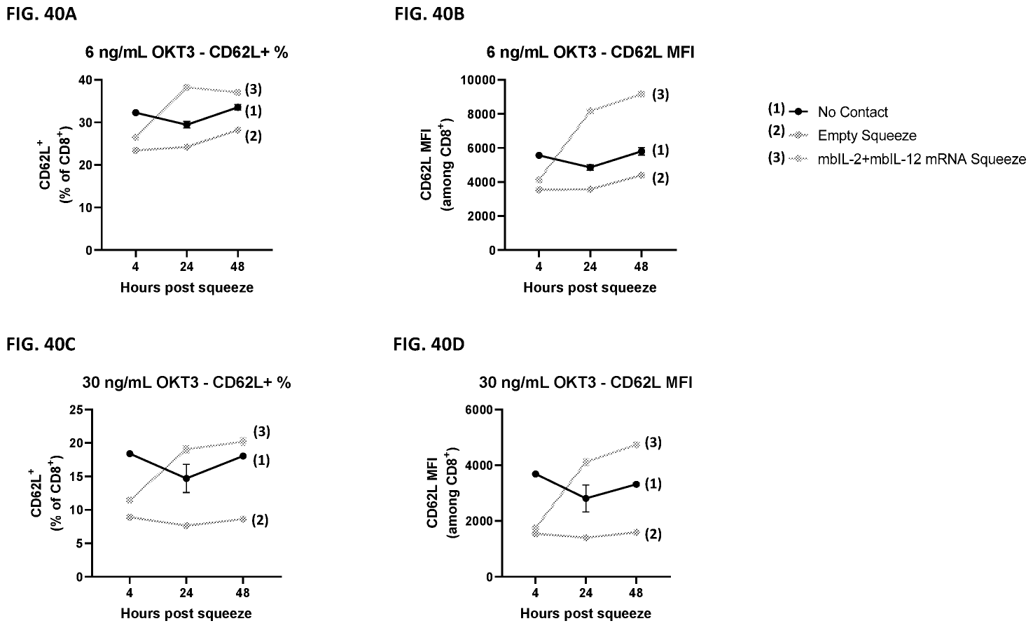

The above results demonstrate that the concentration of the anti-CD3 antibody during the pre-REP phase has minimal impact on the delivery efficiency of the mRNAs to the TILs using squeeze processing. The results also confirm that the squeeze processing methods described herein can alter the T cells to become more central memory like (as evidenced by the increased CD62L expression) (see FIGS. 40A-40D).

### Example 20: Comparison of Microfluidic Chip and Pressure

To further assess the effect that different microfluidic chips and pressures might have on the delivery efficiency of mRNAs to TILs using squeeze processing, the following methods were used. Briefly, lung TILs were collected from the REP culture Following cell harvest, they were resuspended at 200 million live cells/mL cell density as 2× concentration. The cells were mixed with equal volume of 2× mRNA solution and squeezed through 10-4-70 ST or 10-4.5-70 ST at 45 or 60 psi at room temperature. Final concentration of cells was 100 million live cells/mL. Final mRNA concentration for squeeze was 250 mg/mL for each of mbIL-2 and mbIL-12 mRNA. Following squeezing, cells were quenched in CTS OpTmizer media and spun immediately. Squeezed TILs were incubated for 4 hours in a 37° C. incubator in the presence of recombinant IL-2 (500 IU/mL). The squeezed TILs were assessed at 4 hours post squeeze by flow cytometry for viability, cell composition, and surface levels of mbIL-2 and mbIL-12.

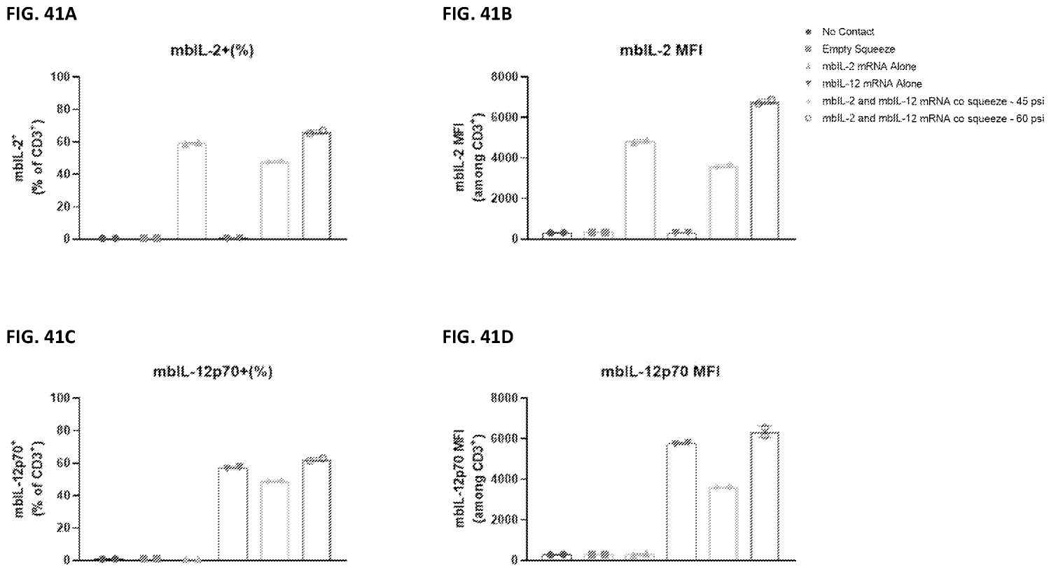

At high cell density, improved delivery of the mRNAs was observed with 10-4.5-70 ST chip as compared to the 10-4-70 ST chip. As to pressure, much better delivery efficiency (as evidenced by mbIL-2 and mbIL-12 expression both as percentage and as MFI) was observed with 60 psi as compared to 45 psi (see, e.g., last two columns in FIGS. 41A-41D).

The above results further demonstrate that by varying certain delivery parameters (e.g., microfluidic chip and pressure) the delivery efficiency of squeeze processing can be modulated.

### Example 21: Comparison of Cell Density

To assess whether the cell density of the cell suspension that is passed through the constriction has an effect, the following methods were used Briefly, cryopreserved melanoma REP TILs were thawed and resuspended in AIM V media containing 6000 IU/mL recombinant IL-2. These cells were incubated for 5 days at 37° C. incubator 5 days later, the TILs were harvested and prepared for squeezing. The TIL suspension was split equally into half, and each was adjusted to a cell density of 40 million live cells and 200 million live cells per mL. The cells were mixed with equal volume of 2× mRNA solution and squeezed through 10-4-70 ST chip at 60 psi at room temperature. Final concentration of cells was 20 M and 100 million live cells/mL for respective groups. Final mRNA concentration for squeeze was 250 mg/mL for each of mbIL-2 and mbIL-12 mRNA. Following squeezing, cells were quenched in CTS OpTmizer media and spun immediately Squeezed TILs were incubated for 4, 24, 48 hours, and 8 days in a 37° C. incubator in the absence or presence of recombinant IL-2 (500 IU/mL). The squeezed TILs were assessed at each time point by flow cytometry for viability, surface levels of mbIL-2 and mbIL-12, and phenotypic markers such as CCR7, CXCR3, CD45RO, CD62L, CD127, and CD25.

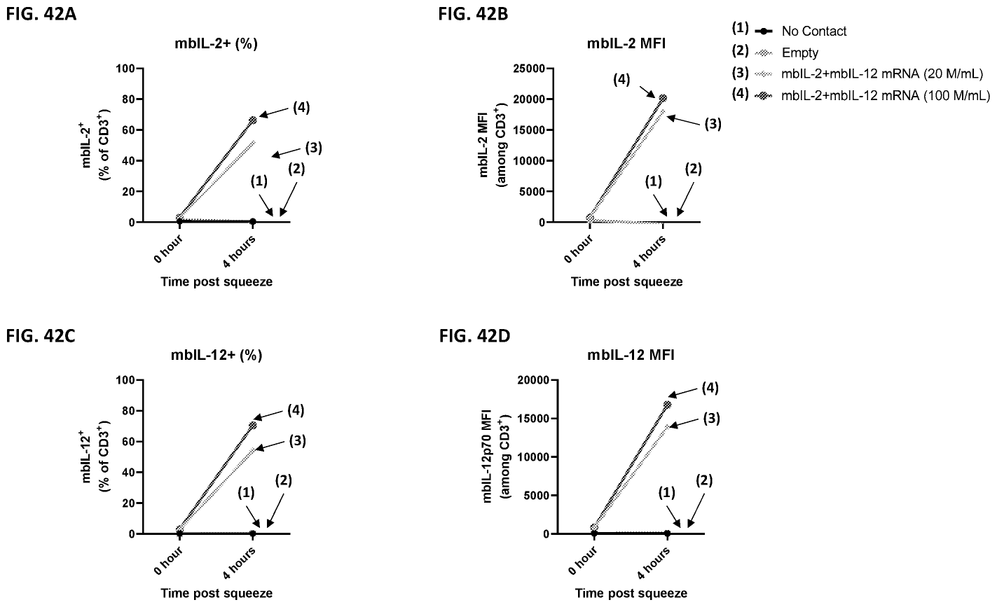

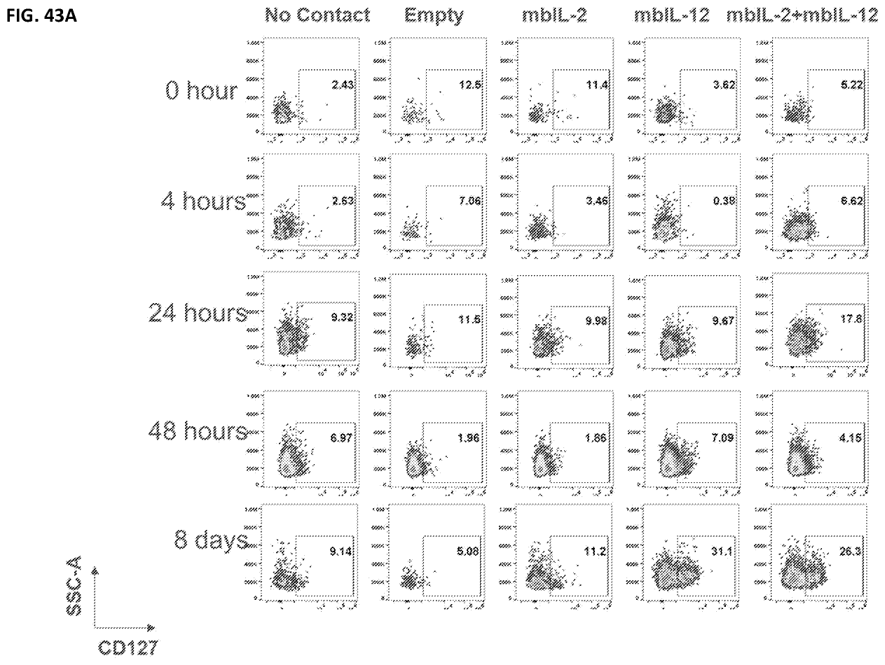

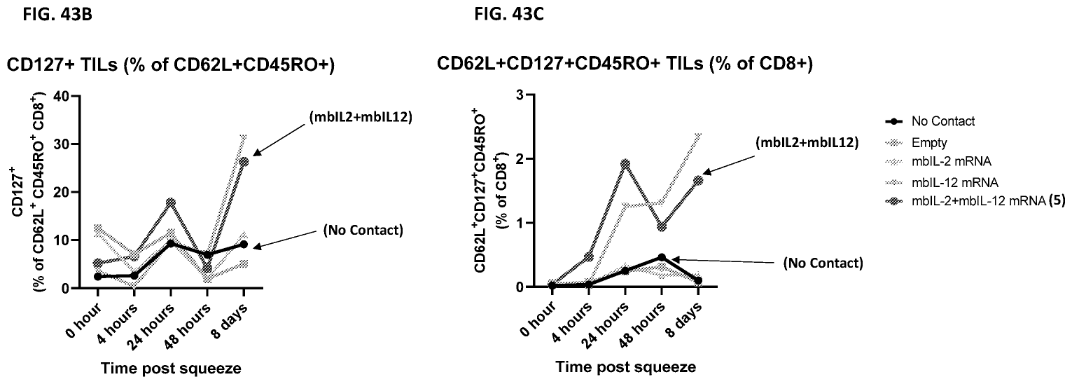

As shown in FIGS. 42A-42D, higher mbIL-2 and mbIL-12 expression was observed in the TILs (both as a percentage and as MFI) with the higher cell density (i.e., 100 million cells/mL). And, as shown in FIGS. 43A-43C, in squeeze-processed TILs expressing mbIL-12, there was a significant increase in the percentage of TILs with central memory phenotype (as evidenced by the positive-expression of CD62L, CD127, and CD45RO), which was observed as early as day 1 post squeeze processing and further increased by day 8 post squeeze processing.

The above results demonstrate that higher cell density during the squeeze processing could be useful in improving the delivery efficiency of mRNAs into TILs using squeeze processing. Additionally, the above results also confirm some of the data described in the earlier Examples, suggesting that the squeeze processing methods provided herein could be useful in producing TILs with a central memory phenotype (particularly where the TILs are modified to express membrane-bound IL-12).

### Example 22: Viability and Expression Analysis

To further assess the squeeze processing methods provided herein on viability of the modified TILs, the following methods were used. REP TILs were harvested and resuspended at 200 million live cells/mL cell density as 2× concentration mRNA solutions were also prepared at 1 mg/mL concentration each for mbIL-2 and mbIL-12 mRNA as 2× concentration. Both cell suspension and the mRNA solutions were pre-chilled separately in a Cool Rack at 4° C. for 20 minutes. After 20 minutes, both cells and mRNA solutions were mixed and squeezed via 10-4.5-70 ST chip at 60 psi at RT. Final concentration of cells was 100 million live cells/mL. Final mRNA concentration for squeeze was 500 mg/mL for each of mbIL-2 and mbIL-12 mRNA. Following squeezing, the squeezed cells were quenched into media that was kept in a 37° C. incubator for 2 hours prior to this step. The squeezed cells were held in the quench media for 20 minutes at room temperature. Cells were spun down and resuspended in various cryo media formulations. They were held in cryo media for 20 minutes at RT. Following the hold, cryovials were filled and the cells were cryopreserved for future use.

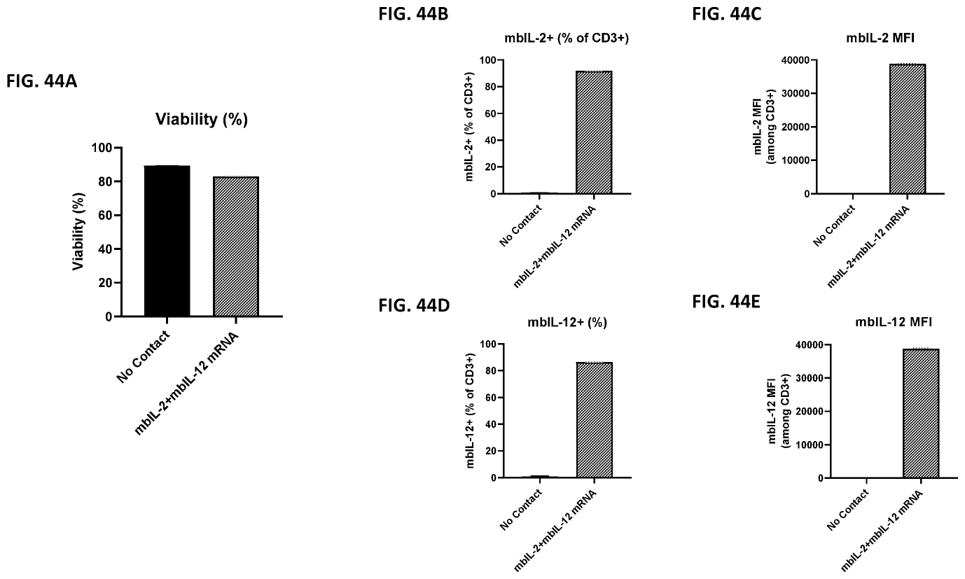

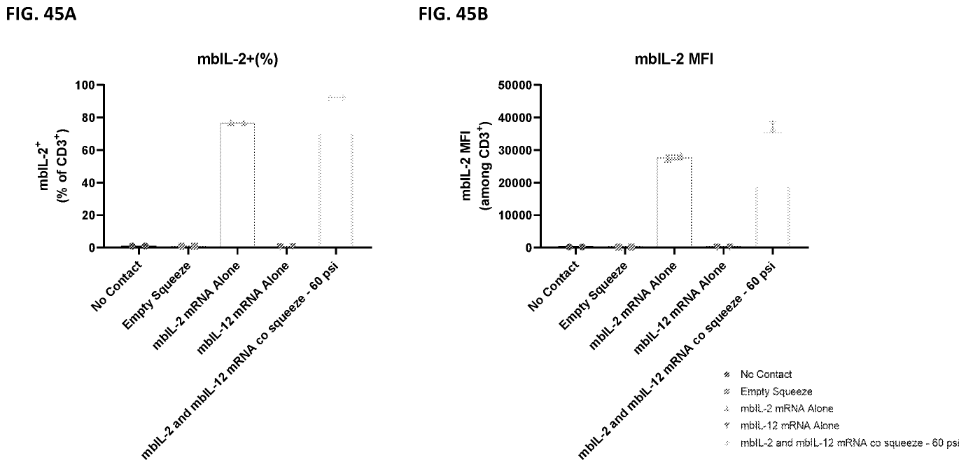

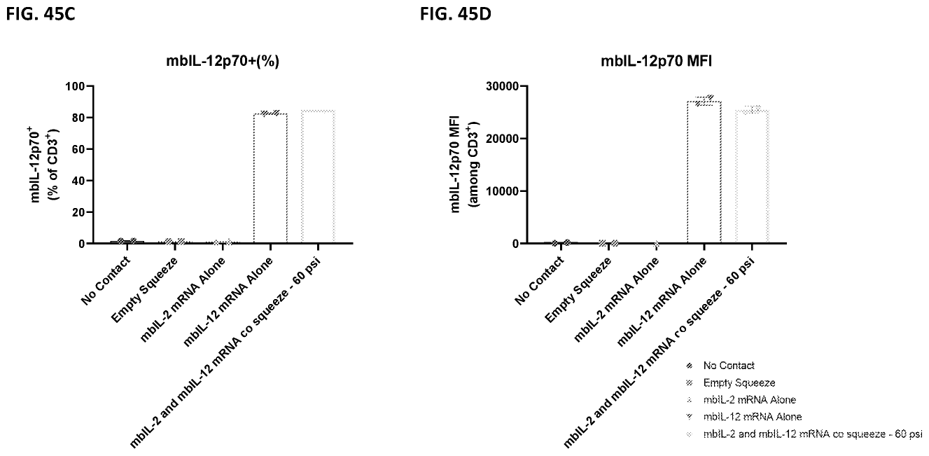

Using the 10-4.5-70 ST microfluidic chip at a pressure of 60 psi resulted in high mbIL-2 and mbIL-12 expression on the squeeze processed TILs (both as percentage and as MFI) (see FIGS. 44B-44E; and 45A-45D). As to viability, no significant differences were observed between the squeezed processed TILs and unprocessed TILs (FIG. 44A). See also FIGS. 45A-45). These results further demonstrate the effectiveness of the squeeze processing methods provided herein in delivering mRNAs (e.g., encoding membrane-bound cytokines) to TILs.

### Example 23: In Vivo Analysis in Autologous PDX Tumor Model

To assess the anti-tumor effects of the modified TILs described herein, an autologous PDX tumor animal model will be used. The animals will receive one of the following: (i) TILs squeeze processed with mRNA encoding membrane-bound IL-2 and mRNA encoding membrane-bound IL-12, (ii) TILs squeeze processed with no mRNA (i.e., empty squeeze), and (iii) unprocessed TILs (i.e., no squeeze processing) that were cultured in recombinant human IL-2. One or more the following parameters will be assessed: (1) tumor killing capacity, e.g., assessed by measuring effect on tumor progression and time to endpoint; (2) infiltration of TILs into tumor, e.g., tumors will be harvested, and TIL infiltration measured by flow cytometry; (3) phenotypic characterization of TILs, e.g., measure activation markers by flow cytometry; and (4) persistence of TILs in vivo, e.g., persistence of TILs in circulation will be assessed by flow cytometry.

### Example 24: In Vivo Analysis in Syngeneic Tumor Model

To further asses the anti-tumor effects of the modified TILs described herein, a syngeneic tumor animal model will be used. Briefly, B16-F10 tumors will be implanted into mice. Once optimal tumor size has been reached, the mice will receive one of the following. (i) transgenic pmel-1 T cells squeeze delivered with mRNA encoding membrane-bound IL-2, (ii) transgenic pmel-1 T cells squeeze delivered with mRNA encoding membrane-bound IL-12, and/or (iii) transgenic pmel-1 T cells squeeze delivered with mRNA encoding membrane-bound IL-2 and mRNA encoding membrane-bound IL-12. In some aspects, control animals will receive unprocessed T cells and/or T cells squeeze processed with no mRNA. Then, anti-tumor immune response will be assessed (e.g., measure T cell response and/or observe tumor growth).

### Example 25: In Vivo Analysis in Syngeneic Tumor Model

To further asses the anti-tumor effects of the modified TILs described herein, a syngeneic tumor animal model will be used. Briefly, B16-F10 tumors will be implanted into mice. Once optimal tumor size has been reached, the mice will receive one of the following: (i) transgenic pmel-1 T cells squeeze delivered with mRNA encoding membrane-bound IL-2, (ii) transgenic pmel-1 T cells squeeze delivered with mRNA encoding membrane-bound IL-12, and/or (iii) transgenic pmel-1 T cells squeeze delivered with mRNA encoding membrane-bound IL-2 and mRNA encoding membrane-bound IL-12. In some aspects, control animals will receive unprocessed T cells and/or T cells squeeze processed with no mRNA. Then, anti-tumor immune response will be assessed (e.g., measure T cell response and/or observe tumor growth).

### Example 26: Effect of Anti-CD3 Antibody on TIL Expansion

Further to the data provided in Example 22, the effect of anti-CD3 antibody on TIL expansion was further assessed. Briefly, freshly resected melanoma tumor was rinsed in 1× Hank's Balanced Salt Solution (HBSS) with 10 μg/mL gentamicin and dissected into 8-27 mm³ fragments using a forceps and scalpel. Duplicate wells of a G-Rex6M plate were filled with either 0, 6, or 30 ng/mL OKT3 antibody in 20 mL of CTS OpTmizer media with 100 U/mL penicillin, 100 μg/mL streptomycin, 1× GlutaMAX Supplement, and 6,000 IU/mL recombinant IL-2. Five tumor fragments, 8-27 mm³ in size, per well were added to the G-Rex6M plate. The G-Rex6M plate was then placed in a 37° C., 5% CO₂ incubator for 4 days.

On day 4, the G-Rex6M plate was removed from the incubator. 20 mL of CTS OpTmizer media with 100 U/mL penicillin, 100 μg/mL streptomycin, lx GlutaMAX Supplement, and 6,000 IU/mL recombinant IL-2 were added on top of the 20 mL of media in each of the wells of the G-Rex6M plate. The G-Rex6M plate was returned to the 37° C., 5% CO₂ incubator for an additional 9 days.

On day 13, the TILs were harvested and transferred to a 50 mL conical tube. The number and viability of the TILs was assessed using a NucleoCounter NC-200.

As shown in Table 20 (below), melanoma tumor fragments cultured with either 6 or 30 ng/mL of OKT3 resulted in greater TIL expansion by day 13 compared to the culture without OKT3 This result demonstrates that addition of OKT3 to the initial TIL culture can increase the proliferation of the TILs.

TABLE 20
Anti-CD3 Antibody Concentration on Cell Count and Viability

| Culture | Replicate | Cell Count | Viability |
|---|---|---|---|
| Condition | Well | on Day 13 | on Day 13 |
| No OKT3 | #1 | 1.50 × 10⁸ | 88.7% |
| | #2 | 5.72 × 10⁷ | 82.5% |
| 6 ng/mL OKT3 | #1 | 2.35 × 10⁸ | 90.7% |
| | #2 | 2.43 × 10⁸ | 91.1% |
| 20 ng/mL OKT3 | #1 | 2.00 × 10⁸ | 90.5% |
| | #2 | 2.17 × 10⁸ | 91.6% |

### Example 27: Effect of Tumor Fragment Size on TIL Expansion

To further assess the different variables that might have an effect on producing the modified TILs described herein, tumor fragment size during the initial culturing process of TILs was assessed. Briefly, freshly resected lung tumor was rinsed in 1× Hank's Balanced Salt Solution (H-BSS) with 10 μg/mL gentamicin and dissected into 1 or 27 mm³ fragments using a forceps and scalpel. Duplicate wells of a G-Rex6M plate were filled with CTS OpTmizer media with 100 U/mL penicillin, 100 μg/mL streptomycin, and 6,000 IU/mL recombinant IL-2 Five tumor fragments, either 1 or 27 mm³ in size, per well were added to the G-Rex6M plate. The G-Rex6M plate was then placed in a 37° C., 5% CO₂ incubator for 4 days.

On day 3, the G-Rex6M plate was removed from the incubator. 20 mL of CTS OpTmizer media with 100 U/mL penicillin, 100 μg/mL streptomycin, and 6,000 IU/mL recombinant IL-2 were added on top of the 20 mL of media in each of wells of the G-Rex6M plate. The G-Rex6M plate was returned to the 37° C., 5% CO₂ incubator. On day 7 and 10, the G-Rex6M plate was removed from the incubator 20 mL of culture media were removed from each well and 20 mL of CTS OpTmizer media with 100 U/mL penicillin, 100 μg/mL streptomycin, and 6,000 IU/mL recombinant IL-2 were added back. The G-Rex6M plate was returned to the 37° C., 5%/o CO₂ incubator.

On day 13, the TILs were harvested and transferred to a 50 mL conical tube. The number and viability of the TILs was assessed using a NucleoCounter NC-200.

As shown in Table 21 (below), 27 mm³ lung tumor fragments resulted in greater TIL expansion by day 13 compared to the cultures with 1 mm³ fragments. This result demonstrates that culturing 27 mm³ tumor fragments results in an increased output of TILs.

TABLE 21
Tumor Fragment Size on Cell Count and Viability

| Culture | Replicate | Cell Count | Viability |
|---|---|---|---|
| Condition | Well | on Day 13 | on Day 13 |
| 1 mm³ fragments | #1 | 4.00 × 10⁵ | 885.8% |
| | #2 | 2.42 × 10⁵ | 78.5% |
| 27 mm³ fragments | #1 | 5.79 × 10⁶ | 94.0% |
| | #2 | 2.48 × 10⁷ | 91.5% |

### Example 28: Effect of Cytokine During TIL Expansion

To assess the effect of different cytokines (IL-2 or IL-15) during the initial TIL expansion and culturing, the following methods were used. Briefly, freshly resected colorectal tumor was rinsed in 1× Hank's Balanced Salt Solution (HBSS) with 10 g/mL gentamicin and dissected into 8-27 mm³ fragments using a forceps and scalpel. Duplicate wells of a G-Rex6M plate were filled with CTS OpTmizer media with 100 U/mL penicillin, 100 μg/mL streptomycin, and either 6,000 IU/mL recombinant IL-2, 500 U/mL recombinant IL-15, or 3,000 U/mL recombinant IL-15 Five tumor fragments, 8-27 mm³ in size, per well were added to the G-Rex6M plate. The G-Rex6M plate was then placed in a 37° C., 5% CO₂ incubator for 3 days.

On day 3, the G-Rex6M plate was removed from the incubator. 20 mL of CTS OpTmizer media with 100 U/mL penicillin, 100 μg/mL streptomycin, and either 6,000 IU/mL of IL-2, 500 U/mL IL-15, or 3,000 U/mL IL-15 were added on top of the 20 mL of media in each of wells of the G-Rex6M plate. The G-Rex6M plate was returned to the 37° C., 5% CO₂ incubator. On day 7 and 10, the G-Rex6M plate was removed from the incubator. 20 mL of culture media were removed from each well and 20 mL of CTS OpTmizer media with 100 U/mL penicillin, 100 μg/mL streptomycin, and either 6,000 IU/mL of IL-2, 500 U/mL IL-15, or 3,000 U/mL IL-15 were added back. The G-Rex6M plate was returned to the 37° C., 5% CO₂ incubator.

On day 14, the TILs were harvested and transferred to a 50 mL conical tube. The number and viability of the TILs was assessed using a NucleoCounter NC-200.

As shown in Table 22 (below), TIL expansion was observed in all conditions. This result demonstrates that culturing tumor fragments in IL-2 or IL-15 can both promote TIL expansion in culture.

TABLE 22
IL-2 and IL-15 on Cell Count and Viability

| Culture | Replicate | Cell Count | Viability |
|---|---|---|---|
| Condition | Well | on Day 13 | on Day 13 |
| 6000 IU/mL IL-2 | #1 | 2.11 × 10⁷ | 87.2% |
| | #2 | 4.00 × 10⁷ | 95.5% |
| 500 U/mL IL-15 | #1 | 1.92 × 10⁷ | 96.5% |
| | #2 | 8.32 × 10⁶ | 93.3% |
| 3,000 U/mL IL-15 | #1 | 3.34 × 10⁶ | 94.8% |
| | #2 | 2.96 × 10⁷ | 91.3% |

### Example 29: Effect of Anti-CD28 on TIL Expansion

To assess the effect that anti-CD28 stimulation has on during the initial TIL expansion and culturing, the following methods were used. Briefly, freshly resected melanoma tumor was rinsed in 1× Hank's Balanced Salt Solution (HBSS) with 10 μg/mL gentamicin and dissected into 8-27 mm³ fragments using a forceps and scalpel. Duplicate wells of a G-Rex6M plate were filled with CTS OpTmizer media with 100 U/mL penicillin, 100 μg/mL streptomycin, and either 6,000 IU/mL recombinant IL-2, 3,000 U/mL recombinant IL-15+0.05 μg/mL anti-CD28, or 3,000 U/mL recombinant IL-15+0.5 μg/mL anti-CD28. Five tumor fragments, 8-27 mm³ in size, per well were added to the G-Rex6M plate. The G-Rex6M plate was then placed in a 37° C., 5% CO₂ incubator for 5 days.

On day 5, the G-Rex6M plate was removed from the incubator 20 mL of CTS OpTmizer media with 100 U/mL penicillin, 100 mg/mL streptomycin, and either 6,000 IU/mL of IL-2 or 3,000 U/mL IL-15 were added on top of the 20 mL of media in each of wells of the G-Rex6M plate. The G-Rex6M plate was returned to the 37° C., 5% CO₂ incubator. On day 9, the G-Rex6M plate was removed from the incubator. 20 mL of culture media were removed from each well and 20 mL of CTS OpTmizer media with 100 U/mL penicillin, 100 μg/mIL streptomycin, and either 6,000 IU/mL of IL-2 or 3,000 U/mL IL-15 were added back. The G-Rex6M plate was returned to the 37° C., 5% CO₂ incubator.

On day 13, the TILs were harvested and transferred to a 50 mL conical tube. The number and viability of the TILs was assessed using a NucleoCounter NC-200.

As shown in Table 23, TIL expansion was observed in all conditions. This result demonstrates that culturing tumor fragments in IL-2 or IL-15+anti-CD28 can both promote TIL expansion in culture.

TABLE 23
Anti-CD28 on Cell Count and Viability

| | Replicate | Cell Count | Viability |
|---|---|---|---|
| Culture Condition | Well | on Day 13 | on Day 13 |
| 6000 IU/mL IL-2 | #1 | 1.06 × 10⁶ | 97.6% |
| | #2 | 5.22 × 10⁷ | 94.4% |
| 3,000 U/mL IL-15 + | #1 | 2.13 × 10⁷ | 72.4% |
| 0.05 μg/mL anti-CD28 | #2 | 4.32 × 10⁶ | 80.0% |
| 3,000 U/mL IL-15 + | #1 | 1.06 × 10⁷ | 91.3% |
| 0.5 μg/mL anti-CD28 | #2 | 1.56 × 10⁷ | 92.5% |

### Example 30: Post Thaw Analysis

To evaluate whether freeze-thaw (including any effect from one or more of the cryopreservatives) has an effect on the squeeze processed TILs described herein, the following methods were used and the mRNA translation and phenotypic expression were assessed. Briefly, cryopreserved, squeezed melanoma and colorectal TILs were thawed and resuspended in CTS OpTmizer media. Post thaw cell recovery and viability were measured via NC-200. Cells were plated in tissue culture-treated plates and incubated for 4, 24 hours, and 8 days in a 37° C. incubator. The TILs were squeezed were assessed at each time point including t=0 (immediately post thaw) by flow cytometry for viability, surface levels of mbIL-2 and mbIL-12, and phenotypic markers such as CCR7, CD45RO, CD62L, CD127, KLRG-1, CD25, CD69, CD39, CD103, and CD25.

### Effect of DMSO

To assess the potential impact from DMSO, the following TILs were formulated in varying concentrations of DMSO (i.e., 0%, 1.25%, 2.5%, and 5%): (1) TILs squeeze processed with mRNA encoding membrane-bound LL-2 and mRNA encoding IL-12; and (2) non-squeeze processed TILs. Each of the formulated samples also contained 5% human serum albumin. Then, the TILs were cryopreserved and subsequently thawed as described above.

### Effect of HSA

To assess the potential impact from HSA, the above-described squeeze-processed and unprocessed TILs were separately formulated in varying concentrations of HSA (i.e., 0%, 1.25%, 2.5%, 5%, and 10%). Each of these formulated samples contained 5% DMSO. Then, the TILs were cryopreserved and subsequently thawed as described above.

### Results

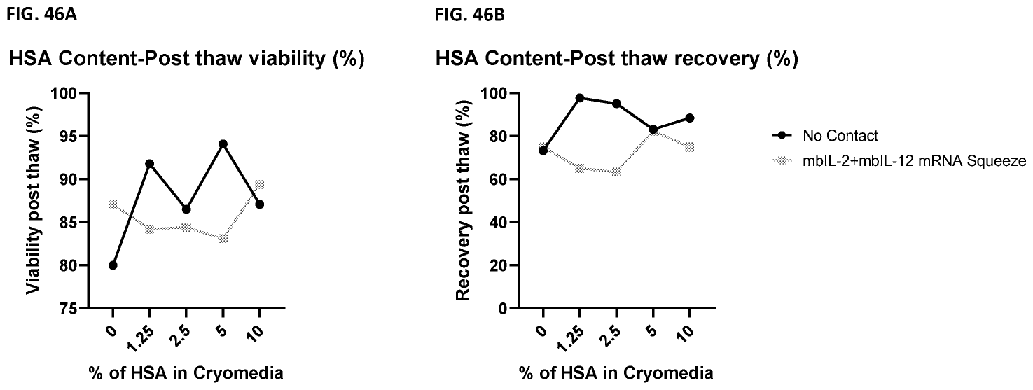

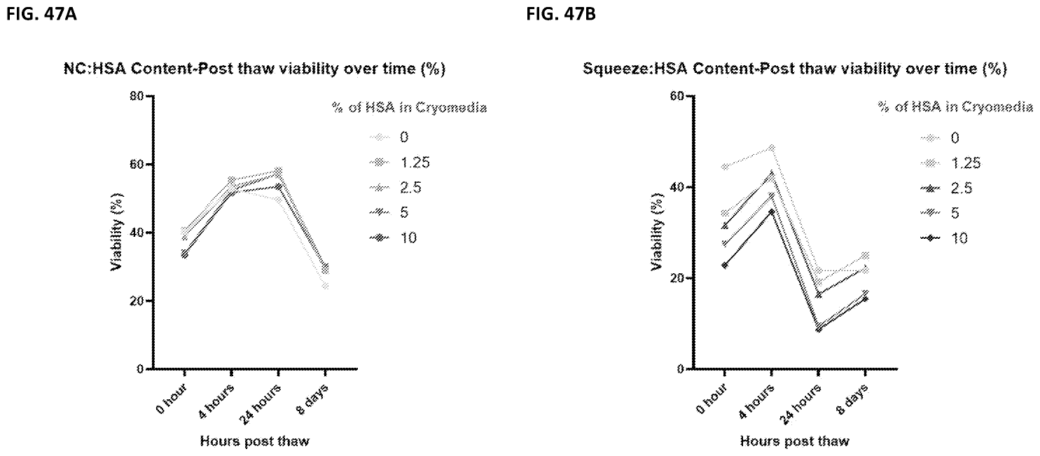

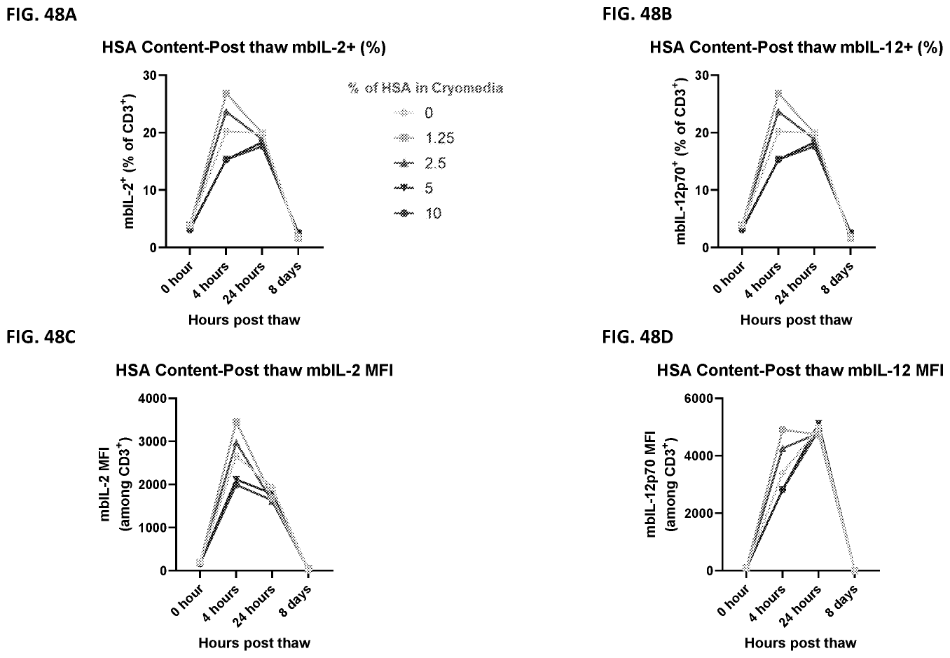

As shown in FIGS. 46A and 46B, HSA concentration had minimal impact on viability and recovery post-thaw. Viability data at various time points post-thaw did suggest that less HSA could result in improved viability (see FIGS. 47A and 47B) As to mRNA translation, greatest membrane-bound IL-2 and membrane-bound IL-12 expression was observed with reduced HSA concentration, at least at 4 hours post-thaw (see FIGS. 48A-48D).

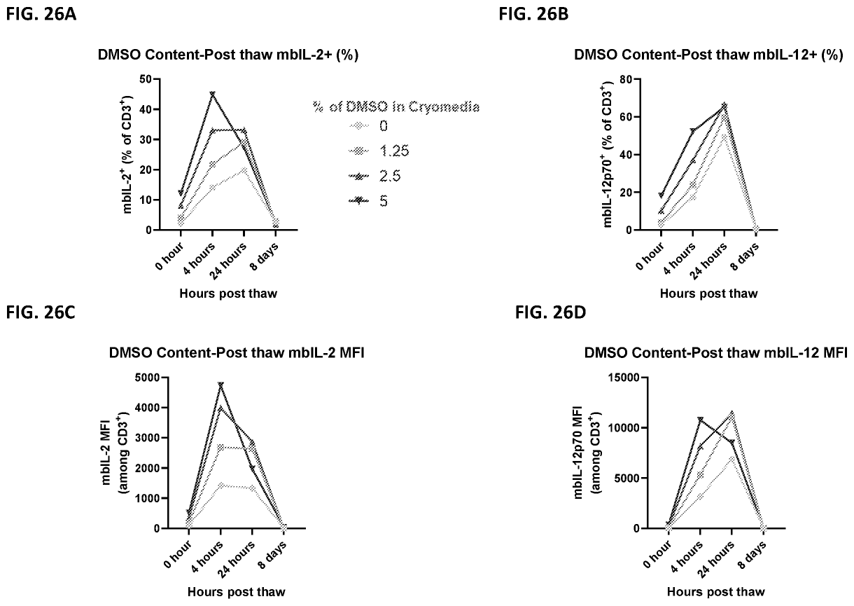

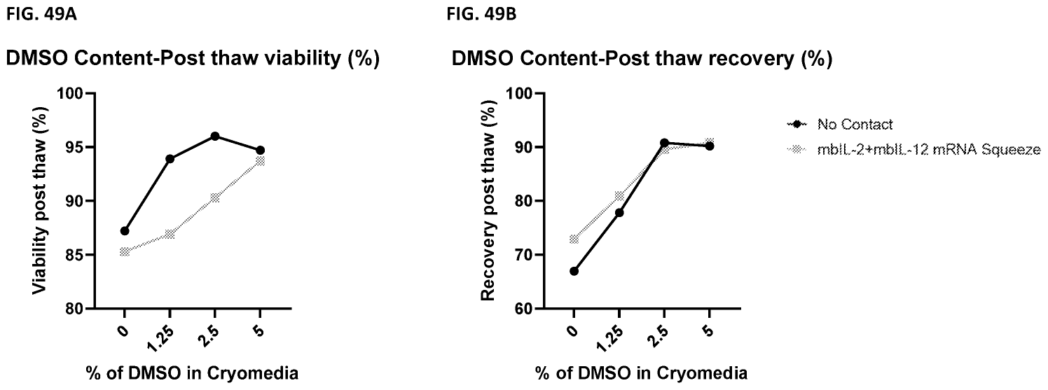

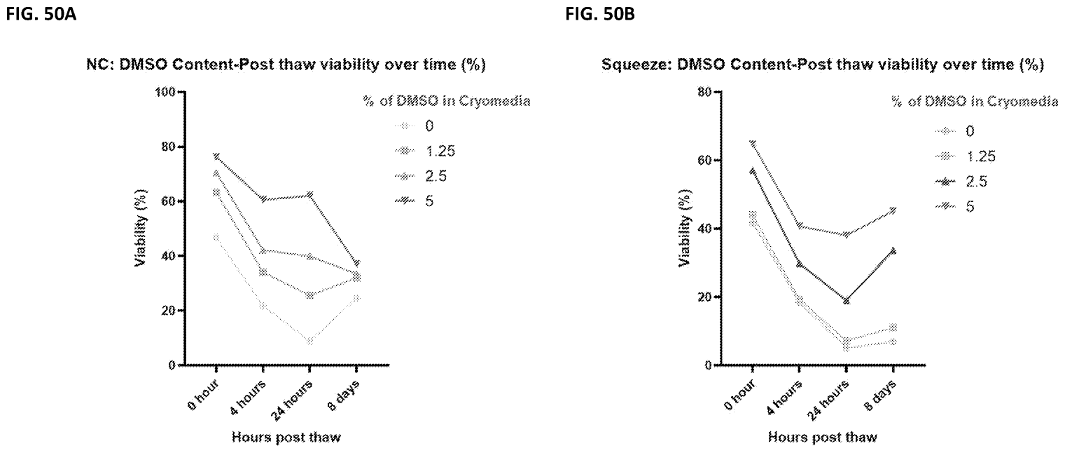

In contrast, with DMSO, increased concentration of DMSO generally resulted in improved effects post-thaw. As shown in FIGS. 49A and 49B, there was a dose-dependent effect of DMSO on viability and recovery post-thaw. Generally, higher DMSO percentage resulted in higher viability and recovery. Additionally, for both squeeze-processed and unprocessed TILs, less DMSO also led to lower viability over time after thaw (see FIGS. 50A and 50B). In addition to improved viability and recovery post-thaw, the presence of DMSO did not have a negative impact on the translation of the delivered mRNAs. As shown in FIGS. 26A-26D, greatest translation of the mRNAs was observed in samples with the greatest about of DMS concentration, as evidenced by increased expression of membrane-bound IL-2 and membrane-bound IL-12 (both as percentage of total cells expressing the membrane-bound IL-2 or IL-12 and as the geometric mean intensity of the expression level). As to phenotypic expression, post-thaw (under both conditions), TILs squeezed with mbIL-2 and mbIL-12 mRNA together exerted upregulation of CD39+CD103+ double positive population, CD69+ population, CD62L+ CD127+ double positive population compared to unprocessed (no contact) TIL controls. These TILs also did not show any KLRG-1 expression which indicated absence of terminally differentiated cells.

Collectively, the above results demonstrate that the TILs described herein (e.g., squeeze-processed and exhibiting increased expression of a co-stimulatory molecule, anti-apoptotic factor, and/or cytokine) are suitable for cryopreservation, particularly in formulations comprising low concentrations of HSA and/or high concentrations of DMSO.

### INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes.

### INCORPORATION BY REFERENCE

While various specific aspects have been illustrated and described, the above specification is not restrictive. It will be appreciated that various changes can be made without departing from the spirit and scope of the disclosure(s) Many variations will become apparent to those skilled in the art upon review of this specification.

Sequence Listing

| | | |
|---|---|---|
| 1 | ATGATGGTGGACGGCGACAACAGCCACGTGGAAATGAAGCTG | TFRC_G4S_IFN-a2a |
| | GCCGTGGACGAGGAAGAGAACGCCGACAACAACACCAAGGCC | mRNA |
| | AACGTGACCAAGCCTAAGAGATGCAGCGGCAGCATCTGCTAC | |
| | GGCACAATCGCCGTGATCGTGTTCTTCCTGATCGGCTTTATGAT | |
| | CGGCTACCTGGGCTACTGCAAGAGCAGTGATGGACCTGGCGAA | |
| | ACAGGCGGAGGCGGAGGATCTGGTGGCGGAGGAAGCGGTGGC | |
| | GGCGGATCTTGTGATCTGCCTCAGACACACAGCCTGGGCAGCA | |
| | GACGAACACTGATGCTGCTGGCCCAGATGCGGAAGATCAGCCT | |
| | GTTCAGCTGCCTGAAGGACCGGCACGATTTCGGCTTCCCTCAA | |
| | GAGGAATTCGGCAACCAGTTCCAGAAGGCCGAGACAATCCCT | |
| | GTGCTGCACGAGATGATCCAGCAGATCTTCAACCTGTTCTCCA | |
| | CCAAGGACAGCAGCGCCGCCTGGGATGAGACACTGCTGGACA | |
| | AGTTCTACACCGAGCTGTACCAGCAGCTGAATGACCTGGAAGC | |
| | CTGCGTGATCCAAGGCGTGGGAGTGACAGAGACACCCCTGATG | |
| | AAGGAAGATAGCATCCTGGCCGTGCGCAAGTACTTCCAGCGGA | |
| | TCACCCTGTACCTGAAAGAGAAGAAGTACAGCCCCTGCGCCTG | |
| | GGAAGTCGTGCGGGCCGAAATCATGAGAAGCTTCAGCCTGAG | |
| | CACCAACCTGCAAGAGAGCCTGCGGAGCAAAGAGTGA | |
| 2 | ATGATGGTGGACGGCGACAACAGCCACGTGGAAATGAAGCTG | TFRC_G4S_IL-12 |
| | GCCGTGGACGAGGAAGAGAACGCCGACAACAACACCAAGGCC | mRNA |
| | AACGTGACCAAGCCTAAGAGATGCAGCGGCAGCATCTGCTAC | |
| | GGCACAATCGCCGTGATCGTGTTCTTCCTGATCGGCTTTATGAT | |
| | CGGCTACCTGGGCTACTGCAAGAGCAGTGATGGACCTGGCGAA | |
| | ACAGGCGGAGGCGGAGGATCTGGTGGCGGAGGAAGTGGCGGC | |
| | GGAGGTTCTATTTGGGAGCTGAAGAAAGACGTGTACGTGGTGG | |
| | AACTGGACTGGTATCCCGATGCTCCTGGCGAGATGGTGGTGCT | |
| | GACCTGCGATACCCCTGAAGAGGACGGCATCACCTGGACACTG | |
| | GATCAGTCTAGCGAGGTGCTCGGCAGCGGCAAGACCCTGACCA | |
| | TCCAAGTGAAAGAGTTTGGCGACGCCGGCCAGTACACCTGTCA | |
| | CAAAGGCGGAGAAGTGCTGAGCCACAGCCTGCTGCTGCTCCAC | |
| | AAGAAAGAGGATGGCATTTGGAGCACCGACATCCTGAAGGAC | |
| | CAGAAAGAGCCCAAGAACAAGACCTTCCTGAGATGCGAGGCC | |
| | AAGAACTACAGCGGCCGGTTCACATGTTGGTGGCTGACCACCA | |
| | TCAGCACCGACCTGACCTTCAGCGTGAAGTCCAGCAGAGGCAG | |
| | CAGTGATCCTCAGGGCGTTACATGTGGCGCCGCTACACTGTCT | |
| | GCCGAAAGAGTGCGGGGCGATAACAAAGAATACGAGTACAGC | |
| | GTGGAATGCCAAGAGGACAGCGCCTGTCCAGCCGCCGAAGAG | |
| | TCTCTGCCTATCGAAGTGATGGTCGACGCCGTGCACAAGCTGA | |
| | AGTACGAGAACTACACCAGCAGCTTTTTCATCCGGGACATCAT | |
| | CAAGCCCGATCCTCCAAAGAACCTGCAGCTGAAGCCTCTGAAG | |
| | AACAGCAGACAGGTGGAAGTGTCCTGGGAGTACCCCGACACC | |
| | TGGTCTACACCCCACAGCTACTTCAGCCTGACCTTTTGCGTGCA | |
| | AGTGCAGGGCAAGTCCAAGCGCGAGAAAAAGGACCGGGTGTT | |
| | CACCGACAAGACCAGCGCCACCGTGATCTGCAGAAAGAACGC | |
| | CAGCATCAGCGTCAGAGCCCAGGACCGGTACTACAGCAGCTCT | |
| | TGGAGCGAATGGGCCAGCGTGCCATGTAGCGGAGGTGGTGGT | |
| | AGCGGAGGCGGCGGAAGCGGCGGTGGTGGATCAGGTGGTGGT | |
| | GGCTCTAGAAACCTGCCAGTGGCTACCCCTGATCCTGGCATGT | |
| | TCCCTTGTCTGCACCACAGCCAGAACCTGCTGAGAGCCGTGTC | |
| | CAACATGCTGCAGAAGGCCAGACAGACCCTGGAATTCTACCCC | |
| | TGCACCAGCGAGGAAATCGACCACGAGGACATCACCAAGGAT | |
| | AAGACCAGCACCGTGGAAGCCTGCCTGCCTCTGGAACTGACCA | |
| | AGAACGAGAGCTGCCTGAACAGCCGGGAAACCTCCTTCATCAC | |
| | CAACGGCTCTTGCCTGGCCAGCAGAAAGACAAGCTTCATGATG | |
| | GCCCTGTGCCTGAGCAGCATCTACGAGGACCTGAAGATGTACC | |
| | AGGTGGAATTCAAGACCATGAACGCCAAGCTGCTGATGGACCC | |
| | CAAGCGGCAGATCTTCCTGGACCAGAACATGCTGGCTGTGATC | |
| | GACGAGCTGATGCAGGCCCTGAACTTCAACAGCGAGACAGTG | |
| | CCCCAGAAGTCTAGCCTGGAAGAACCCGACTTCTACAAGACCA | |
| | AGATCAAGCTGTGCATCCTGCTGCACGCCTTCCGGATCAGAGC | |
| | CGTGACCATCGACAGAGTGATGAGCTACCTGAACGCCTCCTGA | |
| 3 | (G₄S)₃ | G₄S Linker |
| 4 | (EAAAK)3 | EAAAK linker |
| 5 | (G₄S)ₙ | G₄S Linker |
| 6 | (EAAAK)n | EAAAK linker |
| 7 | MMVDGDNSHVEMKLAVDEEENADNNTKANVTKPKRCSGSICYG | TFRC_G4S_IL-2 |
| | TIAVIVFFLIGFMIGYLGYCKSSDGPGETGGGGGSGGGGSGGGGS | |
| | APTSSSTKKTQLQLEHLLLDLQMILNGINNYKNPKLTRMLTFKFY | |
| | MPKKATELKHLQCLEEELKPLEEVLNLAQSKNFHLRPRDLISNINV | |
| | IVLELKGSETTFMCEYADETATIVEFLNRWITFCQSIISTLT | |
| 8 | MLKKRGNHSTGLCLLVMFFMVLVALVGLGLGMFQLFHLQKETG | FasL_G4S_IL-2 |
| | GGGGSGGGGSGGGGS | |
| | APTSSSTKKTQLQLEHLLLDLQMILNGINNYKNPKLTRMLTFKFY | |
| | MPKKATELKHLQCLEEELKPLEEVLNLAQSKNFHLRPRDLISNINV | |
| | IVLELKGSETTFMCEYADETATIVEFLNRWITFCQSIISTLT | |
| 9 | MMVDGDNSHVEMKLAVDEEENADNNTKANVTKPKRCSGSICYG | TFRC_G4S_IL-12 |
| | TIAVIVFFLIGFMIGYLGYCKSSDGPGETGGGGGSGGGGSGGGGS | |
| | IWELKKDVYVVELDWYPDAPGEMVVLTCDTPEEDGITWTLDQSS | |
| | EVLGSGKTLTIQVKEFGDAGQYTCHKGGEVLSHSLLLLHKKEDGI | |
| | WSTDILKDQKEPKNKTFLRCEAKNYSGRFTCWWLTTISTDLTFSV | |
| | KSSRGSSDPQGVTCGAATLSAERVRGDNKEYEYSVECQEDSACP | |
| | AAEESLPIEVMVDAVHKLKYENYTSSFFIRDIIKPDPPKNLQLKPL | |
| | KNSRQVEVSWEYPDTWSTPHSYFSLTFCVQVQGKSKREKKDRVF | |
| | TDKTSATVICRKNASISVRAQDRYYSSSWSEWASVPCSGGGGSGG | |
| | GGSGGGGSGGGGSRNLPVATPDPGMFPCLHHSQNLLRAVSNMLQ | |
| | KARQTLEFYPCTSEEIDHEDITKDKTSTVEACLPLELTKNESCLNS | |
| | RETSFITNGSCLASRKTSFMMALCLSSIYEDLKMYQVEFKTMNAK | |
| | LLMDPKRQIFLDQNMLAVIDELMQALNFNSETVPQKSSLEEPDFY | |
| | KTKIKLCILLHAFRIRAVTIDRVMSYLNAS | |
| 10 | MMVDGDNSHVEMKLAVDEEENADNNTKANVTKPKRCSGSICYG | TFRC_G4S_IFN-a2a |
| | TIAVIVFFLIGFMIGYLGYCKSSDGPGETGGGGGSGGGGSGGGGS | |
| | CDLPQTHSLGSRRTLMLLAQMRKISLFSCLKDRHDFGFPQEEFGN | |
| | QFQKAETIPVLHEMIQQIFNLFSTKDSSAAWDETLLDKFYTELYQQ | |
| | LNDLEACVIQGVGVTETPLMKEDSILAVRKYFQRITLYLKEKKYS | |
| | PCAWEVVRAEIMRSFSLSTNLQESLRSKE | |
| 11 | MMVDGDNSHVEMKLAVDEEENADNNTKANVTKPKRCSGSICYG | TFRC domain |
| | TIAVIVFFLIGFMIGYLGYCKSSDGPGETG | |
| 12 | MLKKRGNHSTGLCLLVMFFMVLVALVGLGLGMFQLFHLQKETG | FasL domain |
| 13 | ATGATGGTGGACGGCGACAACAGCCACGTGGAGATGAAGCTG | membrane-bound IL-7 |
| | GCCGTGGACGAGGAGGAGAACGCCGACAACAACACCAAGGCC | mRNA |
| | AACGTGACCAAGCCCAAGCGCTGCAGCGGCAGCATCTGCTACG | |
| | GCACCATCGCCGTGATCGTGTTCTTCCTGATCGGCTTCATGATC | |
| | GGCTACCTGGGCTACTGCAAGAGCAGCGACGGCCCCGGCGAG | |
| | ACCGGCGGaGGCGGCGGaAGCGGCGGCGGaGGCAGCGGCGGaG | |
| | GCGGaAGCGACTGCGACATCGAGGGCAAGGACGGCAAGCAGT | |
| | ACGAGAGCGTGCTGATGGTGAGCATCGACCAGCTGCTGGACA | |
| | GCATGAAGGAGATCGGCAGCAACTGCCTGAACAACGAGTTCA | |
| | ACTTCTTCAAGCGCCACATCTGCGACGCCAACAAGGAGGGCAT | |
| | GTTCCTGTTCCGCGCCGCCCGCAAGCTGCGCCAGTTCCTGAAG | |
| | ATGAACAGCACCGGCGACTTCGACCTGCACCTGCTGAAGGTGA | |
| | GCGAGGGCACCACCATCCTGCTGAACTGCACCGGCCAGGTGAA | |
| | GGGCCGCAAGCCCGCCGCCCTGGGCGAGGCCCAGCCCACCAA | |
| | GAGCCTGGAGGAGAACAAGAGCCTGAAGGAGCAGAAGAAGCT | |
| | GAACGACCTGTGCTTCCTGAAGCGCCTGCTGCAGGAGATCAAG | |
| | ACCTGCTGGAACAAGATCCTGATGGGCACCAAGGAGCACtaa | |

1. A method of modulating the activity and/or proliferative capacity of tumor-infiltrating lymphocytes (TILs), comprising modifying the TILs to increase expression of one or more co-stimulatory molecules, one or more anti-apoptotic factors, and/or one or more cytokines.

2.-5. (canceled)

6. The method of claim 1, wherein the one or more cytokines comprise a chimeric membrane-bound cytokine, and wherein the cytokine is a Type I cytokine and/or the cytokine is IL-15, IL-12, IL-7, IL-2, IFN-α, IFN-β, or IL-21 or functional variant thereof.

7.-11. (canceled)

12. The method of claim 1, wherein the cytokine is IL-2 or a functional variant thereof, IL-7 or a functional variant thereof, IL-15 or a functional variant thereof, and/or IL-12 or a functional variant thereof.

13. The method of claim 12, wherein the cytokine or the functional variant thereof is a membrane-bound IL-2, a membrane-bound IL-7, a membrane-bound IL-15, and/or a membrane-bound IL-12.

14.-18. (canceled)

19. The method of claim 1, wherein the anti-apoptotic factor comprise Bcl-2 and/or the co-stimulatory molecule is B7-H2 (ICOSL), B7-1 (CD80), B7-2 (CD86), CD70, LIGHT, HVEM, CD40, 4-1BBL, OX40L, TL1A, GITRL, CD30L, TIM4, SLAM, CD48, CD58, CD155, or CD112.

20. The method of claim 1, wherein modifying the TILs to increase expression of one or more co-stimulatory molecules, one or more anti-apoptotic factors, and/or one or more cytokines comprises
(i) passing a cell suspension comprising the TILs through a cell-deforming constriction, thereby causing perturbations of the TILs such that one or more nucleic acids encoding the one or more co-stimulatory molecules, one or more anti-apoptotic factors, and/or one or more nucleic acids encoding the one or more cytokines enter the TILs through the perturbations when contacted with the TILs; and
(ii) contacting the TILs with the one or more nucleic acids encoding the one or more co-stimulatory molecules, the one or more nucleic acids encoding the one or more anti-apoptotic factors, and/or the one or more nucleic acids encoding the one or more cytokines before, during, and/or after passing the cell suspension through the cell-deforming constriction.

21.-31. (canceled)

32. The method of claim 1, wherein after the modifying, the TILs;
(i) exhibit increased survival as compared to reference TILs, which comprise corresponding TILs that have not been modified;
(ii) produce greater amount of IFN-γ upon anti-CD3 stimulation as compared to reference TILs, which comprise corresponding TILs that have not been modified; and/or
(iii) exhibit increased viability as compared to reference TILs, which comprise corresponding TILs that have not been modified; and/or
(iv) exhibit increased in vivo persistence as compared to reference TILs, which comprise corresponding TILs that have not been modified; and/or
(v) exhibit increased proliferation as compared to reference TILs, which comprise corresponding TILs that have not been modified; and/or
(vi) have increased expression of a marker selected from T-bet, EOMES, TCF1, CD127, CD103, CD45RO, CCR5, CD62L, or combinations thereof, as compared to reference TILs, which comprise corresponding TILs that have not been modified.

33. The method of claim 32, wherein after the modifying, the TILs exhibit the increased survival, produce the greater amount of IFN-γ, exhibit the increased viability, exhibit the increased in vivo persistence, exhibit the increased proliferation, and/or have the increased expression of the marker in the absence of an exogenous cytokine as compared to the reference TILs.

34.-40. (canceled)

41. The method of claim 1, further comprising expanding the TILs prior to the modifying, wherein expanding the TILs comprise culturing the TILs with one or more agents, wherein the agents are capable of expanding the TILs such that the number of TILs is increased as compared to the number of the TILs Prior to the expansion.

42.-47. (canceled)

48. The method of claim 41, wherein expanding the TILs comprise a first expansion phase and a second expansion phase,
wherein the first expansion phase comprises culturing the TILs in the presence of an exogenous cytokine and/or an anti-CD3 antibody; and/or
wherein the second expansion phase comprises culturing the TILs in the presence of an exogenous cytokine, an anti-CD3 antibody, a feeder cell, or a combination thereof.

49.-64. (canceled)

65. A composition comprising modified TILs, wherein the modified TILs exhibit increased expression of one or more co-stimulatory molecules, one or more anti-apoptotic factors, and/or one or more cytokines as compared to reference TILs, which comprise corresponding non-modified TILs.

66. (canceled)

67. The composition of claim 65, wherein:
(i) the co-stimulatory molecule is B7-H2 (ICOSL), B7-I (CD80), B7-2 (CD86), CD70, LIGHT, HVEM, CD40, 4-1BBL, OX40L, TL1A, GITRL, CD30L, TIM4, SLAM, CD48, CD58, CD155, or CD112; and/or
(ii) the anti-apoptotic factor comprise Bcl-2.

68.-69. (canceled)

70. The composition claim 65, wherein the one or more cytokines comprise a chimeric membrane-bound cytokine, and/or wherein the cytokine is a Type I cytokine, and/or the cytokine is IL-15, IL-12, IL-7, IL-2, IFN-α, IFN-β, or IL-21 or functional variant thereof.

71.-76. (canceled)

76. The composition of claim 74, wherein the cytokine is IL-2 or a functional variant thereof, IL-7 or a functional variant thereof, IL-15 or a functional variant thereof, and/or IL-12 or a functional variant thereof.

77. The composition of claim 74, wherein the cytokine or the functional variant thereof is a membrane-bound IL-2, a membrane-bound IL-7, a membrane-bound IL-15, and/or a membrane-bond IL-12.

78.-83. (canceled)

84. The composition of claim 65, wherein the modified TILs have been passed through a cell-deforming constriction, thereby causing perturbations of the TILs such that one or more nucleic acids encoding the one or more co-stimulatory molecules, one or more nucleic acids encoding the one or more anti-apoptotic factors, and/or one or more nucleic acids encoding the one or more cytokines entered the TILs through the perturbations when contacted with the TILs.

85.-90. (canceled)

91. The composition of claim 65, wherein the modified TILs;
(a) have increased expression of a marker selected from T-bet, EOMES, TCFI, CD127, CD103, CD45RO, CCR5, CD62L, or combinations thereof, as compared to reference TILs, which comprise corresponding non-modified TILs; and/or
(b) exhibit increased proliferation as compared to reference TILs, which comprise corresponding non-modified TILs; and/or
(c) exhibit one or more of the following properties as compared to reference TILs, which comprise corresponding non-modified TILs: (i) increased in vivo persistence, (ii) increased viability, (iii) increased ability to produce IFN-γ upon anti-CD3 stimulation, (iv) increased ability to kill tumor cells, (v) increased survival, or (vi) any combination of (i) to (v).

92. The composition of claim 91, wherein the modified TILs have increased expression of the marker, exhibit increased proliferation, and/or exhibit one or more of the properties in the absence of an exogenous cytokine.

93. (canceled)

94. The composition of claim 65, wherein the modified TILs exhibit increased expression of one or more of the following: (i) membrane-bound IL-2, (ii) membrane-bound IL-12, (iii) CD86, (iv) membrane-bound IL-7, (v) membrane-bound IL-15, (vi) Bcl-2, or (vii) any combination of (i) to (vi).

95.-115. (canceled)

116. A method of producing TILs which exhibit one or more improved properties, comprising intracellularly delivering one or more nucleic acids encoding a cytokine, one or more nucleic acids encoding an anti-apoptotic factor, and/or one or more nucleic acids encoding a co-stimulatory molecule, wherein the cytokine, anti-apoptotic factor, and/or the co-stimulatory molecule is capable of improving one or more properties of the TILs, wherein the one or more improved properties comprise: (i) increased Proliferation; (ii) increased in vivo persistence, (iii) increased viability, (iv) increased ability to produce IFN-γ upon anti-CD3 stimulation, (v) increased ability to kill tumor cells, (vi) increased survival, or (vii) any combination of (i) to (vi).

117. (canceled)

118. The method of claim 116, wherein the cytokine comprises IL-15, IL-12, IL-7, IL-2, IFN-α, IFN-β, or IL-21 or functional variant thereof; and/or the cytokine or functional variant thereof comprises a membrane-bound cytokine.

119. (canceled)

120. The method of claim 118, wherein the cytokine or functional variant thereof is a membrane-bound IL-2, a membrane-bound IL-7, a membrane-bound IL-15, and/or a membrane-bound IL-12.

121-123. (canceled)

124. The method of claim 116, wherein:
(i) the co-stimulatory molecule comprises B7-H2 (ICOSL), B7-1 (CD80), B7-2 (CD86), CD70, LIGHT, HVEM, CD40, 4-1BBL, OX40L, TLIA, GITRL, CD30L, TIM4, SLAM, CD48, CD58, CD155, or CD112; and/or
(ii) the anti-apoptotic factor comprises Bcl-2.

125.-126. (canceled)

127. The method of claim 116, wherein intracellularly delivering the one or more nucleic acids comprise passing the TILs through a cell-deforming constriction, thereby causing perturbations in the TILs such that the one or more nucleic acids enter the TILs through the perturbations when contacted with the TILs and contacting the TILs with the one or more nucleic acids.

128.-135. (canceled)


SEQUENCE LISTING (13 sequences)

SEQ ID NO: 1 - DNA, 762 bp, synthetic construct
  misc_feature 1..762: note = TFRC_G4S_IFN-a2a mRNA
  source 1..762: mol_type = other DNA
atgatggtggacggcgacaacagccacgtggaaatgaagctggccgtggacgaggaagagaacgccgacaacaacaccaaggccaacgtgaccaagcctaagagatgcagcggcagcatctgctacggcacaatcgccgtgatcgtgttcttcctgatcggctttatgatcggctacctgggctactgcaagagcagtgatggacctggcgaaacaggcggaggcggaggatctggtggcggaggaagcggtggcggcggatcttgtgatctgcctcagacacacagcctgggcagcagacgaacactgatgctgctggcccagatgcggaagatcagcctgttcagctgcctgaaggaccggcacgatttcggcttccctcaagaggaattcggcaaccagttccagaaggccgagacaatccctgtgctgcacgagatgatccagcagatcttcaacctgttctccaccaaggacagcagcgccgcctgggatgagacactgctggacaagttctacaccgagctgtaccagcagctgaatgacctggaagcctgcgtgatccaaggcgtgggagtgacagagacacccctgatgaaggaagatagcatcctggccgtgcgcaagtacttccagcggatcaccctgtacctgaaagagaagaagtacagcccctgcgcctgggaagtcgtgcgggccgaaatcatgagaagcttcagcctgagcaccaacctgcaagagagcctgcggagcaaagagtga

SEQ ID NO: 2 - DNA, 1836 bp, synthetic construct
  misc_feature 1..1836: note = TFRC_G4S_IL-12 mRNA
  source 1..1836: mol_type = other DNA
atgatggtggacggcgacaacagccacgtggaaatgaagctggccgtggacgaggaagagaacgccgacaacaacaccaaggccaacgtgaccaagcctaagagatgcagcggcagcatctgctacggcacaatcgccgtgatcgtgttcttcctgatcggctttatgatcggctacctgggctactgcaagagcagtgatggacctggcgaaacaggcggaggcggaggatctggtggcggaggaagtggcggcggaggttctatttgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgaccatccaagtgaaagagtttggcgacgccggccagtacacctgtcacaaaggcggagaagtgctgagccacagcctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgccgctacactgtctgccgaaagagtgcggggcgataacaaagaatacgagtacagcgtggaatgccaagaggacagcgcctgtccagccgccgaagagtctctgcctatcgaagtgatggtcgacgccgtgcacaagctgaagtacgagaactacaccagcagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggaccggtactacagcagctcttggagcgaatgggccagcgtgccatgtagcggaggtggtggtagcggaggcggcggaagcggcggtggtggatcaggtggtggtggctctagaaacctgccagtggctacccctgatcctggcatgttcccttgtctgcaccacagccagaacctgctgagagccgtgtccaacatgctgcagaaggccagacagaccctggaattctacccctgcaccagcgaggaaatcgaccacgaggacatcaccaaggataagaccagcaccgtggaagcctgcctgcctctggaactgaccaagaacgagagctgcctgaacagccgggaaacctccttcatcaccaacggctcttgcctggccagcagaaagacaagcttcatgatggccctgtgcctgagcagcatctacgaggacctgaagatgtaccaggtggaattcaagaccatgaacgccaagctgctgatggaccccaagcggcagatcttcctggaccagaacatgctggctgtgatcgacgagctgatgcaggccctgaacttcaacagcgagacagtgccccagaagtctagcctggaagaacccgacttctacaagaccaagatcaagctgtgcatcctgctgcacgccttccggatcagagccgtgaccatcgacagagtgatgagctacctgaacgcctcctga

SEQ ID NO: 3 - protein, 15 aa, synthetic construct
  VARIANT 1..15
GGGGSGGGGSGGGGS

SEQ ID NO: 4 - protein, 15 aa, synthetic construct
  VARIANT 1..15
EAAAKEAAAKEAAAK

SEQ ID NO: 5 - protein, 6 aa, synthetic construct
  VARIANT 1..5
GGGGSX

SEQ ID NO: 6 - protein, 6 aa, synthetic construct
  VARIANT 1..5: note = The entire sequence of amino acids 1-5 can be repeated one or more times
EAAAKX

SEQ ID NO: 7 - protein, 221 aa, synthetic construct
  VARIANT 1..220
MMVDGDNSHVEMKLAVDEEENADNNTKANVTKPKRCSGSICYGTIAVIVFFLIGFMIGYLGYCKSSDGPGETGGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 8 - protein, 191 aa, synthetic construct
  VARIANT 1..191
MLKKRGNHSTGLCLLVMFFMVLVALVGLGLGMFQLFHLQKETGGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 9 - protein, 611 aa, synthetic construct
  VARIANT 1..611
MMVDGDNSHVEMKLAVDEEENADNNTKANVTKPKRCSGSICYGTIAVIVFFLIGFMIGYLGYCKSSDGPGETGGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 10 - protein, 253 aa, synthetic construct
  VARIANT 1..253
MMVDGDNSHVEMKLAVDEEENADNNTKANVTKPKRCSGSICYGTIAVIVFFLIGFMIGYLGYCKSSDGPGETGGGGGSGGGGSGGGGSCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 11 - protein, 73 aa, synthetic construct
  VARIANT 1..73
MMVDGDNSHVEMKLAVDEEENADNNTKANVTKPKRCSGSICYGTIAVIVFFLIGFMIGYLGYCKSSDGPGETG

SEQ ID NO: 12 - protein, 43 aa, synthetic construct
  VARIANT 1..43
MLKKRGNHSTGLCLLVMFFMVLVALVGLGLGMFQLFHLQKETG

SEQ ID NO: 13 - DNA, 723 bp, synthetic construct
  misc_feature 1..723: note = membrane-bound IL-7 mRNA
  source 1..723: mol_type = other DNA
atgatggtggacggcgacaacagccacgtggagatgaagctggccgtggacgaggaggagaacgccgacaacaacaccaaggccaacgtgaccaagcccaagcgctgcagcggcagcatctgctacggcaccatcgccgtgatcgtgttcttcctgatcggcttcatgatcggctacctgggctactgcaagagcagcgacggccccggcgagaccggcggaggcggcggaagcggcggcggaggcagcggcggaggcggaagcgactgcgacatcgagggcaaggacggcaagcagtacgagagcgtgctgatggtgagcatcgaccagctgctggacagcatgaaggagatcggcagcaactgcctgaacaacgagttcaacttcttcaagcgccacatctgcgacgccaacaaggagggcatgttcctgttccgcgccgcccgcaagctgcgccagttcctgaagatgaacagcaccggcgacttcgacctgcacctgctgaaggtgagcgagggcaccaccatcctgctgaactgcaccggccaggtgaagggccgcaagcccgccgccctgggcgaggcccagcccaccaagagcctggaggagaacaagagcctgaaggagcagaagaagctgaacgacctgtgcttcctgaagcgcctgctgcaggagatcaagacctgctggaacaagatcctgatgggcaccaaggagcactaa